(19) United States
(12) Patent Application Publication
(10) Pub. No.: US 2024/0000926 A1
(43) Pub. Date: Jan. 4, 2024

(54) PURIFICATION PROCESSES FOR POLYSACCHARIDES AND POLYPEPTIDE CONJUGATES THEREOF

(71) Applicant: Vaxcyte, Inc., San Carlos, CA (US)

(72) Inventors: Christopher R. BEHRENS, San Francisco, CA (US); Paresh AGARWAL, Millbrae, CA (US); Lu YIN, Santa Cruz, CA (US); Peter T. DAVEY, Foster City, CA (US); Angie A. SEDRA, Foster City, CA (US)

(21) Appl. No.: 18/447,146

(22) Filed: Aug. 9, 2023

Related U.S. Application Data
(63) Continuation of application No. PCT/US22/16630, filed on Feb. 16, 2022.
(60) Provisional application No. 63/150,516, filed on Feb. 17, 2021.
(60) Provisional application No. 63/288,387, filed on Dec. 10, 2021.

(51) Int. Cl. A61K 39/385; C07K 14/315; C07K 14/34; C12N 9/52; A61K 39/09; A61P 37/04; C12P 19/04; C12P 19/14; A61K 47/61; A61K 47/64

(52) U.S. Cl. CPC A61K 39/385; C07K 14/315; C07K 14/34; C12N 9/52; C12Y 304/2111; A61K 39/092; A61P 37/04; C12P 19/04; C12P 19/14; A61K 47/61; A61K 47/646; A61K 2039/6087

(57) ABSTRACT

The present disclosure provides a process for purifying high-molecular weight bacterial polysaccharides (e.g., cell wall polysaccharides), and polypeptide-polysaccharide conjugates thereof, suitable for use in immunogenic compositions.

### CROSS-REFERENCE TO RELATED APPLICATIONS

This application is a continuation of International Application No. PCT/US2022/016630, filed Feb. 16, 2022, which claims the benefit of U.S. Provisional Application No. 63/150,516, filed Feb. 17, 2021, and U.S. Provisional Application No. 63/288,387 filed Dec. 10, 2021, the disclosure of each of which are hereby incorporated by reference in their entireties.

### STATEMENT OF GOVERNMENT INTEREST

This invention was made with the support of the United States government under grant number 93.360, subaward 4500003905, awarded by the Health and Human Services Office of the Assistant Secretary for Preparedness and Response (HHS/ASPR) under the CARB-X Pass Through Entity. The government has certain rights in the invention.

### REFERENCE TO AN ELECTRONIC SEQUENCE LISTING

The contents of the electronic sequence listing (STRO_015_02US_SeqList_ST26.xml; Size: 226,834 bytes; and Date of Creation: Aug. 9, 2023) are herein incorporated by reference in their entirety.

### TECHNICAL FIELD

This invention relates generally to the field of bacterial polysaccharides, their purification, their conjugation to polypeptides, and immunogenic compositions comprising such polysaccharide-polypeptide conjugates.

### BACKGROUND

Bacterial cell walls and cell membranes are associated with a number of polymeric molecules, including glycoconjugates and polysaccharides (PSs), which fill various structural and functional roles in the bacterium. In gram-negative bacteria, the outer membrane largely comprises lipopolysaccharides (LPSs). Gram-positive bacteria lack an outer membrane but have a thick peptidoglycan layer with specialized polysaccharides. Some strains of both gram-negative and gram-negative bacteria additionally contain peptidoglycan-bound capsular polysaccharides which aid in virulence.

Inducing immunity against many PSs, including those of bacteria such as meningococcus, HiB, and pneumococcus, confers protection against disease, and a number of effective vaccines have been developed. However, large-scale vaccine manufacture requires equally large-scale and cost-effective supplies of pure, homogenous polysaccharide free of other cell components. Some traditional methods of purifying polysaccharides have relied upon harsh conditions (e.g., concentrated hydrofluoric acid), which pose issues with respect to safety and limit production scale-up. Further, many purification methods degrade the target polysaccharide to an extent, resulting in low yields, or polysaccharides of low molecular weight with limited or reduced immunogenicity. Accordingly, there is a need for, safer, higher-yielding, and more efficient methods of polysaccharide production.

Group A Streptococcus (GAS) is a preeminent human pathogen causing 700 million cases of pharyngitis (‘strep throat’) annually worldwide and increasing cases of severe invasive infections, sepsis, necrotizing fasciitis, otitis media, and toxic shock syndrome. GAS is also responsible for post-infectious immune-mediated rheumatic heart disease (RHD), a leading cause of mortality in the developing world. Some 30 million people are currently affected by RHD, with over 300,000 deaths annually (60%<age 70) and 11.5 million disability-adjusted life years lost. Despite high global demand, there is no safe and efficacious commercial vaccine against GAS. Features of the pathogen pose particular challenges to vaccination, including its invariant capsule of hyaluronic acid, an immunologically inert carbohydrate ubiquitous in connective tissues. Furthermore, the immunodominant surface-anchored GAS M proteins are highly polymorphic (>200 emm types), and regions of their dimeric coiled-coil structure may provoke an autoimmune response against cardiac tissue in RHD. Thus, there is a need for immunogenic compositions that can prevent or treat GAS infection. Such compositions might utilize protein-antigen conjugates, and there is therefore a need for conjugates with sufficient immunogenicity.

### SUMMARY

Disclosed herein are polypeptide-polysaccharide conjugates comprising: (a) a GAS polypeptide antigen or a non-GAS carrier polypeptide comprising at least one non-natural amino acid (nnAA); and (b) a purified cell wall polysaccharide or a peptidoglycan-bound capsular polysaccharide with a molecular weight of at least about 10 kDa to at least about 40 kDa.

Immunogenic compositions comprising a Group A Streptococcus (GAS) C5a peptidase polypeptide antigen, a GAS streptolysin O (SLO) polypeptide antigen, and a polypeptide-polysaccharide conjugate are described herein. The polypeptide-polysaccharide conjugate may comprise (i) a Streptococcus pyogenes Adhesion and Division (SpyAD) conjugate polypeptide, or a fragment thereof, comprising at least one non-natural amino acid (nnAA), wherein the at least one nnAA comprises a click chemistry reactive group; and (ii) a GAS polysaccharide, or a variant thereof, that lacks an immunodominant N-acetyl Glucosamine (GlcNAc) side chain. Between about 8 mol % and about 20 mol % of the polysaccharide repeat units of the GAS polysaccharide, or a variant thereof, may be derivatized by a linker. The average molecular weight of the polypeptide-polysaccharide conjugate may be between about 185 kDa and about 700 kDa or between about 190 kDa and about 700 kDa.

Also disclosed herein are processes for purifying cell wall polysaccharides or peptidoglycan-bound capsular polysaccharides from a bacterial cell, the process comprising: (a) hydrolyzing the bacterial cell in a solution comprising base and a reducing agent to form a lysate comprising polysaccharide; and (b) incubating the lysate comprising polysaccharide with a muralytic enzyme to form a free polysaccharide solution.

### BRIEF DESCRIPTION OF THE DRAWINGS

FIG. 1 shows a flowchart of outlining an exemplary polysaccharide purification method.

FIG. 2A, FIG. 2B, and FIG. 2C show NMR analysis of purified polysaccharide originating from a GAS bacterial strain expressing PS lacking GlcNAc.

FIG. 3 shows a western blot demonstrating M-protein is removed from polysaccharide when purified as described in Example 1.

FIGS. 4A and 4B show flowcharts outlining alternative exemplary polysaccharide purification methods.

FIG. 5 shows a scheme outlining the derivatization of a GAS polysaccharide with DBCO-(PEG)₄-NH2 and subsequent conjugation to pAMF-derivatized SpyAD.

FIG. 6 shows SEC MALS analysis of a SpyAD[4pAMF] polypeptide-polysaccharide conjugate after 3.5 hours of conjugation reaction and post-dialysis, prepared as described in Example 1.

FIG. 7 shows the SEC-MALS analysis of a SpyAD[4pAMF] polypeptide-polysaccharide conjugate where a hydrofluoric acid-based PS purification method was used (Peak 1: 2070.5 kDa: Peak 2: 135,200 Da).

FIG. 8A shows a schematic of 22 SLO(ΔC101) variants (var1-var22) and their pAMF incorporation sites.

FIG. 8B and FIG. 8C show the expression levels of 22 SLO variants with 3-8 pAMF, and the corresponding gels for purified 5-8 pAMF containing variants.

FIG. 8D shows Safe Blue-stained and DBCO-TAMRA-labeled gels which show successful pAMF incorporation in a subset of purified variants (var1, 5, 6, 10, 11, 12, 14, and 15).

FIG. 8E shows SEC-MALS analysis providing molecular mass estimates for a subset of the purified variants (varn, 5, 6, 10, 11, 12, 14, and 15).

FIG. 9A shows SDS-PAGE analysis of 5- and 6-pAMF variant SLO conjugates with long GAC. FIG. 9B shows SEC-MALS analysis of purified conjugates, showing average molar mass of 97 or 116 kDa for conjugates generated using SLO(ΔC101) variants containing 3 pAMFs (inset molar masses for GAC^{PR}: 6.2 kDa and GAC^{PR}-DBCO: 6.8 kDa), FIG. 9C show SEC-MALS analysis of purified conjugates, showing average molar mass 68 or 74 kDa for conjugates generated using SLO(ΔC101) variants containing 4 pAMFs.

FIG. 10 shows SEC-MALS data for 5- and 6-pAMF variant SLO conjugates with the long GAC polysaccharide.

FIG. 11A shows the antibody titers of mice post-immunization using conjugates made with select SLO(ΔC101) variants, and using the long GAC polysaccharide as a coating antigen in the ELISA.

FIG. 11B shows the antibody titers of another cohort of mice, post-immunization, using conjugates made with select SLO(ΔC101) variants, and using the long GAC polysaccharide as a coating antigen in the ELISA.

FIG. 12 shows the results of in vivo mouse immunization study with mock, SLO(ΔC101)var1-GAC conjugate or a combination vaccine [SLO(ΔC101)var1+eCRM-GACPR], post challenge with GAS serotype M1.

FIG. 13 shows a gel comparing conjugates CNJ-AE, CNJ-AF, and CNJ-AG to native SpyAD, showing that the conditions used in the production of CNJ-AG resulted in a higher molecular weight conjugate.

FIG. 14 shows the results of an autoradiography experiment (gel and plot showing estimated expression titer), demonstrating that SpyAD fragments (e.g., SEQ ID NOs: 77, 78, 79, and 81) result in fewer unwanted fragments during expression, allowing for easier purification and better conjugation to activated polysaccharide.

FIG. 15, FIG. 16, FIG. 17, FIG. 18, FIG. 19 show the results (antibody titers via ELISA) of immunization studies comparing SpyAD-GAC conjugates of different molecular weights and that were produced with different conjugation conditions.

### DETAILED DESCRIPTION

Described herein are methods for purifying high-molecular weight polysaccharides PSs (referred to herein as long polysaccharides) from a bacterial cell. These PSs have a higher average molecular weight than those isolated using methods known in the art, and therefore may be used in the production of vaccines, immunogenic compositions, and the like, with increased immunogenicity and/or stability. Such immunogenic compositions are described herein. In addition to the increased size of the PSs, the processes described herein may result in higher yields than traditional methods of purification. As a result, these methods allow for more efficient production of purified PS.

### Purification of Polysaccharides

The processes described herein are useful for purifying polysaccharides from a bacterial cell. The bacterial cells may, for instance, be isolated as a pellet from culture using standard techniques known in the art. In some embodiments, the present disclosure provides a process for purifying cell wall polysaccharides or peptidoglycan-bound capsular polysaccharides from a bacterial cell, including, for example, purification of pellets of bacterial cells.

“Purifying”, as used herein, refers to removing or partially removing polysaccharides from non-polysaccharide components present a bacterial cell. Purifying may mean removing at least about 85% of impurities and/or byproducts. In some embodiments, a “purified” polysaccharide solution or preparation contains less than about 15%, about 14%, about 13%, about 12%, about 11%, about 10%, about 9%, about 8%, about 7%, about 6%, about 5%, about 4%, about 3%, about 2%, about 1%, about 0.9%, about 0.8%, about 0.7%, about 0.6%, about 0.5%, about 0.4%, about 0.3%, about 0.2%, about 0.1%, about 0.09%, about 0.08%, about 0.07%. about 0.06%, about 0.05%, about 0.04%, about 0.03%, about 0.02%, or about 0.01% impurities and/or byproducts.

A “cell wall polysaccharide”, as used herein, refers to any PS found in the cell wall of a bacterium, distinct from those found, for instance, in a bacterial capsule. The cell wall polysaccharide may be bound to the cell via peptidoglycan. The cell wall polysaccharide may come from a gram-positive or gram-negative bacterial cell. An exemplary cell wall polysaccharide is the Group A streptococcus (GAS) PS.

A “peptidoglycan-bound capsular polysaccharide”, as used herein, refers to any PS from a bacterial capsule that is bound to the cell via peptidoglycan and is resistant to degradation under the conditions of the processes described herein. These include certain PS containing some combination of monosaccharides including glucose, rhamnose, galactose, mannose, ribose, glucuronic acid, galacturonic acid, and 2-acetamido-4-amino-2,4,6-trideoxygalactos (AATGal). The peptidoglycan-bound capsular polysaccharide may come from a gram-positive or gram-negative bacterial cell. Peptidoglycan-bound capsular polysaccharides suitable for purification by the described methods may also be characterized by little or no O-Acetylation or few or no phosphodiester bonds.

Generally, the processes for purifying cell wall polysaccharides or peptidoglycan-bound capsular polysaccharides from a bacterial cell described herein comprise hydrolyzing the bacterial cell in a solution comprising base and a reducing agent and incubating polysaccharide-containing composition with a muralytic enzyme to form a free polysaccharide solution. In some embodiments, the process comprises (a) hydrolyzing the bacterial cell in a solution comprising base and a reducing agent to form a lysate comprising polysaccharide; and (b) incubating the lysate comprising polysaccharide with a muralytic enzyme to form a free polysaccharide solution. In some embodiments, the process for producing a purified cell wall polysaccharide or a peptidoglycan-bound capsular polysaccharide from a bacterial cell comprises (a) incubating the bacterial cell with a muralytic enzyme to form a cleaved polysaccharide solution; and (b) incubating the cleaved polysaccharide solution with a base to form a free polysaccharide solution.

In some embodiments, the processes described herein may be suitable for the purification of polysaccharides that lack O-acetyl groups. The purification processes may be used for PS with N-acetylated glucose, rhamnose, mannose, ribose, or galactose monomers. In some embodiments of the processes described herein, the bacterial cell is a Pseudomonas bacterial cell, a Streptococcus bacterial cell, a Staphylococcus bacterial cell, a Neisseria bacterial cell, a Haemophilus bacterial cell, a Listeria bacterial cell, an Enterococcus bacterial cell, or a Clostridium bacterial cell. In certain embodiments, the bacterial cell is selected from the group consisting of Pseudomonas aeruginosa, Streptococcus viridans, Streptococcus mutans, and Streptococcus pyogenes.

In certain embodiments, the bacterial cell is Streptococcus pyogenes, also known as Group A streptococcus (GAS). GAS is a Gram-positive, beta-hemolytic coccus in chains. It is responsible for a range of diseases in humans. These diseases include strep throat (acute pharyngitis), otitis media, and skin and soft tissue infections such impetigo and cellulitis. These can also include rare cases of invasive (serious) illnesses such as necrotizing fasciitis (flesh eating disease) and toxic shock syndrome (TSS). Several virulence factors contribute to the pathogenesis of GAS, such as M protein, hemolysins, and extracellular enzymes. In certain embodiments, the Streptococcus pyogenes bacterial cell is of a serotype selected from M1, M2, M3, M4, M5, M6, M9, M11, M12, M13, M18, M22, M25, M28, M41, M43, M44, M62, M71, M72, M74, M75, M77, M80, M81, M83, M87, M89, and M92, M94, M110, or a mutant of any of the foregoing. In some embodiments of the processes described herein, M protein (e.g., M1) is effectively removed during purification while maintaining good yields of the isolated long polysaccharide.

In some embodiments, a Streptococcus pyogenes bacterial cell may be engineered to produce a polysaccharide or a variant thereof that lacks an immunodominant N-acetyl Glucosamine (GLcNAc) side chain (See e.g., International PCT Publication No. WO 2013/020090, U.S. Pat. No. 10,780,155, International PCT Publication No. WO 2021/167996, and Gao, N. J. et al., Dec. 29, 2020. Infectious Microbes and Diseases, doi: 10.1097/IM9.0000000000000044).

In some embodiments, the processes described herein may be suitable for purifying a peptidoglycan-bound capsular polysaccharide from a bacterial cell

In the processes of the present disclosure, treatment of bacterial cells with base may serve to release polysaccharide from other cellular components (e.g., the cell wall, etc). Unlike known methods of PS purification that utilize acid treatment, base used as described herein may avoid hydrolyzing glycosidic bonds contained in the native polysaccharide. Specifically, the process for purifying cell wall polysaccharides or peptidoglycan-bound capsular polysaccharides from a bacterial cell comprises hydrolyzing the bacterial cell in a solution comprising base and a reducing agent to form a lysate comprising polysaccharide. Non-limiting examples of bases that are suitable for the disclosed process are alkali metal hydroxides such as NaOH, KOH, LiOH, carbonates such as Na₂CO₃ and K₂CO₃, organic amines such as Et₃N and NH₃ and alkoxides such as NaOMe, NaOEt, and KOtBu. In some embodiments, the base is NaOH, KOH, or LiOH. In certain embodiments, the base is NaOH. In certain embodiments, the base is KOH. In some embodiments, the base is LiOH.

In some embodiments, the concentration of base is between about 2M to about 8M. In certain embodiments, the concentration of base is between about 2M and about 6M. In certain embodiments, the concentration of base is between about 2M and about 4M. In certain embodiments, the concentration of base is between about 4M and about 8M. In certain embodiments, the concentration of base is between about 4M and about 6M. In certain embodiments, the concentration of base is between about 6M and about 8M. In some embodiments of the present disclosure, the solution comprising base and a reducing agent is greater than about pH 7. In certain embodiments, the solution comprising base and a reducing agent is about pH 8, about pH 9, about pH 10, about pH 11, about pH 12, about pH 13, or about pH 14.

PS can undergo degradation under high pH through a process called a “peeling reaction,” and a reducing agent may be added to prevent this from occurring. Any suitable reducing agent may be chosen to counteract the peeling reaction. In some embodiments, the reducing agent is selected from sodium borohydride, sodium cyanoborohydride, sodium triacetoxyborohydride, dithiothreitol, or beta-mercaptoethanol. In certain embodiments, the reducing agent is sodium borohydride. The concentration of the reducing agent may be between about 1 mM and about 500 mM. In some embodiments, the concentration of the reducing agent is between about 1 mM and about 500 mM. In certain embodiments, the concentration of the reducing agent is between about 1 mM and about 400 mM, about 1 mM and about 300 mM, about 1 mM and about 200 mM, about 1 mM and about 100 mM, about 1 mM and about 50 mM, about 1 mM and about 10 mM, about 10 mM and about 100 mM, about 100 mM and about 400 mM, about 100 mM and about 300 mM, about 100 mM and about 200 mM, about 200 mM and about 400 mM, about 200 mM and about 300 mM, and about 300 mM and about 400 mM.

When hydrolyzing the bacterial cell in a solution comprising base and a reducing agent to form a lysate comprising polysaccharide, the temperature may be increased in order to achieve higher molecular weight PS and greater yield thereof. In some embodiments, the hydrolysis step further comprises incubating the solution between about 30° C. and about 100° C. In certain embodiments, the hydrolysis step further comprises incubating the solution between about 30° C. and about 100° C.; about 30° C. and about 90° C.; about 30° C. and about 80° C.; about 30° C. and about 70° C.; about 30° C. and about 60° C.; about 30° C. and about 50° C.; about 30° C. and about 40° C.; about 40° C. and about 100° C.; about 40° C. and about 90° C.; about 40° C. and about 80° C.; about 40° C. and about 70° C.; about 40° C. and about 60° C.; about 40° C. and about 50° C.; about 50° C. and about 100° C.; about 50° C. and about 90° C.; about 50° C. and about 80° C.; about 50° C. and about 70° C.; about 50° C. and about 60° C.; about 60° C. and about 100° C.; about 60° C. and about 90° C.; about 60° C. and about 80° C.; about 60° C. and about 70° C.; about 70° C. and about 100° C.; about 70° C. and about 90° C.; about 70° C. and about 80° C.; about 80° C. and about 100° C.; about 80° C. and about 90° C.; or about 90° C. and about 100° C.

The solution comprising base and a reducing agent to form a lysate comprising polysaccharide can be incubated for an amount of time sufficient for completion of the reaction. For instance, in some embodiments, the hydrolysis step further comprises incubating the solution between about 0.5 hours and about 20 hours. In certain embodiments, the hydrolysis step further comprises incubating the solution between about 0.5 hours and about 1 hour; about 1 hour and about 5 hours; about 5 hours and about 20 hours; about 5 hours and about 15 hours; about 5 hours and about 10 hours; about 10 hours and about 20 hours; about 10 hours and about 15 hours; or about 15 hours and about 20 hours. Completion of the hydrolysis can be monitored, for example, by determining the yield of polysaccharide by an appropriate analytical method.

Because hydrolyzing the bacterial cell in a solution comprising base and a reducing agent to form a lysate comprising polysaccharide results in the formation of non-PS byproducts that must be removed, the process may comprise one or more pH adjustment steps to assist in the removal of those byproducts. In one embodiment, the one or more pH adjustment steps are independently selected from: (i) raising the lysate comprising polysaccharide pH, or (ii) lowering the lysate comprising polysaccharide pH.

In some embodiments, the process comprises lowering the lysate comprising polysaccharide pH to between about 3 and about 7. In certain embodiments, the process comprises lowering the lysate comprising polysaccharide pH to between about 3 and about 6; about 3 and about 5; about 3 and about 4; about 4 and about 7; about 4 and about 6; about 4 and about 5; about 5 and about 7; or about 5 and about 6. In some embodiments, the process comprises lowering the lysate comprising polysaccharide pH to about 3, about 3.5, about 4, about 4.5, about 5, about 5.5, about 6, about 6.5, or about 7. In some embodiments, the process comprises: (i) lowering the lysate comprising polysaccharide pH to between about 5.5 and 7.0; (ii) lowering the lysate comprising polysaccharide pH to about 3; and (iii) raising the lysate comprising polysaccharide pH to between about 5.5 and 7.0. In certain embodiments, the process comprises: (i) lowering the lysate comprising polysaccharide pH to between about 6.5; (ii) lowering the lysate comprising polysaccharide pH to about 3; and (iii) raising the lysate comprising polysaccharide pH to between about 6.5.

In the processes disclosed herein, the lysate comprising polysaccharide may be incubated at room temperature after the one or more pH adjustment steps. “Room temperature” is defined as the ambient temperature in a laboratory setting, and is typically between about 20° C. and about 25° C. In some embodiments, the lysate comprising polysaccharide is incubated at between about 4° C. and about 30° C. after the one or more pH adjustment steps. In certain embodiments, the lysate comprising polysaccharide is incubated at between about 4° C. and about 30° C.; about 4° C. and about 25° C.; about 4° C. and about 20° C.; about 4° C. and about 15° C.; about 4° C. and about 10° C.; about 10° C. and about 30° C.; about 10° C. and about 25° C.; about 10° C. and about 20° C.; about 10° C. and about 15° C.; about 15° C. and about 30° C.; about 15° C. and about 25° C.; about 15° C. and about 20° C.; about 20° C. and about 30° C.; about 20° C. and about 25° C.; or about 25° C. and about 30° C. after the one or more pH adjustment steps.

It may be helpful to remove solid byproducts formed during the hydrolysis step. Therefore, in some embodiments, the processes disclosed herein may further comprise removing solids from the lysate comprising polysaccharide. In some embodiments, removing solids from the lysate comprising polysaccharide comprises filtration, centrifugation, or a combination thereof. In some embodiments, the filtration comprises depth filtration, tangential flow filtration (TFF), sterile filtration, membrane filtration, or a combination of the foregoing. In certain embodiments, solids are removed from the lysate comprising polysaccharide by centrifugation. Various methods of removing solids can be applied in combination and in any order. In some embodiments, filtration comprises depth filtration followed by TFF.

In the processes of the present disclosure, treatment of bacterial cells or lysate comprising polysaccharide with a muralytic enzyme may serve to cleave the glycosidic bonds within a peptidoglycan, releasing the PS from their amino acid framework. The muralytic enzyme, may for instance, cleave the bond between N-acetylmuramic acid (NAM) and N-acetylglucosamine (NAG). Specifically, after hydrolysis, the enzymatic cleavage step may comprise incubating the lysate comprising polysaccharide with a muralytic enzyme to form a free polysaccharide solution. In some embodiments, the muralytic enzyme is mutanolysin, lysozyme, or a bacteriophage hydrolase. To further assist in purification, in certain embodiments, incubating the lysate comprising polysaccharide with a muralytic enzyme to form a free polysaccharide solution further comprises incubating with a protease. In some embodiments, the protease is proteinase K. In certain embodiments, the protease is proteinase K, trypsin, chymotrypsin, endoproteinase Asp-N, endoproteinase Arg-C, endoproteinase Glu-C, endoproteinase Lys-C, pepsin, thermolysin, elastase, papain, substilisin, clostripain, carboxypeptidase A, carboxypeptidase B, carboxypeptidase P, carboxypeptidase Y, cathepsin C, acylamino-acid releasing enzyme, or pyroglutamate.

In some embodiments of the processes disclosed herein incubating the lysate comprising polysaccharide with a muralytic enzyme to form a free polysaccharide solution further comprises warming the lysate comprising polysaccharide with the muralytic enzyme to between about 30° C. and about 65° C. In some embodiments, the lysate comprising polysaccharide with the muralytic enzyme is warmed to between about 30° C. and about 60° C.; about 30° C. and about 55° C.; about 30° C. and about 50° C.; about 30° C. and about 45° C.; about 30° C. and about 40° C.; about 30° C. and about 35° C.; about 40° C. and about 60° C.; about 40° C. and about 55° C.; about 40° C. and about 50° C.; about 40° C. and about 60° C.; about 40° C. and about 55° C.; about 40° C. and about 50° C.; about 45° C. and about 60° C.; about 45° C. and about 55° C.; about 45° C. and about 50° C.; about 50° C. and about 60° C.; or about 55° C. and about 60° C.

In some embodiments, the lysate comprising polysaccharide with the protease is warmed. In some embodiments, the lysate comprising polysaccharide with the protease is warmed to between about 45° C. and about 55° C.; about 45° C. and about 50° C.; or about 50° C. and about 55° C.

In the case of both the lysate comprising PS with the muralytic acid or the lysate comprising PS with the protease, the lysate may be warmed for at least 2 hours. In some embodiments the lysate is warmed between about 6 hours and about 20 hours; about 6 hours and about 18 hours; about 6 hours and about 16 hours; about 6 hours and about 14 hours; about 6 hours and about 12 hours; about 6 hours and about 10 hours; about 6 hours and about 8 hours; about 8 hours and about 20 hours; about 8 hours and about 18 hours; about 8 hours and about 16 hours; about 8 hours and about 14 hours; about 8 hours and about 12 hours; about 8 hours and about 10 hours; about 10 hours and about 20 hours; about 10 hours and about 18 hours; about 10 hours and about 16 hours; about 10 hours and about 14 hours; about 10 hours and about 12 hours; about 12 hours and about 20 hours; about 12 hours and about 18 hours; about 12 hours and about 16 hours; about 12 hours and about 14 hours; about 14 hours and about 20 hours; about 14 hours and about 18 hours; about 14 hours and about 16 hours; about 16 hours and about 20 hours; about 16 hours and about 18 hours; of about 118 hours and about 20 hours.

To further aid in the separation of the PS from byproducts, the free polysaccharide solution produced during the step of incubating the lysate comprising polysaccharide with a muralytic enzyme is further purified. In some embodiments, the free polysaccharide solution is further purified to reduce the concentration of nucleic acids, enzymes, host cell proteins (HCPs), or a combination of the foregoing. In certain embodiments, the free polysaccharide solution is further purified by precipitation, anion-exchange chromatography, cation exchange chromatography, hydrophobic interaction chromatography, ceramic hydroxyapatite-type chromatography, or a combination thereof.

In some embodiments, the free polysaccharide solution is further purified by precipitation. This precipitation may be induced by a number of chemical or physical means. In some embodiments, the free polysaccharide solution is treated with further purified to reduce the concentration of nucleic acids, enzymes, host cell proteins (HCPs), or a combination of the foregoing. In some embodiments, the free polysaccharide solution is treated with a surfactant. In some embodiments, the surfactant is cetyltrimethylammonium bromide (CTAB). In certain embodiments, the concentration of CTAB in the free polysaccharide solution is about 0.10% to about 10%. In some embodiments, the concentration of CTAB in the free polysaccharide solution is about 0.1% to about 0.5%; about 0.5% to about 3%; about 0.5% to about 2%; about 0.5% to about 1%; about 1% to about 10%; about 1% to about 9%; about 1% to about 8%; about 1% to about 7%; about 1% to about 6%; about 1% to about 5%; about 1% to about 4%; about 1% to about 3%; about 1% to about 2%; about 2% to about 10%; about 2% to about 9%; about 2% to about 8%; about 2% to about 7%; about 2% to about 6%; about 2% to about 5%; about 2% to about 4%; about 2% to about 3%; about 3% to about 10%; about 3% to about 9%; about 3% to about 8%; about 3% to about 7%; about 3% to about 6%; about 3% to about 5%; about3% to about 4%; about 4% to about 10%; about 4% to about 9%; about 4% to about 8%; about 4% to about 7%; about 4% to about 6%; about 4% to about 5%; about 5% to about 10%; about 5% to about 9%; about 5% to about 8%; about 5% to about 7%; about 5% to about 6%; about 6% to about 10%; about 6% to about 9%; about 6% to about 8%; about 6% to about 7%; about 7% to about 10%; about 7% to about 9%; about 7% to about 8%; about 8% to about 10%; about 8% to about 9%; or about 9% to about 10%.

Potassium iodide (KI) or another suitable salt may be utilized to remove excess CTAB from solution. In some embodiments, the free polysaccharide solution is treated with potassium KI. In some embodiments, the concentration of KI in the free polysaccharide solution is between about 20 mM to about 400 mM. In certain embodiments, the concentration of KI is between about 20 mM and about 300 mM; about 20 mM and about 200 mM; about 20 mM and about 100 mM; about 20 mM and about 50 mM; about 50 mM and about 300 mM; about 50 mM and about 200 mM; about 50 mM and about 100 mM; about 100 mM and about 300 mM; about 100 mM and about 200 mM; or about 200 mM and about 300 mM.

The free polysaccharide solution produced during the process for producing a purified cell wall polysaccharide or a peptidoglycan-bound capsular polysaccharide from a bacterial cell may be further purified, at any point, by filtration, centrifugation, chromatography, or a combination of the foregoing. In some embodiments, filtration is used. In certain embodiments, the filtration comprises depth filtration, tangential flow filtration (TFF), sterile filtration, or a combination of the foregoing. In some embodiments, the chromatography comprises hydrophobic interaction chromatography (HIC), anion exchange chromatography (AEX), ceramic hydroxyapatite-type chromatography, or cation exchange chromatography (CEX).

### Polypeptide-polysaccharide Conjugates

As discussed previously, the high-molecular weight polysaccharides produced by the methods described herein, are suitable for the formation of polypeptide-polysaccharide conjugates for use in vaccines and the like.

The high-molecular weight polysaccharides of the present disclosure may also be referred to as “long polysaccharides.”

Thus, disclosed herein is a polypeptide-polysaccharide conjugate comprising: (a) a polypeptide antigen (e.g., a GAS polypeptide antigen) or a non-GAS carrier polypeptide comprising at least one non-natural amino acid (nnAA); and (b) a purified cell wall polysaccharide or a peptidoglycan-bound capsular polysaccharide with a molecular weight of at least about 10 kDa to at least about 40 kDa. As described in greater detail herein, the nnAA comprise a click chemistry reactive group to facilitate conjugation between the polypeptide antigen and the PS.

In some embodiments, the polysaccharide used in the conjugate is a purified cell wall polysaccharide or a peptidoglycan-bound capsular polysaccharide.

As described previously, GAS is an important bacteria in human health, causing a number of diseases. In some embodiments, the purified cell wall polysaccharide is a GAS polysaccharide. One GAS polysaccharide, group A carbohydrate (GAC) is composed of a polyrhamnose backbone with an immunodominant N-acetyl Glucosamine (GlcNAc) side chain and is present on the surface of strains of all GAS serotypes irrespective of M type and has been examined as a GAS vaccine candidate. Affinity-purified anti-GAC antibodies successfully opsonized three tested GAS serotypes (Salvadori et al., 1995. J. Infect. Dis. 171:593-600.), and mice immunized with GAC were protected against both intraperitoneal and intranasal GAS challenge (Sabharwal et al., 2006. J. Infect. Dis. 193:129-135). However, immunological cross-reactivity between anti-GAC antibodies and host heart valve proteins (Goldstein et al., 1967. Nature 213:44-47) and cytoskeletal proteins, such as actin, keratin, myosin, and vimentin, raises important potential safety concerns regarding the use of GAC as a GAS vaccine constituent. Therefore, in some embodiments, the purified cell wall polysaccharide is a variant of the GAC that lacks the immunodominant GlcNAc side chain (see e.g., International PCT Publication No. WO 2013/020090 U.S. Pat. No. 10,780,155, and Gao, N. J. et al., Infectious Microbes and Diseases, 2021, 3(2), 87-100).

In some embodiments, the purified cell wall polysaccharide or peptidoglycan-bound capsular polysaccharide has an average molecular weight of about 10 kDa to about 45 kDa; about 10 kDa to about 40 kDa; about 10 kDa to about 35 kDa; about 10 kDa to about 30 kDa; about 10 kDa to about 25 kDa; about 10 kDa to about 20 kDa; about 10 kDa to about 15 kDa; 15 kDa to about 40 kDa; about 15 kDa to about 35 kDa; about 15 kDa to about 30 kDa; about 15 kDa to about 25 kDa; about 15 kDa to about 20 kDa; 20 kDa to about 40 kDa; about 20 kDa to about 35 kDa; about 20 kDa to about 30 kDa; about 20 kDa to about 25 kDa; 25 kDa to about 40 kDa; about 25 kDa to about 35 kDa; about 25 kDa to about 30 kDa; about 30 kDa to about 40 kDa; about 30 kDa to about 35 kDa; or about 35 kDa to about 40 kDa. In some embodiments, the purified cell wall polysaccharide or peptidoglycan-bound capsular polysaccharide has an average molecular weight of about 10 kDa, about 15 kDa, about 20 kDa, about 25 kDa, about 30 kDa, about 35 kDa, about 40 kDa, or about 45 kDa.

In some embodiments, the purified cell wall polysaccharides or peptidoglycan-bound capsular polysaccharides are modified with a click chemistry reactive group to facilitate conjugation to the conjugate protein. Examples of click chemistry reactive groups can be found, for instance, in International PCT Publication No. WO 2018/126229, and International PCT Publication No. WO 2021/167996, each of which is incorporated by reference herein in its entirety. For example, in some embodiments, the c purified cell wall polysaccharides or peptidoglycan-bound capsular polysaccharides are modified with DBCO or DBCO-PEG (e.g., DBCO-PEG-NH₂). In some embodiments, the purified cell wall polysaccharides or peptidoglycan-bound capsular polysaccharides are modified with DBCO-(PEG)₄-NH₂.

The degree to which the purified cell wall polysaccharides or peptidoglycan-bound capsular polysaccharides are modified with a click chemistry reactive group or a linker comprising a click chemistry reactive group may be defined by how many, or what percent (e.g., mol %) of, the polysaccharide repeat units (PSRU) have been modified. Thus, in some embodiments of the purified cell wall polysaccharide or a peptidoglycan-bound capsular polysaccharides described herein, between about 8 mol % and 20 mol % of the polysaccharide repeat units are derivatized by a click chemistry reactive group or linker comprising a click chemistry reactive group. In some embodiments, between about 8 mol % and about 20 mol %, about 8 mol % and about 19 mol %, about 8 mol % and about 18 mol %, about 8 mol % and about 17 mol %, about 8 mol % and about 16 mol %, about 8 mol % and about 15 mol %, about 8 mol % and about 14 mol %, 8 mol % and about 13 mol %, about 8 mol % and about 12 mol %, about 8 mol % and about 11 mol %, 8 mol % and about 10 mol %, about 8 mol % and about 9 mol %, about 9 mol % and about 20 mol %, about 9 mol % and about 19 mol %, about 9 mol % and about 18 mol %, about 9 mol % and about 17 mol %, about 9 mol % and about 16 mol %, about 9 mol % and about 15 mol %, about 9 mol % and about 14 mol %, 9 mol % and about 13 mol %, about 9 mol % and about 12 mol %, about 9 mol % and about 11 mol %, 9 mol % and about 10 mol %, about 10 mol % and about 20 mol %, about 10 mol % and about 19 mol %, about 10 mol % and about 18 mol %, about 10 mol % and about 17 mol %, about 10 mol % and about 16 mol %, about 10 mol % and about 15 mol %, about 10 mol % and about 14 mol %, 10 mol % and about 13 mol %, about 10 mol % and about 12 mol %, about 10 mol % and about 11 mol %, about 11 mol % and about 20 mol %, about 11 mol % and about 19 mol %, about 11 mol % and about 18 mol %, about 11 mol % and about 17 mol %, about 11 mol % and about 16 mol %, about 11 mol % and about 15 mol %, about 11 mol % and about 14 mol %, 11 mol % and about 13 mol %, about 11 mol % and about 12 mol %, about 12 mol % and about 20 mol %, about 12 mol % and about 19 mol %, about 12 mol % and about 18 mol %, about 12 mol % and about 17 mol %, about 12 mol % and about 16 mol %, about 12 mol % and about 15 mol %, about 12 mol % and about 14 mol %, 12 mol % and about 13 mol %, about 13 mol % and about 20 mol %, about 13 mol % and about 19 mol %, about 13 mol % and about 18 mol %, about 13 mol % and about 17 mol %, about 13 mol % and about 16 mol %, about 13 mol % and about 15 mol %, about 13 mol % and about 14 mol %, about 14 mol % and about 20 mol %, about 14 mol % and about 19 mol %, about 14 mol % and about 18 mol %, about 14 mol % and about 17 mol %, about 14 mol % and about 16 mol %, about 14 mol % and about 15 mol %, about 15 mol % and about 20 mol %, about 15 mol % and about 19 mol %, about 15 mol % and about 18 mol %, about 15 mol % and about 17 mol %, about 15 mol % and about 16 mol %, about 16 mol % and about 20 mol %, about 16 mol % and about 19 mol %, about 16 mol % and about 18 mol %, about 16 mol % and about 17 mol %, about 17 mol % and about 20 mol %, about 17 mol % and about 19 mol %, about 17 mol % and about 18 mol %, about 18 mol % and about 20 mol %, about 18 mol % and about 19 mol %, or about 19 mol % and about 20 mol % of the PS repeat units of a polysaccharide are derivatized by a click chemistry reactive group or a linker comprising a click chemistry reactive group. In some embodiments, the polysaccharide repeat units of a polysaccharide are polysaccharide repeat units of a GAS polysaccharide, or a variant thereof. In some embodiments of the purified cell wall polysaccharide or a peptidoglycan-bound capsular polysaccharides described herein, between about 8, 9, 10, 11, 12, 13, 14, 15, 16, 17, 18, 19, or 20 mol % of the polysaccharide repeat units are derivatized by a click chemistry reactive group or linker comprising a click chemistry reactive group. In some embodiments, the GAS polysaccharide, or a variant thereof, lacks an immunodominant N-acetyl Glucosamine (GlcNAc) side chain.

In general, increased linker incorporation (mol %) into the polysaccharide repeat units of a polysaccharides may allow for the production of polypeptide-polysaccharide conjugates of increased molecular weight. As discussed further herein, polypeptide-polysaccharide conjugates formed using polysaccharides with greater linker incorporation may exhibit greater immunogenicity than those with a lower mole percentage of linker incorporation. Additionally, polysaccharides with increased linker incorporation may also result in lower amounts of free polysaccharide present post-conjugation, as discussed further herein.

In reference to the polypeptide-polysaccharide conjugates and the immunogenic compositions described herein, “free polysaccharide” refers to a polysaccharide, polysaccharide derivatized with a linker and/or a click chemistry reactive group, or fragment of a polysaccharide that is not conjugated to a polypeptide. For instance, a free polysaccharide may be present in a mixture of a polypeptide-polysaccharide conjugate after running the conjugation reaction between a derivatized polysaccharide and a polypeptide. Fee polysaccharide concentration may be measured, for example, by the method described in Synthetic Example 6.

The polypeptide antigens used in the conjugates described herein may be GAS or non-GAS polypeptide antigens. In some embodiments, the polypeptide antigen is a full-length GAS polypeptide antigen or a fragment of a full-length GAS polypeptide antigen, comprising at least one non-natural amino acid (nnAA). In some embodiments, the polypeptide antigens are selected from C5a peptidase (UniProt P15926), streptolysin O (SLO, UniProt POC0I3), streptococcal immunoglobulin-binding protein 35 (Sib35, UniProt (Q1XG74), and Fibronectin binding protein F1 (Sfb1, UniProt Q48VN7), and Adhesion and Division polypeptide (SpyAD, UniProt Q9A1H3).

The non-GAS carrier polypeptides used in the conjugates described herein may be be based on full-length non-GAS carrier polypeptides or a fragment of a full-length non-GAS carrier polypeptide, comprising at least one non-natural amino acid. In some embodiments, the non-GAS carrier polypeptide is selected from arginine deiminase (ADI, UniProt POCOB3), CRM197 (1272033-67-6), ferritin (UniProt P02792), and Protein D (UniProt R4R7Q5).

In some embodiments, the polypeptide antigen or the non-GAS carrier protein comprises one or more amino acid mutations in the wild-type amino acid sequence. For example, in some embodiments, the polypeptide antigen is SpyAD, wherein the SpyAD polypeptide comprises one or more amino acid mutations. In some embodiments, the polypeptide antigen is ADI, wherein the ADI polypeptide comprises one or more amino acid mutations. In some embodiments, the amino acid mutation is D277A (e.g., SEQ ID NO: 36).

In some embodiments, the polypeptide antigen or the non-GAS carrier protein comprises at least one non-natural amino acids (nnAA). In some embodiments, the at least one nnAA is substituted for a lysine, a leucine, an arginine, or an isoleucine in the polypeptide antigen or the non-GAS carrier polypeptide. In some embodiments, the one or more nnAA comprise a click chemistry reactive group. Herein, a “click chemistry reactive group” refers to a moiety, such as an azide or an alkyne, capable of undergoing a click chemistry reaction with a second click chemistry reactive group. In some embodiments, one click chemistry reactive group reacts with a second click chemistry reactive group to form a substituted triazole. Examples of this type of click reaction can be found, for instance, in International PCT Publication No. WO 2018/126229. General examples of metal-free click reactions used in biomedical applications can be found, for instance, in Kim, et al., Chemical Science, 2019, 10, 7835-7851. Examples of nnAAs comprising click chemistry reactive groups include 2-amino-3-(4-azidophenyl)propanoic acid (pAF), 2-amino-4-azidobutanoic acid, 2-azido-3-phenylpropionic acid, 2-amino-3-azidopropanoic acid, 2-amino-3-(4-(azidomethyl)phenyl)propanoic acid (pAMF), 2-amino-3-(5-(azidomethyl)pyridin-2-yl)propanoic acid, 2-amino-3-(4-(azidomethyl)pyridin-2-yl)propanoic acid, 2-amino-3-(6-(azidomethyl)pyridin-3-yl)propanoic acid, and 2-amino-5-azidopentanoic acid. In some embodiments, the conjugate polypeptide comprises one or more nnAAs, wherein each of the nnAAs are pAMF.

The polypeptide-polysaccharide conjugates described herein may be characterized by their molecular weight (e.g., average molecular weight), as defined by any reasonable characterization method (e.g., SEC-MALS). The polypeptide-polysaccharide conjugates may, for instance, have a molecular weight or average molecular weight greater than about 185 kDa or greater than 190 kDa. In some embodiments, the polypeptide-polysaccharide conjugates may have a molecular weight or average molecular weight between about 185 kDa and about 700 kDa, about 185 kDa and about 600 kDa, about 185 kDa and about 500 kDa, about 185 kDa and about 400 kDa, about 185 kDa and about 300 kDa, and about 185 kDa and about 200 kDa. In some embodiments, the polypeptide-polysaccharide conjugates may have a molecular weight or average molecular weight between about 185 kDa and about 700 kDa, about 185 kDa and about 650 kDa, about 185 kDa and about 600 kDa, about 185 kDa and about 550 kDa, about 185 kDa and about 500 kDa, about 185 kDa and about 450 kDa, about 185 kDa and about 400 kDa, about 185 kDa and about 350 kDa, about 185 kDa and about 300 kDa, about 185 kDa and about 250 kDa, about 185 kDa and about 200 kDa, between about 190 kDa and about 700 kDa, about 190 kDa and about 650 kDa, about 190 kDa and about 600 kDa, about 190 kDa and about 550 kDa, about 190 kDa and about 500 kDa, about 190 kDa and about 450 kDa, about 190 kDa and about 400 kDa, about 190 kDa and about 350 kDa, about 190 kDa and about 300 kDa, about 190 kDa and about 250 kDa, about 190 kDa and about 200 kDa, about 200 kDa and about 700 kDa, about 200 kDa and about 650 kDa, about 200 kDa and about 600 kDa, about 200 kDa and about 550 kDa, about 200 kDa and about 500 kDa, about 200 kDa and about 450 kDa, about 200 kDa and about 400 kDa, about 200 kDa and about 350 kDa, about 200 kDa and about 300 kDa, about 200 kDa and about 250 kDa, about 250 kDa and about 700 kDa, about 250 kDa and about 650 kDa, about 250 kDa and about 600 kDa, about 250 kDa and about 550 kDa, about 250 kDa and about 500 kDa, about 250 kDa and about 450 kDa, about 250 kDa and about 400 kDa, about 250 kDa and about 350 kDa, about 250 kDa and about 300 kDa, about 300 kDa and about 700 kDa, about 300 kDa and about 650 kDa, about 300 kDa and about 600 kDa, about 300 kDa and about 550 kDa, about 300 kDa and about 500 kDa, about 300 kDa and about 450 kDa, about 300 kDa and about 400 kDa, about 300 kDa and about 350 kDa, about 350 kDa and about 700 kDa, about 350 kDa and about 650 kDa, about 350 kDa and about 600 kDa, about 350 kDa and about 550 kDa, about 350 kDa and about 500 kDa, about 350 kDa and about 450 kDa, about 350 kDa and about 400 kDa, about 400 kDa and about 700 kDa, about 400 kDa and about 650 kDa, about 400 kDa and about 600 kDa, about 400 kDa and about 550 kDa, about 400 kDa and about 500 kDa, about 400 kDa and about 450 kDa, about 450 kDa and about 700 kDa, about 450 kDa and about 650 kDa, about 450 kDa and about 600 kDa, about 450 kDa and about 550 kDa, about 450 kDa and about 500 kDa, about 500 kDa and about 700 kDa, about 500 kDa and about 650 kDa, about 500 kDa and about 600 kDa, about 500 kDa and about 550 kDa, about 550 kDa and about 700 kDa, about 550 kDa and about 650 kDa, about 550 kDa and about 600 kDa, about 600 kDa and about 700 kDa, about 600 kDa and about 650 kDa, or about 650 kDa and about 700 kDa. In some embodiments, the polypeptide-polysaccharide conjugates may have a molecular weight or average molecular weight of about 185, 190, 200, 250, 300, 350, 400, 450, 500, 550, 600, 650, or 700 kDa.

In some embodiments, the polypeptide-polysaccharide conjugates described herein may be characterized by their molecular weight. In some embodiments, the polypeptide-polysaccharide conjugates described herein may be characterized by the mol % of the polysaccharide repeat units of the GAS polysaccharide, or variant thereof, derivatized by a linker. In some embodiments, the polypeptide-polysaccharide conjugates described herein may be characterized by their molecular weight and by the mol % of the polysaccharide repeat units of the GAS polysaccharide, or variant thereof, derivatized by a linker.

In some embodiments, a polypeptide-polysaccharide conjugate described herein comprises: (a) a GAS polypeptide antigen or a non-GAS carrier polypeptide comprising at least one non-natural amino acid (nnAA), wherein the nnAA comprises a click chemistry reactive group; and (b) a purified cell wall polysaccharide or a peptidoglycan-bound capsular polysaccharide with a molecular weight of at least about 10 kDa to at least about 40 kDa..

In some embodiments, a polypeptide-polysaccharide conjugate described herein comprises: (a) a GAS polypeptide antigen or a non-GAS carrier polypeptide comprising at least one non-natural amino acid (nnAA), wherein the nnAA comprises a click chemistry reactive group; and (b) a purified cell wall polysaccharide or a peptidoglycan-bound capsular polysaccharide with a molecular weight of at least about 10 kDa to at least about 40 kDa; wherein between about 10 mol % o and about 18 mol % o of the polysaccharide repeat units of the GAS polysaccharide, or a variant thereof, are derivatized by a linker

Table 1 contains exemplary sequences for select polypeptide antigens and non-GAS carrier proteins, including native, variant, truncated, and nnAA-containing versions of the same.

Table TA contains exemplary sequences for select polypeptide antigens containing rmAAs.

TABLE 1
Exemplary Polypeptide Sequences

| Seq | | |
|---|---|---|
| ID | Name/description | Amino acid sequence |
| 1 | C5a [90-1035 | MHHHHHHGSGENLYFQGKTADTPVTSKATIRDLNDPSQVKTLQE |
| | fragment] WT w/ | KAGKGAGTVVAVIDAGEDKNHEAWRLTDKTKARYQSKEDLEKAK |
| | leader | KEHGITYGEWVNDKVAYYHDYSKDGKTAVDQEHGTHVSGILSGN |
| | | APSETKEPYRLEGAMPEAQLLLMRVEIVNGLADYARNYAQAIRD |
| | | AVNLGAKVINMSFGNAALAYANLPDETKKAFDYAKSKGVSIVTS |
| | | AGNDSSFGGKTRLPLADHPDYGVVGTPAAADSTLTVASYSPDKQ |
| | | LTETATVKTADKQDKEMPVLSTNRFEPNKAYDYAYANRGMKEDD |
| | | FKDVKGKIALIERGDIDFKDKIANAKKAGAVGVLIYDNQDKGFP |
| | | IELPNVDQMPAAFISRKDGLLLKDNPKKTITFNATPKVLPTASG |
| | | TKLSRESSWGLTADGNIKPDIAAPGQDILSSVANNKYAKLSGTS |
| | | MSAPLVAGIMGLLQKQYETQYPDMTPSERLDLAKKVLMSSATAL |
| | | YDEDEKAYFSPRQQGAGAVDAKKASAATMYVTDKDNTSSKVHLN |
| | | NVSDTFEVTVTVHNKSDKPQELYYQATVQTDKVDGKHFALAPKA |
| | | LYETSWQKITIPANSSKQVTVPIDASRESKDLLAQMKNGYFLEG |
| | | FVRFKQDPTKEELMSIPYIGFRGDEGNLSALEKPIYDSKDGSSY |
| | | YHEANSDAKDQLDGDGLQFYALKNNFTALTTESNPWTIIKAVKE |
| | | GVENIEDIESSEITETIFAGTFAKQDDDSHYYIHRHANGKPYAA |
| | | ISPNGDGNRDYVQFQGTFLRNAKNLVAEVLDKEGNVVWTSEVTE |
| | | QVVKNYNNDLASTLGSTRFEKTRWDGKDKDGKVVANGTYTYRVR |
| | | YTPISSGAKEQHTDFDVIVDNTTPEVATSATESTEDRRLTLASK |
| | | PKTSQPVYRERIAYTYMDEDLPTTEYISPNEDGTFTLPEEAETM |
| | | EGATVPLKMSDFTYVVEDMAGNITYTPVTKLLEGHSNK |
| 2 | C5a [90-1035 | KTADTPVTSKATIRDLNDPSQVKTLQEKAGKGAGTVVAVIDAGF |
| | fragment] WT w/o | DKNHEAWRLTDKTKARYQSKEDLEKAKKEHGITYGEWVNDKVAY |
| | leader | YHDYSKDGKTAVDQEHGTHVSGILSGNAPSETKEPYRLEGAMPE |
| | | AQLLLMRVEIVNGLADYARNYAQAIRDAVNLGAKVINMSFGNAA |
| | | LAYANLPDETKKAFDYAKSKGVSIVTSAGNDSSFGGKTRLPLAD |
| | | HPDYGVVGTPAAADSTLTVASYSPDKQLTETATVKTADKQDKEM |
| | | PVLSTNRFEPNKAYDYAYANRGMKEDDFKDVKGKIALIERGDID |
| | | FKDKIANAKKAGAVGVLIYDNQDKGFPIELPNVDQMPAAFISRK |
| | | DGLLLKDNPKKTITFNATPKVLPTASGTKLSRESSWGLTADGNI |
| | | KPDIAAPGQDILSSVANNKYAKLSGTSMSAPLVAGIMGLLQKQY |
| | | ETQYPDMTPSERLDLAKKVLMSSATALYDEDEKAYESPRQQGAG |
| | | AVDAKKASAATMYVTDKDNTSSKVHLNNVSDTFEVTVTVHNKSD |
| | | KPQELYYQATVQTDKVDGKHFALAPKALYETSWQKITIPANSSK |
| | | QVTVPIDASRFSKDLLAQMKNGYFLEGFVRFKQDPTKEELMSIP |
| | | YIGFRGDFGNLSALEKPIYDSKDGSSYYHEANSDAKDQLDGDGL |
| | | QFYALKNNFTALTTESNPWTIIKAVKEGVENIEDIESSEITETI |
| | | FAGTFAKQDDDSHYYIHRHANGKPYAAISPNGDGNRDYVQFQGT |
| | | FLRNAKNLVAEVLDKEGNVVWTSEVTEQVVKNYNNDLASTLGST |
| | | RFEKTRWDGKDKDGKVVANGTYTYRVRYTPISSGAKEQHTDFDV |
| | | IVDNTTPEVATSATFSTEDRRLTLASKPKTSQPVYRERIAYTYM |
| | | DEDLPTTEYISPNEDGTFTLPEEAETMEGATVPLKMSDFTYVVE |
| | | DMAGNITYTPVTKLLEGHSNK |
| 3 | C5a [90-1035 | MHHHHHHGSGENLYFQGKTADTPVTSKATIRDLNDPSQVKTLQE |
| | fragment] | KAGKGAGTVVAVIAAGFDKNHEAWRLTDKTKARYQSKEDLEKAK |
| | D131A/S513A w/ | KEHGITYGEWVNDKVAYYHDYSKDGKTAVDQEHGTHVSGILSGN |
| | leader | APSETKEPYRLEGAMPEAQLLLMRVEIVNGLADYARNYAQAIRD |
| | | AVNLGAKVINMSFGNAALAYANLPDETKKAFDYAKSKGVSIVTS |
| | | AGNDSSFGGKTRLPLADHPDYGVVGTPAAADSTLTVASYSPDKQ |
| | | LTETATVKTADKQDKEMPVLSTNRFEPNKAYDYAYANRGMKEDD |
| | | FKDVKGKIALIERGDIDFKDKIANAKKAGAVGVLIYDNQDKGFP |
| | | IELPNVDQMPAAFISRKDGLLLKDNPKKTITFNATPKVLPTASG |
| | | TKLSRFSSWGLTADGNIKPDIAAPGQDILSSVANNKYAKLSGTA |
| | | MSAPLVAGIMGLLQKQYETQYPDMTPSERLDLAKKVLMSSATAL |
| | | YDEDEKAYESPRQQGAGAVDAKKASAATMYVTDKDNTSSKVHLN |
| | | NVSDTFEVTVTVHNKSDKPQELYYQATVQTDKVDGKHFALAPKA |
| | | LYETSWQKITIPANSSKQVTVPIDASRFSKDLLAQMKNGYFLEG |
| | | FVRFKQDPTKEELMSIPYIGFRGDEGNLSALEKPIYDSKDGSSY |
| | | YHEANSDAKDQLDGDGLQFYALKNNFTALTTESNPWTIIKAVKE |
| | | GVENIEDIESSEITETIFAGTFAKQDDDSHYYIHRHANGKPYAA |
| | | ISPNGDGNRDYVQFQGTFLRNAKNLVAEVLDKEGNVVWTSEVTE |
| | | QVVKNYNNDLASTLGSTRFEKTRWDGKDKDGKVVANGTYTYRVR |
| | | YTPISSGAKEQHTDEDVIVDNTTPEVATSATFSTEDRRLTLASK |
| | | PKTSQPVYRERIAYTYMDEDLPTTEYISPNEDGTFTLPEEAETM |
| | | EGATVPLKMSDFTYVVEDMAGNITYTPVTKLLEGHSNK |
| 4 | C5a [90-1035 | KTADTPVTSKATIRDLNDPSQVKTLQEKAGKGAGTVVAVIAAGF |
| | fragment] | DKNHEAWRLTDKTKARYQSKEDLEKAKKEHGITYGEWVNDKVAY |
| | D131A/S513A w/o | YHDYSKDGKTAVDQEHGTHVSGILSGNAPSETKEPYRLEGAMPE |
| | leader | AQLLLMRVEIVNGLADYARNYAQAIRDAVNLGAKVINMSEGNAA |
| | | LAYANLPDETKKAFDYAKSKGVSIVTSAGNDSSFGGKTRLPLAD |
| | | HPDYGVVGTPAAADSTLTVASYSPDKQLTETATVKTADKQDKEM |
| | | PVLSTNRFEPNKAYDYAYANRGMKEDDFKDVKGKIALIERGDID |
| | | FKDKIANAKKAGAVGVLIYDNQDKGFPIELPNVDQMPAAFISRK |
| | | DGLLLKDNPKKTITFNATPKVLPTASGTKLSRFSSWGLTADGNI |
| | | KPDIAAPGQDILSSVANNKYAKLSGTAMSAPLVAGIMGLLQKQY |
| | | ETQYPDMTPSERLDLAKKVLMSSATALYDEDEKAYESPRQQGAG |
| | | AVDAKKASAATMYVTDKDNTSSKVHLNNVSDTFEVTVTVHNKSD |
| | | KPQELYYQATVQTDKVDGKHFALAPKALYETSWQKITIPANSSK |
| | | QVTVPIDASRFSKDLLAQMKNGYFLEGFVRFKQDPTKEELMSIP |
| | | YIGFRGDEGNLSALEKPIYDSKDGSSYYHEANSDAKDQLDGDGL |
| | | QFYALKNNFTALTTESNPWTIIKAVKEGVENIEDIESSEITETI |
| | | FAGTFAKQDDDSHYYIHRHANGKPYAAISPNGDGNRDYVQFQGT |
| | | FLRNAKNLVAEVLDKEGNVVWTSEVTEQVVKNYNNDLASTLGST |
| | | RFEKTRWDGKDKDGKVVANGTYTYRVRYTPISSGAKEQHTDEDV |
| | | IVDNTTPEVATSATFSTEDRRLTLASKPKTSQPVYRERIAYTYM |
| | | DEDLPTTEYISPNEDGTFTLPEEAETMEGATVPLKMSDFTYVVE |
| | | DMAGNITYTPVTKLLEGHSNK |
| 5 | SLO [79-571 | MHHHHHHGSGENLYFQGAPKEMPLESAEKEEKKSEDKKKSEEDH |
| | fragment] WT w/ | TEEINDKIYSLNYNELEVLAKNGETIENFVPKEGVKKADKFIVI |
| | His tag and TEV | ERKKKNINTTPVDISIIDSVTDRTYPAALQLANKGFTENKPDAV |
| | sequence | VTKRNPQKIHIDLPGMGDKATVEVNDPTYANVSTAIDNLVNQWH |
| | | DNYSGGNTLPARTQYTESMVYSKSQIEAALNVNSKILDGTLGID |
| | | FKSISKGEKKVMIAAYKQIFYTVSANLPNNPADVEDKSVTEKEL |
| | | QRKGVSNEAPPLEVSNVAYGRTVFVKLETSSKSNDVEAAFSAAL |
| | | KGTDVKTNGKYSDILENSSFTAVVLGGDAAEHNKVVTKDEDVIR |
| | | NVIKDNATFSRKNPAYPISYTSVFLKNNKIAGVNNRTEYVETTS |
| | | TEYTSGKINLSHQGAYVAQYEILWDEINYDDKGKEVITKRRWDN |
| | | NWYSKTSPFSTVIPLGANSRNIRIMARECTGLAWEWWRKVIDER |
| | | DVKLSKEINVNISGSTLSPYGSITYK |
| 6 | SLO [79-571 | APKEMPLESAEKEEKKSEDKKKSEEDHTEEINDKIYSLNYNELE |
| | fragment] WT w/o | VLAKNGETIENFVPKEGVKKADKFIVIERKKKNINTTPVDISII |
| | leader | DSVTDRTYPAALQLANKGFTENKPDAVVTKRNPQKIHIDLPGMG |
| | | DKATVEVNDPTYANVSTAIDNLVNQWHDNYSGGNTLPARTQYTE |
| | | SMVYSKSQIEAALNVNSKILDGTLGIDFKSISKGEKKVMIAAYK |
| | | QIFYTVSANLPNNPADVEDKSVTFKELQRKGVSNEAPPLFVSNV |
| | | AYGRTVFVKLETSSKSNDVEAAFSAALKGTDVKINGKYSDILEN |
| | | SSFTAVVLGGDAAEHNKVVTKDEDVIRNVIKDNATFSRKNPAYP |
| | | ISYTSVFLKNNKIAGVNNRTEYVETTSTEYTSGKINLSHQGAYV |
| | | AQYEILWDEINYDDKGKEVITKRRWDNNWYSKTSPESTVIPLGA |
| | | NSRNIRIMARECTGLAWEWWRKVIDERDVKLSKEINVNISGSTL |
| | | SPYGSITYK |
| 7 | SLO [79-571 | MHHHHHHGSGENLYFQGAPKEMPLESAEKEEKKSEDKKKSEEDH |
| | fragment] W535A | TEEINDKIYSLNYNELEVLAKNGETIENFVPKEGVKKADKFIVI |
| | w/ leader | ERKKKNINTTPVDISIIDSVTDRTYPAALQLANKGFTENKPDAV |
| | | VTKRNPQKIHIDLPGMGDKATVEVNDPTYANVSTAIDNLVNQWH |
| | | DNYSGGNTLPARTQYTESMVYSKSQIEAALNVNSKILDGTLGID |
| | | FKSISKGEKKVMIAAYKQIFYTVSANLPNNPADVEDKSVTFKEL |
| | | QRKGVSNEAPPLEVSNVAYGRTVFVKLETSSKSNDVEAAFSAAL |
| | | KGTDVKINGKYSDILENSSFTAVVLGGDAAEHNKVVTKDEDVIR |
| | | NVIKDNATFSRKNPAYPISYTSVFLKNNKIAGVNNRTEYVETTS |
| | | TEYTSGKINLSHQGAYVAQYEILWDEINYDDKGKEVITKRRWDN |
| | | NWYSKTSPFSTVIPLGANSRNIRIMARECTGLAAEWWRKVIDER |
| | | DVKLSKEINVNISGSTLSPYGSITYK |
| 8 | SLO [79-571 | APKEMPLESAEKEEKKSEDKKKSEEDHTEEINDKIYSLNYNELE |
| | fragment] W535A | VLAKNGETIENFVPKEGVKKADKFIVIERKKKNINTTPVDISII |
| | w/o leader | DSVTDRTYPAALQLANKGFTENKPDAVVTKRNPQKIHIDLPGMG |
| | | DKATVEVNDPTYANVSTAIDNLVNQWHDNYSGGNTLPARTQYTE |
| | | SMVYSKSQIEAALNVNSKILDGTLGIDFKSISKGEKKVMIAAYK |
| | | QIFYTVSANLPNNPADVFDKSVTFKELQRKGVSNEAPPLFVSNV |
| | | AYGRTVFVKLETSSKSNDVEAAFSAALKGTDVKINGKYSDILEN |
| | | SSFTAVVLGGDAAEHNKVVTKDFDVIRNVIKDNATFSRKNPAYP |
| | | ISYTSVFLKNNKIAGVNNRTEYVETTSTEYTSGKINLSHQGAYV |
| | | AQYEILWDEINYDDKGKEVITKRRWDNNWYSKTSPESTVIPLGA |
| | | NSRNIRIMARECTGLAAEWWRKVIDERDVKLSKEINVNISGSTL |
| | | SPYGSITYK |
| 9 | Spy AD [33-849 | MHHHHHHGSGENLYFQGQVKADDRASGETKASNTHDDSLPKPET |
| | fragment] WT w/ | IQEAKATIDAVEKTLSQQKAELTELATALTKTTAEINHLKEQQD |
| | leader | NEQKALTSAQEIYTNTLASSEETLLAQGAEHQRELTATETELHN |
| | | AQADQHSKETALSEQKASISAETTRAQDLVEQVKTSEQNIAKLN |
| | | AMISNPDAITKAAQTANDNTKALSSELEKAKADLENQKAKVKKQ |
| | | LTEELAAQKAALAEKEAELSRLKSSAPSTQDSIVGNNTMKAPQG |
| | | YPLEELKKLEASGYIGSASYNNYYKEHADQIIAKASPGNQLNQY |
| | | QDIPADRNRFVDPDNLTPEVQNELAQFAAHMINSVRRQLGLPPV |
| | | TVTAGSQEFARLLSTSYKKTHGNTRPSFVYGQPGVSGHYGVGPH |
| | | DKTIIEDSAGASGLIRNDDNMYENIGAFNDVHTVNGIKRGIYDS |
| | | IKYMLFTDHLHGNTYGHAINFLRVDKHNPNAPVYLGESTSNVGS |
| | | LNEHFVMFPESNIANHQRFNKTPIKAVGSTKDYAQRVGTVSDTI |
| | | AAIKGKVSSLENRLSAIHQEADIMAAQAKVSQLQGKLASTLKQS |
| | | DSLNLQVRQLNDTKGSLRTELLAAKAKQAQLEATRDQSLAKLAS |
| | | LKAALHQTEALAEQAAARVTALVAKKAHLQYLRDFKLNPNRLQV |
| | | IRERIDNTKQDLAKTTSSLLNAQEALAALQAKQSSLEATIATTE |
| | | HQLTLLKTLANEKEYRHLDEDIATVPDLQVAPPLTGVKPLSYSK |
| | | IDTTPLVQEMVKETKQLLEASARLAAENTSLVAEALVGQTSEMV |
| | | ASNAIVSKITSSITQPSSKTSYGSGSSTTSNLISDVDESTQR |
| 10 | Spy AD [33-849 | QVKADDRASGETKASNTHDDSLPKPETIQEAKATIDAVEKTLSQ |
| | fragment] WT w/o | QKAELTELATALTKTTAEINHLKEQQDNEQKALTSAQEIYTNTL |
| | leader | ASSEETLLAQGAEHQRELTATETELHNAQADQHSKETALSEQKA |
| | | SISAETTRAQDLVEQVKTSEQNIAKLNAMISNPDAITKAAQTAN |
| | | DNTKALSSELEKAKADLENQKAKVKKQLTEELAAQKAALAEKEA |
| | | ELSRLKSSAPSTQDSIVGNNTMKAPQGYPLEELKKLEASGYIGS |
| | | ASYNNYYKEHADQIIAKASPGNQLNQYQDIPADRNRFVDPDNLT |
| | | PEVQNELAQFAAHMINSVRRQLGLPPVTVTAGSQEFARLLSTSY |
| | | KKTHGNTRPSFVYGQPGVSGHYGVGPHDKTIIEDSAGASGLIRN |
| | | DDNMYENIGAFNDVHTVNGIKRGIYDSIKYMLFTDHLHGNTYGH |
| | | AINFLRVDKHNPNAPVYLGFSTSNVGSLNEHFVMFPESNIANHQ |
| | | RFNKTPIKAVGSTKDYAQRVGTVSDTIAAIKGKVSSLENRLSAI |
| | | HQEADIMAAQAKVSQLQGKLASTLKQSDSLNLQVRQLNDTKGSL |
| | | RTELLAAKAKQAQLEATRDQSLAKLASLKAALHQTEALAEQAAA |
| | | RVTALVAKKAHLQYLRDFKLNPNRLQVIRERIDNTKQDLAKTTS |
| | | SLLNAQEALAALQAKQSSLEATIATTEHQLTLLKTLANEKEYRH |
| | | LDEDIATVPDLQVAPPLTGVKPLSYSKIDTTPLVQEMVKETKQL |
| | | LEASARLAAENTSLVAEALVGQTSEMVASNAIVSKITSSITQPS |
| | | SKTSYGSGSSTTSNLISDVDESTQR |
| 11 | Spy AD [33-849 | MHHHHHHGSGENLYFQGQVKADDRASGETKASNTHDDSLPKPET |
| | fragment] nnAA | IQEAXATIDAVEKTLSQQKAELTELATALTKTTAEINHLKEQQD |
| | (e.g., pAMF) | NEQKALTSAQEIYTNTLASSEETLLAQGAEHQRELTATETELHN |
| | mutant w/ | AQADQHSKETALSEQKASISAETTRAQDLVEQVKTSEQNIAKLN |
| | leader | AMISNPDAITKAAQTANDNTKALSSELEKAKADLENQKAKVKKQ |
| | X = nnAA (e.g., | LTEELAAQKAALAEKEAELSRLKSSAPSTQDSIVGNNTMKAPQG |
| | pAMF) positions | YPLEELKXLEASGYIGSASYNNYYKEHADQIIAKASPGNQLNQY |
| | K64/K287/K386/ | QDIPADRNRFVDPDNLTPEVQNELAQFAAHMINSVRRQLGLPPV |
| | K657 - numbered | TVTAGSQEFARLLSTSYKXTHGNTRPSFVYGQPGVSGHYGVGPH |
| | according to full | DKTIIEDSAGASGLIRNDDNMYENIGAFNDVHTVNGIKRGIYDS |
| | length sequence | IKYMLFTDHLHGNTYGHAINFLRVDKHNPNAPVYLGESTSNVGS |
| | | LNEHFVMFPESNIANHQRENKTPIKAVGSTKDYAQRVGTVSDTI |
| | | AAIKGKVSSLENRLSAIHQEADIMAAQAKVSQLQGKLASTLKQS |
| | | DSLNLQVRQLNDTKGSLRTELLAAKAKQAQLEATRDQSLAKLAS |
| | | LKAALHQTEALAEQAAARVTALVAKXAHLQYLRDFKLNPNRLQV |
| | | IRERIDNTKQDLAKTTSSLLNAQEALAALQAKQSSLEATIATTE |
| | | HQLTLLKTLANEKEYRHLDEDIATVPDLQVAPPLTGVKPLSYSK |
| | | IDTTPLVQEMVKETKQLLEASARLAAENTSLVAEALVGQTSEMV |
| | | ASNAIVSKITSSITQPSSKTSYGSGSSTTSNLISDVDESTQR |
| 12 | Spy AD [33-849 | QVKADDRASGETKASNTHDDSLPKPETIQEAXATIDAVEKTLSQ |
| | fragment] nnAA | QKAELTELATALTKTTAEINHLKEQQDNEQKALTSAQEIYTNTL |
| | (e.g., pAMF) | ASSEETLLAQGAEHQRELTATETELHNAQADQHSKETALSEQKA |
| | mutant w/o leader | SISAETTRAQDLVEQVKTSEQNIAKLNAMISNPDAITKAAQTAN |
| | X = nnAA (e.g., | DNTKALSSELEKAKADLENQKAKVKKQLTEELAAQKAALAEKEA |
| | pAMF) | ELSRLKSSAPSTQDSIVGNNTMKAPQGYPLEELKXLEASGYIGS |
| | K64/K287/K386/ | ASYNNYYKEHADQIIAKASPGNQLNQYQDIPADRNRFVDPDNLT |
| | K657 - numbered | PEVQNELAQFAAHMINSVRRQLGLPPVTVTAGSQEFARLLSTSY |
| | according to full- | KXTHGNTRPSFVYGQPGVSGHYGVGPHDKTIIEDSAGASGLIRN |
| | length sequence | DDNMYENIGAFNDVHTVNGIKRGIYDSIKYMLFTDHLHGNTYGH |
| | | AINFLRVDKHNPNAPVYLGFSTSNVGSLNEHFVMFPESNIANHQ |
| | | RFNKTPIKAVGSTKDYAQRVGTVSDTIAAIKGKVSSLENRLSAI |
| | | HQEADIMAAQAKVSQLQGKLASTLKQSDSLNLQVRQLNDTKGSL |
| | | RTELLAAKAKQAQLEATRDQSLAKLASLKAALHQTEALAEQAAA |
| | | RVTALVAKXAHLQYLRDFKLNPNRLQVIRERIDNTKQDLAKTTS |
| | | SLLNAQEALAALQAKQSSLEATIATTEHQLTLLKTLANEKEYRH |
| | | LDEDIATVPDLQVAPPLTGVKPLSYSKIDTTPLVQEMVKETKQL |
| | | LEASARLAAENTSLVAEALVGQTSEMVASNAIVSKITSSITQPS |
| | | SKTSYGSGSSTTSNLISDVDESTQR |
| 13 | ADI [full length] | MHHHHHHGSGENLYFQGTAQTPIHVYSEIGKLKKVLLHRPGKEI |
| | WT w/ leader | ENLMPDYLERLLEDDIPFLEDAQKEHDAFAQALRDEGIEVLYLE |
| | | TLAAESLVTPEIREAFIDEYLSEANIRGRATKKAIRELLMAIED |
| | | NQELIEKTMAGVQKSELPEIPASEKGLTDLVESNYPFAIDPMPN |
| | | LYFTRDPFATIGTGVSLNHMFSETRNRETLYGKYIFTHHPIYGG |
| | | GKVPMVYDRNETTRIEGGDELVLSKDVLAVGISQRTDAASIEKL |
| | | LVNIFKQNLGFKKVLAFEFANNRKFMHLDTVFTMVDYDKFTIHP |
| | | EIEGDLRVYSVTYDNEELHIVEEKGDLAELLAANLGVEKVDLIR |
| | | CGGDNLVAAGREQWNDGSNTLTIAPGVVVVYNRNTITNAILESK |
| | | GLKLIKIHGSELVRGRGGPRCMSMPFEREDI |
| 14 | ADI [full length] | TAQTPIHVYSEIGKLKKVLLHRPGKEIENLMPDYLERLLEDDIP |
| | WT w/o leader | FLEDAQKEHDAFAQALRDEGIEVLYLETLAAESLVTPEIREAFI |
| | | DEYLSEANIRGRATKKAIRELLMAIEDNQELIEKTMAGVQKSEL |
| | | PEIPASEKGLTDLVESNYPFAIDPMPNLYFTRDPFATIGTGVSL |
| | | NHMESETRNRETLYGKYIFTHHPIYGGGKVPMVYDRNETTRIEG |
| | | GDELVLSKDVLAVGISQRTDAASIEKLLVNIFKQNLGFKKVLAF |
| | | EFANNRKFMHLDTVFTMVDYDKFTIHPEIEGDLRVYSVTYDNEE |
| | | LHIVEEKGDLAELLAANLGVEKVDLIRCGGDNLVAAGREQWNDG |
| | | SNTLTIAPGVVVVYNRNTITNAILESKGLKLIKIHGSELVRGRG |
| | | GPRCMSMPFEREDI |
| 15 | ADI [full length] | MHHHHHHGSGENLYFQGTAQTPIHVYSEIGKLKKVLLHRPGKEI |
| | D277A w/ leader | ENLMPDYLERLLEDDIPFLEDAQKEHDAFAQALRDEGIEVLYLE |
| | | TLAAESLVTPEIREAFIDEYLSEANIRGRATKKAIRELLMAIED |
| | | NQELIEKTMAGVQKSELPEIPASEKGLTDLVESNYPFAIDPMPN |
| | | LYFTRDPFATIGTGVSLNHMFSETRNRETLYGKYIFTHHPIYGG |
| | | GKVPMVYDRNETTRIEGGDELVLSKDVLAVGISQRTDAASIEKL |
| | | LVNIFKQNLGFKKVLAFEFANNRKFMHLATVFTMVDYDKFTIHP |
| | | EIEGDLRVYSVTYDNEELHIVEEKGDLAELLAANLGVEKVDLIR |
| | | CGGDNLVAAGREQWNDGSNTLTIAPGVVVVYNRNTITNAILESK |
| | | GLKLIKIHGSELVRGRGGPRCMSMPFEREDI |
| 16 | ADI [full length] | TAQTPIHVYSEIGKLKKVLLHRPGKEIENLMPDYLERLLEDDIP |
| | D277A w/o leader | FLEDAQKEHDAFAQALRDEGIEVLYLETLAAESLVTPEIREAFI |
| | | DEYLSEANIRGRATKKAIRELLMAIEDNQELIEKTMAGVQKSEL |
| | | PEIPASEKGLTDLVESNYPFAIDPMPNLYFTRDPFATIGTGVSL |
| | | NHMESETRNRETLYGKYIFTHHPIYGGGKVPMVYDRNETTRIEG |
| | | GDELVLSKDVLAVGISQRTDAASIEKLLVNIFKQNLGFKKVLAF |
| | | EFANNRKFMHLATVFTMVDYDKFTIHPEIEGDLRVYSVTYDNEE |
| | | LHIVEEKGDLAELLAANLGVEKVDLIRCGGDNLVAAGREQWNDG |
| | | SNTLTIAPGVVVVYNRNTITNAILESKGLKLIKIHGSELVRGRG |
| | | GPRCMSMPFEREDI |
| 17 | ADI [full length] | MHHHHHHGSGENLYFQGTAQTPIHVYSEIGXLKKVLLHRPGKEI |
| | nnAA (e.g., | ENLMPDYLERLLEDDIPFLEDAQKEHDAFAQALRDEGIEVLYLE |
| | pAMF) w/ leader | TLAAESLVTPEIREAFIDEYLSEANIRGRATKKAIRELLMAIED |
| | X = nnAA (e.g., | NQELIEKTMAGVQKSELPEIPASEKGLTDLVESNYPFAIDPMPN |
| | pAMF) positions - | LYFTRDPFATIGTGVSLNHMFSETRNRETLYGXYIFTHHPIYGG |
| | K15, K193, K316 | GKVPMVYDRNETTRIEGGDELVLSKDVLAVGISQRTDAASIEKL |
| | | LVNIFKQNLGFKKVLAFEFANNRKFMHLDTVFTMVDYDKFTIHP |
| | | EIEGDLRVYSVTYDNEELHIVEEXGDLAELLAANLGVEKVDLIR |
| | | CGGDNLVAAGREQWNDGSNTLTIAPGVVVVYNRNTITNAILESK |
| | | GLKLIKIHGSELVRGRGGPRCMSMPFEREDI |
| 18 | ADI [full length] | TAQTPIHVYSEIGXLKKVLLHRPGKEIENLMPDYLERLLEDDIP |
| | nnAA (e.g., | FLEDAQKEHDAFAQALRDEGIEVLYLETLAAESLVTPEIREAFI |
| | pAMF) mutant w/o | DEYLSEANIRGRATKKAIRELLMAIEDNQELIEKTMAGVQKSEL |
| | leader | PEIPASEKGLTDLVESNYPFAIDPMPNLYFTRDPFATIGTGVSL |
| | X = nnAA (e.g., | NHMESETRNRETLYGXYIFTHHPIYGGGKVPMVYDRNETTRIEG |
| | pAMF) positions - | GDELVLSKDVLAVGISQRTDAASIEKLLVNIFKQNLGFKKVLAF |
| | K15, K193, K316 | EFANNRKFMHLDTVFTMVDYDKFTIHPEIEGDLRVYSVTYDNEE |
| | | LHIVEEXGDLAELLAANLGVEKVDLIRCGGDNLVAAGREQWNDG |
| | | SNTLTIAPGVVVVYNRNTITNAILESKGLKLIKIHGSELVRGRG |
| | | GPRCMSMPFEREDI |
| 19 | ADI (mut5) [full | MHHHHHHGSGENLYFQGTAQTPIHVYSEIGXLKKVLLHRPGKEI |
| | length] D277A | ENLMPDYLERLLEDDIPFLEDAQKEHDAFAQALRDEGIEVLYLE |
| | nnAA (e.g., | TLAAESLVTPEIREAFIDEYLSEANIRGRATKKAIRELLMAIED |
| | pAMF) w/ leader | NQELIEKTMAGVQKSELPEIPASEKGLTDLVESNYPFAIDPMPN |
| | X = nnAA (e.g., | LYFTRDPFATIGTGVSLNHMFSETRNRETLYGXYIFTHHPIYGG |
| | pAMF) positions - | GKVPMVYDRNETTRIEGGDELVLSKDVLAVGISQRTDAASIEKL |
| | K15, K193, K316 | LVNIFKQNLGFKKVLAFEFANNRKFMHLATVFTMVDYDKFTIHP |
| | | EIEGDLRVYSVTYDNEELHIVEEXGDLAELLAANLGVEKVDLIR |
| | | CGGDNLVAAGREQWNDGSNTLTIAPGVVVVYNRNTITNAILESK |
| | | GLKLIKIHGSELVRGRGGPRCMSMPFEREDI |
| 20 | ADI (mut5) [full | TAQTPIHVYSEIGXLKKVLLHRPGKEIENLMPDYLERLLEDDIP |
| | length] D277A | FLEDAQKEHDAFAQALRDEGIEVLYLETLAAESLVTPEIREAFI |
| | nnAA (e.g., | DEYLSEANIRGRATKKAIRELLMAIEDNQELIEKTMAGVQKSEL |
| | pAMF) w/o leader | PEIPASEKGLTDLVESNYPFAIDPMPNLYFTRDPFATIGTGVSL |
| | X = nnAA (e.g., | NHMESETRNRETLYGXYIFTHHPIYGGGKVPMVYDRNETTRIEG |
| | pAMF) positions - | GDELVLSKDVLAVGISQRTDAASIEKLLVNIFKQNLGFKKVLAF |
| | K15, K193, K316 | EFANNRKFMHLATVFTMVDYDKFTIHPEIEGDLRVYSVTYDNEE |
| | | LHIVEEXGDLAELLAANLGVEKVDLIRCGGDNLVAAGREQWNDG |
| | | SNTLTIAPGVVVVYNRNTITNAILESKGLKLIKIHGSELVRGRG |
| | | GPRCMSMPFEREDI |
| 21 | hFL [full length] | MHHHHHHGSGSSQIRQNYSTDVEAAVNSLVNLYLQASYTYLSLG |
| | WT w/ leader | FYFDRDDVALEGVSHFFRELAEEKREGYERLLKMQNQRGGRALF |
| | | QDIKKPAEDEWGKTPDAMKAAMALEKKLNQALLDLHALGSARTD |
| | | PHLCDFLETHFLDEEVKLIKKMGDHLTNLHRLGGPEAGLGEYLF |
| | | ERLTLKHD |
| 22 | hFL [full length] | SSQIRQNYSTDVEAAVNSLVNLYLQASYTYLSLGFYFDRDDVAL |
| | WT w/o leader | EGVSHFFRELAEEKREGYERLLKMQNQRGGRALFQDIKKPAEDE |
| | | WGKTPDAMKAAMALEKKLNQALLDLHALGSARTDPHLCDFLETH |
| | | FLDEEVKLIKKMGDHLTNLHRLGGPEAGLGEYLFERLTLKHD |
| 23 | hFL [full length] | MHHHHHHGSGSSQXRQNYSTDVEAAVNSLVNLYLQASYTYLSLG |
| | nnAA (e.g., | FYFDRDDVALEGVSHFFRELAEEKREGYERLLKMQNQRGGRALF |
| | pAMF) w/ leader | QDIKKPAEDEWGKTPDAMKAAMALEKKLNQALLDLHALGSARTD |
| | X = nnAA (e.g., | PHLCDFLETHFLDEEVKLIKKMGDHLTNLHRLGGPEAGLGEYLF |
| | pAMF) position - | ERLTLKHD |
| | I5 | |
| 24 | hFL [full length] | SSQXRQNYSTDVEAAVNSLVNLYLQASYTYLSLGFYFDRDDVAL |
| | nnAA (e.g., | EGVSHFFRELAEEKREGYERLLKMQNQRGGRALFQDIKKPAEDE |
| | pAMF) w/o leader | WGKTPDAMKAAMALEKKLNQALLDLHALGSARTDPHLCDFLETH |
| | X = nnAA (e.g., | FLDEEVKLIKKMGDHLTNLHRLGGPEAGLGEYLFERLTLKHD |
| | pAMF) position - | |
| | I5 | |
| 25 | eCRM-pAMF6 | MGADDVVDSSKSFVMENFSSYHGTKPGYVDSIQXGIQKPKSGTQ |
| | mutant | GNYDDDWKEFYSTDNKYDAAGYSVDNENPLSGKAGGVVKVTYPG |
| | | LTKVLALKVDNAETIKKELGLSLTEPLMEQVGTEEFIKRFGDGA |
| | | SRVVLSLPFAEGSSSVEYINNWEQAKALSVELEINFETRGKRGQ |
| | | DAMYEYMAQACAGNRVRNSVGSSLSCINLDWDVIRDXTKTKIES |
| | | LKEHGPIKNKMSESPNKTVSEEKAXQYLEEFHQTALEHPELSEL |
| | | XTVTGTNPVFAGANYAAWAVNVAQVIDSETADNLEKTTAALSIL |
| | | PGIGSVMGIADGAVHHNTEEIVAQSIALSSLMVAQAIPLVGELV |
| | | DIGFAAYNFVESIINLFQVVHNSYNRPAYSPGHXTQPFLHDGYA |
| | | VSWNTVEDSIIRTGFQGESGHDIKITAENTPLPIAGVLLPTIPG |
| | | KLDVNKSKTHISVNGRKIRMRCRAIDGDVTFCRPKSPVYVGNGV |
| | | HANLHVAFHRSSSEKIHSNEISSDSIGVLGYQKTVDHTKVNSXL |
| | | SLFFEIKS |
| 26 | ADI (mut2) [full | MHHHHHHGSGENLYFQGTAQTPIHVYSEIGKLKKVLLHRPGKEI |
| | length] D277A | ENLMPDYLERLLEDDIPFLEDAQKEHDAFAQALRDEGIEVLYLE |
| | nnAA (e.g., | TLAAESLVTPEIREAFIDEYLSEANIRGRATKKAIRELLMAIED |
| | pAMF) w/ leader | NQELIEXTMAGVQKSELPEIPASEKGLTDLVESNYPFAIDPMPN |
| | X = nnAA (e.g., | LYFTRDPFATIGTGVSLNHMESETRNRETLYGKYIFTHHPIYGG |
| | pAMF) positions - | GKVPMVYDRNETTRIEGGDELVLSKDVLAVGISQRTDAASIEXL |
| | K123, K247, K316 | LVNIFKQNLGFKKVLAFEFANNRKFMHLATVFTMVDYDKFTIHP |
| | | EIEGDLRVYSVTYDNEELHIVEEXGDLAELLAANLGVEKVDLIR |
| | | CGGDNLVAAGREQWNDGSNTLTIAPGVVVVYNRNTITNAILESK |
| | | GLKLIKIHGSELVRGRGGPRCMSMPFEREDI |
| 27 | ADI (mut2) [full | GTAQTPIHVYSEIGKLKKVLLHRPGKEIENLMPDYLERLLEDDI |
| | length] D277A | PFLEDAQKEHDAFAQALRDEGIEVLYLETLAAESLVTPEIREAF |
| | nnAA (e.g., | IDEYLSEANIRGRATKKAIRELLMAIEDNQELIEXTMAGVQKSE |
| | pAMF) w/o leader, | LPEIPASEKGLTDLVESNYPFAIDPMPNLYFTRDPFATIGTGVS |
| | with G overhang | LNHMESETRNRETLYGKYIFTHHPIYGGGKVPMVYDRNETTRIE |
| | from cleavage | GGDELVLSKDVLAVGISQRTDAASIEXLLVNIFKQNLGFKKVLA |
| | X = nnAA (e.g., | FEFANNRKFMHLATVFTMVDYDKFTIHPEIEGDLRVYSVTYDNE |
| | pAMF) positions - | ELHIVEEXGDLAELLAANLGVEKVDLIRCGGDNLVAAGREQWND |
| | K123, K247, K316 | GSNTLTIAPGVVVVYNRNTITNAILESKGLKLIKIHGSELVRGR |
| | | GGPRCMSMPFEREDI |
| 28 | Sfb1 repeat region | MHHHHHHGSGVETEDTKEPGVLMGGQSESVEFTKDTQTGMSGQT |
| | with leader | TPQVETEDTKEPGVLMGGQSESVEFTKDTQTGMSGQTASQVETE |
| | | DTKEPGVLMGGQSESVEFTKDTQTGMSGQTTPQVETEDTKEPGV |
| | | LMGGQSESVEFTKDTQTGMSGFSET |
| 29 | C5a [90-1035 | GKTADTPVTSKATIRDLNDPSQVKTLQEKAGKGAGTVVAVIDAG |
| | fragment] WT w/o | FDKNHEAWRLTDKTKARYQSKEDLEKAKKEHGITYGEWVNDKVA |
| | leader, with G | YYHDYSKDGKTAVDQEHGTHVSGILSGNAPSETKEPYRLEGAMP |
| | overhang from | EAQLLLMRVEIVNGLADYARNYAQAIRDAVNLGAKVINMSEGNA |
| | cleavage | ALAYANLPDETKKAFDYAKSKGVSIVTSAGNDSSFGGKTRLPLA |
| | | DHPDYGVVGTPAAADSTLTVASYSPDKQLTETATVKTADKQDKE |
| | | MPVLSTNRFEPNKAYDYAYANRGMKEDDFKDVKGKIALIERGDI |
| | | DFKDKIANAKKAGAVGVLIYDNQDKGFPIELPNVDQMPAAFISR |
| | | KDGLLLKDNPKKTITFNATPKVLPTASGTKLSRESSWGLTADGN |
| | | IKPDIAAPGQDILSSVANNKYAKLSGTSMSAPLVAGIMGLLQKQ |
| | | YETQYPDMTPSERLDLAKKVLMSSATALYDEDEKAYESPRQQGA |
| | | GAVDAKKASAATMYVTDKDNTSSKVHLNNVSDTFEVTVTVHNKS |
| | | DKPQELYYQATVQTDKVDGKHFALAPKALYETSWQKITIPANSS |
| | | KQVTVPIDASRFSKDLLAQMKNGYFLEGFVRFKQDPTKEELMSI |
| | | PYIGFRGDEGNLSALEKPIYDSKDGSSYYHEANSDAKDQLDGDG |
| | | LQFYALKNNFTALTTESNPWTIIKAVKEGVENIEDIESSEITET |
| | | IFAGTFAKQDDDSHYYIHRHANGKPYAAISPNGDGNRDYVQFQG |
| | | TFLRNAKNLVAEVLDKEGNVVWTSEVTEQVVKNYNNDLASTLGS |
| | | TRFEKTRWDGKDKDGKVVANGTYTYRVRYTPISSGAKEQHTDED |
| | | VIVDNTTPEVATSATFSTEDRRLTLASKPKTSQPVYRERIAYTY |
| | | MDEDLPTTEYISPNEDGTFTLPEEAETMEGATVPLKMSDFTYVV |
| | | EDMAGNITYTPVTKLLEGHSNK |
| 30 | C5a [90-1035 | GKTADTPVTSKATIRDLNDPSQVKTLQEKAGKGAGTVVAVIAAG |
| | fragment] | FDKNHEAWRLTDKTKARYQSKEDLEKAKKEHGITYGEWVNDKVA |
| | D131A/S513A w/o | YYHDYSKDGKTAVDQEHGTHVSGILSGNAPSETKEPYRLEGAMP |
| | leader, with G | EAQLLLMRVEIVNGLADYARNYAQAIRDAVNLGAKVINMSFGNA |
| | overhang from | ALAYANLPDETKKAFDYAKSKGVSIVTSAGNDSSFGGKTRLPLA |
| | cleavage | DHPDYGVVGTPAAADSTLTVASYSPDKQLTETATVKTADKQDKE |
| | | MPVLSTNRFEPNKAYDYAYANRGMKEDDFKDVKGKIALIERGDI |
| | | DFKDKIANAKKAGAVGVLIYDNQDKGFPIELPNVDQMPAAFISR |
| | | KDGLLLKDNPKKTITFNATPKVLPTASGTKLSRFSSWGLTADGN |
| | | IKPDIAAPGQDILSSVANNKYAKLSGTAMSAPLVAGIMGLLQKQ |
| | | YETQYPDMTPSERLDLAKKVLMSSATALYDEDEKAYESPRQQGA |
| | | GAVDAKKASAATMYVTDKDNTSSKVHLNNVSDTFEVTVTVHNKS |
| | | DKPQELYYQATVQTDKVDGKHFALAPKALYETSWQKITIPANSS |
| | | KQVTVPIDASRFSKDLLAQMKNGYFLEGFVRFKQDPTKEELMSI |
| | | PYIGFRGDEGNLSALEKPIYDSKDGSSYYHEANSDAKDQLDGDG |
| | | LQFYALKNNFTALTTESNPWTIIKAVKEGVENIEDIESSEITET |
| | | IFAGTFAKQDDDSHYYIHRHANGKPYAAISPNGDGNRDYVQFQG |
| | | TFLRNAKNLVAEVLDKEGNVVWTSEVTEQVVKNYNNDLASTLGS |
| | | TRFEKTRWDGKDKDGKVVANGTYTYRVRYTPISSGAKEQHTDED |
| | | VIVDNTTPEVATSATESTEDRRLTLASKPKTSQPVYRERIAYTY |
| | | MDEDLPTTEYISPNEDGTFTLPEEAETMEGATVPLKMSDFTYVV |
| | | EDMAGNITYTPVTKLLEGHSNK |
| 31 | SLO [79-571 | GAPKEMPLESAEKEEKKSEDKKKSEEDHTEEINDKIYSLNYNEL |
| | fragment] WT w/o | EVLAKNGETIENFVPKEGVKKADKFIVIERKKKNINTTPVDISI |
| | leader, with G | IDSVTDRTYPAALQLANKGFTENKPDAVVTKRNPQKIHIDLPGM |
| | overhang from | GDKATVEVNDPTYANVSTAIDNLVNQWHDNYSGGNTLPARTQYT |
| | cleavage | ESMVYSKSQIEAALNVNSKILDGTLGIDFKSISKGEKKVMIAAY |
| | | KQIFYTVSANLPNNPADVEDKSVTFKELQRKGVSNEAPPLEVSN |
| | | VAYGRTVFVKLETSSKSNDVEAAFSAALKGTDVKINGKYSDILE |
| | | NSSFTAVVLGGDAAEHNKVVTKDFDVIRNVIKDNATFSRKNPAY |
| | | PISYTSVFLKNNKIAGVNNRTEYVETTSTEYTSGKINLSHQGAY |
| | | VAQYEILWDEINYDDKGKEVITKRRWDNNWYSKTSPESTVIPLG |
| | | ANSRNIRIMARECTGLAWEWWRKVIDERDVKLSKEINVNISGST |
| | | LSPYGSITYK |
| 32 | SLO [79-571 | GAPKEMPLESAEKEEKKSEDKKKSEEDHTEEINDKIYSLNYNEL |
| | fragment] W535A | EVLAKNGETIENFVPKEGVKKADKFIVIERKKKNINTTPVDISI |
| | w/o leader, with G | IDSVTDRTYPAALQLANKGFTENKPDAVVTKRNPQKIHIDLPGM |
| | overhang from | GDKATVEVNDPTYANVSTAIDNLVNQWHDNYSGGNTLPARTQYT |
| | cleavage | ESMVYSKSQIEAALNVNSKILDGTLGIDFKSISKGEKKVMIAAY |
| | | KQIFYTVSANLPNNPADVEDKSVTFKELQRKGVSNEAPPLEVSN |
| | | VAYGRTVFVKLETSSKSNDVEAAFSAALKGTDVKINGKYSDILE |
| | | NSSFTAVVLGGDAAEHNKVVTKDFDVIRNVIKDNATFSRKNPAY |
| | | PISYTSVFLKNNKIAGVNNRTEYVETTSTEYTSGKINLSHQGAY |
| | | VAQYEILWDEINYDDKGKEVITKRRWDNNWYSKTSPFSTVIPLG |
| | | ANSRNIRIMARECTGLAAEWWRKVIDERDVKLSKEINVNISGST |
| | | LSPYGSITYK |
| 33 | Spy AD [33-849 | GQVKADDRASGETKASNTHDDSLPKPETIQEAKATIDAVEKTLS |
| | fragment] WT w/o | QQKAELTELATALTKTTAEINHLKEQQDNEQKALTSAQEIYTNT |
| | leader, with G | LASSEETLLAQGAEHQRELTATETELHNAQADQHSKETALSEQK |
| | overhang from | ASISAETTRAQDLVEQVKTSEQNIAKLNAMISNPDAITKAAQTA |
| | cleavage | NDNTKALSSELEKAKADLENQKAKVKKQLTEELAAQKAALAEKE |
| | | AELSRLKSSAPSTQDSIVGNNTMKAPQGYPLEELKKLEASGYIG |
| | | SASYNNYYKEHADQIIAKASPGNQLNQYQDIPADRNRFVDPDNL |
| | | TPEVQNELAQFAAHMINSVRRQLGLPPVTVTAGSQEFARLLSTS |
| | | YKKTHGNTRPSFVYGQPGVSGHYGVGPHDKTIIEDSAGASGLIR |
| | | NDDNMYENIGAFNDVHTVNGIKRGIYDSIKYMLFTDHLHGNTYG |
| | | HAINFLRVDKHNPNAPVYLGFSTSNVGSLNEHFVMFPESNIANH |
| | | QRFNKTPIKAVGSTKDYAQRVGTVSDTIAAIKGKVSSLENRLSA |
| | | IHQEADIMAAQAKVSQLQGKLASTLKQSDSLNLQVRQLNDTKGS |
| | | LRTELLAAKAKQAQLEATRDQSLAKLASLKAALHQTEALAEQAA |
| | | ARVTALVAKKAHLQYLRDFKLNPNRLQVIRERIDNTKQDLAKTT |
| | | SSLLNAQEALAALQAKQSSLEATIATTEHQLTLLKTLANEKEYR |
| | | HLDEDIATVPDLQVAPPLTGVKPLSYSKIDTTPLVQEMVKETKQ |
| | | LLEASARLAAENTSLVAEALVGQTSEMVASNAIVSKITSSITQP |
| | | SSKTSYGSGSSTTSNLISDVDESTQR |
| 34 | Spy AD [33-849 | GQVKADDRASGETKASNTHDDSLPKPETIQEAXATIDAVEKTLS |
| | fragment] nnAA | QQKAELTELATALTKTTAEINHLKEQQDNEQKALTSAQEIYTNT |
| | (e.g., pAMF) w/o | LASSEETLLAQGAEHQRELTATETELHNAQADQHSKETALSEQK |
| | leader, with G | ASISAETTRAQDLVEQVKTSEQNIAKLNAMISNPDAITKAAQTA |
| | overhang from | NDNTKALSSELEKAKADLENQKAKVKKQLTEELAAQKAALAEKE |
| | cleavage | AELSRLKSSAPSTQDSIVGNNTMKAPQGYPLEELKXLEASGYIG |
| | X = nnAA (e.g., | SASYNNYYKEHADQIIAKASPGNQLNQYQDIPADRNRFVDPDNL |
| | pAMF) positions | TPEVQNELAQFAAHMINSVRRQLGLPPVTVTAGSQEFARLLSTS |
| | K64/K287/K386/ | YKXTHGNTRPSFVYGQPGVSGHYGVGPHDKTIIEDSAGASGLIR |
| | K657 - numbered | NDDNMYENIGAFNDVHTVNGIKRGIYDSIKYMLFTDHLHGNTYG |
| | | HAINFLRVDKHNPNAPVYLGFSTSNVGSLNEHFVMFPESNIANH |
| | | QRFNKTPIKAVGSTKDYAQRVGTVSDTIAAIKGKVSSLENRLSA |
| | | IHQEADIMAAQAKVSQLQGKLASTLKQSDSLNLQVRQLNDTKGS |
| | | LRTELLAAKAKQAQLEATRDQSLAKLASLKAALHQTEALAEQAA |
| | | ARVTALVAKXAHLQYLRDFKLNPNRLQVIRERIDNTKQDLAKTT |
| | | SSLLNAQEALAALQAKQSSLEATIATTEHQLTLLKTLANEKEYR |
| | | HLDEDIATVPDLQVAPPLTGVKPLSYSKIDTTPLVQEMVKETKQ |
| | | LLEASARLAAENTSLVAEALVGQTSEMVASNAIVSKITSSITQP |
| | | SSKTSYGSGSSTTSNLISDVDESTQR |
| 35 | ADI [full length] | GTAQTPIHVYSEIGKLKKVLLHRPGKEIENLMPDYLERLLEDDI |
| | WT w/o leader, | PFLEDAQKEHDAFAQALRDEGIEVLYLETLAAESLVTPEIREAF |
| | with G overhang | IDEYLSEANIRGRATKKAIRELLMAIEDNQELIEKTMAGVQKSE |
| | from cleavage | LPEIPASEKGLTDLVESNYPFAIDPMPNLYFTRDPFATIGTGVS |
| | | LNHMFSETRNRETLYGKYIFTHHPIYGGGKVPMVYDRNETTRIE |
| | | GGDELVLSKDVLAVGISQRTDAASIEKLLVNIFKQNLGFKKVLA |
| | | FEFANNRKFMHLDTVFTMVDYDKFTIHPEIEGDLRVYSVTYDNE |
| | | ELHIVEEKGDLAELLAANLGVEKVDLIRCGGDNLVAAGREQWND |
| | | GSNTLTIAPGVVVVYNRNTITNAILESKGLKLIKIHGSELVRGR |
| | | GGPRCMSMPFEREDI |
| 36 | ADI [full length] | GTAQTPIHVYSEIGKLKKVLLHRPGKEIENLMPDYLERLLEDDI |
| | D277A w/o leader, | PFLEDAQKEHDAFAQALRDEGIEVLYLETLAAESLVTPEIREAF |
| | with G overhang | IDEYLSEANIRGRATKKAIRELLMAIEDNQELIEKTMAGVQKSE |
| | from cleavage | LPEIPASEKGLTDLVESNYPFAIDPMPNLYFTRDPFATIGTGVS |
| | | LNHMESETRNRETLYGKYIFTHHPIYGGGKVPMVYDRNETTRIE |
| | | GGDELVLSKDVLAVGISQRTDAASIEKLLVNIFKQNLGFKKVLA |
| | | FEFANNRKFMHLATVFTMVDYDKFTIHPEIEGDLRVYSVTYDNE |
| | | ELHIVEEKGDLAELLAANLGVEKVDLIRCGGDNLVAAGREQWND |
| | | GSNTLTIAPGVVVVYNRNTITNAILESKGLKLIKIHGSELVRGR |
| | | GGPRCMSMPFEREDI |
| 37 | ADI [full length] | GTAQTPIHVYSEIGXLKKVLLHRPGKEIENLMPDYLERLLEDDI |
| | nnAA (e.g., | PFLEDAQKEHDAFAQALRDEGIEVLYLETLAAESLVTPEIREAF |
| | pAMF) w/o leader, | IDEYLSEANIRGRATKKAIRELLMAIEDNQELIEKTMAGVQKSE |
| | with G overhang | LPEIPASEKGLTDLVESNYPFAIDPMPNLYFTRDPFATIGTGVS |
| | from cleavage | LNHMFSETRNRETLYGXYIFTHHPIYGGGKVPMVYDRNETTRIE |
| | X = nnAA (e.g., | GGDELVLSKDVLAVGISQRTDAASIEKLLVNIFKQNLGFKKVLA |
| | pAMF) positions - | FEFANNRKFMHLDTVFTMVDYDKFTIHPEIEGDLRVYSVTYDNE |
| | K15, K193, K316 | ELHIVEEXGDLAELLAANLGVEKVDLIRCGGDNLVAAGREQWND |
| | | GSNTLTIAPGVVVVYNRNTITNAILESKGLKLIKIHGSELVRGR |
| | | GGPRCMSMPFEREDI |
| 38 | ADI (mut5) [full | GTAQTPIHVYSEIGXLKKVLLHRPGKEIENLMPDYLERLLEDDI |
| | length] D277A | PFLEDAQKEHDAFAQALRDEGIEVLYLETLAAESLVTPEIREAF |
| | nnAA (e.g., | IDEYLSEANIRGRATKKAIRELLMAIEDNQELIEKTMAGVQKSE |
| | pAMF) w/o leader, | LPEIPASEKGLTDLVESNYPFAIDPMPNLYFTRDPFATIGTGVS |
| | with G overhang | LNHMFSETRNRETLYGXYIFTHHPIYGGGKVPMVYDRNETTRIE |
| | from cleavage | GGDELVLSKDVLAVGISQRTDAASIEKLLVNIFKQNLGFKKVLA |
| | X = nnAA (e.g., | FEFANNRKFMHLATVFTMVDYDKFTIHPEIEGDLRVYSVTYDNE |
| | pAMF) positions - | ELHIVEEXGDLAELLAANLGVEKVDLIRCGGDNLVAAGREQWND |
| | K15, K193, K316 | GSNTLTIAPGVVVVYNRNTITNAILESKGLKLIKIHGSELVRGR |
| | | GGPRCMSMPFEREDI |
| 39 | hFL [full length] | MHHHHHHGSGSSQIRQNYSTDVEAAVNSLVNLYLQASYTYLSLG |
| | WT w/ leader | FYFDRDDVALEGVSHFFRELAEEKREGYERLLKMQNQRGGRALF |
| | | QDIKKPAEDEWGKTPDAMKAAMALEKKLNQALLDLHALGSARTD |
| | | PHLCDFLETHFLDEEVKLIKKMGDHLTNLHRLGGPEAGLGEYLE |
| | | ERLTLKHD |
| 40 | hFL [full length] | SSQIRQNYSTDVEAAVNSLVNLYLQASYTYLSLGFYFDRDDVAL |
| | WT w/o leader | EGVSHFFRELAEEKREGYERLLKMQNQRGGRALFQDIKKPAEDE |
| | | WGKTPDAMKAAMALEKKLNQALLDLHALGSARTDPHLCDFLETH |
| | | FLDEEVKLIKKMGDHLTNLHRLGGPEAGLGEYLFERLTLKHD |
| 41 | hFL [full length] | MHHHHHHGSGSSQXRQNYSTDVEAAVNSLVNLYLQASYTYLSLG |
| | nnAA (e.g., | FYFDRDDVALEGVSHFFRELAEEKREGYERLLKMQNQRGGRALF |
| | pAMF) w/ leader | QDIKKPAEDEWGKTPDAMKAAMALEKKLNQALLDLHALGSARTD |
| | X = nnAA (e.g., | PHLCDFLETHFLDEEVKLIKKMGDHLTNLHRLGGPEAGLGEYLE |
| | pAMF) position - | ERLTLKHD |
| | I5 | |
| 42 | hFL [full length] | SSQXRQNYSTDVEAAVNSLVNLYLQASYTYLSLGFYFDRDDVAL |
| | nnAA (e.g., | EGVSHFFRELAEEKREGYERLLKMQNQRGGRALFQDIKKPAEDE |
| | pAMF) w/o leader | WGKTPDAMKAAMALEKKLNQALLDLHALGSARTDPHLCDFLETH |
| | X = nnAA (e.g., | FLDEEVKLIKKMGDHLTNLHRLGGPEAGLGEYLFERLTLKHD |
| | pAMF) position - | |
| | I5 | |
| 43 | eCRM-pAMF6 | GADDVVDSSKSFVMENFSSYHGTKPGYVDSIQXGIQKPKSGTQG |
| | mutant | NYDDDWKEFYSTDNKYDAAGYSVDNENPLSGKAGGVVKVTYPGL |
| | | TKVLALKVDNAETIKKELGLSLTEPLMEQVGTEEFIKRFGDGAS |
| | | RVVLSLPFAEGSSSVEYINNWEQAKALSVELEINFETRGKRGQD |
| | | AMYEYMAQACAGNRVRNSVGSSLSCINLDWDVIRDXTKTKIESL |
| | | KEHGPIKNKMSESPNKTVSEEKAXQYLEEFHQTALEHPELSELX |
| | | TVTGTNPVFAGANYAAWAVNVAQVIDSETADNLEKTTAALSILP |
| | | GIGSVMGIADGAVHHNTEEIVAQSIALSSLMVAQAIPLVGELVD |
| | | IGFAAYNFVESIINLFQVVHNSYNRPAYSPGHXTQPFLHDGYAV |
| | | SWNTVEDSIIRTGFQGESGHDIKITAENTPLPIAGVLLPTIPGK |
| | | LDVNKSKTHISVNGRKIRMRCRAIDGDVTFCRPKSPVYVGNGVH |
| | | ANLHVAFHRSSSEKIHSNEISSDSIGVLGYQKTVDHTKVNSXLS |
| | | LFFEIKS |
| 44 | SLO(ΔC11) | GAPKEMPLESAEKEEKKSEDKKKSEEDHTEEINDKIYSLNYNEL |
| | W535A w/o leader, | EVLAKNGETIENFVPKEGVKKADKFIVIERKKKNINTTPVDISI |
| | with G overhang | IDSVTDRTYPAALQLANKGFTENKPDAVVTKRNPQKIHIDLPGM |
| | from cleavage | GDKATVEVNDPTYANVSTAIDNLVNQWHDNYSGGNTLPARTQYT |
| | | ESMVYSKSQIEAALNVNSKILDGTLGIDFKSISKGEKKVMIAAY |
| | | KQIFYTVSANLPNNPADVFDKSVTFKELQRKGVSNEAPPLEVSN |
| | | VAYGRTVFVKLETSSKSNDVEAAFSAALKGTDVKINGKYSDILE |
| | | NSSFTAVVLGGDAAEHNKVVTKDEDVIRNVIKDNATFSRKNPAY |
| | | PISYTSVFLKNNKIAGVNNRTEYVETTSTEYTSGKINLSHQGAY |
| | | VAQYEILWDEINYDDKGKEVITKRRWDNNWYSKTSPFSTVIPLG |
| | | ANSRNIRIMARECTGLAAEWWRKVIDERDVKLSKEINVNISGS |
| 45 | SLO(ΔC21) | GAPKEMPLESAEKEEKKSEDKKKSEEDHTEEINDKIYSLNYNEL |
| | fragment of | EVLAKNGETIENFVPKEGVKKADKFIVIERKKKNINTTPVDISI |
| | W535A w/o leader, | IDSVTDRTYPAALQLANKGFTENKPDAVVTKRNPQKIHIDLPGM |
| | with G overhang | GDKATVEVNDPTYANVSTAIDNLVNQWHDNYSGGNTLPARTQYT |
| | from cleavage | ESMVYSKSQIEAALNVNSKILDGTLGIDFKSISKGEKKVMIAAY |
| | | KQIFYTVSANLPNNPADVEDKSVTFKELQRKGVSNEAPPLEVSN |
| | | VAYGRTVFVKLETSSKSNDVEAAFSAALKGTDVKINGKYSDILE |
| | | NSSFTAVVLGGDAAEHNKVVTKDFDVIRNVIKDNATFSRKNPAY |
| | | PISYTSVFLKNNKIAGVNNRTEYVETTSTEYTSGKINLSHQGAY |
| | | VAQYEILWDEINYDDKGKEVITKRRWDNNWYSKTSPFSTVIPLG |
| | | ANSRNIRIMARECTGLAAEWWRKVIDERDVKLS |
| 46 | SLO(ΔC31) | GAPKEMPLESAEKEEKKSEDKKKSEEDHTEEINDKIYSLNYNEL |
| | fragment of | EVLAKNGETIENFVPKEGVKKADKFIVIERKKKNINTTPVDISI |
| | W535A w/o leader, | IDSVTDRTYPAALQLANKGFTENKPDAVVTKRNPQKIHIDLPGM |
| | with G overhang | GDKATVEVNDPTYANVSTAIDNLVNQWHDNYSGGNTLPARTQYT |
| | from cleavage | ESMVYSKSQIEAALNVNSKILDGTLGIDFKSISKGEKKVMIAAY |
| | | KQIFYTVSANLPNNPADVEDKSVTFKELQRKGVSNEAPPLEVSN |
| | | VAYGRTVFVKLETSSKSNDVEAAFSAALKGTDVKINGKYSDILE |
| | | NSSFTAVVLGGDAAEHNKVVTKDEDVIRNVIKDNATFSRKNPAY |
| | | PISYTSVFLKNNKIAGVNNRTEYVETTSTEYTSGKINLSHQGAY |
| | | VAQYEILWDEINYDDKGKEVITKRRWDNNWYSKTSPESTVIPLG |
| | | ANSRNIRIMARECTGLAAEWWRK |
| 47 | SLO(ΔC41) | GAPKEMPLESAEKEEKKSEDKKKSEEDHTEEINDKIYSLNYNEL |
| | fragment of | EVLAKNGETIENFVPKEGVKKADKFIVIERKKKNINTTPVDISI |
| | SLO(ΔC11) w/o | IDSVTDRTYPAALQLANKGFTENKPDAVVTKRNPQKIHIDLPGM |
| | leader, with G | GDKATVEVNDPTYANVSTAIDNLVNQWHDNYSGGNTLPARTQYT |
| | overhang from | ESMVYSKSQIEAALNVNSKILDGTLGIDFKSISKGEKKVMIAAY |
| | cleavage | KQIFYTVSANLPNNPADVEDKSVTFKELQRKGVSNEAPPLEVSN |
| | | VAYGRTVFVKLETSSKSNDVEAAFSAALKGTDVKTNGKYSDILE |
| | | NSSFTAVVLGGDAAEHNKVVTKDFDVIRNVIKDNATFSRKNPAY |
| | | PISYTSVFLKNNKIAGVNNRTEYVETTSTEYTSGKINLSHQGAY |
| | | VAQYEILWDEINYDDKGKEVITKRRWDNNWYSKTSPFSTVIPLG |
| | | ANSRNIRIMAREC |
| 48 | SLO(ΔC51) | GAPKEMPLESAEKEEKKSEDKKKSEEDHTEEINDKIYSLNYNEL |
| | fragment of | EVLAKNGETIENFVPKEGVKKADKFIVIERKKKNINTTPVDISI |
| | SLO(ΔC11) w/o | IDSVTDRTYPAALQLANKGFTENKPDAVVTKRNPQKIHIDLPGM |
| | leader, with G | GDKATVEVNDPTYANVSTAIDNLVNQWHDNYSGGNTLPARTQYT |
| | overhang from | ESMVYSKSQIEAALNVNSKILDGTLGIDFKSISKGEKKVMIAAY |
| | cleavage | KQIFYTVSANLPNNPADVEDKSVTFKELQRKGVSNEAPPLEVSN |
| | | VAYGRTVFVKLETSSKSNDVEAAFSAALKGTDVKINGKYSDILE |
| | | NSSFTAVVLGGDAAEHNKVVTKDEDVIRNVIKDNATFSRKNPAY |
| | | PISYTSVFLKNNKIAGVNNRTEYVETTSTEYTSGKINLSHQGAY |
| | | VAQYEILWDEINYDDKGKEVITKRRWDNNWYSKTSPESTVIPLG |
| | | ANS |
| 49 | SLO(ΔC61) | GAPKEMPLESAEKEEKKSEDKKKSEEDHTEEINDKIYSLNYNEL |
| | fragment of | EVLAKNGETIENFVPKEGVKKADKFIVIERKKKNINTTPVDISI |
| | SLO(ΔC11) w/o | IDSVTDRTYPAALQLANKGFTENKPDAVVTKRNPQKIHIDLPGM |
| | leader, with G | GDKATVEVNDPTYANVSTAIDNLVNQWHDNYSGGNTLPARTQYT |
| | overhang from | ESMVYSKSQIEAALNVNSKILDGTLGIDFKSISKGEKKVMIAAY |
| | cleavage | KQIFYTVSANLPNNPADVEDKSVTFKELQRKGVSNEAPPLEVSN |
| | | VAYGRTVFVKLETSSKSNDVEAAFSAALKGTDVKINGKYSDILE |
| | | NSSFTAVVLGGDAAEHNKVVTKDFDVIRNVIKDNATFSRKNPAY |
| | | PISYTSVFLKNNKIAGVNNRTEYVETTSTEYTSGKINLSHQGAY |
| | | VAQYEILWDEINYDDKGKEVITKRRWDNNWYSKTSPF |
| 50 | SLO(ΔC71) | GAPKEMPLESAEKEEKKSEDKKKSEEDHTEEINDKIYSLNYNEL |
| | fragment of | EVLAKNGETIENFVPKEGVKKADKFIVIERKKKNINTTPVDISI |
| | SLO(ΔC11) w/o | IDSVTDRTYPAALQLANKGFTENKPDAVVTKRNPQKIHIDLPGM |
| | leader, with G | GDKATVEVNDPTYANVSTAIDNLVNQWHDNYSGGNTLPARTQYT |
| | overhang from | ESMVYSKSQIEAALNVNSKILDGTLGIDFKSISKGEKKVMIAAY |
| | cleavage | KQIFYTVSANLPNNPADVEDKSVTFKELQRKGVSNEAPPLEVSN |
| | | VAYGRTVFVKLETSSKSNDVEAAFSAALKGTDVKINGKYSDILE |
| | | NSSFTAVVLGGDAAEHNKVVTKDEDVIRNVIKDNATFSRKNPAY |
| | | PISYTSVELKNNKIAGVNNRTEYVETTSTEYTSGKINLSHQGAY |
| | | VAQYEILWDEINYDDKGKEVITKRRWD |
| 51 | SLO(ΔC81) | GAPKEMPLESAEKEEKKSEDKKKSEEDHTEEINDKIYSLNYNEL |
| | fragment of | EVLAKNGETIENFVPKEGVKKADKFIVIERKKKNINTTPVDISI |
| | SLO(ΔC11) w/o | IDSVTDRTYPAALQLANKGFTENKPDAVVTKRNPQKIHIDLPGM |
| | leader, with G | GDKATVEVNDPTYANVSTAIDNLVNQWHDNYSGGNTLPARTQYT |
| | overhang from | ESMVYSKSQIEAALNVNSKILDGTLGIDFKSISKGEKKVMIAAY |
| | cleavage | KQIFYTVSANLPNNPADVEDKSVTFKELQRKGVSNEAPPLEVSN |
| | | VAYGRTVFVKLETSSKSNDVEAAFSAALKGTDVKINGKYSDILE |
| | | NSSFTAVVLGGDAAEHNKVVTKDFDVIRNVIKDNATFSRKNPAY |
| | | PISYTSVFLKNNKIAGVNNRTEYVETTSTEYTSGKINLSHQGAY |
| | | VAQYEILWDEINYDDKG |
| 52 | SLO(ΔC91) | GAPKEMPLESAEKEEKKSEDKKKSEEDHTEEINDKIYSLNYNEL |
| | fragment of | EVLAKNGETIENFVPKEGVKKADKFIVIERKKKNINTTPVDISI |
| | SLO(ΔC11) w/o | IDSVTDRTYPAALQLANKGFTENKPDAVVTKRNPQKIHIDLPGM |
| | leader, with G | GDKATVEVNDPTYANVSTAIDNLVNQWHDNYSGGNTLPARTQYT |
| | overhang from | ESMVYSKSQIEAALNVNSKILDGTLGIDFKSISKGEKKVMIAAY |
| | cleavage | KQIFYTVSANLPNNPADVEDKSVTFKELQRKGVSNEAPPLEVSN |
| | | VAYGRTVFVKLETSSKSNDVEAAFSAALKGTDVKINGKYSDILE |
| | | NSSFTAVVLGGDAAEHNKVVTKDFDVIRNVIKDNATFSRKNPAY |
| | | PISYTSVFLKNNKIAGVNNRTEYVETTSTEYTSGKINLSHQGAY |
| | | VAQYEIL |
| 53 | SLO(ΔC101) | GAPKEMPLESAEKEEKKSEDKKKSEEDHTEEINDKIYSLNYNEL |
| | fragment of | EVLAKNGETIENFVPKEGVKKADKFIVIERKKKNINTTPVDISI |
| | SLO(ΔC11) w/o | IDSVTDRTYPAALQLANKGFTENKPDAVVTKRNPQKIHIDLPGM |
| | leader, with G | GDKATVEVNDPTYANVSTAIDNLVNQWHDNYSGGNTLPARTQYT |
| | overhang from | ESMVYSKSQIEAALNVNSKILDGTLGIDFKSISKGEKKVMIAAY |
| | cleavage | KQIFYTVSANLPNNPADVEDKSVTFKELQRKGVSNEAPPLEVSN |
| | | VAYGRTVFVKLETSSKSNDVEAAFSAALKGTDVKINGKYSDILE |
| | | NSSFTAVVLGGDAAEHNKVVTKDEDVIRNVIKDNATFSRKNPAY |
| | | PISYTSVFLKNNKIAGVNNRTEYVETTSTEYTSGKINLSHQ |
| 54 | SLO(ΔC101) | GAPKEMPLESAEKEEKKSEDXKKSEEDHTEEINDXIYSLNYNEL |
| | fragment of | EVLAKNGETIENFVPKEGVKKADKFIVIEXKKKNINTTPVDISI |
| | SLO(ΔC11) w/o | IDSVTDRTYPAALQLANKGFTENXPDAVVTKRNPQKIHIDLPGM |
| | leader, with G | GDKATVEVNDPTYANVSTAIDNLVNQWHDNYSGGNTLPARTQYT |
| | overhang from | ESMVYSKSQIEAALNVNSXILDGTLGIDFKSISKGEKKVMIAAY |
| | cleavage | KQIFYTVSANLPNNPADVEDKSVTEXELQRKGVSNEAPPLEVSN |
| | X = nnAA (e.g., | VAYGRTVFVKLETSSXSNDVEAAFSAALKGTDVXINGKYSDILE |
| | pAMF) or native | NSSFTAVVLGGDAAEHNKVVTXDFDVIRNVIKDNATFSRKNPAY |
| | amino acid | PISYTSVELKNNKIAGVNNRTEYVETTSTEYTSGXINLSHQ |
| | (e.g .: K98, K112, | |
| | R151, K189, K272, | |
| | K323, K357, | |
| | K375, K407, | |
| | K464; or K or R.) | |
| 55 | SLO(ΔC101) | GAPKEMPLESAEKEEKKSEDXKKSEEDHTEEINDKIYSLNYNEL |
| | fragment of | EVLAKNGETIENFVPKEGVKKADKFIVIEXKKKNINTTPVDISI |
| | SLO(ΔC11) - | IDSVTDRTYPAALQLANKGFTENKPDAVVTKRNPQKIHIDLPGM |
| | nnAA (e.g., | GDKATVEVNDPTYANVSTAIDNLVNQWHDNYSGGNTLPARTQYT |
| | pAMF) 1 - w/o | ESMVYSKSQIEAALNVNSXILDGTLGIDFKSISKGEKKVMIAAY |
| | leader, with G | KQIFYTVSANLPNNPADVEDKSVTFKELQRKGVSNEAPPLEVSN |
| | overhang from | VAYGRTVFVKLETSSXSNDVEAAFSAALKGTDVKTNGKYSDILE |
| | cleavage (420995) | NSSFTAVVLGGDAAEHNKVVTKDEDVIRNVIKDNATFSRKNPAY |
| | X = nnAA (e.g., | PISYTSVFLKNNKIAGVNNRTEYVETTSTEYTSGKINLSHQ |
| | pAMF) positions - | |
| | K98, R151 , K272, | |
| | K357 | |
| 56 | SLO(ΔC101) | GAPKEMPLESAEKEEKKSEDKKKSEEDHTEEINDXIYSLNYNEL |
| | fragment of | EVLAKNGETIENFVPKEGVKKADKFIVIERKKKNINTTPVDISI |
| | SLO(ΔC11) - | IDSVTDRTYPAALQLANKGFTENXPDAVVTKRNPQKIHIDLPGM |
| | nnAA (e.g., | GDKATVEVNDPTYANVSTAIDNLVNQWHDNYSGGNTLPARTQYT |
| | pAMF) 2 - w/o | ESMVYSKSQIEAALNVNSKILDGTLGIDFKSISKGEKKVMIAAY |
| | leader, with G | KQIFYTVSANLPNNPADVEDKSVTEXELQRKGVSNEAPPLEVSN |
| | overhang from | VAYGRTVFVKLETSSKSNDVEAAFSAALKGTDVXTNGKYSDILE |
| | cleavage (420996) | NSSFTAVVLGGDAAEHNKVVTKDEDVIRNVIKDNATFSRKNPAY |
| | X = nnAA (e.g., | PISYTSVFLKNNKIAGVNNRTEYVETTSTEYTSGKINLSHQ |
| | pAMF) positions - | |
| | K112, K189, | |
| | K323, K375 | |
| 57 | SLO(ΔC101) | GAPKEMPLESAEKEEKKSEDKKKSEEDHTEEINDKIYSLNYNEL |
| | fragment of | EVLAKNGETIENFVPKEGVKKADKFIVIEXKKKNINTTPVDISI |
| | SLO(ΔC11) - | IDSVTDRTYPAALQLANKGFTENKPDAVVTKRNPQKIHIDLPGM |
| | nnAA (e.g., | GDKATVEVNDPTYANVSTAIDNLVNQWHDNYSGGNTLPARTQYT |
| | pAMF) 3 - w/o | ESMVYSKSQIEAALNVNSXILDGTLGIDFKSISKGEKKVMIAAY |
| | leader, with G | KQIFYTVSANLPNNPADVEDKSVTFKELQRKGVSNEAPPLEVSN |
| | overhang from | VAYGRTVFVKLETSSXSNDVEAAFSAALKGTDVKINGKYSDILE |
| | cleavage (420997) | NSSFTAVVLGGDAAEHNKVVTXDEDVIRNVIKDNATFSRKNPAY |
| | X = nnAA (e.g., | PISYTSVFLKNNKIAGVNNRTEYVETTSTEYTSGKINLSHQ |
| | pAMF) positions - | |
| | R151, K272, K357, | |
| | K407 | |
| 58 | SLO(ΔC101) | GAPKEMPLESAEKEEKKSEDKKKSEEDHTEEINDKIYSLNYNEL |
| | fragment of | EVLAKNGETIENFVPKEGVKKADKFIVIEXKKKNINTTPVDISI |
| | SLO(ΔC11) - | IDSVTDRTYPAALQLANKGFTENKPDAVVTKRNPQKIHIDLPGM |
| | nnAA (e.g., | GDKATVEVNDPTYANVSTAIDNLVNQWHDNYSGGNTLPARTQYT |
| | pAMF) 4 - w/o | ESMVYSKSQIEAALNVNSXILDGTLGIDFKSISKGEKKVMIAAY |
| | leader, with G | KQIFYTVSANLPNNPADVEDKSVTFKELQRKGVSNEAPPLEVSN |
| | overhang from | VAYGRTVFVKLETSSKSNDVEAAFSAALKGTDVXINGKYSDILE |
| | cleavage (420998) | NSSFTAVVLGGDAAEHNKVVTKDFDVIRNVIKDNATFSRKNPAY |
| | X = nnAA (e.g., | PISYTSVFLKNNKIAGVNNRTEYVETTSTEYTSGXINLSHQ |
| | pAMF) positions - | |
| | R151, K272, K375, | |
| | K464 | |
| 59 | SLO(ΔC101) | GAPKEMPLESAEKEEKKSEDKKKSEEDHTEEINDXIYSLNYNEL |
| | fragment of | EVLAKNGETIENFVPKEGVKKADKFIVIERKKKNINTTPVDISI |
| | SLO(ΔC11) - | IDSVTDRTYPAALQLANKGFTENKPDAVVTKRNPQKIHIDLPGM |
| | nnAA (e.g., | GDKATVEVNDPTYANVSTAIDNLVNQWHDNYSGGNTLPARTQYT |
| | pAMF)5 - w/o | ESMVYSKSQIEAALNVNSXILDGTLGIDFKSISKGEKKVMIAAY |
| | leader, with G | KQIFYTVSANLPNNPADVEDKSVTFKELQRKGVSNEAPPLEVSN |
| | overhang from | VAYGRTVFVKLETSSXSNDVEAAFSAALKGTDVKINGKYSDILE |
| | cleavage (420999) | NSSFTAVVLGGDAAEHNKVVTKDFDVIRNVIKDNATFSRKNPAY |
| | X = nnAA (e.g., | PISYTSVELKNNKIAGVNNRTEYVETTSTEYTSGXINLSHQ |
| | pAMF) positions - | |
| | K112, K272, | |
| | K357, K464 | |
| 60 | SLO(ΔC101) | GAPKEMPLESAEKEEKKSEDXKKSEEDHTEEINDKIYSLNYNEL |
| | fragment of | EVLAKNGETIENFVPKEGVKKADKFIVIERKKKNINTTPVDISI |
| | SLO(ΔC11) - | IDSVTDRTYPAALQLANKGFTENXPDAVVTKRNPQKIHIDLPGM |
| | nnAA (e.g., | GDKATVEVNDPTYANVSTAIDNLVNQWHDNYSGGNTLPARTQYT |
| | pAMF) 6 - w/o | ESMVYSKSQIEAALNVNSKILDGTLGIDFKSISKGEKKVMIAAY |
| | leader, with G | KQIFYTVSANLPNNPADVEDKSVTFKELQRKGVSNEAPPLEVSN |
| | overhang from | VAYGRTVFVKLETSSXSNDVEAAFSAALKGTDVKTNGKYSDILE |
| | cleavage (421000) | NSSFTAVVLGGDAAEHNKVVTKDFDVIRNVIKDNATFSRKNPAY |
| | X = nnAA (e.g., | PISYTSVFLKNNKIAGVNNRTEYVETTSTEYTSGKINLSHQ |
| | pAMF) positions - | |
| | K98, K189, K357 | |
| 61 | SLO(ΔC101) | GAPKEMPLESAEKEEKKSEDXKKSEEDHTEEINDXIYSLNYNEL |
| | fragment of | EVLAKNGETIENFVPKEGVKKADKFIVIERKKKNINTTPVDISI |
| | SLO(ΔC11) - | IDSVTDRTYPAALQLANKGFTENXPDAVVTKRNPQKIHIDLPGM |
| | nnAA (e.g., | GDKATVEVNDPTYANVSTAIDNLVNQWHDNYSGGNTLPARTQYT |
| | pAMF) 7 - w/o | ESMVYSKSQIEAALNVNSKILDGTLGIDFKSISKGEKKVMIAAY |
| | leader, with G | KQIFYTVSANLPNNPADVEDKSVTEXELQRKGVSNEAPPLEVSN |
| | overhang from | VAYGRTVFVKLETSSKSNDVEAAFSAALKGTDVKINGKYSDILE |
| | cleavage (421001) | NSSFTAVVLGGDAAEHNKVVTKDEDVIRNVIKDNATFSRKNPAY |
| | X = nnAA (e.g., | PISYTSVFLKNNKIAGVNNRTEYVETTSTEYTSGKINLSHQ |
| | pAMF) positions - | |
| | K112, K189, K323 | |
| 62 | SLO(ΔC101) | GAPKEMPLESAEKEEKKSEDXKKSEEDHTEEINDKIYSLNYNEL |
| | fragment of | EVLAKNGETIENFVPKEGVKKADKFIVIEXKKKNINTTPVDISI |
| | SLO(ΔC11) - | IDSVTDRTYPAALQLANKGFTENKPDAVVTKRNPQKIHIDLPGM |
| | nnAA (e.g., | GDKATVEVNDPTYANVSTAIDNLVNQWHDNYSGGNTLPARTQYT |
| | pAMF) 8 - w/o | ESMVYSKSQIEAALNVNSXILDGTLGIDFKSISKGEKKVMIAAY |
| | leader, with G | KQIFYTVSANLPNNPADVEDKSVTFKELQRKGVSNEAPPLEVSN |
| | overhang from | VAYGRTVFVKLETSSKSNDVEAAFSAALKGTDVKINGKYSDILE |
| | cleavage (421002) | NSSFTAVVLGGDAAEHNKVVTKDFDVIRNVIKDNATFSRKNPAY |
| | X = nnAA (e.g., | PISYTSVELKNNKIAGVNNRTEYVETTSTEYTSGKINLSHQ |
| | pAMF) positions - | |
| | K98, R151, K272 | |
| 63 | SLO(ΔC101) | GAPKEMPLESAEKEEKKSEDKKKSEEDHTEEINDXIYSLNYNEL |
| | fragment of | EVLAKNGETIENFVPKEGVKKADKFIVIERKKKNINTTPVDISI |
| | SLO(ΔC11) - | IDSVTDRTYPAALQLANKGFTENKPDAVVTKRNPQKIHIDLPGM |
| | nnAA (e.g., | GDKATVEVNDPTYANVSTAIDNLVNQWHDNYSGGNTLPARTQYT |
| | pAMF) 9 - w/o | ESMVYSKSQIEAALNVNSXILDGTLGIDFKSISKGEKKVMIAAY |
| | leader, with G | KQIFYTVSANLPNNPADVEDKSVTFKELQRKGVSNEAPPLEVSN |
| | overhang from | VAYGRTVFVKLETSSKSNDVEAAFSAALKGTDVXINGKYSDILE |
| | cleavage (421003) | NSSFTAVVLGGDAAEHNKVVTKDEDVIRNVIKDNATESRKNPAY |
| | X = nnAA (e.g., | PISYTSVFLKNNKIAGVNNRTEYVETTSTEYTSGKINLSHQ |
| | pAMF) positions - | |
| | K112, K272, K375 | |
| 64 | SLO(ΔC101) | GAPKEMPLESAEKEEKKSEDKKKSEEDHTEEINDXIYSLNYNEL |
| | fragment of | EVLAKNGETIENFVPKEGVKKADKFIVIERKKKNINTTPVDISI |
| | SLO(ΔC11) - | IDSVTDRTYPAALQLANKGFTENKPDAVVTKRNPQKIHIDLPGM |
| | nnAA (e.g., | GDKATVEVNDPTYANVSTAIDNLVNQWHDNYSGGNTLPARTQYT |
| | pAMF) 10 - w/o | ESMVYSKSQIEAALNVNSKILDGTLGIDFKSISKGEKKVMIAAY |
| | leader, with G | KQIFYTVSANLPNNPADVEDKSVTEXELQRKGVSNEAPPLFVSN |
| | overhang from | VAYGRTVFVKLETSSKSNDVEAAFSAALKGTDVKINGKYSDILE |
| | cleavage (421004) | NSSFTAVVLGGDAAEHNKVVTXDEDVIRNVIKDNATFSRKNPAY |
| | X = nnAA (e.g., | PISYTSVFLKNNKIAGVNNRTEYVETTSTEYTSGKINLSHQ |
| | pAMF) positions - | |
| | K112, K323, K407 | |
| 65 | SLO(ΔC101) | GAPKEMPLESAEKEEKKSEDXKKSEEDHTEEINDKIYSLNYNEL |
| | fragment of | EVLAKNGETIENFVPKEGVKKADKFIVIEXKKKNINTTPVDISI |
| | SLO(ΔC11) - | IDSVTDRTYPAALQLANKGFTENKPDAVVTKRNPQKIHIDLPGM |
| | nnAA (e.g., | GDKATVEVNDPTYANVSTAIDNLVNQWHDNYSGGNTLPARTQYT |
| | pAMF) 10 - w/o | ESMVYSKSQIEAALNVNSXILDGTLGIDFKSISKGEKKVMIAAY |
| | leader, with G | KQIFYTVSANLPNNPADVFDKSVTFKELQRKGVSNEAPPLFVSN |
| | overhang from | VAYGRTVFVKLETSSXSNDVEAAFSAALKGTDVKINGKYSDILE |
| | cleavage (426507) | NSSFTAVVLGGDAAEHNKVVTXDFDVIRNVIKDNATFSRKNPAY |
| | X = nnAA (e.g., | PISYTSVFLKNNKIAGVNNRTEYVETTSTEYTSGKINLSHQ |
| | pAMF) positions - | |
| | K98, R151, K272, | |
| | K357, K407 | |
| 66 | SLO(ΔC101) | GAPKEMPLESAEKEEKKSEDKKKSEEDHTEEINDXIYSLNYNEL |
| | fragment of | EVLAKNGETIENFVPKEGVKKADKFIVIERKKKNINTTPVDISI |
| | SLO(ΔC11) - | IDSVTDRTYPAALQLANKGFTENXPDAVVTKRNPQKIHIDLPGM |
| | nnAA (e.g., | GDKATVEVNDPTYANVSTAIDNLVNQWHDNYSGGNTLPARTQYT |
| | pAMF) 10 - w/o | ESMVYSKSQIEAALNVNSKILDGTLGIDFKSISKGEKKVMIAAY |
| | leader, with G | KQIFYTVSANLPNNPADVEDKSVTEXELQRKGVSNEAPPLFVSN |
| | overhang from | VAYGRTVFVKLETSSKSNDVEAAFSAALKGTDVXTNGKYSDILE |
| | cleavage (426508) | NSSFTAVVLGGDAAEHNKVVTKDFDVIRNVIKDNATFSRKNPAY |
| | X = nnAA (e.g., | PISYTSVFLKNNKIAGVNNRTEYVETTSTEYTSGXINLSHQ |
| | pAMF) positions - | |
| | K112, K189, | |
| | K323, K375, K464 | |
| 67 | SLO(ΔC101) | GAPKEMPLESAEKEEKKSEDKKKSEEDHTEEINDXIYSLNYNEL |
| | fragment of | EVLAKNGETIENFVPKEGVKKADKFIVIEXKKKNINTTPVDISI |
| | SLO(ΔC11) - | IDSVTDRTYPAALQLANKGFTENKPDAVVTKRNPQKIHIDLPGM |
| | nnAA (e.g., | GDKATVEVNDPTYANVSTAIDNLVNQWHDNYSGGNTLPARTQYT |
| | pAMF) 10 - w/o | ESMVYSKSQIEAALNVNSXILDGTLGIDFKSISKGEKKVMIAAY |
| | leader, with G | KQIFYTVSANLPNNPADVEDKSVTFKELQRKGVSNEAPPLEVSN |
| | overhang from | VAYGRTVFVKLETSSXSNDVEAAFSAALKGTDVKINGKYSDILE |
| | cleavage (426509) | NSSFTAVVLGGDAAEHNKVVTXDEDVIRNVIKDNATFSRKNPAY |
| | X = nnAA (e.g., | PISYTSVFLKNNKIAGVNNRTEYVETTSTEYTSGKINLSHQ |
| | pAMF) positions - | |
| | K112, R151, K272, | |
| | K357, K407 | |
| 68 | SLO(ΔC101) | GAPKEMPLESAEKEEKKSEDXKKSEEDHTEEINDKIYSLNYNEL |
| | fragment of | EVLAKNGETIENFVPKEGVKKADKFIVIEXKKKNINTTPVDISI |
| | SLO(ΔC11) - | IDSVTDRTYPAALQLANKGFTENKPDAVVTKRNPQKIHIDLPGM |
| | nnAA (e.g., | GDKATVEVNDPTYANVSTAIDNLVNQWHDNYSGGNTLPARTQYT |
| | pAMF) 10 - w/o | ESMVYSKSQIEAALNVNSXILDGTLGIDFKSISKGEKKVMIAAY |
| | leader, with G | KQIFYTVSANLPNNPADVFDKSVTFKELQRKGVSNEAPPLEVSN |
| | overhang from | VAYGRTVFVKLETSSXSNDVEAAFSAALKGTDVKINGKYSDILE |
| | cleavage (426510) | NSSFTAVVLGGDAAEHNKVVTXDFDVIRNVIKDNATFSRKNPAY |
| | X = nnAA (e.g., | PISYTSVELKNNKIAGVNNRTEYVETTSTEYTSGXINLSHQ |
| | pAMF) positions - | |
| | K98, R151, K272, | |
| | K357, K407, K464 | |
| 69 | SLO(ΔC101) | GAPKEMPLESAEKEEKKSEDKKKSEEDHTEEINDXIYSLNYNEL |
| | fragment of | EVLAKNGETIENFVPKEGVKKADKFIVIEXKKKNINTTPVDISI |
| | SLO(ΔC11) - | IDSVTDRTYPAALQLANKGFTENXPDAVVTKRNPQKIHIDLPGM |
| | nnAA (e.g., | GDKATVEVNDPTYANVSTAIDNLVNQWHDNYSGGNTLPARTQYT |
| | pAMF) 10 - w/o | ESMVYSKSQIEAALNVNSKILDGTLGIDFKSISKGEKKVMIAAY |
| | leader, with G | KQIFYTVSANLPNNPADVEDKSVTEXELQRKGVSNEAPPLFVSN |
| | overhang from | VAYGRTVFVKLETSSKSNDVEAAFSAALKGTDVXINGKYSDILE |
| | cleavage (426511) | NSSFTAVVLGGDAAEHNKVVTKDEDVIRNVIKDNATESRKNPAY |
| | X = nnAA (e.g., | PISYTSVFLKNNKIAGVNNRTEYVETTSTEYTSGXINLSHQ |
| | pAMF) positions - | |
| | K112, R151, K189, | |
| | K323, K375, K464 | |
| 70 | SLO(ΔC101) | GAPKEMPLESAEKEEKKSEDXKKSEEDHTEEINDXIYSLNYNEL |
| | fragment of | EVLAKNGETIENFVPKEGVKKADKFIVIERKKKNINTTPVDISI |
| | SLO(ΔC11) - | IDSVTDRTYPAALQLANKGFTENXPDAVVTKRNPQKIHIDLPGM |
| | nnAA (e.g., | GDKATVEVNDPTYANVSTAIDNLVNQWHDNYSGGNTLPARTQYT |
| | pAMF) 10 - w/o | ESMVYSKSQIEAALNVNSKILDGTLGIDFKSISKGEKKVMIAAY |
| | leader, with G | KQIFYTVSANLPNNPADVEDKSVTEXELQRKGVSNEAPPLEVSN |
| | overhang from | VAYGRTVFVKLETSSKSNDVEAAFSAALKGTDVXTNGKYSDILE |
| | cleavage (426512) | NSSFTAVVLGGDAAEHNKVVTKDFDVIRNVIKDNATFSRKNPAY |
| | X = nnAA (e.g., | PISYTSVFLKNNKIAGVNNRTEYVETTSTEYTSGXINLSHQ |
| | pAMF) positions - | |
| | K98, K112, K189, | |
| | K323, K375, K464 | |
| 71 | SLO(ΔC101) | GAPKEMPLESAEKEEKKSEDKKKSEEDHTEEINDXIYSLNYNEL |
| | fragment of | EVLAKNGETIENFVPKEGVKKADKFIVIEXKKKNINTTPVDISI |
| | SLO(ΔC11) - | IDSVTDRTYPAALQLANKGFTENXPDAVVTKRNPQKIHIDLPGM |
| | nnAA (e.g., | GDKATVEVNDPTYANVSTAIDNLVNQWHDNYSGGNTLPARTQYT |
| | pAMF) 10 - w/o | ESMVYSKSQIEAALNVNSXILDGTLGIDEKSISKGEKKVMIAAY |
| | leader, with G | KQIFYTVSANLPNNPADVEDKSVTFKELQRKGVSNEAPPLEVSN |
| | overhang from | VAYGRTVFVKLETSSXSNDVEAAFSAALKGTDVKINGKYSDILE |
| | cleavage (426513) | NSSFTAVVLGGDAAEHNKVVTXDFDVIRNVIKDNATFSRKNPAY |
| | X = nnAA (e.g., | PISYTSVFLKNNKIAGVNNRTEYVETTSTEYTSGXINLSHQ |
| | pAMF) positions - | |
| | K112, R151, K189, | |
| | K272, K357, | |
| | K407, K464 | |
| 72 | SLO(ΔC101) | GAPKEMPLESAEKEEKKSEDXKKSEEDHTEEINDKIYSLNYNEL |
| | fragment of | EVLAKNGETIENFVPKEGVKKADKFIVIEXKKKNINTTPVDISI |
| | SLO(ΔC11) - | IDSVTDRTYPAALQLANKGFTENXPDAVVTKRNPQKIHIDLPGM |
| | nnAA (e.g., | GDKATVEVNDPTYANVSTAIDNLVNQWHDNYSGGNTLPARTQYT |
| | pAMF) 10 - w/o | ESMVYSKSQIEAALNVNSKILDGTLGIDFKSISKGEKKVMIAAY |
| | leader, with G | KQIFYTVSANLPNNPADVEDKSVTEXELQRKGVSNEAPPLEVSN |
| | overhang from | VAYGRTVFVKLETSSKSNDVEAAFSAALKGTDVXTNGKYSDILE |
| | cleavage (426514) | NSSFTAVVLGGDAAEHNKVVTXDEDVIRNVIKDNATFSRKNPAY |
| | X = nnAA (e.g., | PISYTSVFLKNNKIAGVNNRTEYVETTSTEYTSGXINLSHQ |
| | pAMF) positions - | |
| | K98, R151, K189, | |
| | K323, K375, | |
| | K407, K464 | |
| 73 | SLO(ΔC101) | GAPKEMPLESAEKEEKKSEDKKKSEEDHTEEINDXIYSLNYNEL |
| | fragment of | EVLAKNGETIENFVPKEGVKKADKFIVIERKKKNINTTPVDISI |
| | SLO(ΔC11) - | IDSVTDRTYPAALQLANKGFTENXPDAVVTKRNPQKIHIDLPGM |
| | nnAA (e.g., | GDKATVEVNDPTYANVSTAIDNLVNQWHDNYSGGNTLPARTQYT |
| | pAMF) 10 - w/o | ESMVYSKSQIEAALNVNSXILDGTLGIDFKSISKGEKKVMIAAY |
| | leader, with G | KQIFYTVSANLPNNPADVEDKSVTFKELQRKGVSNEAPPLFVSN |
| | overhang from | VAYGRTVFVKLETSSXSNDVEAAFSAALKGTDVXTNGKYSDILE |
| | cleavage (426515) | NSSFTAVVLGGDAAEHNKVVTXDEDVIRNVIKDNATFSRKNPAY |
| | X = nnAA (e.g., | PISYTSVFLKNNKIAGVNNRTEYVETTSTEYTSGXINLSHQ |
| | pAMF) positions - | |
| | K112, K189, | |
| | K272, K357, | |
| | K375, K407, K464 | |
| 74 | SLO(ΔC101) | GAPKEMPLESAEKEEKKSEDXKKSEEDHTEEINDXIYSLNYNEL |
| | fragment of | EVLAKNGETIENFVPKEGVKKADKFIVIEXKKKNINTTPVDISI |
| | SLO(ΔC11) - | IDSVTDRTYPAALQLANKGFTENXPDAVVTKRNPQKIHIDLPGM |
| | nnAA (e.g., | GDKATVEVNDPTYANVSTAIDNLVNQWHDNYSGGNTLPARTQYT |
| | pAMF) 10 - w/o | ESMVYSKSQIEAALNVNSXILDGTLGIDFKSISKGEKKVMIAAY |
| | leader, with G | KQIFYTVSANLPNNPADVEDKSVTEXELQRKGVSNEAPPLEVSN |
| | overhang from | VAYGRTVFVKLETSSXSNDVEAAFSAALKGTDVXTNGKYSDILE |
| | cleavage (426516) | NSSFTAVVLGGDAAEHNKVVTKDEDVIRNVIKDNATFSRKNPAY |
| | X = nnAA (e.g., | PISYTSVFLKNNKIAGVNNRTEYVETTSTEYTSGKINLSHQ |
| | pAMF) positions - | |
| | K98, K112, R151, | |
| | K189, K272, | |
| | K323, K357, | |
| | K375, | |
| 75 | SLO(ΔC101) | GAPKEMPLESAEKEEKKSEDXKKSEEDHTEEINDKIYSLNYNEL |
| | fragment of | EVLAKNGETIENFVPKEGVKKADKFIVIEXKKKNINTTPVDISI |
| | SLO(ΔC11) - | IDSVTDRTYPAALQLANKGFTENXPDAVVTKRNPQKIHIDLPGM |
| | nnAA (e.g., | GDKATVEVNDPTYANVSTAIDNLVNQWHDNYSGGNTLPARTQYT |
| | pAMF) 10 - w/o | ESMVYSKSQIEAALNVNSXILDGTLGIDFKSISKGEKKVMIAAY |
| | leader, with G | KQIFYTVSANLPNNPADVFDKSVTFXELQRKGVSNEAPPLEVSN |
| | overhang from | VAYGRTVFVKLETSSXSNDVEAAFSAALKGTDVKINGKYSDILE |
| | cleavage (426510) | NSSFTAVVLGGDAAEHNKVVTXDEDVIRNVIKDNATESRKNPAY |
| | X = nnAA (e.g., | PISYTSVFLKNNKIAGVNNRTEYVETTSTEYTSGXINLSHQ |
| | pAMF) positions - | |
| | K98, R151, K189, | |
| | K272, K323, | |
| | K357, K407, K464 | |
| 76 | SLO(ΔC101) | GAPKEMPLESAEKEEKKSEDKKKSEEDHTEEINDXIYSLNYNEL |
| | fragment of | EVLAKNGETIENFVPKEGVKKADKFIVIERKKKNINTTPVDISI |
| | SLO(ΔC11) - | IDSVTDRTYPAALQLANKGFTENXPDAVVTKRNPQKIHIDLPGM |
| | nnAA (e.g., | GDKATVEVNDPTYANVSTAIDNLVNQWHDNYSGGNTLPARTQYT |
| | pAMF) 10 - w/o | ESMVYSKSQIEAALNVNSXILDGTLGIDFKSISKGEKKVMIAAY |
| | leader, with G | KQIFYTVSANLPNNPADVEDKSVTEXELQRKGVSNEAPPLEVSN |
| | overhang from | VAYGRTVFVKLETSSXSNDVEAAFSAALKGTDVXINGKYSDILE |
| | cleavage (426515) | NSSFTAVVLGGDAAEHNKVVTXDEDVIRNVIKDNATFSRKNPAY |
| | X = nnAA (e.g., | PISYTSVELKNNKIAGVNNRTEYVETTSTEYTSGXINLSHQ |
| | pAMF) positions - | |
| | K112, K189, | |
| | K272, K323, | |
| | K357, K375, | |
| | K407, K464 | |

TABLE 1A
Exemplary Polypeptide Sequences

| Seq | | |
|---|---|---|
| ID | Name/description | Amino acid sequence |
| 77 | Spy AD [33-714 | GQVKADDRASGETKASNTHDDSLPKPETIQEAXATIDAVEKTLS |
| | fragment] nnAA | QQKAELTELATALTKTTAEINHLKEQQDNEQKALTSAQEIYTNT |
| | (e.g., pAMF) | LASSEETLLAQGAEHQRELTATETELHNAQADQHSKETALSEQK |
| | mutant w/o leader | ASISAETTRAQDLVEQVKTSEQNIAKLNAMISNPDAITKAAQTA |
| | Fragment of SEQ | NDNTKALSSELEKAKADLENQKAKVKKQLTEELAAQKAALAEKE |
| | ID NO: 34 | AELSRLKSSAPSTQDSIVGNNTMKAPQGYPLEELKXLEASGYIG |
| | X = nnAA (e.g., | SASYNNYYKEHADQIIAKASPGNQLNQYQDIPADRNRFVDPDNL |
| | pAMF) | TPEVQNELAQFAAHMINSVRRQLGLPPVTVTAGSQEFARLLSTS |
| | K64/K287/K386/ | YKXTHGNTRPSFVYGQPGVSGHYGVGPHDKTIIEDSAGASGLIR |
| | K657 - numbered | NDDNMYENIGAFNDVHTVNGIKRGIYDSIKYMLFTDHLHGNTYG |
| | according to full- | HAINFLRVDKHNPNAPVYLGFSTSNVGSLNEHFVMFPESNIANH |
| | length sequence | QRFNKTPIKAVGSTKDYAQRVGTVSDTIAAIKGKVSSLENRLSA |
| | | IHQEADIMAAQAKVSQLQGKLASTLKQSDSLNLQVRQLNDTKGS |
| | | LRTELLAAKAKQAQLEATRDQSLAKLASLKAALHQTEALAEQAA |
| | | ARVTALVAKXAHLQYLRDFKLNPNRLQVIRERIDNTKQDLAKTT |
| | | SSLLNAQEALAALQAKQSSLEAT |
| 78 | Spy AD [33-699 | GQVKADDRASGETKASNTHDDSLPKPETIQEAXATIDAVEKTLS |
| | fragment] nnAA | QQKAELTELATALTKTTAEINHLKEQQDNEQKALTSAQEIYTNT |
| | (e.g., pAMF) | LASSEETLLAQGAEHQRELTATETELHNAQADQHSKETALSEQK |
| | mutant w/o leader | ASISAETTRAQDLVEQVKTSEQNIAKLNAMISNPDAITKAAQTA |
| | Fragment of SEQ | NDNTKALSSELEKAKADLENQKAKVKKQLTEELAAQKAALAEKE |
| | ID NO: 34 | AELSRLKSSAPSTQDSIVGNNTMKAPQGYPLEELKXLEASGYIG |
| | X = nnAA (e.g., | SASYNNYYKEHADQIIAKASPGNQLNQYQDIPADRNRFVDPDNL |
| | pAMF) | TPEVQNELAQFAAHMINSVRRQLGLPPVTVTAGSQEFARLLSTS |
| | K64/K287/K386/ | YKXTHGNTRPSFVYGQPGVSGHYGVGPHDKTIIEDSAGASGLIR |
| | K657 - numbered | NDDNMYENIGAFNDVHTVNGIKRGIYDSIKYMLFTDHLHGNTYG |
| | according to full- | HAINFLRVDKHNPNAPVYLGFSTSNVGSLNEHFVMFPESNIANH |
| | length sequence | QRENKTPIKAVGSTKDYAQRVGTVSDTIAAIKGKVSSLENRLSA |
| | | IHQEADIMAAQAKVSQLQGKLASTLKQSDSLNLQVRQLNDTKGS |
| | | LRTELLAAKAKQAQLEATRDQSLAKLASLKAALHQTEALAEQAA |
| | | ARVTALVAKXAHLQYLRDFKLNPNRLQVIRERIDNTKQDLAKTT |
| | | SSLLNAQE |
| 79 | SpyAD [33-684 | GQVKADDRASGETKASNTHDDSLPKPETIQEAXATIDAVEKTLS |
| | fragment] nnAA | QQKAELTELATALTKTTAEINHLKEQQDNEQKALTSAQEIYTNT |
| | (e.g., pAMF) | LASSEETLLAQGAEHQRELTATETELHNAQADQHSKETALSEQK |
| | mutant w/o leader | ASISAETTRAQDLVEQVKTSEQNIAKLNAMISNPDAITKAAQTA |
| | Fragment of SEQ | NDNTKALSSELEKAKADLENQKAKVKKQLTEELAAQKAALAEKE |
| | ID NO: 34 | AELSRLKSSAPSTQDSIVGNNTMKAPQGYPLEELKXLEASGYIG |
| | X = nnAA (e.g., | SASYNNYYKEHADQIIAKASPGNQLNQYQDIPADRNRFVDPDNL |
| | pAMF) | TPEVQNELAQFAAHMINSVRRQLGLPPVTVTAGSQEFARLLSTS |
| | K64/K287/K386/ | YKXTHGNTRPSFVYGQPGVSGHYGVGPHDKTIIEDSAGASGLIR |
| | K657 - numbered | NDDNMYENIGAFNDVHTVNGIKRGIYDSIKYMLFTDHLHGNTYG |
| | according to full- | HAINFLRVDKHNPNAPVYLGFSTSNVGSLNEHFVMFPESNIANH |
| | length sequence | QRFNKTPIKAVGSTKDYAQRVGTVSDTIAAIKGKVSSLENRLSA |
| | | IHQEADIMAAQAKVSQLQGKLASTLKQSDSLNLQVRQLNDTKGS |
| | | LRTELLAAKAKQAQLEATRDQSLAKLASLKAALHQTEALAEQAA |
| | | ARVTALVAKXAHLQYLRDFKLNPNRLQVIRERIDNTK |
| 80 | Spy AD [33-849 | MHHHHHHHHHHHHGSGENLYFQGQVKADDRASGETKASNTHDDS |
| | fragment] nnAA | LPKPETIQEAXATIDAVEKTLSQQKAELTELATALTKTTAEINH |
| | (e.g., | LKEQQDNEQKALTSAQEIYTNTLASSEETLLAQGAEHQRELTAT |
| | pAMF)mutant w/ | ETELHNAQADQHSKETALSEQKASISAETTRAQDLVEQVKTSEQ |
| | long leader | NIAKLNAMISNPDAITKAAQTANDNTKALSSELEKAKADLENQK |
| | X = nnAA (e.g., | AKVKKQLTEELAAQKAALAEKEAELSRLKSSAPSTQDSIVGNNT |
| | pAMF) positions | MKAPQGYPLEELKXLEASGYIGSASYNNYYKEHADQIIAKASPG |
| | K64/K287/K386/ | NQLNQYQDIPADRNRFVDPDNLTPEVQNELAQFAAHMINSVRRQ |
| | K657 - numbered | LGLPPVTVTAGSQEFARLLSTSYKXTHGNTRPSFVYGQPGVSGH |
| | according to full | YGVGPHDKTIIEDSAGASGLIRNDDNMYENIGAFNDVHTVNGIK |
| | length sequence | RGIYDSIKYMLFTDHLHGNTYGHAINFLRVDKHNPNAPVYLGES |
| | | TSNVGSLNEHFVMFPESNIANHQRENKTPIKAVGSTKDYAQRVG |
| | | TVSDTIAAIKGKVSSLENRLSAIHQEADIMAAQAKVSQLQGKLA |
| | | STLKQSDSLNLQVRQLNDTKGSLRTELLAAKAKQAQLEATRDQS |
| | | LAKLASLKAALHQTEALAEQAAARVTALVAKXAHLQYLRDEKLN |
| | | PNRLQVIRERIDNTKQDLAKTTSSLLNAQEALAALQAKQSSLEA |
| | | TIATTEHQLTLLKTLANEKEYRHLDEDIATVPDLQVAPPLTGVK |
| | | PLSYSKIDTTPLVQEMVKETKQLLEASARLAAENTSLVAEALVG |
| | | QTSEMVASNAIVSKITSSITQPSSKTSYGSGSSTTSNLISDVDE |
| | | STQR |
| 81 | Spy AD [33-594 | QVKADDRASGETKASNTHDDSLPKPETIQEAXATIDAVEKTLSQ |
| | fragment] nnAA | QKAELTELATALTKTTAEINHLKEQQDNEQKALTSAQEIYTNTL |
| | (e.g., | ASSEETLLAQGAEHQRELTATETELHNAQADQHSKETALSEQKA |
| | pAMF)mutant w/ | SISAETTRAQDLVEQVKTSEQNIAKLNAMISNPDAITKAAQTAN |
| | long leader | DNTKALSSELEKAKADLENQKAKVKKQLTEELAAQKAALAEKEA |
| | Fragment of SEQ | ELSRLKSSAPSTQDSIVGNNTMKAPQGYPLEELKXLEASGYIGS |
| | ID NO: 34 | ASYNNYYKEHADQIIAKASPGNQLNQYQDIPADRNRFVDPDNLT |
| | X = nnAA (e.g., | PEVQNELAQFAAHMINSVRRQLGLPPVTVTAGSQEFARLLSTSY |
| | pAMF) positions | KXTHGNTRPSFVYGQPGVSGHYGVGPHDKTIIEDSAGASGLIRN |
| | K64/K287/K386 - | DDNMYENIGAFNDVHTVNGIKRGIYDSIKYMLFTDHLHGNTYGH |
| | numbered | AINFLRVDKHNPNAPVYLGFSTSNVGSLNEHFVMFPESNIANHQ |
| | according to full | RFNKTPIKAVGSTKDYAQRVGTVSDTIAAIKGKVSSLENRLSAI |
| | length sequence | HQEADIMAAQAKVSQLQGKLASTLKQSDSLNLQV |
| 82 | SpyAD [33-657 | QVKADDRASGETKASNTHDDSLPKPETIQEAXATIDAVEKTLSQ |
| | fragment] nnAA | QKAELTELATALTKTTAEINHLKEQQDNEQKALTSAQEIYTNTL |
| | (e.g., | ASSEETLLAQGAEHQRELTATETELHNAQADQHSKETALSEQKA |
| | pAMF)mutant w/ | SISAETTRAQDLVEQVKTSEQNIAKLNAMISNPDAITKAAQTAN |
| | long leader | DNTKALSSELEKAKADLENQKAKVKKQLTEELAAQKAALAEKEA |
| | Fragment of SEQ | ELSRLKSSAPSTQDSIVGNNTMKAPQGYPLEELKXLEASGYIGS |
| | ID NO: 34 | ASYNNYYKEHADQIIAKASPGNQLNQYQDIPADRNRFVDPDNLT |
| | X = nnAA (e.g., | PEVQNELAQFAAHMINSVRRQLGLPPVTVTAGSQEFARLLSTSY |
| | pAMF) positions | KXTHGNTRPSFVYGQPGVSGHYGVGPHDKTIIEDSAGASGLIRN |
| | K64/K287/K386/ | DDNMYENIGAFNDVHTVNGIKRGIYDSIKYMLFTDHLHGNTYGH |
| | K657 - numbered | AINFLRVDKHNPNAPVYLGFSTSNVGSLNEHFVMFPESNIANHQ |
| | according to full | RFNKTPIKAVGSTKDYAQRVGTVSDTIAAIKGKVSSLENRLSAI |
| | length sequence | HQEADIMAAQAKVSQLQGKLASTLKQSDSLNLQVRQLNDTKGSL |
| | | RTELLAAKAKQAQLEATRDQSLAKLASLKAALHQTEALAEQAAA |
| | | RVTALVAKX |
| 83 | Spy AD [33-667 | QVKADDRASGETKASNTHDDSLPKPETIQEAXATIDAVEKTLSQ |
| | fragment] nnAA | QKAELTELATALTKTTAEINHLKEQQDNEQKALTSAQEIYTNTL |
| | (e.g., | ASSEETLLAQGAEHQRELTATETELHNAQADQHSKETALSEQKA |
| | pAMF)mutant w/ | SISAETTRAQDLVEQVKTSEQNIAKLNAMISNPDAITKAAQTAN |
| | long leader | DNTKALSSELEKAKADLENQKAKVKKQLTEELAAQKAALAEKEA |
| | Fragment of SEQ | ELSRLKSSAPSTQDSIVGNNTMKAPQGYPLEELKXLEASGYIGS |
| | ID NO: 34 | ASYNNYYKEHADQIIAKASPGNQLNQYQDIPADRNRFVDPDNLT |
| | X = nnAA (e.g., | PEVQNELAQFAAHMINSVRRQLGLPPVTVTAGSQEFARLLSTSY |
| | pAMF) positions | KXTHGNTRPSFVYGQPGVSGHYGVGPHDKTIIEDSAGASGLIRN |
| | K64/K287/K386/ | DDNMYENIGAFNDVHTVNGIKRGIYDSIKYMLFTDHLHGNTYGH |
| | K657 - numbered | AINFLRVDKHNPNAPVYLGESTSNVGSLNEHFVMFPESNIANHQ |
| | according to full | RFNKTPIKAVGSTKDYAQRVGTVSDTIAAIKGKVSSLENRLSAI |
| | length sequence | HQEADIMAAQAKVSQLQGKLASTLKQSDSLNLQVRQLNDTKGSL |
| | | RTELLAAKAKQAQLEATRDQSLAKLASLKAALHQTEALAEQAAA |
| | | RVTALVAKXAHLQYLRDFK |

In some embodiments, the polypeptide antigen is SLO. In some embodiments, the polypeptide antigen is SLO(ΔC101) and comprises three or four nnAAs substituted at positions selected from selected from K98, K112, R151, K189, K272, K323, K357, K375, K407, and K464 of SEQ ID NO: 53. In some embodiments, the polypeptide antigen is SLO(ΔC101) and comprises or consists of nnAAs substituted at positions K98, R151, K272, and K357; positions K112, K189, K323, and K375; positions R151, K272, K357, and K407; positions R151, K272, K375, and K464; positions K112, K272, K357, and K464; positions K98, K189, and K357; positions K112, K189, and K323; positions K98, R151, and K272; positions K112, K272, and K375; or positions K112, K323, and K407. In some embodiments, the three or four nnAAs are each pAMF. In some embodiments, the polypeptide antigen is a SLO polypeptide and comprises an amino acid sequence that is at least 95%, 96%, 97%, 98%, or 99% identical to any one of SEQ ID NOs: 55, 56, 57, 58, 59, 60, 61, 62, 63, or 64. In some embodiments, the conjugate polypeptide is a SLO polypeptide and comprises or consists of the amino acid sequence of any one of SEQ ID NOs: 55, 56, 57, 58, 59, 60, 61, 62, 63, or 64.

In some embodiments, the polypeptide antigen is a SLO polypeptide. In some embodiments, the polypeptide antigen is SLO(ΔC101) and comprises five, six, seven, or eight nnAAs substituted at positions selected from selected from K98, K112, R151, K189, K272, K323, K357, K375, K407, and K464 of SEQ ID NO: 53. In some embodiments, the polypeptide antigen is SLO(ΔC101) and comprises or consists of nnAAs substituted at positions K98, R151, K272, K357, and K407; positions K112, K189, K323, K375, and K464; positions K112, R151, K272, K357, and K407; positions K98, R151, K272, K357, K407, and K464; positions K112, R151, K189, K323, K375, and K464; positions K98, K112, K189, K323, K375, and K464; positions K112, R151, K189, K272, K357, K407, and K464; positions K98, R151, K189, K323, K375, K407, and K464; positions K112, K189, K272, K357, K375, K407, and K464; positions K98, K112, R151, K189, K272, K323, K357, and K375; positions K98, R151, K189, K272, K323, K357, K407, and K464; or positions K112, K189, K272, K323, K357, K375, K407, and K464. In some embodiments, the five, six, seven, or eight nnAAs are each pAMF. In some embodiments, the polypeptide antigen is a SLO polypeptide and comprises an amino acid sequence that is at least 95%, 96%, 97%, 98%, or 99% identical to any one of SEQ ID NOs: 65, 66, 67, 68, 69, 70, 71, 72, 73, 74, 75, or 76. In some embodiments, the polypeptide antigen is a SLO polypeptide and comprises or consists of the amino acid sequence of any one of SEQ ID NOs: 65, 66, 67, 68, 69, 70, 71, 72, 73, 74, 75, or 76.

In some embodiments, the polypeptide antigen is SpyAD and comprises four nnAAs substituted at positions K64, K287, K396, and K657 of SEQ ID NO: 9. In some embodiments, the polypeptide antigen is SpyAD and comprises four nnAAs substituted at positions K64, K287, K396, and K657 of SEQ ID NO: 33. In some embodiments, the four nnAAs are each pAMF. In some embodiments, the polypeptide antigen is SpyAD and comprises an amino acid sequence that is at least 95%, 96%, 97%, 98%, or 99% identical to SEQ ID NO: 11 or SEQ ID NO: 12. In some embodiments, the four nnAAs are each pAMF. In some embodiments, the polypeptide antigen is SpyAD and comprises an amino acid sequence that is at least 95%, 96%, 97%, 98%, or 99% identical to SEQ ID NO: 34. In some embodiments, the polypeptide antigen is SpyAD and comprises or consists of the amino acid sequence of SEQ ID NO: 11 or SEQ ID NO: 12. In some embodiments, the polypeptide antigen is SpyAD and comprises or consists of the amino acid sequence of SEQ ID NO: 34.

In some embodiments, the polypeptide antigen is SpyAD and comprises an amino acid sequence that is at least 95%, 96%, 97%, 98%, or 99% identical to SEQ ID NO: 11, SEQ ID NO: 12, or SEQ ID NO: 80. In some embodiments, the polypeptide antigen is SpyAD and comprises or consists of the amino acid sequence of SEQ ID NO: 11, SEQ ID NO: 12, or SEQ ID NO: 80.

In some embodiments, the polypeptide antigen is SpyAD and comprises an amino acid sequence that is at least 95%, 96%, 97%, 98%, or 99% identical to SEQ ID NO: 77, SEQ ID NO: 78, SEQ ID NO: 79, SEQ ID NO: 81, SEQ ID NO: 82, or SEQ ID NO: 83. In some embodiments, the polypeptide antigen is SpyAD and comprises or consists of SEQ ID NO: 77, SEQ ID NO: 78, or SEQ ID NO: 79, SEQ ID NO: 81, SEQ ID NO: 82, or SEQ ID NO: 83.

In some embodiments, the non-GAS carrier polypeptide comprises of consists of eCRM197. In certain embodiments, the eCRM197 has the sequence of SEQ ID NO: 25.

In some embodiments, the GAS polypeptide antigen or anon-GAS carrier polypeptide comprises or consists of the amino acid sequence of a polypeptide listed in Table 1. In some embodiments, the GAS polypeptide antigen or a non-GAS carrier polypeptide comprises an amino acid sequence that is at least 95%, 96%, 97%, 98%, or 99% identical to any one of SEQ ID NOs: 1-76.

In some embodiments, the GAS polypeptide antigen or anon-GAS carrier polypeptide comprises or consists of the amino acid sequence of a polypeptide listed in Table 1 or 1A. In some embodiments, the GAS polypeptide antigen or a non-GAS carrier polypeptide comprises an amino acid sequence that is at least 95%, 96%, 97%, 98%, or 99% identical to any one of SEQ ID NOs: 1-80.

In some embodiments, the GAS polypeptide antigen or anon-GAS carrier polypeptide comprises or consists of the amino acid sequence of a polypeptide listed in Table 1. In some embodiments, the GAS polypeptide antigen or a non-GAS carrier polypeptide comprises an amino acid sequence that is at least 95%, 96%, 97%, 98%, or 99% identical to any one of SEQ ID NOs: 1-76

In some embodiments, the GAS polypeptide antigen or anon-GAS carrier polypeptide comprises or consists of the amino acid sequence of a polypeptide listed in Table 1A. In some embodiments, the GAS polypeptide antigen or a non-GAS carrier polypeptide comprises an amino acid sequence that is at least 95%, 96%, 97%, 98%, or 99% identical to any one of SEQ ID NOs: 77-80.

### Immunogenic Compositions

As discussed previously, the long polysaccharides produced by the methods described herein and polysaccharide-polypeptide conjugates prepared using the long polysaccharides are suitable for use in immunogenic compositions (e.g., vaccines for treating or preventing illness). For example, in some embodiments, the immunogenic compositions may induce a protective immune response against a GAS bacterium in a subject. In some embodiments, provided herein are the use of the immunogenic compositions described herein in the manufacture of a medicament for inducing a protective immune response against a GAS bacterium in a subject.

Herein, the term “subject” refers to a mammal. In some embodiments, the subject is a mouse, a rat, a dog, a guinea pig, a sheep, a non-human primate, or a human. In some embodiments, the subject is a human. In some embodiments, the human subjects are 18 years of age or older. In some embodiments, the human subjects are less than 18 years of age.

In some embodiments, the human subjects are between 6 months of age and 17 years of age. In some embodiments, the human subjects are between 6 months of age and 9 years of age, between 6 months of age and 8 years of age, between 6 months of age and 7 years of age, between 6 months of age and 6 years of age, between 6 months of age and 5 years of age, between 6 months of age and 4 years of age, between 6 months of age and 3 years of age, between 6 months of age and 2 years of age, or between 6 months of age and 1 year of age. In some embodiments, the human subjects are between 5 years of age and 17 years of age, between 7 years of age and 17 years of age, between 9 years of age and 17 years of age, between 11 years of age and 17 years of age, between 13 years of age and 17 years of age, or between 15 years of age and 17 years of age. In some embodiments, the human subjects are 6 months, 1 year, 2 years, 3 years, 4 years, 5 years, 6 years, 7 years, 8 years, 9 years, 10 years, 11 years, 12 years, 13 years, 14 years, 15 years, 16 years, 17 years, or 18 years of age.

Herein, the term “protective immune response” encompasses eliciting an anti-GAS antibody response in the subject. Antibody titers generated after administration of the immunogenic compositions described herein can be determined by means known in the art, for example by ELISA assays of serum samples derived from immunized subjects. In some embodiments, the immunogenic compositions described herein elicit antibody responses in treated subjects, wherein the antibodies generated bind to multiple (i.e., two or more) GAS serotypes. In some embodiments, the immunogenic compositions described herein elicit antibody responses in treated subjects, wherein the antibodies generated bind to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or more GAS serotypes. In some embodiments, the immunogenic compositions described herein do not elicit antibody responses against human proteins or tissue.

In some embodiments, the immunogenic compositions described herein elicit antibody responses in treated subjects, wherein the antibodies generated bind to at least one GAS serotype selected from M1, M2, M3, M4, M5, M6, M9, M11, M12, M13, M18, M22, M25, M28, M62, M71, M72, M74, M75, M77, M80, M81, M83, M87, M89, and M92. In some embodiments, the immunogenic compositions described herein elicit antibody responses in treated subjects, wherein the antibodies generated bind to two or more GAS serotypes selected from M1, M2, M3, M4, M5, M6, M9, M11, M12, M13, M18, M22, M25, M28, M62, M71, M72, M74, M75, M77, M80, M81, M83, M87, M89, and M92.

In some embodiments, the immunogenic compositions described herein elicit antibody responses in treated subjects, wherein the antibodies generated bind to at least one GAS serotype selected from M1, M2, M3, M4, M6, M11, M12, M22, M28, M75, and M89. In some embodiments, the immunogenic compositions described herein elicit antibody responses in treated subjects, wherein the antibodies generated bind to two or more GAS serotypes selected from M1, M2, M3, M4, M6, M11, M12, M22, M28, M75, and M89.

In some embodiments, the immunogenic compositions described herein elicit antibody responses in treated subjects, wherein the antibodies generated bind to at least one GAS serotype selected from M1, M3, M5, M9, M12, M18, M22, M25, M28, M71, M72, and M74. In some embodiments, the immunogenic compositions described herein elicit antibody responses in treated subjects, wherein the antibodies generated bind to two or more GAS serotypes selected from M1, M3, M5, M9, M12, M18, M22, M25, M28, M71, M72, and M74.

In some embodiments, the immunogenic compositions described herein elicit antibody responses in treated subjects, wherein the antibodies generated bind to at least one GAS serotype selected from M1, M4, M6, M11, M12, M22, M44, M75, M77, M77, and M81. In some embodiments, the immunogenic compositions described herein elicit antibody responses in treated subjects, wherein the antibodies generated bind to two or more GAS serotypes selected from M1, M4, M6, M11, M12, M22, M44, M75, M77, M77, and M81.

In some embodiments, the immunogenic compositions described herein elicit antibody responses in treated subjects, wherein the antibodies generated bind to at least one GAS serotype selected from M1, M2, M3, M4, M6, M9, M12, M18, M22, M75, M77, M89, and M92. In some embodiments, the immunogenic compositions described herein elicit antibody responses in treated subjects, wherein the antibodies generated bind to two or more GAS serotypes selected from M1, M2, M3, M4, M6, M9, M12, M18, M22, M75, M77, M89, and M92.

In some embodiments, the immunogenic compositions described herein elicit antibody responses in treated subjects, wherein the antibodies generated bind to at least one GAS serotype selected from M1, M2, M3, M4, M5, M6, M9, M11, M12, M13, M28, M62, and M89. In some embodiments, the immunogenic compositions described herein elicit antibody responses in treated subjects, wherein the antibodies generated bind to two or more GAS serotypes selected from M1, M2, M3, M4, M5, M6, M9, M11, M12, M13, M28, M62, and M89.

In some embodiments, the immunogenic compositions described herein elicit antibody responses in treated subjects, wherein the antibodies generated bind to at least one GAS serotype selected from M1, M2, M3, M4, M6, M12, M22, M28, M49, M53, M68, M77, M80, M83, M87, M89, and M92. In some embodiments, the immunogenic compositions described herein elicit antibody responses in treated subjects, wherein the antibodies generated bind to two or more GAS serotypes selected from M1, M2, M3, M4, M6, M12, M22, M28, M49, M53, M68, M77, M80, M83, M87, M89, and M92.

In some embodiments, the immunogenic compositions described herein elicit antibody responses in treated subjects that bind to one or more Shigella serotypes. In some embodiments, the immunogenic compositions described herein elicit antibody responses in treated subjects that bind to one or more GAS serotypes and also bind to one or more Shigella serotypes. In some embodiments, the Shigella serotypes comprise a polysaccharide comprising a polyrhamanose backbone. Exemplary Shigella serotypes comprising such polysaccharides include S. flexneri such as S. flexneri 2A and 3A and S. flexneri 6.

Such immunogenic compositions may, generally, comprise (a) a Group A Streptococcus (GAS) C5a peptidase polypeptide antigen; (b) a GAS streptolysin O (SLO) polypeptide antigen; and (c) a polypeptide-polysaccharide conjugate.

In some embodiments, a Group A Streptococcus (GAS) C5a peptidase polypeptide antigen may be a full-length, native C5a peptidase polypeptide, or a fragment thereof. In some embodiments, the C5a peptidase polypeptide antigen, or a fragment thereof, comprises or consists of the amino acid sequence of SEQ ID NO: 1, SEQ ID NO: 2, SEQ ID NO: 3, SEQ ID NO: 4, SEQ ID NO: 29, or SEQ ID NO: 30. In some embodiments, the C5a peptidase polypeptide antigen, or a fragment thereof, comprises or consists of the amino acid sequence of SEQ ID NO: 29 or SEQ ID NO: 30. In some embodiments, the C5a peptidase polypeptide antigen comprises or consists of the amino acid sequence of SEQ ID NO: 30. In some embodiments, the C5a peptidase polypeptide antigen, or a fragment thereof, comprises or consists of an amino acid sequence that is at least 95%, 96%, 97%, 98%, or 99% identical to SEQ ID NO: 1, SEQ ID NO: 2, SEQ ID NO: 3, SEQ ID NO: 4, SEQ ID NO: 29, or SEQ ID NO: 30. In some embodiments, the C5a peptidase polypeptide antigen, or a fragment thereof, comprises an amino acid sequence that is at least 95%, 96%, 97%, 98%, or 99% identical to SEQ ID NO: 29, or SEQ ID NO: 30. In some embodiments the C5a peptidase polypeptide antigen, or a fragment thereof, has the amino acid sequence of SEQ ID NO: 1, SEQ ID NO: 2, SEQ ID NO: 3, SEQ ID NO: 4, SEQ ID NO: 29, or SEQ ID NO: 30. In some embodiments the C5a peptidase polypeptide antigen, or a fragment thereof, has the amino acid sequence of SEQ ID NO: 29 or SEQ ID NO: 30.

Likewise, the immunogenic compositions described herein may comprise a GAS streptolysin O (SLO) polypeptide antigen, or a fragment thereof. In some embodiments, the SLO polypeptide antigen, or a fragment thereof, comprises or consists of the amino acid sequence of SEQ ID NO: 5, SEQ ID NO: 6, SEQ ID NO: 7, SEQ ID NO: 8, SEQ ID NO: 31, SEQ ID NO: 32, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53. In certain embodiments, the SLO polypeptide antigen, or a fragment thereof, comprises or consists of the amino acid sequence of SEQ ID NO: 31, SEQ ID NO: 32, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53. In certain embodiments, the SLO antigen comprises or consists of the amino acid sequence of SEQ ID NO: 53. In some embodiments described herein, the SLO antigen, or a fragment thereof, comprises or consists of an amino acid sequence that is at least 95%, 96%, 97%, 98%, or 99% identical to SEQ ID NO: 5, SEQ ID NO: 6, SEQ ID NO: 7, SEQ ID NO: 8, SEQ ID NO: 31, SEQ ID NO: 32, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53. In some embodiments described herein, the SLO antigen, or a fragment thereof, comprises or consists of an amino acid sequence that is at least 95%, 96%, 97%, 98%, or 99% identical to SEQ ID NO: 31, SEQ ID NO: 32, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53. In some embodiments described herein, the SLO antigen, or a fragment thereof comprises or consists of an amino acid sequence that is at least 95%, 96%, 97%, 98%, or 99% identical to SEQ ID NO: 53. In some embodiments, the SLO antigen, or a fragment thereof, has the amino acid sequence of SEQ ID NO: 5, SEQ ID NO: 6, SEQ ID NO: 7, SEQ ID NO: 8, SEQ ID NO: 31, SEQ ID NO: 32, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53. In some embodiments, the SLO antigen, or a fragment thereof, has the amino acid sequence of SEQ ID NO: 31, SEQ ID NO: 32, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53. In some embodiments, the SLO antigen, or a fragment thereof, has the amino acid sequence of SEQ ID NO: 53.

The immunogenic compositions described herein may comprise a polypeptide-polysaccharide conjugate. In some embodiments, the polypeptide-polysaccharide conjugate comprises: (i) a Streptococcus pyogenes Adhesion and Division (SpyAD) conjugate polypeptide, or a fragment thereof, comprising at least one non-natural amino acid (nnAA), wherein the at least one nnAA comprises a click chemistry reactive group; and (ii) a GAS polysaccharide, or a variant thereof, that lacks an immunodominant N-acetyl Glucosamine (GlcNAc) side chain.

As discussed above, the SpyAD conjugate polypeptide, or a fragment thereof, of the immunogenic compositions comprise at least one nnAA comprising a click chemistry reactive group, enabling its conjugation to a GAS polysaccharide or variant thereof. In some embodiments, the at least one nnAA may be selected from 2-amino-3-(4-azidophenyl)propanoic acid (pAF), 2-amino-4-azidobutanoic acid, 2-azido-3-phenylpropionic acid, 2-amino-3-azidopropanoic acid, 2-amino-3-(4-(azidomethyl)phenyl)propanoic acid (pAMF), 2-amino-3-(5-(azidomethyl)pyridin-2-yl)propanoic acid, 2-amino-3-(4-(azidomethyl)pyridin-2-yl)propanoic acid, 2-amino-3-(6-(azidomethyl)pyridin-3-yl)propanoic acid, and 2-amino-5-azidopentanoic acid. In some embodiments, the at least one nnAA is pAMF.

The SpyAD conjugate polypeptide may be a native or full-length SpyAD conjugate polypeptide, or a fragment thereof. In some embodiments, the SpyAD conjugate polypeptide, or fragment thereof, comprises or consists of an amino acid sequence that is at least 95%, 96%, 97%, 98%, or 99% identical to SEQ ID NO: 33. In some embodiments, the SpyAD conjugate polypeptide comprises or consists of an amino acid sequence that is a fragment of SEQ ID NO: 33. In some embodiments, the SpyAD conjugate polypeptide has an amino acid sequence that is a fragment of SEQ ID NO: 33.

In some embodiments, the SpyAD conjugate polypeptide, or a fragment thereof, comprises a pAMF substitution at positions K64, K287, K386, and K657 of SEQ ID NO: 33 (note: numbering herein is based on the full-length native sequences). Thus, in some embodiments, the SpyAD conjugate polypeptide, or a fragment thereof, comprises or consists of the amino acid sequence of SEQ ID NO: 34, SEQ ID NO: 77, SEQ ID NO: 78, SEQ ID NO: 79, SEQ ID NO: 81, SEQ ID NO: 82, or SEQ ID NO: 83. In some embodiments, the SpyAD conjugate polypeptide, or a fragment thereof, comprises or consists of an amino acid sequence that is at least 95%, 96%, 97%, 98%, or 99% identical to SEQ ID NO: 34, SEQ ID NO: 77, SEQ ID NO: 78, SEQ ID NO: 79, SEQ ID NO: 81, SEQ ID NO: 82, or SEQ ID NO: 83. In some embodiments, the SpyAD conjugate polypeptide, or a fragment thereof, comprises or consists of an amino acid sequence that is at least 95%, 96%, 97%, 98%, or 99% identical to SEQ ID NO: 34. In certain embodiments, the SpyAD conjugate polypeptide, or a fragment thereof, has the amino acid sequence of SEQ ID NO: 34, SEQ ID NO: 77, SEQ ID NO: 78, SEQ ID NO: 79, SEQ ID NO: 81, SEQ ID NO: 82, or SEQ ID NO: 83. In certain embodiments, the SpyAD conjugate polypeptide, or a fragment thereof, has the amino acid sequence of SEQ ID NO: 34.

In some embodiments of the immunogenic compositions described herein, the C5a peptidase polypeptide antigen, or a fragment thereof, comprises an amino acid sequence that is at least 95% identical to SEQ ID NO: 30; the SLO polypeptide antigen, or a fragment thereof, comprises an amino acid sequence that is at least 95% identical to SEQ ID NO: 53; and the SpyAD conjugate polypeptide, or a fragment thereof, comprises an amino acid sequence that is at least 95% identical to SEQ ID NO: 34. In certain embodiments, the C5a peptidase polypeptide antigen, or a fragment thereof, comprises the amino acid sequence of SEQ ID NO: 30; the SLO polypeptide antigen, or a fragment thereof, comprises the amino acid sequence of SEQ ID NO: 53; and the SpyAD conjugate polypeptide, or a fragment thereof, comprises the amino acid sequence of SEQ ID NO: 34. In some embodiments, the C5a peptidase polypeptide antigen, or a fragment thereof, has the amino acid sequence of SEQ ID NO: 30; the SLO polypeptide antigen, or a fragment thereof, has the amino acid sequence of SEQ ID NO: 53; and the SpyAD conjugate polypeptide, or a fragment thereof, has the amino acid sequence of SEQ ID NO: 34.

In some embodiments of any of the immunogenic compositions described herein, between about 8 mol % and 20 mol % of the polysaccharide repeat units in the GAS polysaccharide, or variant thereof, of the polypeptide-polysaccharide conjugate are derivatized by a click chemistry reactive group or linker comprising a click chemistry reactive group. In some embodiments, between about 8 mol % and about 20 mol %, about 8 mol % and about 19 mol %, about 8 mol % and about 18 mol %, about 8 mol % and about 17 mol %, about 8 mol % and about 16 mol %, about 8 mol % and about 15 mol %, about 8 mol % and about 14 mol %, 8 mol % and about 13 mol %, about 8 mol % and about 12 mol %, about 8 mol % and about 11 mol %, 8 mol % and about 10 mol %, about 8 mol % and about 9 mol %, about 9 mol % and about 20 mol %, about 9 mol % and about 19 mol %, about 9 mol % and about 18 mol %, about 9 mol % and about 17 mol %, about 9 mol % and about 16 mol %, about 9 mol % and about 15 mol %, about 9 mol % and about 14 mol %, 9 mol % and about 13 mol %, about 9 mol % and about 12 mol %, about 9 mol % and about 11 mol %, 9 mol % and about 10 mol %, about 10 mol % and about 20 mol %, about 10 mol % and about 19 mol %, about 10 mol % and about 18 mol %, about 10 mol % and about 17 mol %, about 10 mol % and about 16 mol %, about 10 mol % and about 15 mol %, about 10 mol % and about 14 mol %, 10 mol % and about 13 mol %, about 10 mol % and about 12 mol %, about 10 mol % and about 11 mol %, about 11 mol % and about 20 mol %, about 11 mol % and about 19 mol %, about 11 mol % and about 18 mol %, about 11 mol % and about 17 mol %, about 11 mol % and about 16 mol %, about 11 mol % and about 15 mol %, about 11 mol % and about 14 mol %, 11 mol % and about 13 mol %, about 11 mol % and about 12 mol %, about 12 mol % and about 20 mol %, about 12 mol % and about 19 mol %, about 12 mol % and about 18 mol %, about 12 mol % and about 17 mol %, about 12 mol % and about 16 mol %, about 12 mol % and about 15 mol %, about 12 mol % and about 14 mol %, 12 mol % and about 13 mol %, about 13 mol % and about 20 mol %, about 13 mol % and about 19 mol %, about 13 mol % and about 18 mol %, about 13 mol % and about 17 mol %, about 13 mol % and about 16 mol %, about 13 mol % and about 15 mol %, about 13 mol % and about 14 mol %, about 14 mol % and about 20 mol %, about 14 mol % and about 19 mol %, about 14 mol % and about 18 mol %, about 14 mol % and about 17 mol %, about 14 mol % and about 16 mol %, about 14 mol % and about 15 mol %, about 15 mol % and about 20 mol %, about 15 mol % and about 19 mol %, about 15 mol % and about 18 mol %, about 15 mol % and about 17 mol %, about 15 mol % and about 16 mol %, about 16 mol % and about 20 mol %, about 16 mol % and about 19 mol %, about 16 mol % and about 18 mol %, about 16 mol % and about 17 mol %, about 17 mol % and about 20 mol %, about 17 mol % and about 19 mol %, about 17 mol % and about 18 mol %, about 18 mol % and about 20 mol %, about 18 mol % and about 19 mol %, or about 19 mol % and about 20 mol % of the PS repeat units of a polysaccharide are derivatized by a click chemistry reactive group or a linker comprising a click chemistry reactive group. In some embodiments, the polysaccharide repeat units of a polysaccharide are polysaccharide repeat units of a GAS polysaccharide, or a variant thereof. In some embodiments of the immunogenic compositions described herein, between about 8, 9, 10, 11, 12, 13, 14, 15, 16, 17, 18, 19, or 20 mol % of the polysaccharide repeat units in the GAS polysaccharide, or a variant thereof, of the polypeptide-polysaccharide conjugate are derivatized by a click chemistry reactive group or linker comprising a click chemistry reactive group. In some embodiments, the GAS polysaccharide, or a variant thereof, of the polypeptide-polysaccharide conjugate, lacks an immunodominant N-acetyl Glucosamine (GlcNAc) side chain.

In some embodiments of the present immunogenic compositions, the linker, prior to reaction with the click chemistry reactive group of the nnAA, comprises a structure of Formula I:

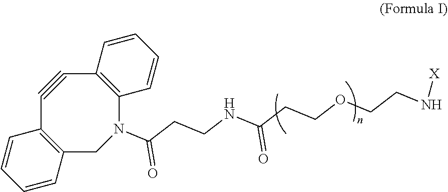

wherein, X is at least one polysaccharide repeat unit of polysaccharide and n is at least 1. In some embodiments, the polysaccharide is a GAS polysaccharide, or a variant thereof. In some embodiments, n is at least 1, at least 2, at least 3, at least 4, or at least 5. In some embodiments, in is 1, 2, 3, 4, or 5. Where X is described, here or elsewhere, as being attached to a polysaccharide repeat unit of a GAS polysaccharide, or a variant thereof, this can refer to a chemical attachment to or via any suitable functional group within the polysaccharide repeat unit (e.g., reacted with an aldehyde, which may arise from oxidation of a vicinal diol, via reductive amination). In some embodiments, X, and the —NH— of Formula I, are part of an isourea moiety.

In some embodiments, the SpyAD conjugate polypeptide is linked to the GAS polysaccharide, or a variant thereof, according to Formula II:

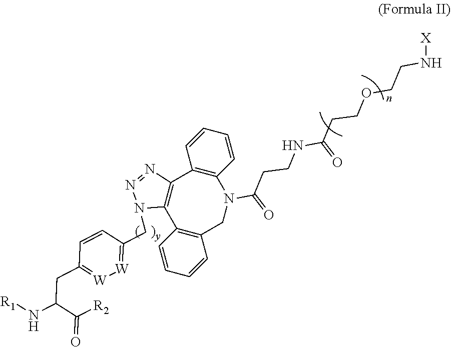

wherein, R₁ is H, formyl, or at least one amino acid of the SpyAD conjugate polypeptide, or fragment thereof; R₂ is OH or at least one amino acid of the SpyAD conjugate polypeptide, or fragment thereof; W is CH or N; y is at least 1; n is at least 1; and X is at least one polysaccharide repeat unit of a GAS polysaccharide or variant thereof. In some embodiments R₁ and R₂ are both amino acids of the SpyAD conjugate polypeptide, or fragment thereof. In some embodiments, y is at least 1, at least 2, or at least 3. In certain embodiments, y is 1, 2, or 3. In some embodiments, both of W are CH. In some embodiments, both of W are N. In some embodiments, one W is N and one W is CH. In some embodiments, n is at least 1, at least 2, at least 3, at least 4, or at least 5. In certain embodiments, n is 1, 2, 3, 4, or 5. In some embodiments, X, and the —NH— of Formula I, are part of an isourea moiety.

In some embodiments of the immunogenic compositions described herein, the GAS polysaccharide, or a variant thereof, has a molecular weight of at least about 10 kDa to at least about 40 kDa. In certain embodiments, the purified cell wall polysaccharide or peptidoglycan-bound capsular polysaccharide has an average molecular weight of about 10 kDa to about 40 kDa; about 10 kDa to about 35 kDa; about 10 kDa to about 30 kDa; about 10 kDa to about 25 kDa; about 10 kDa to about 20 kDa; about 10 kDa to about 15 kDa; 15 kDa to about 40 kDa; about 15 kDa to about 35 kDa; about 15 kDa to about 30 kDa; about 15 kDa to about 25 kDa; about 15 kDa to about 20 kDa; 20 kDa to about 40 kDa; about 20 kDa to about 35 kDa; about 20 kDa to about 30 kDa; about 20 kDa to about 25 kDa; 25 kDa to about 40 kDa; about 25 kDa to about 35 kDa; about 25 kDa to about 30 kDa; about 30 kDa to about 40 kDa; about 30 kDa to about 35 kDa; or about 35 kDa to about 40 kDa. In some embodiments, the purified cell wall polysaccharide or peptidoglycan-bound capsular polysaccharide has an average molecular weight of about 10 kDa, about 15 kDa, about 20 kDa, about 25 kDa, about 30 kDa, about 35 kDa, or about 40 kDa. In some embodiments, the GAS polysaccharide, or a variant thereof, lacks an immunodominant N-acetyl Glucosamine (GlcNAc) side chain.

In addition to having longer polysaccharides, the polypeptide-polysaccharide conjugates of the immunogenic compositions described herein may have increased average molecular weight. In some embodiments of any of the polypeptide-polysaccharide conjugates described herein, the average molecular weight is greater than about 185 kDa or 190 kDa. In some embodiments, the average molecular weight is between about 185 kDa and about 700 kDa, about 185 kDa and about 600 kDa, about 185 kDa and about 500 kDa, about 185 kDa and about 400 kDa, about 185 kDa and about 300 kDa, and about 185 kDa and about 200 kDa. In some embodiments, the polypeptide-polysaccharide conjugates may have a molecular weight or average molecular weight between about 185 kDa and about 700 kDa, about 185 kDa and about 650 kDa, about 185 kDa and about 600 kDa, about 185 kDa and about 550 kDa, about 185 kDa and about 500 kDa, about 185 kDa and about 450 kDa, about 185 kDa and about 400 kDa, about 185 kDa and about 350 kDa, about 185 kDa and about 300 kDa, about 185 kDa and about 250 kDa, about 185 kDa and about 200 kDa, about 200 kDa and about 700 kDa, about 200 kDa and about 650 kDa, about 200 kDa and about 600 kDa, about 200 kDa and about 550 kDa, about 200 kDa and about 500 kDa, about 200 kDa and about 450 kDa, about 200 kDa and about 400 kDa, about 200 kDa and about 350 kDa, about 200 kDa and about 300 kDa, about 200 kDa and about 250 kDa, about 250 kDa and about 700 kDa, about 250 kDa and about 650 kDa, about 250 kDa and about 600 kDa, about 250 kDa and about 550 kDa, about 250 kDa and about 500 kDa, about 250 kDa and about 450 kDa, about 250 kDa and about 400 kDa, about 250 kDa and about 350 kDa, about 250 kDa and about 300 kDa, about 300 kDa and about 700 kDa, about 300 kDa and about 650 kDa, about 300 kDa and about 600 kDa, about 300 kDa and about 550 kDa, about 300 kDa and about 500 kDa, about 300 kDa and about 450 kDa, about 300 kDa and about 400 kDa, about 300 kDa and about 350 kDa, about 350 kDa and about 700 kDa, about 350 kDa and about 650 kDa, about 350 kDa and about 600 kDa, about 350 kDa and about 550 kDa, about 350 kDa and about 500 kDa, about 350 kDa and about 450 kDa, about 350 kDa and about 400 kDa, about 400 kDa and about 700 kDa, about 400 kDa and about 650 kDa, about 400 kDa and about 600 kDa, about 400 kDa and about 550 kDa, about 400 kDa and about 500 kDa, about 400 kDa and about 450 kDa, about 450 kDa and about 700 kDa, about 450 kDa and about 650 kDa, about 450 kDa and about 600 kDa, about 450 kDa and about 550 kDa, about 450 kDa and about 500 kDa, about 500 kDa and about 700 kDa, about 500 kDa and about 650 kDa, about 500 kDa and about 600 kDa, about 500 kDa and about 550 kDa, about 550 kDa and about 700 kDa, about 550 kDa and about 650 kDa, about 550 kDa and about 600 kDa, about 600 kDa and about 700 kDa, about 600 kDa and about 650 kDa, or about 650 kDa and about 700 kDa. In some embodiments, the polypeptide-polysaccharide conjugates may have a molecular weight or average molecular weight of about 185, 200, 250, 300, 350, 400, 450, 500, 550, 600, 650, or 700 kDa. In some embodiments, the GAS polysaccharide, or a variant thereof, of the polypeptide-polysaccharide conjugates lacks an immunodominant N-acetyl Glucosamine (GlcNAc) side chain.

Using the long polysaccharides described herein may allow for the production of immunogenic compositions comprising reduced amounts of free GAS polysaccharides, or variants thereof. For instance, the immunogenic compositions may further comprise less than about 60%, 55%, 50%, 45%, 40%, 35, 30%, 25%, 20%, 15%, 10%, or 5% free GAS polysaccharide, or a variant thereof. In some embodiments, the immunogenic composition further comprises about 60%, 55%, 50%, 45%, 40%, 35, 30%, 25%, 20%, 15%, 10%, or 5% free GAS polysaccharide, or a variant thereof.

The immunogenic compositions described herein may be suitable for inducing a protective immune response against a Group A Streptococcus (GAS) bacterium in a subject comprising administering any of the immunogenic compositions described herein. In some embodiments, the immunogenic composition may induce an antibody response in a subject against the Group A Streptococcus (GAS) bacterium and does not induce an antibody response in the subject against human tissue. In some embodiments, the present disclosure provides for the use of the immunogenic compositions described herein in the manufacture of a medicament for inducing a protective immune response against a GAS bacterium in a subject. Also provided is the use of any of the immunogenic compositions described herein for inducing a protective immune response against a GAS bacterium in a subject. In some embodiments, the subject is 18 years or older. In some embodiments, the subject is less than 18 years old. In some embodiments, the subject is between 5 years and 17 years old, between 6 months and 9 years old, or between 5 years and 9 years old.

In some embodiments, the immunogenic compositions described herein comprise: (i) a Streptococcus pyogenes Adhesion and Division (SpyAD) conjugate polypeptide, or a fragment thereof, comprising at least one non-natural amino acid (nnAA), wherein the at least one nnAA comprises a click chemistry reactive group, and (ii) a GAS polysaccharide, or a variant thereof, that lacks an immunodominant N-acetyl Glucosamine (GlcNAc) side chain. In some embodiments, between about 8 mol % and about 20 mol % of the polysaccharide repeat units of the GAS polysaccharide, or a variant thereof, are derivatized by a linker. Additionally, in some embodiments, the average molecular weight of the polypeptide-polysaccharide conjugate is between about 185 kDa and about 700 kDa. In some embodiments, the average molecular weight of the polypeptide-polysaccharide conjugate is greater than about 185 kDa. In some embodiments, the average molecular weight of the polypeptide-polysaccharide conjugate is greater than about 190 kDa.

In some embodiments, an immunogenic composition may comprise: (a) a Group A Streptococcus (GAS) C5a peptidase polypeptide antigen that comprises or consists of the amino acid sequence of SEQ ID NO: 30, or a fragment thereof; (b) a GAS streptolysin O (SLO) polypeptide antigen that comprises or consists of the amino acid sequence of SEQ ID NO: 53, or a fragment thereof; and (c) a polypeptide-polysaccharide conjugate comprising: (i) a Streptococcus pyogenes Adhesion and Division (SpyAD) conjugate polypeptide that comprises or consists of the amino acid sequence of SEQ ID NO: 34, or a fragment thereof, and (ii) a GAS polysaccharide, or a variant thereof, that lacks an immunodominant N-acetyl Glucosamine (GlcNAc) side chain. In some embodiments, between about 8 mol % and about 20 mol % of the polysaccharide repeat units of the GAS polysaccharide, or a variant thereof, are derivatized by a linker. In some embodiments, the average molecular weight of the polypeptide-polysaccharide conjugate is between about 185 kDa and about 700 kDa. In some embodiments, the average molecular weight of the polypeptide-polysaccharide conjugate is between about 200 kDa and about 700 kDa. In certain embodiments, the average molecular weight is between about 300 kDa and about 600 kDa. In certain embodiments, the average molecular weight is between about 400 kDa and about 500 kDa. In some embodiments, the GAS polysaccharide, or a variant thereof, has a molecular weight of at least about 10 kDa to at least about 40 kDa. In some embodiments, the SpyAD conjugate polypeptide is a fragment of the amino acid sequence of SEQ ID NO: 34, and comprises or consists of the amino acid sequence of SEQ ID NO: 77, SEQ ID NO: 78, SEQ ID NO: 79, SEQ ID NO: 81, SEQ ID NO: 82, or SEQ ID NO: 83.

In some embodiments, an immunogenic composition described herein comprises (a) a Group A Streptococcus (GAS) C5a peptidase polypeptide antigen; (b) a GAS streptolysin O (SLO) polypeptide antigen; and (c) a polypeptide-polysaccharide conjugate comprising: (i) a Streptococcus pyogenes Adhesion and Division (SpyAD) conjugate polypeptide, or a fragment thereof, comprising at least one non-natural amino acid (nnAA), wherein the at least one nnAA comprises a click chemistry reactive group; and (ii) a GAS polysaccharide, or a variant thereof, that lacks an immunodominant N-acetyl Glucosamine (GlcNAc) side chain; wherein the average molecular weight of the polypeptide-polysaccharide conjugate is between about 185 kDa and about 700 kDa.

In some embodiments, an immunogenic composition comprises (a) a Group A Streptococcus (GAS) C5a peptidase polypeptide antigen; (b) a GAS streptolysin O (SLO) polypeptide antigen; and (c) a polypeptide-polysaccharide conjugate comprising: (i) a Streptococcus pyogenes Adhesion and Division (SpyAD) conjugate polypeptide, or a fragment thereof, comprising at least one non-natural amino acid (nnAA), wherein the at least one nnAA comprises a click chemistry reactive group; and (ii) a GAS polysaccharide, or a variant thereof, that lacks an immunodominant N-acetyl Glucosamine (GlcNAc) side chain; wherein between about 8 mol % and about 20 mol % of the polysaccharide repeat units of the GAS polysaccharide, or a variant thereof, are derivatized by a linker.

In some embodiments, an immunogenic composition described herein comprises (a) a Group A Streptococcus (GAS) C5a peptidase polypeptide antigen; (b) a GAS streptolysin O (SLO) polypeptide antigen; and (c) a polypeptide-polysaccharide conjugate comprising: (i) a Streptococcus pyogenes Adhesion and Division (SpyAD) conjugate polypeptide, or a fragment thereof, comprising at least one non-natural amino acid (nnAA), wherein the at least one nnAA comprises a click chemistry reactive group; and (ii) a GAS polysaccharide, or a variant thereof, that lacks an immunodominant N-acetyl Glucosamine (GlcNAc) side chain; wherein between about 8 mol % and about 20 mol % of the polysaccharide repeat units of the GAS polysaccharide, or a variant thereof, are derivatized by a linker; and wherein the average molecular weight of the polypeptide-polysaccharide conjugate is between about 185 kDa and about 700 kDa.

### ENUMERATED EMBODIMENTS

Embodiment I-1. A process for purifying cell wall polysaccharides or peptidoglycan-bound capsular polysaccharides from a bacterial cell, the process comprising:
(a) hydrolyzing the bacterial cell in a solution comprising base and a reducing agent to form a lysate comprising polysaccharide; and
(b) incubating the lysate comprising polysaccharide with a muralytic enzyme to form a free polysaccharide solution.

Embodiment I-2. The process according to embodiment I-1, wherein the bacterial cell is a Pseudomonas bacterial cell, a Streptococcus bacterial cell, a Staphylococcus bacterial cell, a Neisseria bacterial cell, a Haemophilus bacterial cell, a Listeria bacterial cell, a Enterococcus bacterial cell, or a Clostridium bacterial cell.

Embodiment I-3. The process according to embodiment I-1 or I-2, wherein the bacterial cell is selected from of Pseudomonas aeruginosa, Streptococcus viridans, Streptococcus mutans, or Streptococcus pyogenes, [etc].

Embodiment I-4. The process according to embodiment I-3, wherein the Streptococcus pyogenes bacterial cell is of a serotype selected from M1, M2, M3, M4, M5, M6, M9, M11, M12, M13, M18, M22, M25, M28, M62, M71, M72, M74, M75, M77, M80, M81, M83, M87, M89, or M92.

Embodiment I-5. The process according to embodiment I-3, wherein the Streptococcus pyogenes bacterial cell produces a polysaccharide or a variant thereof that lacks an immunodominant N-acetyl Glucosamine (GlcNAc) side chain.

Embodiment I-6. The process according to embodiment I-1, for purifying a peptidoglycan-bound capsular polysaccharide from a bacterial cell.

Embodiment I-7. The process according to any one of embodiments I-1 to I-6, wherein the base of step (a) is NaOH, KOH, or LiOH.

Embodiment I-8. The process according to any one of embodiments I-1 to I-7, wherein the base of step (a) is NaOH.

Embodiment I-9. The process according to any one of embodiments I-1 to I-8, wherein the concentration of base is between about 2M to about 8M.

Embodiment I-10. The process according to any one of embodiments I-1 to I-9, wherein the solution comprising base and a reducing agent is about pH 14.

Embodiment I-11. The process according to any one of embodiments I-1 to I-10, wherein the reducing agent is sodium borohydride, sodium cyanoborohydride, sodium triacetoxyborohydride, dithiothreitol, or beta-mercaptoethanol.

Embodiment I-12. The process according to any one of embodiments I-1 to I-11, wherein the reducing agent is sodium borohydride.

Embodiment I-13. The process according to any one of embodiments I-1 to I-12, wherein the concentration of the reducing agent is between about 1 mM and 500 mM.

Embodiment I-14. The process according to any one of embodiments I-1 to I-13, wherein step (a) further comprises incubating the solution between about 30° C. and about 100° C.

Embodiment I-15. The process according to any one of embodiments I-1 to I-14, wherein step (a) further comprises incubating the solution for between about 0.5 to about 20 hours.

Embodiment I-16. The process according to any one of embodiments I-1 to I-15, wherein step (a) further comprises one or more pH adjustment steps.

Embodiment I-17. The process according to embodiment I-16, wherein the one or more pH adjustment steps are independently selected from:
(i) raising the lysate comprising polysaccharide pH, or
(ii) lowering the lysate comprising polysaccharide pH.

Embodiment I-18. The process according to embodiment I-17, comprising lowering the lysate comprising polysaccharide pH to between about 3 and 7.0.

Embodiment I-19. The process according to embodiments I-17 or I-18, comprising lowering the lysate comprising polysaccharide pH to about 6.5.

Embodiment I-20. The process according to embodiments I-17 or I-18, comprising lowering the lysate comprising polysaccharide pH to between about 3 and about 4.

Embodiment I-21. The process according to embodiments I-16 or I-17, comprising
(i) lowering the lysate comprising polysaccharide pH to between about 5.5 and 7.0;
(ii) lowering the lysate comprising polysaccharide pH to about 3; and
(iii) raising the lysate comprising polysaccharide pH to between about 5.5 and 7.0.

Embodiment I-22. The process according to any one of embodiments I-16 to I-21, wherein the lysate comprising polysaccharide is incubated at about room temperature (r.t.) after the one or more pH adjustment steps.

Embodiment I-23. The process according to any one of embodiments I-17 to I-20, wherein the lysate comprising polysaccharide is incubated at between about 4° C. and about 30° C. after the one or more pH adjustment steps.

Embodiment I-24. The process according to any one of embodiments I-1 to I-23, further comprising removing solids from the lysate comprising polysaccharide.

Embodiment I-25. The process according to embodiment I-24, wherein removing solids from the lysate comprising polysaccharide comprises filtration, centrifugation, or a combination thereof.

Embodiment I-26. The process according to embodiment I-25, wherein the filtration comprises depth filtration, tangential flow filtration (TFF), sterile filtration, or a combination of the foregoing.

Embodiment I-27. The process according to embodiments I-25 or I-26, wherein the filtration comprises depth filtration followed by TFF.

Embodiment I-28. The process according to embodiments I-24 or I-25, wherein solids are removed from the lysate comprising polysaccharide by centrifugation.

Embodiment I-29. The process according to any one of embodiments I-1 to I-28, wherein the muralytic enzyme of step (b) is mutanolysin, lysozyme, or a bacteriophage hydrolase.

Embodiment I-30. The process according to any one of embodiments I-1 to I-29, wherein step (b) further comprises incubating with a protease.

Embodiment I-31. The process according to embodiment I-30, wherein the protease is proteinase K, trypsin, chymotrypsin, endoproteinase Asp-N, endoproteinase Arg-C, endoproteinase Glu-C, endoproteinase Lys-C, pepsin, thermolysin, elastase, papain, substilisin, clostripain, carboxypeptidase A, carboxypeptidase B, carboxypeptidase P, carboxypeptidase Y, cathepsin C, acylamino-acid releasing enzyme, or pyroglutamate.

Embodiment I-32. The process according to any one of embodiments I-1 to I-31, wherein step (b) further comprises warming the lysate comprising polysaccharide with the muralytic enzyme to between about 30° C. and about 65° C.

Embodiment I-33. The process according to any one of embodiments I-30 to I-32, wherein the lysate comprising polysaccharide with the protease is warmed to between about 45° C. and 55° C.

Embodiment I-34. The process according to embodiments I-32 or I-33, wherein the lysate is warmed between about 6 and about 20 hours.

Embodiment I-35. The process according to any one of embodiments I-1 to I-34, wherein the free polysaccharide solution of step (b) is further purified to reduce the concentration of nucleic acids, enzymes, host cell proteins (HCPs), or a combination of the foregoing.

Embodiment I-36. The process according to any one of embodiments I-1 to I-35, wherein the free polysaccharide solution of step (b) is further purified by precipitation.

Embodiment I-37. The process according to any one of embodiments I-1 to I-36, wherein the free polysaccharide solution of step (b) is treated with cetyltrimethylammonium bromide (CTAB).

Embodiment I-38. The process according to embodiment I-37, wherein the concentration of CTAB in the free polysaccharide solution is about 0.10% to about 10%.

Embodiment I-39. The process according to embodiments I-37 or 38, the concentration of CTAB is between about 0.5% and about 3%.

Embodiment I-40. The process according to any one of embodiments I-37 to I-39, wherein the free polysaccharide solution of step (b) is treated with potassium iodide (KI).

Embodiment I-41. The process according to embodiment I-32, wherein the concentration of KI in the free polysaccharide solution is between about 20 mM to about 400 mM.

Embodiment I-42. The process according to any one of embodiments I-1 to I-41, wherein the free polysaccharide solution is further purified by filtration, centrifugation, chromatography, or a combination of the foregoing.

Embodiment I-43. The process according to embodiment I-42, wherein the filtration comprises depth filtration, tangential flow filtration (TFF), sterile filtration, or a combination of the foregoing.

Embodiment I-44. The process according to embodiment I-42, wherein the chromatography comprises hydrophobic interaction chromatography (HIC), anion-exchange chromatography (AEX), ceramic hydroxyapatite-type chromatography, or cation exchange chromatography (CEX).

Embodiment I-45. A polypeptide-polysaccharide conjugate comprising:
(a) a GAS polypeptide antigen or a non-GAS carrier polypeptide comprising at least one non-natural amino acid (nnAA); and
(b) a purified cell wall polysaccharide or a peptidoglycan-bound capsular polysaccharide with a molecular weight of at least about 10 kDa to at least about 40 kDa.

Embodiment I-46. The polypeptide-polysaccharide conjugate of embodiment I-45, wherein the purified cell wall polysaccharide lacks an immunodominant N-acetyl Glucosamine (GlcNAc) side chain.

Embodiment I-47. The polypeptide-polysaccharide conjugate of embodiments I-45 to I-46, wherein the polypeptide antigen is a full-length GAS polypeptide antigen or a fragment of a full-length GAS polypeptide antigen.

Embodiment I-48. The polypeptide-polysaccharide conjugate of any one of embodiments I-45 to I-47, wherein the at least one nnAA is substituted for a lysine, a leucine, an isoleucine, or an arginine in the polypeptide antigen or the non-GAS carrier polypeptide.

Embodiment I-49. The polypeptide-polysaccharide conjugate of any one of embodiments I-45 to I-48, wherein the nnAA comprises a click chemistry reactive group.

Embodiment I-50. The polypeptide-polysaccharide conjugate of any one of embodiments I-45 to I-49, wherein the nnAA is selected from 2-amino-3-(4-azidophenyl)propanoic acid (pAF), 2-amino-4-azidobutanoic acid, 2-azido-3-phenylpropionic acid, 2-amino-3-azidopropanoic acid, 2-amino-3-(4-(azidomethyl)phenyl)propanoic acid (pAMF), 2-amino-3-(5-(azidomethyl)pyridin-2-yl)propanoic acid, 2-amino-3-(4-(azidomethyl)pyridin-2-yl)propanoic acid, 2-amino-3-(6-(azidomethyl)pyridin-3-yl)propanoic acid, and 2-amino-5-azidopentanoic acid.

Embodiment I-51. The polypeptide-polysaccharide conjugate of any one of embodiments I-45 to I-50, wherein the nnAA is pAMF.

Embodiment I-52. The polypeptide-polysaccharide conjugate of any one of embodiments I-45 to I-51, wherein the polypeptide antigen is selected from C5a peptidase, streptolysin O (SLO), SpyAD, Sib35, and Sfb1.

Embodiment I-53. The polypeptide-polysaccharide conjugate of any one of embodiments I-45 to I-52, wherein the polypeptide antigen is SLO.

Embodiment I-54. The polypeptide-polysaccharide conjugate of embodiment I-53, wherein the SLO polypeptide antigen comprises an amino acid sequence that is at least 95% identical to SEQ ID NO: 31, SEQ ID NO: 32, or SEQ ID NO: 53.

Embodiment I-55. The polypeptide-polysaccharide conjugate of embodiment I-53, wherein the SLO polypeptide antigen is at least 95% identical to SEQ ID NO: 31, SEQ ID NO: 32 or SEQ ID NO: 53.

Embodiment I-56. The polypeptide-polysaccharide conjugate of any one of embodiments I-53 to I-55, wherein the SLO polypeptide comprises 3 or 4 pAMF substitutions at positions selected from K98, K112, R151, K189, K272, K323, K357, K375, K407, or K464.

Embodiment I-57. The polypeptide-polysaccharide conjugate of any one of embodiments I-53 to I-56, wherein the SLO polypeptide comprises the amino acid sequence of SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, or SEQ ID NO: 64.

Embodiment I-58. The polypeptide-polysaccharide conjugate of any one of embodiments I-53 to I-56, wherein the SLO polypeptide has the amino acid sequence of SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, or SEQ ID NO: 64.

Embodiment I-59. The polypeptide-polysaccharide conjugate of any one of embodiment I-53 to I-55, wherein the SLO polypeptide comprises 5, 6, 7, or 8 pAM F substitutions at positions selected from K98, K112, R151, K189, K272, K323, K357, K375, K407, or K464

Embodiment I-60. The polypeptide-polysaccharide conjugate of any one of embodiments I-53 to I-55 or 59, wherein the SLO polypeptide comprises the amino acid sequence of SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, or SEQ ID NO: 76.

Embodiment I-61. The polypeptide-polysaccharide conjugate of any one of embodiments I-53 to I-55 or 59-60, wherein the SLO polypeptide has the amino acid sequence of SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, or SEQ ID NO: 76.

Embodiment I-62. The polypeptide-polysaccharide conjugate of any one of embodiments I-45 to I-52, wherein the polypeptide antigen is a SpyAD polypeptide.

Embodiment I-63. The polypeptide-polysaccharide conjugate of embodiment I-62, wherein the SpyAD polypeptide comprises an amino acid sequence that is at least 95% identical to SEQ ID NO: 33.

Embodiment I-64. The polypeptide-polysaccharide conjugate of embodiment I-62, wherein the SpyAD polypeptide is at least 95% identical to SEQ ID NO: 33.

Embodiment I-65. The polypeptide-polysaccharide conjugate of any one of embodiments I-62 to I-64, wherein the SpyAD polypeptide comprises a pAMF substitution at positions K64, K287, K386, and K657 of SEQ ID NO: 33.

Embodiment I-66. The polypeptide-polysaccharide conjugate of any one of embodiments I-62 to I-65, wherein the SpyAD polypeptide comprises the amino acid sequence of SEQ ID NO: 34.

Embodiment I-67. The polypeptide-polysaccharide conjugate of any one of embodiments I-62 to I-65, wherein the SpyAD polypeptide has the amino acid sequence of SEQ ID NO: 34.

Embodiment I-68. The polypeptide-polysaccharide conjugate of any one of embodiment I-45 to I-45 or 48-51, wherein the non-GAS carrier polypeptide is selected from ADI, ferritin, Protein D, and eCRM197.

Embodiment I-69. The polypeptide-polysaccharide conjugate of embodiment I-68, wherein the non-GAS carrier polypeptide is eCRM197.

Embodiment I-70. The polypeptide-polysaccharide conjugate of embodiment I-69, wherein the eCRM197 has the sequence of SEQ ID NO: 25.

Embodiment I-71. The polypeptide-polysaccharide conjugate of any one of embodiments I-45 to I-50, wherein the GAS polypeptide antigen or a non-GAS carrier polypeptide comprises or consists comprises or consists of the amino acid sequence of a polypeptide listed in Table 1.

Embodiment II-1. A polypeptide-polysaccharide conjugate comprising:
(a) a GAS polypeptide antigen or a non-GAS carrier polypeptide comprising at least one non-natural amino acid (nnAA), wherein the nnAA comprises a click chemistry reactive group; and
(b) a purified cell wall polysaccharide or a peptidoglycan-bound capsular polysaccharide with a molecular weight of at least about 10 kDa to at least about 40 kDa.

Embodiment II-2. The polypeptide-polysaccharide conjugate of embodiment II-1, wherein the purified cell wall polysaccharide lacks an immunodominant N-acetyl Glucosamine (GlcNAc) side chain.

Embodiment II-3. The polypeptide-polysaccharide conjugate of embodiments II-1 to II-2, wherein the GAS polypeptide antigen is a full-length GAS polypeptide antigen or a fragment of a full-length GAS polypeptide antigen.

Embodiment II-4. The polypeptide-polysaccharide conjugate of any one of embodiments II-1 to II-3, wherein the cell wall polysaccharide is a GAS polysaccharide, or a variant thereof.

Embodiment II-5. The polypeptide-polysaccharide conjugate of embodiment II-4, wherein between about 8 mol % and about 20 mol % of the polysaccharide repeat units of the GAS polysaccharide, or a variant thereof, are derivatized by a linker.

Embodiment II-6. The polypeptide-polysaccharide conjugate of embodiments II-4 to II-5, wherein between about 10 mol % and about 20 mol % of the polysaccharide repeat units of the GAS polysaccharide, or a variant thereof, are derivatized by a linker.

Embodiment II-7. The polypeptide-polysaccharide conjugate of embodiments II-4 to II-6, wherein between about 10 mol % and about 18 mol % of the polysaccharide repeat units of the GAS polysaccharide, or a variant thereof, are derivatized by a linker.

Embodiment II-8. The polypeptide-polysaccharide conjugate of any one of embodiments II-1 to II-6, wherein the average molecular weight is between about 185 kDa and about 700.

Embodiment II-9. The polypeptide-polysaccharide conjugate of any one of embodiments II-1 to II-7, wherein the average molecular weight is between about 200 kDa and about 700 kDa.

Embodiment II-10. The polypeptide-polysaccharide conjugate of any one of embodiments II-1 to II-9, wherein the average molecular weight of the polypeptide-polysaccharide conjugate is between about 300 kDa and about 600 kDa.

Embodiment II-11. The polypeptide-polysaccharide conjugate of any one of embodiments II-1 to II-10, wherein the average molecular weight of the polypeptide-polysaccharide conjugate is between about 400 kDa and about 500 kDa.

Embodiment II-12. The polypeptide-polysaccharide conjugate of any one of embodiments II-1 to II-11, wherein the at least one nnAA is substituted for a lysine, a leucine, an isoleucine, or an arginine in the GAS polypeptide antigen or the non-GAS carrier polypeptide.

Embodiment II-13. The polypeptide-polysaccharide conjugate of any one of embodiments 11-1 to 11-12, wherein the at least one nnAA is selected from 2-amino-3-(4-azidophenyl)propanoic acid (pAF), 2-amino-4-azidobutanoic acid, 2-azido-3-phenylpropionic acid, 2-amino-3-azidopropanoic acid, 2-amino-3-(4-(azidomethyl)phenyl)propanoic acid (pAMF), 2-amino-3-(5-(azidomethyl)pyridin-2-yl)propanoic acid, 2-amino-3-(4-(azidomethyl)pyridin-2-yl)propanoic acid, 2-amino-3-(6-(azidomethyl)pyridin-3-yl)propanoic acid, and 2-amino-5-azidopentanoic acid.

Embodiment II-14. The polypeptide-polysaccharide conjugate of any one of embodiments II-1 to II-13, wherein the at least one nnAA is pAMF.

Embodiment II-15. The polypeptide-polysaccharide conjugate of any one of embodiments II-1 to II-14, wherein the GAS polypeptide antigen is selected from C5a peptidase, streptolysin O (SLO), SpyAD, Sib35, and Sfb1.

Embodiment II-15a. The polypeptide-polysaccharide conjugate of any one of embodiments II-1 to II-15, wherein the GAS polypeptide antigen is SLO.

Embodiment II-15b. The polypeptide-polysaccharide conjugate of embodiment II-15a, wherein the SLO polypeptide antigen comprises an amino acid sequence that is at least 95% identical to SEQ ID NO: 31, SEQ ID NO: 32, or SEQ ID NO: 53.

Embodiment II-15c. The polypeptide-polysaccharide conjugate of embodiment II-15b, wherein the SLO polypeptide antigen is at least 95% identical to SEQ ID NO: 31, SEQ ID NO: 32 or SEQ ID NO: 53.

Embodiment II-15d. The polypeptide-polysaccharide conjugate of any one of embodiments II-15a to II-15c, wherein the SLO polypeptide comprises 3 or 4 pAMF substitutions at positions selected from K98, K112, R151, K189, K272, K323, K357, K375, K407, or K464.

Embodiment II-15e. The polypeptide-polysaccharide conjugate of any one of embodiments II-15a-15d, wherein the SLO polypeptide comprises the amino acid sequence of SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, or SEQ ID NO: 64.

Embodiment II-15f. The polypeptide-polysaccharide conjugate of any one of embodiments II-15a to II-15d, wherein the SLO polypeptide has the amino acid sequence of SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, or SEQ ID NO: 64.

Embodiment II-15g. The polypeptide-polysaccharide conjugate of any one of embodiments II-15a to II-15c, wherein the SLO polypeptide comprises 5, 6, 7, or 8 pAMF substitutions at positions selected from K98, K112, R151, K189, K272, K323, K357, K375, K407, or K464

Embodiment II-15h. The polypeptide-polysaccharide conjugate of any one of embodiments II-15a to II-15c or II-22, wherein the SLO polypeptide comprises the amino acid sequence of SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, or SEQ ID NO: 76.

Embodiment II-15i. The polypeptide-polysaccharide conjugate of any one of embodiments II-15a to II-15c or II-15g to II-15f, wherein the SLO polypeptide has the amino acid sequence of SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, or SEQ ID NO: 76.

Embodiment II-16. The polypeptide-polysaccharide conjugate of any one of embodiments II-1 to II-15 or II-15a to II-15i, wherein the GAS polypeptide antigen is a SpyAD polypeptide, or a fragment thereof.

Embodiment II-17. The polypeptide-polysaccharide conjugate of embodiment II-16, wherein the SpyAD polypeptide comprises an amino acid sequence that is at least 95% identical to SEQ ID NO: 33.

Embodiment II-18. The polypeptide-polysaccharide conjugate of embodiment II-16, wherein the SpyAD polypeptide is at least 95% identical to SEQ ID NO: 33.

Embodiment II-19. The polypeptide-polysaccharide conjugate of any one of embodiments II-16 to II-18, wherein the SpyAD polypeptide comprises a pAMF substitution at positions K64, K287, K386, and K657 of SEQ ID NO: 33.

Embodiment II-20. The polypeptide-polysaccharide conjugate of any one of embodiments II-16 to II-19, wherein the SpyAD polypeptide comprises the amino acid sequence of SEQ ID NO: 34.

Embodiment II-21. The polypeptide-polysaccharide conjugate of any one of embodiments II-16 to II-19, wherein the SpyAD polypeptide has the amino acid sequence of SEQ ID NO: 34.

Embodiment II-22. The polypeptide-polysaccharide conjugate of any one of embodiments II-16 to II-19, wherein the SpyAD polypeptide comprises the amino acid sequence of SEQ ID NO: 77, SEQ ID NO: 78, SEQ ID NO: 79, SEQ ID NO: 81, SEQ ID NO: 82, or SEQ ID NO: 83.

Embodiment II-23. The polypeptide-polysaccharide conjugate of any one of embodiments II-16 to II-19, wherein the SpyAD polypeptide has the amino acid sequence of SEQ ID NO: 77, SEQ ID NO: 78, SEQ ID NO: 79, SEQ ID NO: 81, SEQ ID NO: 82, or SEQ ID NO: 83.

Embodiment II-24. The polypeptide-polysaccharide conjugate of any one of embodiments II-1 to II-2 or II-4 to II-14, wherein the non-GAS carrier polypeptide is selected from ADI, ferritin, Protein D, and eCRM197.

Embodiment II-25. The polypeptide-polysaccharide conjugate of embodiment II-24, wherein the non-GAS carrier polypeptide is eCRM197.

Embodiment II-26. The polypeptide-polysaccharide conjugate of embodiment II-25, wherein the eCRM197 has the sequence of SEQ ID NO: 25.

Embodiment II-27. The polypeptide-polysaccharide conjugate of any one of embodiments II-1 to II-13, wherein the GAS polypeptide antigen or a non-GAS carrier polypeptide comprises or consists comprises or consists of the amino acid sequence of a polypeptide listed in Table 1.

Embodiment II-28. The polypeptide-polysaccharide conjugate of any one of embodiments II-1 to II-13, wherein the GAS polypeptide antigen or a non-GAS carrier polypeptide comprises or consists comprises or consists of the amino acid sequence of a polypeptide listed in Table 1A.

Embodiment II-29. An immunogenic composition comprising:
(a) a Group A Streptococcus (GAS) C5a peptidase polypeptide antigen;
(b) a GAS streptolysin O (SLO) polypeptide antigen; and
(c) a polypeptide-polysaccharide conjugate comprising
   a Streptococcus pyogenes Adhesion and Division (SpyAD) conjugate polypeptide, or a fragment thereof, comprising at least one non-natural amino acid (nnAA), wherein the at least one nnAA comprises a click chemistry reactive group, and
   a GAS polysaccharide, or a variant thereof, that lacks an immunodominant N-acetyl Glucosamine (GlcNAc) side chain;
   wherein between about 8 mol % and about 20 mol % of the polysaccharide repeat units of the GAS polysaccharide, or a variant thereof, are derivatized by a linker; and
   wherein the average molecular weight of the polypeptide-polysaccharide conjugate is between about 185 kDa and about 700 kDa.

Embodiment II-30. The immunogenic composition of embodiment II-29, wherein the C5a peptidase polypeptide antigen comprises the amino acid sequence of SEQ ID NO: 29 or SEQ ID NO: 30.

Embodiment II-31. The immunogenic composition of embodiment II-29 or 11-30, wherein the C5a peptidase polypeptide antigen comprises an amino acid sequence that is at least 95% identical to SEQ ID NO: 29 or SEQ ID NO: 30.

Embodiment II-32. The immunogenic composition of any one of embodiments II-29 to II-31, wherein the C5a peptidase polypeptide antigen has the amino acid sequence of SEQ ID NO: 29 or SEQ ID NO: 30.

Embodiment II-33. The immunogenic composition of any one of embodiments II-29 to II-32, wherein the SLO polypeptide antigen comprises the amino acid sequence of SEQ ID NO: 31, SEQ ID NO: 32, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53.

Embodiment II-34. The immunogenic composition of embodiments 11-29 to 11-33, wherein the SLO antigen comprises an amino acid sequence that is at least 95% identical to SEQ ID NO: 31, SEQ ID NO: 32, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53.

Embodiment II-35. The immunogenic composition of any one of embodiments II-29 to II-34, wherein the SLO polypeptide antigen has the amino acid sequence of SEQ ID NO: 31, SEQ ID NO: 32, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53.

Embodiment II-36. The immunogenic composition of any one of embodiments II-29 to 11-35, wherein the at least one nnAA is selected from 2-amino-3-(4-azidophenyl)propanoic acid (pAF), 2-amino-4-azidobutanoic acid, 2-azido-3-phenylpropionic acid, 2-amino-3-azidopropanoic acid, 2-amino-3-(4-(azidomethyl)phenyl)propanoic acid (pAMF), 2-amino-3-(5-(azidomethyl)pyridin-2-yl)propanoic acid, 2-amino-3-(4-(azidomethyl)pyridin-2-yl)propanoic acid, 2-amino-3-(6-(azidomethyl)pyridin-3-yl)propanoic acid, and 2-amino-5-azidopentanoic acid.

Embodiment II-37. The immunogenic composition of any one of embodiments II-29 to II-36, wherein the at least one nnAA is pAMF.

Embodiment II-38. The immunogenic composition of any one of embodiments II-29 to II-37, wherein the SpyAD conjugate polypeptide, or a fragment thereof, comprises an amino acid sequence that is at least 95% identical to SEQ ID NO: 33.

Embodiment II-39. The immunogenic composition of any one of embodiments II-29 to II-38, wherein the SpyAD conjugate polypeptide comprises an amino acid sequence that is a fragment of SEQ ID NO: 33.

Embodiment II-40. The immunogenic composition of any one of embodiments II-29 to II-38, wherein the SpyAD conjugate polypeptide has an amino acid sequence that is a fragment of SEQ ID NO: 33.

Embodiment II-41. The immunogenic composition of any one of embodiments II-29 to II-40, wherein the SpyAD conjugate polypeptide comprises a pAMF substitution at positions K64, K287, K386, and K657 of SEQ ID NO: 33.

Embodiment II-42. The immunogenic composition of any one of embodiments II-29 to II-41, wherein the SpyAD conjugate polypeptide comprises the amino acid sequence of SEQ ID NO: 34, SEQ ID NO: 77, SEQ ID NO: 78, SEQ ID NO: 79, SEQ ID NO: 81, SEQ ID NO: 82, or SEQ ID NO: 83.

Embodiment II-43. The immunogenic composition of any one of embodiments II-29 to II-42, wherein the SpyAD conjugate polypeptide comprises an amino acid sequence that is at least 95% identical to SEQ ID NO: 34, SEQ ID NO: 77, SEQ ID NO: 78, SEQ ID NO: 79, SEQ ID NO: 81, SEQ ID NO: 82, or SEQ ID NO: 83.

Embodiment II-44. The immunogenic composition of any one of embodiments II-29 to II-43, wherein the SpyAD conjugate polypeptide has the amino acid sequence of SEQ ID NO: 34, SEQ ID NO: 77, SEQ ID NO: 78, SEQ ID NO: 79, SEQ ID NO: 81, SEQ ID NO: 82, or SEQ ID NO: 83.

Embodiment II-45. The immunogenic composition of any one of embodiments II-29 to II-44, wherein the C5a peptidase polypeptide antigen comprises an amino acid sequence that is at least 95% identical to SEQ ID NO: 30; the SLO polypeptide antigen comprises an amino acid sequence that is at least 95% identical to SEQ ID NO: 53; and the SpyAD conjugate polypeptide comprises an amino acid sequence that is at least 95% identical to SEQ ID NO: 34.

Embodiment II-46. The immunogenic composition of any one of embodiments II-29 to II-45 wherein the C5a peptidase polypeptide antigen comprises the amino acid sequence of SEQ ID NO: 30; the SLO polypeptide antigen comprises the amino acid sequence of SEQ ID NO: 53; and the SpyAD conjugate polypeptide comprises the amino acid sequence of SEQ ID NO: 34.

Embodiment II-47. The immunogenic composition of any one of embodiments II-29 to II-46, wherein the C5a peptidase polypeptide antigen has the amino acid sequence of SEQ ID NO: 30; the SLO polypeptide antigen has the amino acid sequence of SEQ ID NO: 53; and the SpyAD conjugate polypeptide has the amino acid sequence of SEQ ID NO: 34.

Embodiment II-48. The immunogenic composition of any one of embodiments II-29 to II-47, wherein between about 15 mol % and about 20 mol % of the polysaccharide repeat units of the GAS polysaccharide, or a variant thereof, are derivatized by a linker.

Embodiment II-49. The immunogenic composition of any one of embodiments II-29 to II-48, wherein the linker, prior to reaction with the click chemistry reactive group of the nnAA, comprises a structure of Formula I:

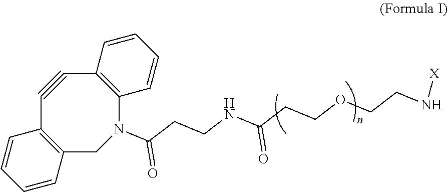

wherein, X is at least one polysaccharide repeat unit of a GAS polysaccharide, or a fragment thereof; and n is at least 1.

Embodiment II-50. The immunogenic composition of any one of embodiments II-29 to II-49, wherein the SpyAD conjugate polypeptide, or fragment thereof, is linked to the GAS polysaccharide according to Formula II:

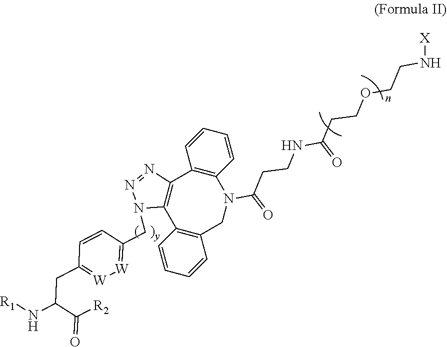

wherein,
R₁ is H, formyl, or at least one amino acid of the SpyAD conjugate polypeptide;
R₂ is OH or at least one amino acid of the SpyAD conjugate polypeptide;
W is CH or N;
y is at least 1;
n is at least 1; and
X is at least one polysaccharide repeat unit of a GAS polysaccharide or variant thereof.

Embodiment II-51. The immunogenic composition of any one of embodiments II-29 to II-50, wherein the GAS polysaccharide, or a variant thereof, has a molecular weight of at least about 10 kDa to at least about 40 kDa.

Embodiment II-52. The immunogenic composition of any one of embodiments II-29 to II-51, wherein the average molecular weight of the polypeptide-polysaccharide conjugate is between about 185 kDa and about 700.

Embodiment II-53. The immunogenic composition of any one of embodiments II-29 to II-52, wherein the average molecular weight of the polypeptide-polysaccharide conjugate is between about 200 kDa and about 700 kDa.

Embodiment II-54. The immunogenic composition of any one of embodiments II-29 to II-53, wherein the average molecular weight of the polypeptide-polysaccharide conjugate is between about 300 kDa and about 700 kDa.

Embodiment II-55. The immunogenic composition of any one of embodiments II-29 to II-54, wherein the average molecular weight of the polypeptide-polysaccharide conjugate is between about 300 kDa and about 600 kDa.

Embodiment II-56. The immunogenic composition of any one of embodiments II-29 to II-55 further comprising less than about 60% free GAS polysaccharide, or a variant thereof.

Embodiment II-57. The immunogenic composition of any one of embodiments II-29 to II-56 further comprising less than about 50% free GAS polysaccharide, or a variant thereof.

Embodiment II-58. The immunogenic composition of any one of embodiments II-29 to II-57 further comprising less than about 25% free GAS polysaccharide, or a variant thereof.

Embodiment II-59. The immunogenic composition of any one of embodiments II-29 to II-58 further comprising less than about 15% free GAS polysaccharide, or a variant thereof.

Embodiment II-60. The immunogenic composition of any one of embodiments II-29 to II-59 further comprising less than about 10% free GAS polysaccharide, or a variant thereof.

Embodiment II-61. A method of inducing a protective immune response against a Group A Streptococcus (GAS) bacterium in a subject comprising administering the immunogenic composition of any one of embodiments II-29 to II-60 to the subject.

Embodiment II-62. The method of embodiment II-61, wherein the immunogenic composition induces an antibody response in the subject against the Group A Streptococcus (GAS) bacterium and does not induce an antibody response in the subject against human tissue.

Embodiment II-63. The use of the immunogenic composition of any one of embodiments II-29 to II-60 in the manufacture of a medicament for inducing a protective immune response against a GAS bacterium in a subject.

Embodiment II-64. Use of the immunogenic composition of any one of embodiments II-29 to II-60 for inducing a protective immune response against a GAS bacterium in a subject.

Embodiment II-65. A process for purifying cell wall polysaccharides or peptidoglycan-bound capsular polysaccharides from a bacterial cell, the process comprising:
(a) hydrolyzing the bacterial cell in a solution comprising base and a reducing agent to form a lysate comprising polysaccharide; and
(b) incubating the lysate comprising polysaccharide with a muralytic enzyme to form a free polysaccharide solution.

Embodiment II-66. The process according to embodiment II-65, wherein the bacterial cell is a Pseudomonas bacterial cell, a Streptococcus bacterial cell, a Staphylococcus bacterial cell, a Neisseria bacterial cell, a Haemophilus bacterial cell, a Listeria bacterial cell, a Enterococcus bacterial cell, or a Clostridium bacterial cell.

Embodiment II-67. The process according to embodiment II-65 or II-66, wherein the bacterial cell is selected from of Pseudomonas aeruginosa, Streptococcus viridans, Streptococcus mutans, and Streptococcus pyogenes.

Embodiment II-68. The process according to embodiment II-67, wherein the Streptococcus pyogenes bacterial cell is of a serotype selected from M1, M2, M3, M4, M5, M6, M9, M11, M12, M13, M18, M22, M25, M28, M62, M71, M72, M74, M75, M77, M80, M81, M83, M87, M89, or M92.

Embodiment II-69. The process according to embodiment II-68, wherein the Streptococcus pyogenes bacterial cell produces a polysaccharide or a variant thereof that lacks an immunodominant N-acetyl Glucosamine (GlcNAc) side chain.

Embodiment II-70. The process according to embodiment II-65, for purifying a peptidoglycan-bound capsular polysaccharide from a bacterial cell.

Embodiment II-71. The process according to any one of embodiments II-65 to II-70, wherein the base of step (a) is NaOH, KOH, or LiOH.

Embodiment II-72. The process according to any one of embodiments II-65 to II-71, wherein the base of step (a) is NaOH.

Embodiment II-73. The process according to any one of embodiments II-65 to II-72, wherein the concentration of base is between about 2M to about 8M.

Embodiment II-74. The process according to any one of embodiments II-65 to II-73, wherein the solution comprising base and a reducing agent is about pH 14.

Embodiment II-75. The process according to any one of embodiments II-65 to II-72, wherein the reducing agent is sodium borohydride, sodium cyanoborohydride, sodium triacetoxyborohydride, dithiothreitol, or beta-mercaptoethanol.

Embodiment II-76. The process according to any one of embodiments II-65 to II-73, wherein the reducing agent is sodium borohydride.

Embodiment II-77. The process according to any one of embodiments II-65 to II-76, wherein the concentration of the reducing agent is between about 1 mM and 500 mM.

Embodiment II-78. The process according to any one of embodiments II-65 to II-75, wherein step (a) further comprises incubating the solution between about 30° C. and about 100° C.

Embodiment II-79. The process according to any one of embodiments II-65 to II-78, wherein step (a) further comprises incubating the solution for between about 0.5 to about 20 hours.

Embodiment II-80. The process according to any one of embodiments II-65 to II-79, wherein step (a) further comprises one or more pH adjustment steps.

Embodiment II-81. The process according to embodiment II-80, wherein the one or more pH adjustment steps are independently selected from:
(i) raising the lysate comprising polysaccharide pH, or
(ii) lowering the lysate comprising polysaccharide pH.

Embodiment II-82. The process according to embodiment II-81, comprising lowering the lysate comprising polysaccharide pH to between about 3 and 7.0.

Embodiment II-83. The process according to embodiment II-81 or II-82, comprising lowering the lysate comprising polysaccharide pH to about 6.5.

Embodiment II-84. The process according to embodiment II-81 or II-82, comprising lowering the lysate comprising polysaccharide pH to between about 3 and about 4.

Embodiment II-85. The process according to embodiment II-80 or II-81, comprising
(i) lowering the lysate comprising polysaccharide pH to between about 5.5 and 7.0;
(ii) lowering the lysate comprising polysaccharide pH to about 3; and
(iii) raising the lysate comprising polysaccharide pH to between about 5.5 and 7.0.

Embodiment II-86. The process according to any one of embodiments II-80 to II-85, wherein the lysate comprising polysaccharide is incubated at about room temperature (r.t.) after the one or more pH adjustment steps.

Embodiment II-87. The process according to any one of embodiments II-81 to II-84, wherein the lysate comprising polysaccharide is incubated at between about 4° C. and about 30° C. after the one or more pH adjustment steps.

Embodiment II-88. The process according to any one of embodiments II-65 to II-87, further comprising removing solids from the lysate comprising polysaccharide.

Embodiment II-89. The process according to embodiment II-88, wherein removing solids from the lysate comprising polysaccharide comprises filtration, centrifugation, or a combination thereof.

Embodiment II-90. The process according to embodiment II-89, wherein the filtration comprises depth filtration, tangential flow filtration (TFF), sterile filtration, or a combination of the foregoing.

Embodiment II-91. The process according to embodiment II-89 or II-90, wherein the filtration comprises depth filtration followed by TFF.

Embodiment II-92. The process according to embodiment II-88 or II-89, wherein solids are removed from the lysate comprising polysaccharide by centrifugation.

Embodiment II-93. The process according to any one of embodiments II-65 to II-92, wherein the muralytic enzyme of step (b) is mutanolysin, lysozyme, or a bacteriophage hydrolase.

Embodiment II-94. The process according to any one of embodiments II-65 to II-93, wherein step (b) further comprises incubating with a protease.

Embodiment II-95. The process according to embodiment II-94, wherein the protease is proteinase K, trypsin, chymotrypsin, endoproteinase Asp-N, endoproteinase Arg-C, endoproteinase Glu-C, endoproteinase Lys-C, pepsin, thermolysin, elastase, papain, substilisin, clostripain, carboxypeptidase A, carboxypeptidase B, carboxypeptidase P, carboxypeptidase Y, cathepsin C, acylamino-acid releasing enzyme, or pyroglutamate.

Embodiment II-96. The process according to any one of embodiments II-65 to II-95, wherein step (b) further comprises warming the lysate comprising polysaccharide with the muralytic enzyme to between about 30° C. and about 65° C.

Embodiment II-97. The process according to any one of embodiments II-94 to II-96, wherein the lysate comprising polysaccharide with the protease is warmed to between about 45° C. and 55° C.

Embodiment II-98. The process according to embodiment II-96 or II-97, wherein the lysate is warmed between about 6 and about 20 hours.

Embodiment II-99. The process according to any one of embodiments II-65 to II-98, wherein the free polysaccharide solution of step (b) is further purified to reduce the concentration of nucleic acids, enzymes, host cell proteins (HCPs), or a combination of the foregoing.

Embodiment II-100. The process according to any one of embodiments II-65 to II-99, wherein the free polysaccharide solution of step (b) is further purified by precipitation.

Embodiment II-101. The process according to any one of embodiments II-65 to II-100, wherein the free polysaccharide solution of step (b) is treated with cetyltrimethylammonium bromide (CTAB).

Embodiment II-102. The process according to embodiment II-101, wherein the concentration of CTAB in the free polysaccharide solution is about 0.10% to about 10%.

Embodiment II-103. The process according to embodiment II-101 or II-102, the concentration of CTAB is between about 0.5% and about 3%.

Embodiment II-104. The process according to any one of embodiments II-101 to II-103, wherein the free polysaccharide solution of step (b) is treated with potassium iodide (KI).

Embodiment II-105. The process according to embodiment II-104, wherein the concentration of KI in the free polysaccharide solution is between about 20 mM to about 400 mM.

Embodiment II-106. The process according to any one of embodiments II-65 to II-105, wherein the free polysaccharide solution is further purified by filtration, centrifugation, chromatography, or a combination of the foregoing.

Embodiment II-107. The process according to embodiment II-106, wherein the filtration comprises depth filtration, tangential flow filtration (TFF), sterile filtration, or a combination of the foregoing.

Embodiment II-108. The process according to embodiment II-106, wherein the chromatography comprises hydrophobic interaction chromatography (HIC), anion-exchange chromatography (AEX), ceramic hydroxyapatite-type chromatography, or cation exchange chromatography (CEX).

Embodiment II-109. An immunogenic composition comprising:
(a) a Group A Streptococcus (GAS) C5a peptidase polypeptide antigen that comprises or consists of the amino acid sequence of SEQ ID NO: 30, or a fragment thereof,
(b) a GAS streptolysin O (SLO) polypeptide antigen that comprises or consists of the amino acid sequence of SEQ ID NO: 53, or a fragment thereof, and
(c) a polypeptide-polysaccharide conjugate comprising
   a Streptococcus pyogenes Adhesion and Division (SpyAD) conjugate polypeptide that comprises or consists of the amino acid sequence of SEQ ID NO: 34, or a fragment thereof, and
   a GAS polysaccharide, or a variant thereof, that lacks an immunodominant N-acetyl Glucosamine (GlcNAc) side chain;
   wherein between about 8 mol % and about 20 mol % of the polysaccharide repeat units of the GAS polysaccharide, or a variant thereof, are derivatized by a linker.

Embodiment II-110. The immunogenic composition of embodiment II-109, wherein the average molecular weight of the polypeptide-polysaccharide conjugate is between about 185 kDa and about 700 kDa.

Embodiment II-111. The immunogenic composition of embodiment II-109 or II-110, wherein the average molecular weight of the polypeptide-polysaccharide conjugate is between about 200 kDa and about 700 kDa.

Embodiment II-112. The immunogenic composition of any one of embodiments II-109 to II-111, wherein the average molecular weight of the polypeptide-polysaccharide conjugate is between about 300 kDa and about 700 kDa.

Embodiment II-113. The polypeptide-polysaccharide conjugate of any one of embodiments II-109 to II-112, wherein the average molecular weight of the polypeptide-polysaccharide conjugate is between about 300 kDa and about 600 kDa.

Embodiment II-114. The polypeptide-polysaccharide conjugate of any one of embodiments II-109 to II-113, wherein the GAS polysaccharide, or a variant thereof, has a molecular weight of at least about 10 kDa to at least about 40 kDa.

Embodiment II-115. The polypeptide-polysaccharide conjugate of any one of embodiments II-109 to II-114, wherein the SpyAD conjugate polypeptide is a fragment of the amino acid sequence of SEQ ID NO: 34, and comprises or consists of the amino acid sequence of SEQ ID NO: 77, SEQ ID NO: 78, SEQ ID NO: 79, SEQ ID NO: 81, SEQ ID NO: 82, or SEQ ID NO: 83.

### EXAMPLES

### Synthetic Example 1: Purification of High Molecular Weight (Long) Polysaccharides

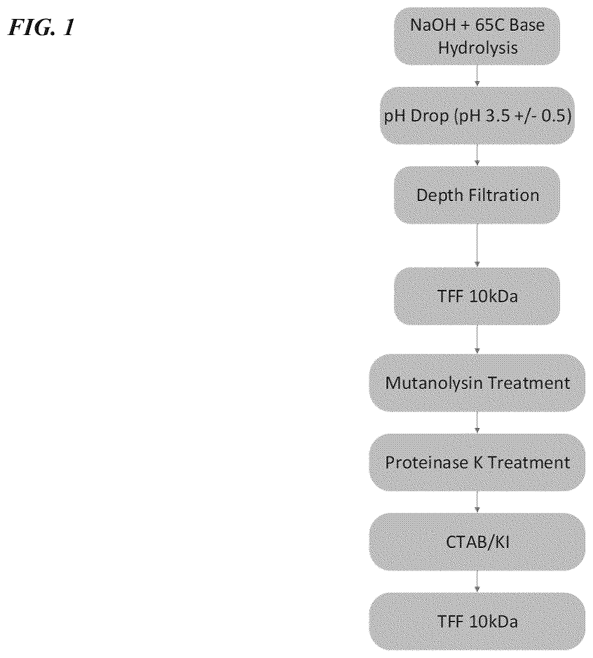

Experiments were performed to extract and purify GAS polysaccharides from GAS bacterial cultures. FIG. 1 shows a simplified flowchart of a sample purification described herein.

Base Hydrolysis: A prepared GAS cell pellet was re-suspended in 50 mM NaCl solution. Using a serological pipette, 50 mL NaCl was added and vortexed until re-suspended. 10N sodium hydroxide & 1M sodium borohydride were added to reach a final concentration of 4N NaOH and 25 mM NaBH₄. The re-suspended pellet was split between centrifuge bottles with a final target volume of 160 mL per 1 L fermentation volume. When splitting the solution, constant swirling was used to ensure homogeneity. Bottles were placed on a shaker in the pre-heated incubator at 65° C. for 2 hours. After incubation, the hydrolysis solution was centrifuged for 30 min at 14,000×g at 25° C. to pellet any cell debris, making sure to let the hydrolysis sample cool down to room temperature prior to centrifugation. The supernatant was collected once centrifugation stopped, in order not to disturb the pellet, and the supernatant was neutralized to pH 6.5±0.3. When neutralizing, the bottle was placed on a 4° C. ice bath, using 37% HCl to adjust pH, with 1M NaOH used for further adjustment if necessary. The sample is then incubated at 4° C. overnight.

Filtration: After incubation, a white precipitate forms. The solution was centrifuged for 30 min at 10,000×g. A bulky white pellet is formed, containing host cell proteins (HCPs). The clear supernatant was collected. The pH was adjusted to 3.0 using 37% HCl, and the solution was incubated for 1 hour at RT. Using a Clarisolve Filter μPod −40 MS, the sample was filtered. For example: at a pump flow rate of 23 mL/min, water was flushed through the filter until primed, and the valve was then opened and flushed with 120 mL volume, and equilibrated using 15 mM NaCl for a volume of 120 mL. The extract was run through the filter at the same pump flow rate of 23 mL/min, and the clear permeate coming from the filter was collected. The filter was washed with 60 mL of 15 mM NaCl, and flushed out the tubes and filter content. The filter was discarded. The sample pH was readjusted to 6.5±0.3. Tangential flow filtration (TFF-10k) was then conducted to remove salts, concentrated (ultrafiltered), then buffer exchanged (diafiltered) in 10 mM NaCl.

Mutanolysin Treatment: The solution was prepared for mutanolysin treatment by adding 1M MgCl₂ to reach a final conc. of 1 mM MgCl₂, and 200 mM sodium phosphate (10×) to reach a final concentration of 20 mM sodium phosphate at pH 6.8. Mutanolysin solution (5000 IU/mL) to reach 120 IU/mL. The sample was incubated at 37° C. overnight with shaking.

Proteinase-K Treatment: The sample was then treated with Proteinase-K by adding Proteinase-K solution to achieve a final concentration of 40 IU/mL (Proteinase-K at 45 u/mg). The mixture was incubated at 50° C. overnight while mixing gently.

Precipitation and Filtration: To precipitate enzymes, nucleic acids and HCPs in the sample, CTAB in 20 mM sodium phosphate buffer at pH 6.8 was added, shaking for 1 hour at 30° C. The solutions used were all pre-warmed: the PS sample, 5% CTAB stock solution and 200 mM Na Phosphate pH 6.8. The PS solution was mixed (magnetic stir bar) while being heated, and the heated stir plate was set up with an internal thermometer in order to monitor temperature in the solution at all times. 200 mM sodium phosphate (pH 6.8) solution was added to the PS solution, to reach a final concentration of 20 mM sodium phosphate. 5% CTAB was added to the PS solution to reach a 1% CTAB concentration. The solution was allowed to mix for 1 hour. Using a 40 MS filter, a depth filtration is conducted over the precipitating solution. For example, the system was flushed with 200 mL MilliQ H20 at a pump flow rate of 23 mL/min, and once water started to come out from the vent, it was closed so the solution is forced to come out from the outlet (priming). The system was then flushed with 75 mL of a solution of 20 mM sodium phosphate (pH 6.8) and 15 mM NaCl. The sample was filtered at 23 mL/min, and flushed with 60 mL of 20 mM sodium phosphate (pH 6.8) and 15 mM NaCl solution at 20 mL/min. The permeate was collected until air bubbles eluted. The filter was discarded.

The PS solution and 274 mM KI was warmed to 30° C. Enough 274 mM KI was added to the mixing PS solution to achieve a final concentration of 27.4 mM KI. The mixture was incubated at 30° C. with mixing for 1 hour. The solution was centrifuged post incubation at 30° C., 10,000×g for 30 minutes. The supernatant was collected and the pellet was discarded, be cautious as the pellet breaks up easily. The supernatant was then vacuum filtered through a 0.45 μm filter.

The sample was first concentrated by TFF-10k, followed by diafiltration with 9 DVs of 350 mM NaCl and finally 2 DVs with MilliQ water. For example, the TFF system volume is around 35 mL, with pump flow set at 200 mL/min, and the diafiltration is conducted with 9 DV (50 mL) of buffer TMP: 7-8 psi

The polysaccharide solution was then purified by hydrophobic interaction chromatography using HiPrep™ Butyl Fast Flow 16/10 pre-equilibrated with 3M sodium chloride and 50 mM sodium phosphate pH 6.8. Sodium chloride and phosphate buffer were added to the polysaccharide solution in order to reach 3M sodium chloride and 50 mM potassium phosphate (pH 6.8). The polysaccharide solution was passed over HIC resin and was operated in flow through mode. The resin is washed with the same equilibration buffer and both the flow-through and wash were collected for further processing.

A final TFF 10 kDa/3 kDa was conducted in order to remove high NaCl content in the PS sample by diafiltration against 9DV of 15 mM NaCl or WFI. The purified PS solution was then 0.22 um filtered.

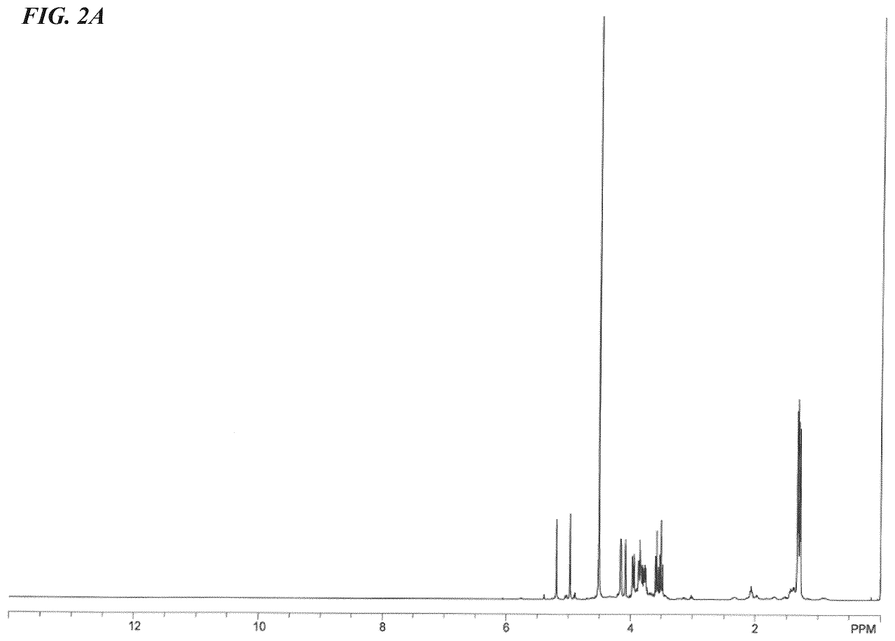

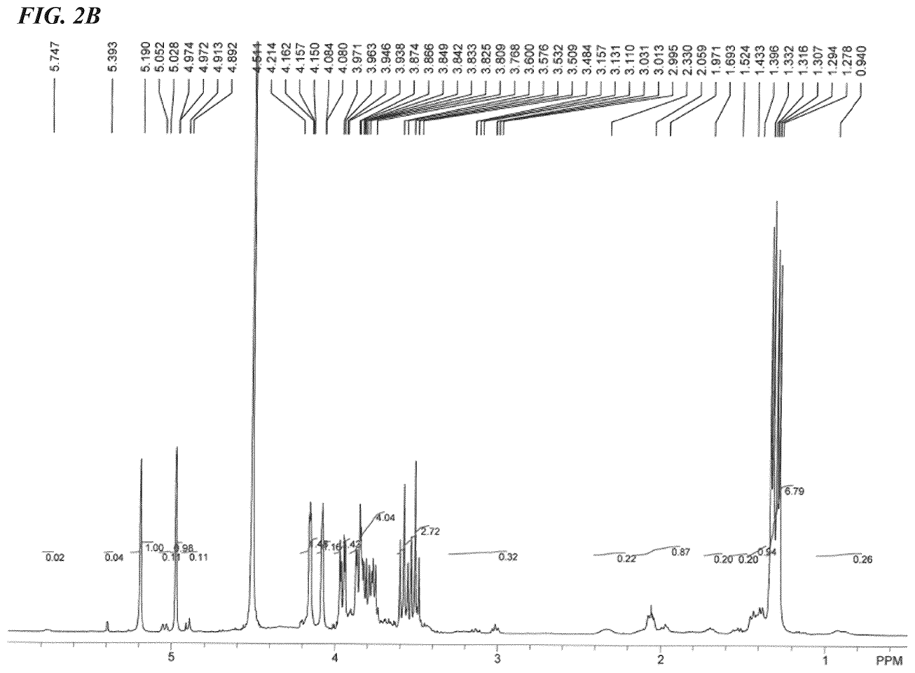

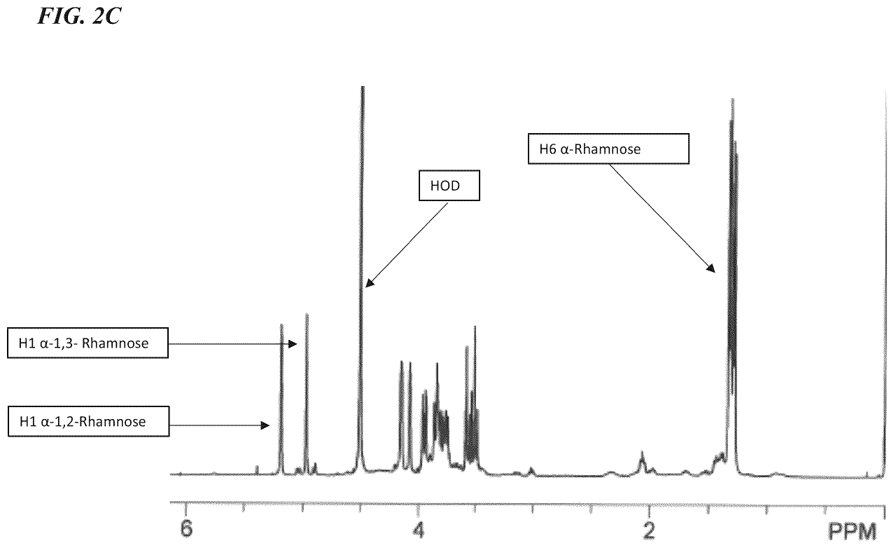

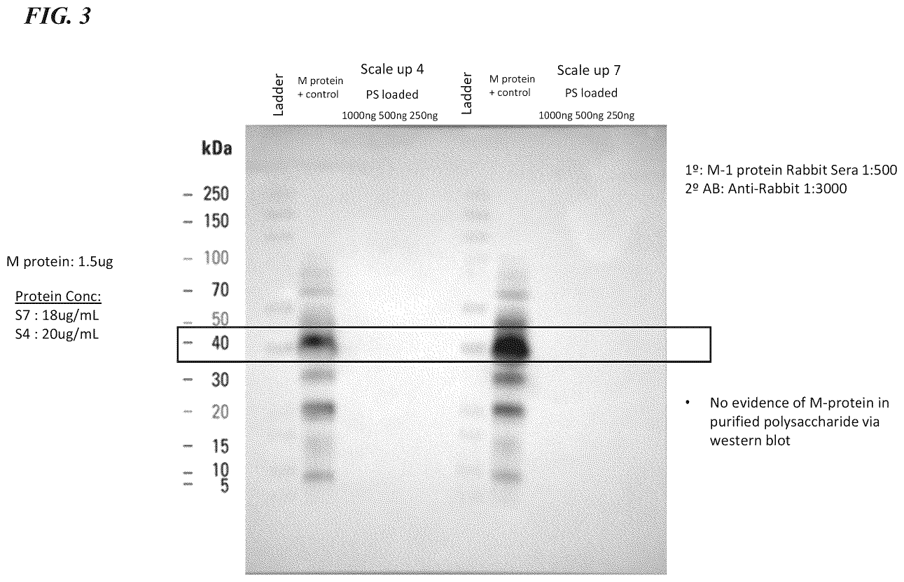

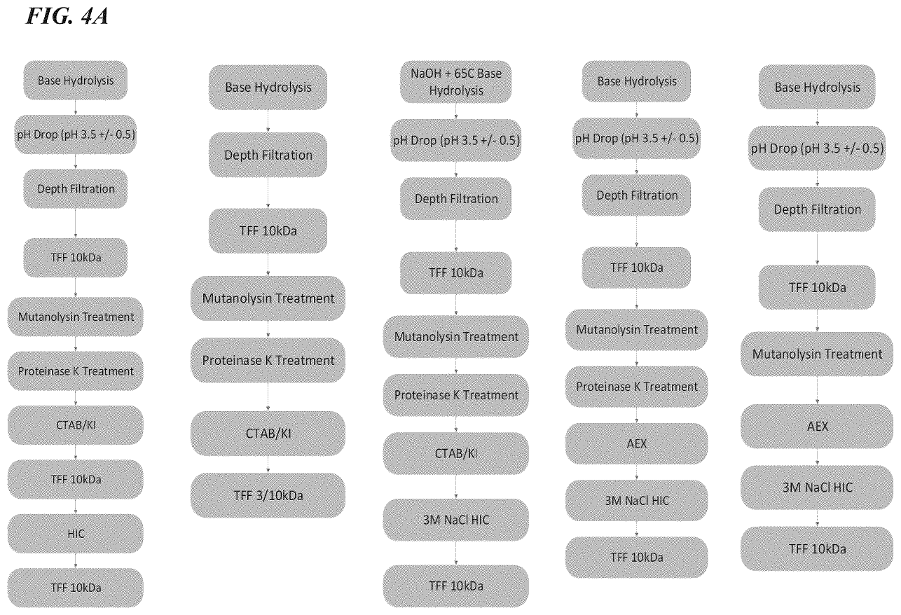

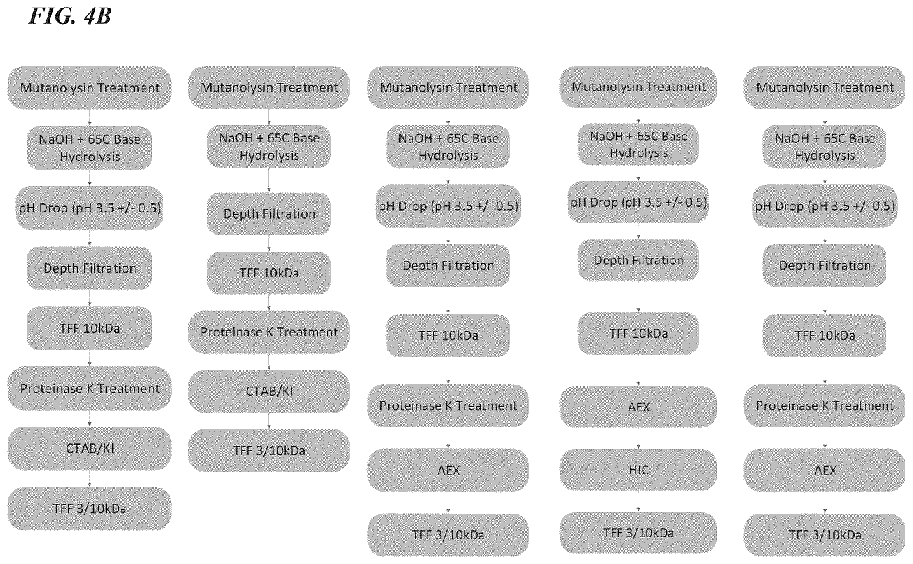

FIG. 2A, FIG. 2B, and FIG. 2C show NMR analysis of purified polysaccharide produced by Synthetic Example 1, originating from a GAS bacterial strain expressing PS lacking GlcNAc (see PCT/US2012/049604). The NMR confirms the presence of polyrhamnose and the absence of GlcNAc. For sample preparation, the purified polysaccharide solution was lyophilized and exchanged into D20 with TSP added as an internal standard. A proton NMR spectrum was obtained at 50° C. on a 400 MHz instrument, with signal averaging over 40 scans. 0.2 Hz of line broadening was applied. The purification protocol was also able to produce purified polysaccharide free of M-protein, as evidenced by the western blot of FIG. 3. FIGS. 4A and 4B show flowcharts outlining alternative exemplary polysaccharide purification methods.

### Synthetic Example 2: General Protocol for the Derivatization of High-Molecular Weight Polysaccharides

Purified polysaccharides, for instance those produced by the methods of Example 1, can be functionalized with a DBCO-PEG linker.

Generally, to a solution of polysaccharide in water (5.5 mM final concentration after all reagents are added), borate buffer (1M, pH 8.5) was added such that the final concentration of borate is 100 mM in the final volume. Water was then added to fill any extra reaction volume. 2.5 equivalents (with respect to the polysaccharide repeating unit) of 1-cyano-4-dimethylaminopyridinium tetra fluoroborate (CDAP; from 100 mg/mL solution in acetonitrile) was added with vigorous stirring. CDAP is stored at −20° C. and solution must be prepared immediately before use. Five minutes after the addition of CDAP (this timing is critical—any longer than 5 min results in reduced DBCO-PEG-Amine incorporation), 0.5 molar equivalents of dibenzocyclooctyne-amine linker (from DMSO stock solution, final concentration of DMSO is 5% v/v) was added. DBCO-PEG4-Amine linker is stored at −20° C. and must be prepared immediately before use. After one hour of further reaction, glycine (2M, pH 8.35) was added 1:10 by volume to give a final concentration of 200 mM glycine to quench any unreacted cyanate esters. After 1 h of quenching, the derivatized polysaccharide was then purified via Zeba spin column. 2-3 mL of solution was added to each 10 mL Zeba column. The purified polysaccharide was analyzed on Bound/Free DBCO HPLC method to determine if residual DBCO-PEG4-Amine linker and DMAP were completely removed by column purification. The material can be further purified if necessary. The polysaccharide concentration was measured using an anthrone assay, and dibenzocyclooctyne concentration was measured using absorbance at 309 nm. These two values were combined to give an estimate of the percentage of polysaccharide derivatized with a dibenzocyclooctyne functional group. Percent DBCO should be between 5-10% for CDAP reactions.

DBCO-PEG4 Derivatization of GAS Polysaccharide: To a 6 mM solution of GAS polysaccharide in 100 mM Borate Buffer pH 8.5, three equivalents (to the polysaccharide repeating unit) of 1-cyano-4-dimethylaminopyridinium tetrafluoroborate (CDAP; from 100 mg/mL solution in acetonitrile) were added with vigorous stirring to facilitate cyanylation at reactive hydroxyl groups. 5 minutes after addition of CDAP, 2 molar equivalents of dibenzocyclooctyne-amine linker stock in DMSO was added such that the final DMSO concentration was 5% (v/v). After DBCO-derivatization, 200 mM glycine was added to the reaction to quench unreacted cyanate esters. The DBCO-derivatized polysaccharide was purified via zeba spin desalting column and the purity of the recovered material was assessed by reverse phase. A single peak in HPLC when absorbance was monitored at 309 nm confirmed complete removal of excess DBCO linker and other reaction byproducts. Finally, the polysaccharide concentration was measured using anthrone assay (see below) and dibenzocyclooctyne concentration was measured using absorbance at 309 nm. These two values were combined to give an estimate of the percentage of polysaccharide derivatized with a dibenzocyclooctyne functional group. For conjugation, % DBCO derivatization of the GAS polysaccharide was kept between 5-10%.

Anthrone assay for total polysaccharide concentration: A stock of 2 mg/ml of the anthrone reagent (Sigma-Aldrich, CAS #90-44-8) was prepared in cold sulfuric acid while a 1 mM stock of polysaccharide repeating unit (PSRU) comprising 2× rhamnose was prepared in water as a standard. In triplicate wells, 100 μl of PSRU stock (serially diluted into reference standards) or the unknown samples (diluted 1:3) were plated (96-well plate) followed by addition of 200 μl/well of the anthrone reagent stock. All reactions were thoroughly mixed and sealed with a plate cover for incubation at 95° C. for 10 min. The plate was briefly placed on ice to cool to ambient temperature before absorbance is measured at 620 nm using a UV/VIS plate reader. To determine concentration of unknown samples, PSRU standard concentrations and absorbances were used to generate a least-square fit regression.

### Synthetic Example 3: Expression and Purification of pAMF-Modified Conjugate Polypeptide

Experiments are performed to express and purify pAMF-modified conjugate polypeptides from a cell free protein synthesis extract.

Polypeptides containing nnAAs (e.g., pAMF) are expressed in a cell free protein synthesis (CFPS) reaction, using an extract (XtractCF⁺) derived from E. coli engineered to produce an orthogonal tRNA for insertion of a nnAA at an amber stop codon. Sample protocols used for cloning, expression, and purification of these modified conjugate polypeptides may be found, for example, in Kapoor et al., Biochemistry, 2018, 57(5), 516-519.

### Synthetic Example 4: General Protocol for the Conjugation of High-Molecular Weight Polysaccharides with Polypeptide Antigens or Non-GAS Carrier Polypeptides to Form Polypeptide-Polysaccharide Conjugates

Generally, polypeptide antigens or a non-GAS carrier polypeptides are conjugated to the purified DBCO-derivatized polysaccharides of Example 2 by reacting the cyclooctyne moiety of the DBCO group with the azide moiety of the nnAA side-chain incorporated into the polypeptide antigen or a non-GAS carrier polypeptide. Sample protocols for the conjugation reaction between the DBCO and azide groups may be found, for example, in Zimmerman et al., Bioconjugate Chemistry, 2014, 25(2), 351-361 and Kapoor et al., Biochemistry, 2018, 57(5), 516-519.

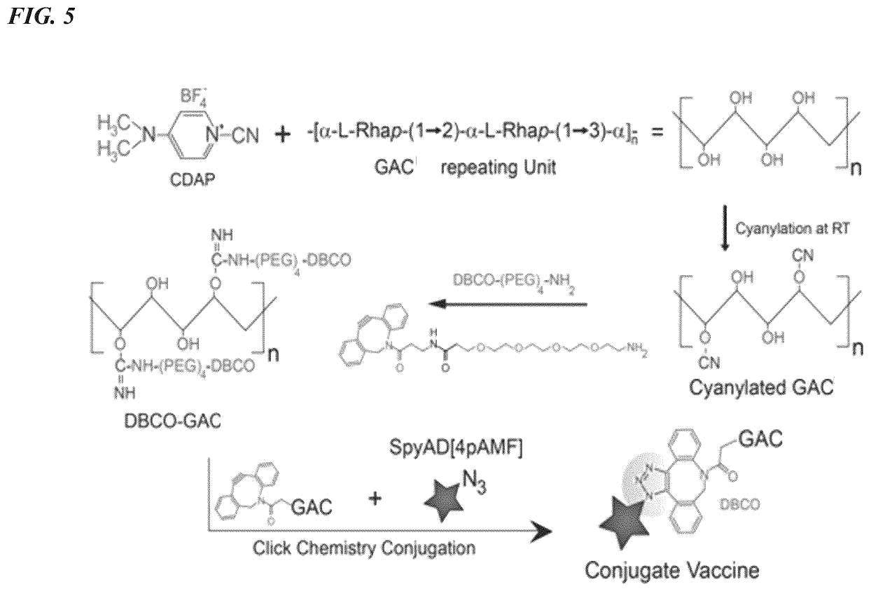

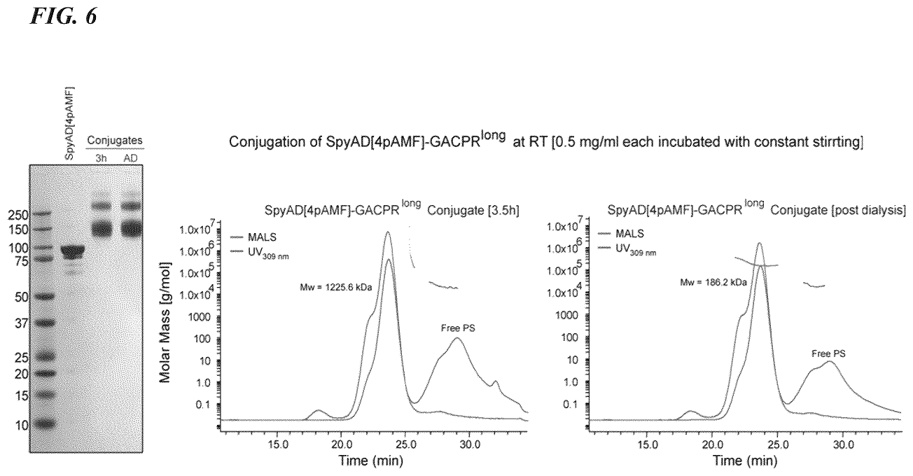

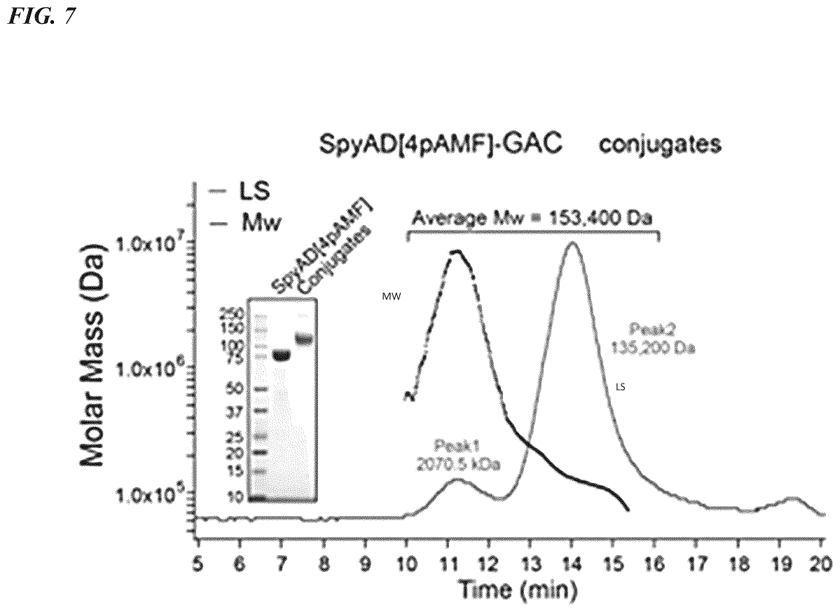

Conjugation of pAMF-derivatized GAS polysaccharide to SpyAD: SpyAD[4pAMF](SEQ ID NO: 34) was mixed with DBCO-derivatized GAS polysaccharide at a 1:1 ratio [0.5 mg/ml each] to facilitate conjugation via click chemistry. Post-conjugation, the reaction mixture was dialyzed against a 50 kDa cutoff membrane to remove excess unreacted free polysaccharide. The recovered conjugates were analyzed by SEC (multi-angle light scattering) MALS and the concentration was estimated using an anthrone assay. FIG. 5 is a scheme outlining the derivatization of GAS polysaccharide with DBCO-(PEG)₄-NH₂ and subsequent conjugation to pAMF-derivatized SpyAD. FIG. 6 shows SEC MALS analysis of the SpyAD[4pAMF] (SEQ ID NO: 34) polypeptide-polysaccharide conjugate after 3.5 hours of conjugation reaction and post-dialysis. After 3.5 hours, conjugates greater than 1 MDa were observed. Post-dialysis, the isolated conjugate size distribution showed a peak at approximately 186 kDa. FIG. 7 shows the SEC-MALS analysis of the polypeptide-polysaccharide conjugate using the same SpyAD polypeptide as the present example, but where a hydrofluoric acid-based PS purification was utilized (see e.g., van Sorge, N. M. et al. 2014, Cell Host Microbe. 15(6): 729-740 for a sample protocol). Using the PS purified by the older method, the peak molecular weight was 153 kDa compared to ˜186 kDa for the presently described purified polysaccharide.

SEC MALS-UV-RI was performed with an Agilent HPLC 1100 degasser, temperature-controlled auto-sampler (4° C.), column compartment (25° C.) and UV-VIS diode array detector (Agilent, Santa Clara, CA) in line with a DAWN-HELEOS multi-angle laser light scattering detector and Optilab T-rEX differential refractive interferometer (Wyatt Technology, Santa Barbara, CA) coupled to three TOSOH columns in series: TSKgel Guard PWXL 6.0 mm ID×4.0 cm long, 12 μm particle; TOSOH TSKgel 6000 PWXL 7.8 mm ID×30 cm long, 13 μm particle; and a TSKgel 3000 PWXL 7.8 mm ID×30 cm long, 7 μm particle. A mobile phase consisting of 0.2 μm filtered 1×PBS with 5% (v/v) acetonitrile was used at a 0.5 mL/min flow rate and 50-100 μg sample was injected for analysis. Agilent Open Lab software was used to control the HPLC, and Wyatt Astra 7 software was used for data collection and molecular weight analysis.

### Synthetic Example 5: Truncated SLO(ΔC101) Polypeptides with 3, 4, 5, 6, 7, or 8 nnAAs are Expressed, Purified, and Conjugated

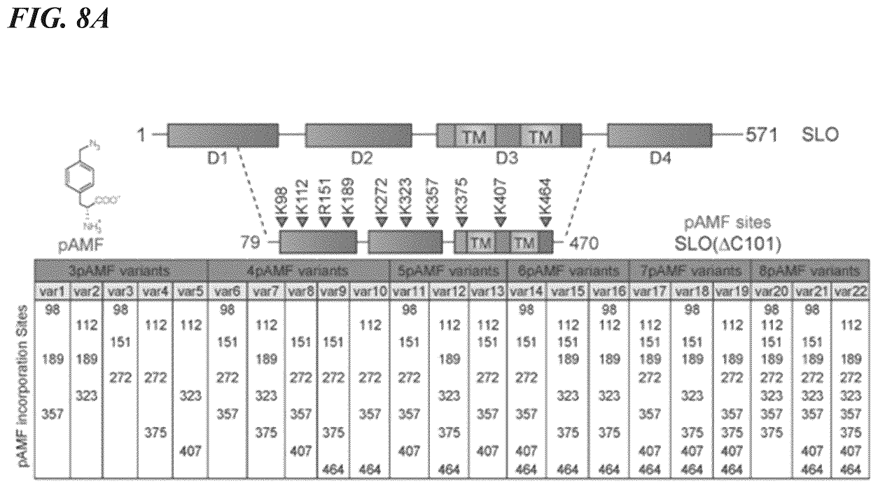

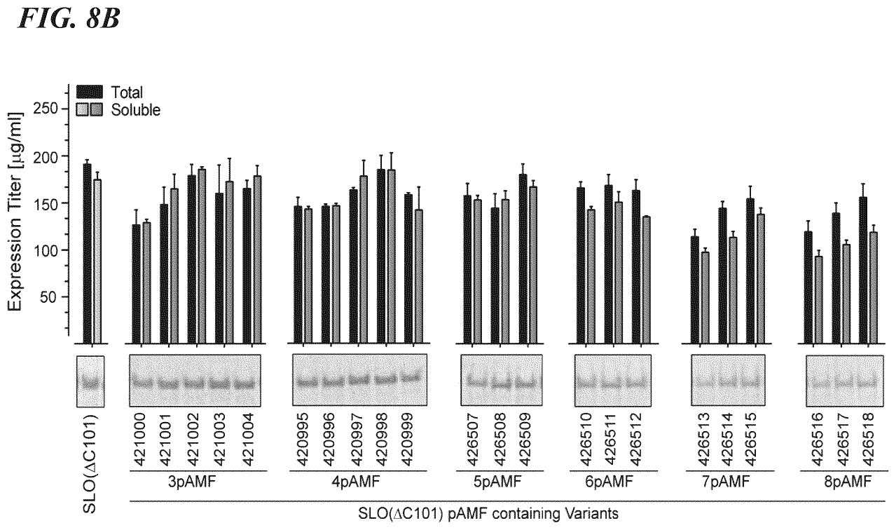

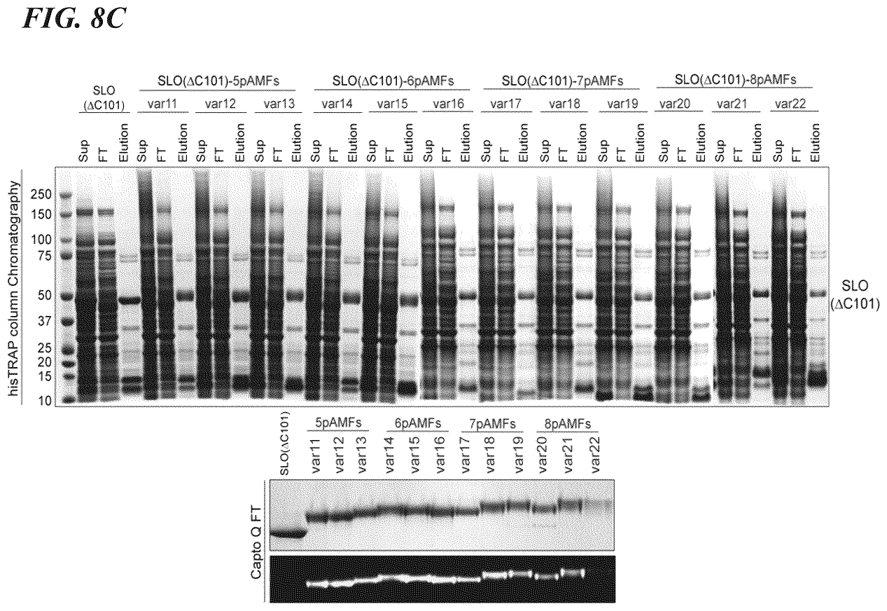

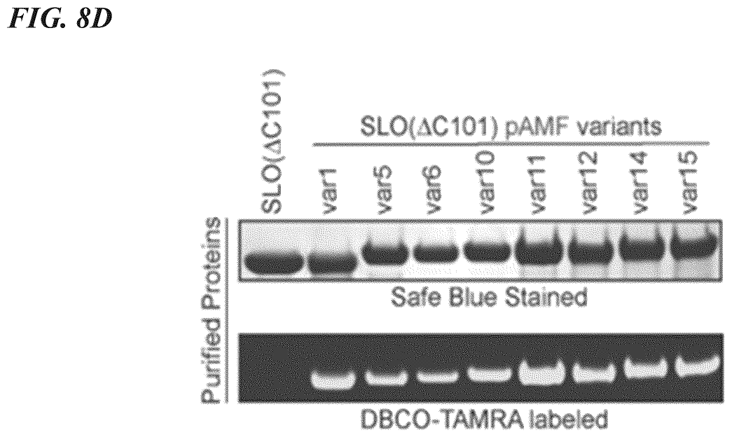

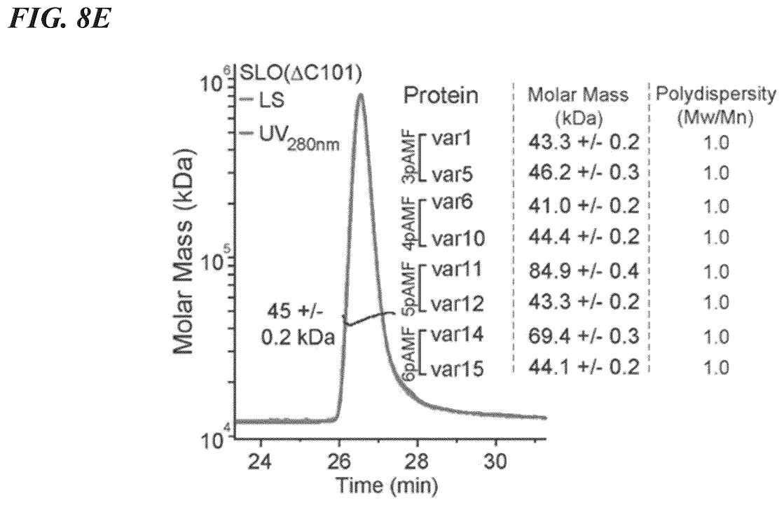

Truncated SLO(ΔC101) variants containing nnAAs are expressed, for instance, according to the above methods (see e.g., Synthetic Example 3). The variants contain 3, 4, 5, 6, 7, or 8 pAMF residues, corresponding to SEQ ID NO: 55, SEQ ID NO: 56, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 65, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, and SEQ ID NO: 76. FIG. 8A shows a schematic of 22 SLO(ΔC101) variants (var1-var22) and their pAMF incorporation sites. FIG. 8B shows the expression levels of these 22 pAMF variants (3pAMF-8pAMF) in both total and soluble protein concentration. FIG. 8C shows the corresponding gels for the expression (SEQ ID NOs: 65-76 correspond to var11-var22). 8D shows gels which reveal successful pAMF incorporation in a subset of purified variants, as confirmed by DBCO-TAMRA labeling (var1, 5, 6, 10, 11, 12, 14, and 15). In short, for purification, the CFPS was harvested, spun down and filtered before loading onto a pre-equilibrated hisTRAP excel 1 ml column at 0.75 ml/min and eluted in a single step of 250 mM imidazole in a 6 ml final volume. 5 μl of each step fraction was analyzed using SDS-PAGE safe blue staining. Thereafter, the elutions were 3× diluted and loaded onto the CaptoQ column for final purification and analyzed using Safe Blue staining and DBCO TAMRA labeling. The FT fractions were concentrated and stored at −80° C. in 200 μl aliquots. SEC-MALS analysis provided molecular mass estimates for a subset of the purified variants (FIG. 8E). This analysis shows that the 5-pAMF (var11) and 6-pAMF (var14) variants showed a higher-order oligomeric state in solution.

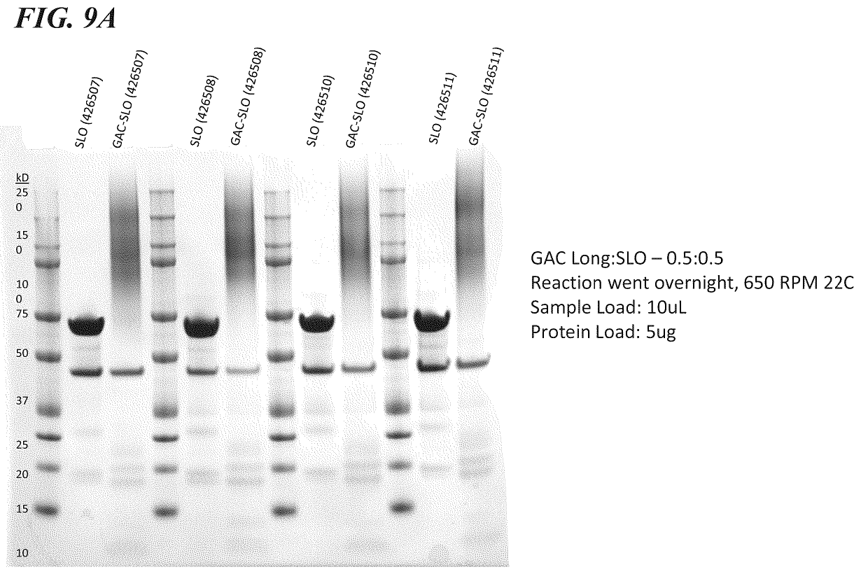

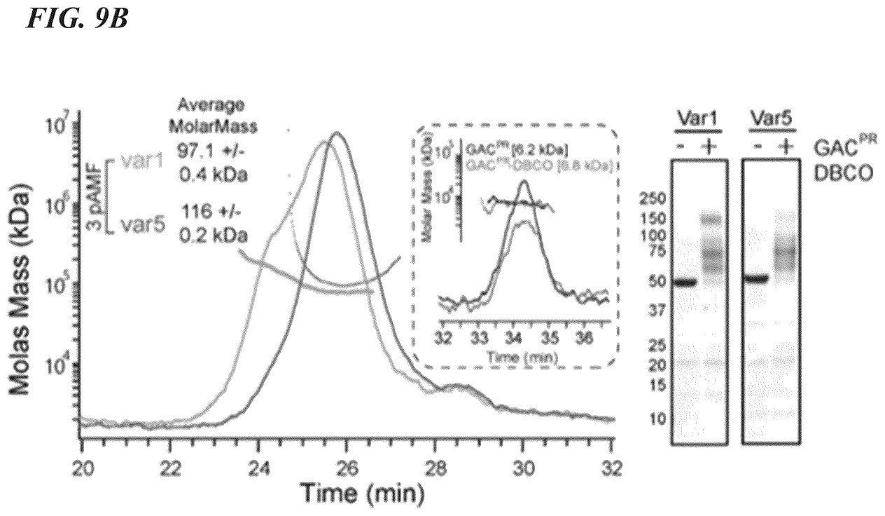

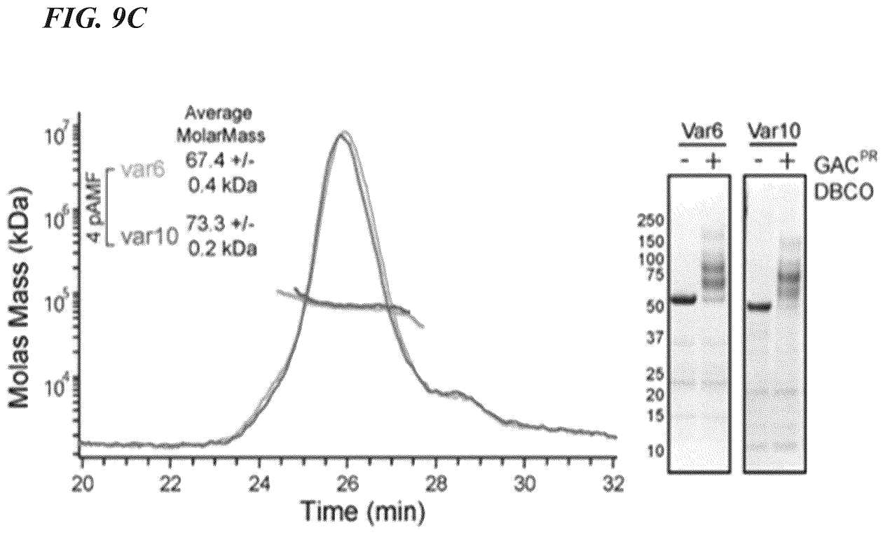

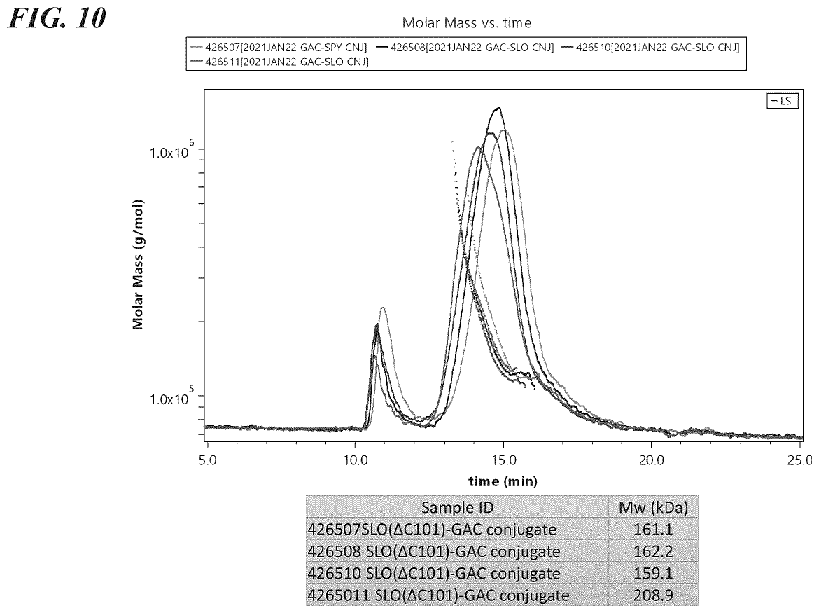

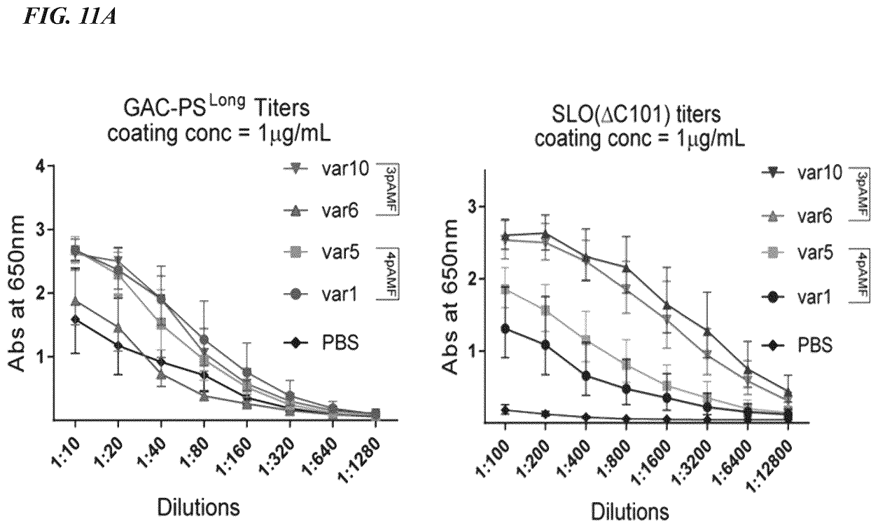

Conjugation of SLO(ΔC101) polypeptides with 5, 6, 7, or 8 nnAAs: The polypeptides containing 5, 6, 7, or 8 nnAAs are conjugated to GAS polysaccharides using the methods described above, including in Synthetic Example 4. In this way, the pAMF-containing SLO (ΔC101) variants of SEQ ID NO: 65, SEQ ID NO: 66, SEQ ID NO: 68, and SEQ ID NO: 69 were conjugated to long DBCO-derivatized GAC (Synthetic Example 1). The conjugation reactions were analyzed by SDS page, as shown in FIG. 9A. The first lane for each variant shows the protein pre-conjugation. The final conjugate sizes were determined by SEC-MALS, the results of which are shown in FIG. 10.

Biophysical characterization and immunogenicity assessment of conjugates generated using 3- and 5-pAMF SLO variants as carrier protein. To generate conjugates using purified SLO(ΔC101) variants, 3 pAMF (var1 and var5) and 4 pAMF (var6 and var10) containing SLO(ΔC101) variants were used in a copper-free click chemistry reaction with DBCO-derivatized polyrhamnose rich core of the species defining membrane-anchored GAS carbohydrate (GAC^{PR}) namely GAC^{PR}-DBCO. Each protein was mixed with DBCO-GAC^{PR} for 4 h at room temperature with constant stirring. Thereafter, the reactions were harvested and the conjugates were dialyzed against buffer to remove excess free PS. Next, SEC-MALS analysis was performed on the purified conjugates, which estimated an average molar mass of 97 or 116 kDa for conjugates generated using SLO(ΔC101) variants containing 3 pAMFs (FIG. 9B) and 68 or 74 kDa for conjugates generated using SLO(ΔC101) variants containing 4 pAMFs (FIG. 9C). Finally, SDS-PAGE analysis of the purified conjugates followed by SafeBlue™ staining corroborated the results and confirmed complete consumption of the carrier proteins for the generation of each conjugate post click chemistry reaction (FIGS. 9B-9C).

### Synthetic Example 6: Optimizing Linker Incorporation into Polysaccharide Repeat Units

As described above, purified polysaccharides (e.g., a GAS polysaccharide), for instance those produced by the methods of Example 1, can be derivatized by a linker (e.g., DBCO-PEG). The mol % of polysaccharide repeat units (PSRU) of the GAS polysaccharide derivatized by DBCO-PEG4-amine can be defined in several related ways (e.g., the % incorporation of DBCO-PEG4-amine as measured by mol of linker per mol PSRU; mol % of PSRU derivatized by a linker).

To an aqueous solution containing Group A Carbohydrate (GAC) polysaccharide lacking an immunodominant N-acetyl Glucosamine side chain, sodium borate pH 8.8, and dimethyl sulfoxide, was added 1-Cyano-4-dimethylaminopyridinium tetrafluoroborate (CDAP, 0.5-4 molar equivalents relative to GAC polysaccharide repeat unit) in acetonitrile. After 5 minutes, a solution of DBCO-PEG4-amine (0.5-2 molar equivalents) in dimethyl sulfoxide was added. The final concentrations of reaction components were as follows: GAC, 4 mg/mL; sodium borate, 0.1 M; dimethyl sulfoxide, 10% v/v. After 1 hour, glycine was added to a final concentration of 0.2 M. After 2 hours, the reaction mixture was purified by gel filtration chromatography or by tangential flow filtration using saline solution and water as diafiltration buffers. Activated GAC (also referred to as APS, activated polysaccharide) was analyzed for polysaccharide concentration and incorporated % DBCO-PEG4-amine.

By altering reaction conditions, GAS polysaccharide was prepared that had 6-15% incorporation of DBCO-PEG4-amine (mol % relative to PSRU) using the above protocol. Table 2 shows examples resulting in the noted % incorporation.

TABLE 2
Sample PS Activation Conditions and Functionalization Outcome

| | CDAP | DBCO | % Incorporated |
|---|---|---|---|
| | molar | molar | DBCO-PEG4-amine |
| APS lot | equivalents | equivalents | (mol DBCO/mol PSRU) |
| A | 1 | 1 | 6 |
| B | 2 | 1 | 11 |
| C | 3 | 1 | 15 |
| D | 1 | 1 | 6 |
| E | 2 | 1 | 12 |
| F | 1.4 | 1 | 7 |

### Synthetic Example 7: Optimizing Conjugation Conditions for Long Polysaccharide and SpyAD

Conjugation efficiency of the long polysaccharides described herein is dependent upon the mol % incorporation of linker (see, for instance, Synthetic Example 6) as well as the reaction concentration of polypeptide and the amount of activated polysaccharide relative to polypeptide.

Sample Conjugation Protocol: To an aqueous solution containing activated polysaccharide (see Synthetic Example 6) and phosphate-buffered saline was added SpyAD-4pAMF (SEQ ID NO: 34). The final concentrations of reaction components were as follows: activated PS with DBCO linker, 0.075-1.35 mg/mL; SpyAD-4pAMF, 0.5-3.7 mg/mL. After 16-20 hours, sodium azide (4 equivalents relative to polysaccharide repeat unit) was added. After 2 hours, the reaction mixture was purified by dialysis or tangential flow filtration using phosphate-buffered saline as the diafiltration buffer. Conjugates were filtered through a 0.22 micron rated filter (Pall KM2EKVS) and then analyzed for polysaccharide concentration, protein concentration, percent free saccharide, and molecular weight. Examples of conjugates produced are shown in Table 3.

TABLE 3
Sample PS Activation Conditions and Functionalization Outcome

| | % | | | | | Output | |
|---|---|---|---|---|---|---|---|
| | Incorporated | | | | | ratio | Output |
| | DBCO- | | Input | | | (PS:Spy | ratio |
| Conjugate | PEG4- | SpyAD | APS:Spy | Conjugate | % Free | AD) in | adjusted |
| lot | amine in | concentration | AD mass | Mw | saccharide | purified | for |
| number | Activated PS | (mg/mL) | ratio | (kDa) | (FS) | conjugate | % FS |
| CNJ-A | 6% | 0.5 | 1 | 144 | 30% | 0.28 | 0.19 |
| CNJ-B | 6% | 0.5 | 0.5 | 166 | 25% | 0.22 | 0.17 |
| CNJ-C | 6% | 0.5 | 0.15 | 157 | 7% | 0.12 | 0.11 |
| CNJ-D | 6% | 1 | 0.3 | 182 | 13% | 0.17 | 0.15 |
| CNJ-E | 11% | 0.5 | 1 | 145 | 35% | 0.31 | 0.20 |
| CNJ-F | 11% | 0.5 | 0.5 | 156 | 14% | 0.18 | 0.16 |
| CNJ-G | 11% | 0.5 | 0.3 | 175 | 5% | 0.17 | 0.16 |
| CNJ-H | 11% | 0.5 | 0.15 | 176 | 8% | 0.14 | 0.13 |
| CNJ-I | 11% | 1 | 0.3 | 205 | 11% | 0.16 | 0.14 |
| CNJ-J | 15% | 0.5 | 1 | 179 | 24% | 0.26 | 0.20 |
| CNJ-K | 15% | 0.5 | 0.5 | 177 | 16% | 0.18 | 0.15 |
| CNJ-L | 15% | 0.5 | 0.3 | 263 | 0% | 0.22 | 0.22 |
| CNJ-M | 15% | 0.5 | 0.15 | 214 | 21% | 0.12 | 0.09 |
| CNJ-N | 15% | 1 | 0.3 | 213 | 13% | 0.22 | 0.19 |
| CNJ-O | 11% | 2.76 | 0.3 | 247 | 49% | 0.21 | 0.11 |
| CNJ-P | 11% | 2.76 | 0.15 | 374 | 14% | 0.14 | 0.12 |
| CNJ-Q | 11% | 2.76 | 0.15 | 381 | 14% | 0.12 | 0.10 |
| CNJ-R | 11% | 2.76 | 0.075 | 319 | 14% | 0.09 | 0.08 |
| CNJ-S | 15% | 2.76 | 0.3 | 273 | 36% | 0.18 | 0.11 |
| CNJ-T | 15% | 2.76 | 0.15 | 412 | 6% | 0.13 | 0.12 |
| CNJ-U | 15% | 2.76 | 0.15 | 372 | 8% | 0.14 | 0.13 |
| CNJ-V | 15% | 2.76 | 0.075 | 445 | 14% | 0.09 | 0.08 |
| CNJ-W | 11% | 2.76 | 0.15 | 342 | 14% | 0.11 | 0.10 |
| CNJ-X | 6% | 3.7 | 0.15 | 246 | 24% | 0.10 | 0.08 |
| CNJ-Y | 11% | 3.7 | 0.15 | 429 | 14% | 0.10 | 0.09 |
| CNJ-Z | 15% | 3.7 | 0.15 | 476 | 15% | 0.12 | 0.10 |
| CNJ-AA | 15% | 3.7 | 0.1 | 556 | 13% | 0.10 | 0.09 |
| CNJ-AB | 6% | 1.8 | 0.75 | 201 | 52% | 0.42 | 0.20 |
| CNJ-AC | 11% | 1.8 | 0.75 | 221 | 52% | 0.34 | 0.17 |
| CNJ-AD | 15% | 1.8 | 0.75 | 244 | 60% | 0.39 | 0.16 |
| CNJ-AE | 6% | 0.5 | 0.5 | 172 | 51% | 0.26 | 0.13 |
| CNJ-AF | 6% | 0.5 | 0.5 | 171 | 15% | 0.19 | 0.16 |
| CNJ-AG | 15% | 3.7 | 0.14 | 469 | 8% | 0.13 | 0.12 |
| CNJ-AH | 7% | 0.5 | 0.5 | 184 | 35% | 0.23 | 0.15 |
| CNJ-AI | 12% | 2.5 | 0.15 | 318 | 10% | 0.10 | 0.09 |

As will be discussed further in the Biological Examples below, of the conjugates made with the long polysaccharides of this disclosure, higher molecular weight SpyAD-GAC conjugates (e.g., those made by the methods in Synthetic Example 7) elicited stronger titers against SpyAD and GAC than low molecular weight conjugates. Several factors are important for the production of high molecular weight conjugates. First, the percent of DBCO-PEG4-amine incorporation into the activated polysaccharide (e.g., Synthetic Example 4) influences conjugate size. Across a range of 6-15% o, a higher degree of DBCO incorporation (mol % o) generally yields larger conjugates. Second, higher mass ratios of GAC:SpyAD generally produce smaller conjugates, while lower ratios result in unconjugated SpyAD polypeptide. Mass ratios of 0.10-0.15 generally limit the amount of unconjugated SpyAD and activated polysaccharides, and result in larger conjugates. Finally, at a given GAC:SpyAD ratio, higher concentrations of both reactants generally result in larger conjugates.

Percent incorporated DBCO-PEG4-amine in the activated polysaccharide was reported as the molar ratio (Total DBCO-PEG4-amine)*((% Bound DBCO-PEG4-amine)/(PSRU concentration). Those values obtained as follows: For total DBCO-PEG4-amine, the absorbance of an APS sample was measured at 307 nm. The total DBCO-PEG4-amine concentration in the sample was determined by using an extinction coefficient determined for DBCO-PEG4-amine. Percent bound DBCO-PEG4-amine was measured by HPLC analysis. Samples were injected on a Sepax Zenix-C SEC-300 column and eluted with a mobile phase containing 50 mM potassium chloride, 15% v/v acetonitrile, and 0.1% v/v trifluoroacetic acid. Percent bound DBCO-PEG4-amine was determined as the peak area ratio of (APS)/(APS+free DBCO-PEG4-amine) at 310 nm.

Polysaccharide concentration was measured using an Anthrone assay as described previously. Samples were assayed for protein concentration using a Pierce Modified Lowry assay kit, following the manufacturer's protocol. To measure free saccharide, a solution of sodium deoxycholate (1% w/v in water) was prepared and the pH was adjusted to 6.8 with HCl. To a solution of conjugate was added 0.1 volumes of deoxycholate stock solution and 0.05 volumes of HCl (1 M). The sample was spun and the supernatant was recovered, and this procedure was repeated once more. The polysaccharide content of the supernatant was measured by the anthrone assay (as described previously), and the free saccharide content of the conjugate was determined as a ratio of polysaccharide concentration of the deoxycholate supernatant to polysaccharide concentration of the conjugate. Conjugate molecular weight was determined by SEC-MALS, as described previously.

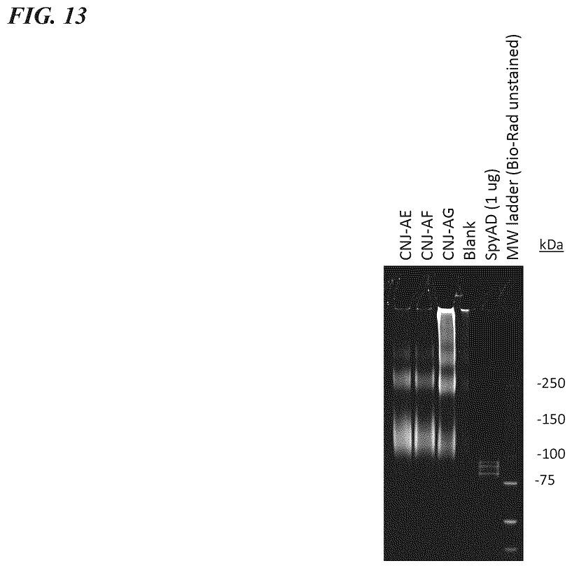

By way of example, SpyAD conjugates CNJ-AE, CNJ-AF, and CNJ-AG were produced by varying the parameters described above. Notably, conjugate CNJ-AG had an average molecular weight (determined by SEC-MALS) of approximately 469 kDa, versus roughly 170 kDa for both CNJ-AE and CNJ-AF (see FIG. 13). This can be attributed to the higher degree of DBCO incorporation in the activated polysaccharide (15% versus 6%), the higher concentration of the SpyAD polypeptide in the reaction mixture, and the lower input mass ratio of activated polysaccharide to polypeptide. Additionally, CNJ-AG was produced with lower amounts of free polysaccharide, which simplifies purification procedures and makes for a more consistent active pharmaceutical ingredient for use in the immunogenic compositions described herein. This also means that production is more efficient, cost-effective, and sparing of reagents.

### Synthetic Example 8: Expression of SpyAD Fragments

SpyAD polypeptide fragments of SEQ ID NO: 34 (SEQ ID NO: 77, SEQ ID NO: 78, SEQ ID NO: 79, and SEQ ID NP: 81) were expressed according to the methods of Synthetic Example 3. A construct encoding for a polypeptide with a 2×6-histag leader sequence (such as that of SEQ ID NO: 80) was used for expressing each fragment, with SEQ ID NOs: 77, 78, 79, and 81 corresponding to the sequence after purification and cleavage of the leader tag.

Fragments of SpyAD may be useful for improving expression, purification, and/or immunogenicity compared to a native or full-length SpyAD sequence. To monitor production of the desired polypeptide fragments, ¹⁴C-leucine (GE Life Sciences, Piscataway, NJ) was added to CFPS reactions to synthesize SEQ ID NOs: 77, 78, 79, and 81, and incorporated into the translating polypeptides. After 10 hours at 23° C. with shaking, the supernatant was recovered by spinning at 4,500 rpm for 10 minutes. Expression titer of total and soluble proteins were estimated by ¹⁴C counts. A total 4 μl of supernatant were loaded to non-reducing 4-12% SDS-PAGE gels. After the protein gels were dehydrated for 2 hours at 80° C., the expression pattern of native SpyAD (SEQ ID NO: 33), SpyAD(4pAMF) (SEQ ID NO: 34), and fragments (SEQ ID NOs: 77, 78, 79, and 81) were analyzed by autoradiography using a Storm 820 PhosphoImager.

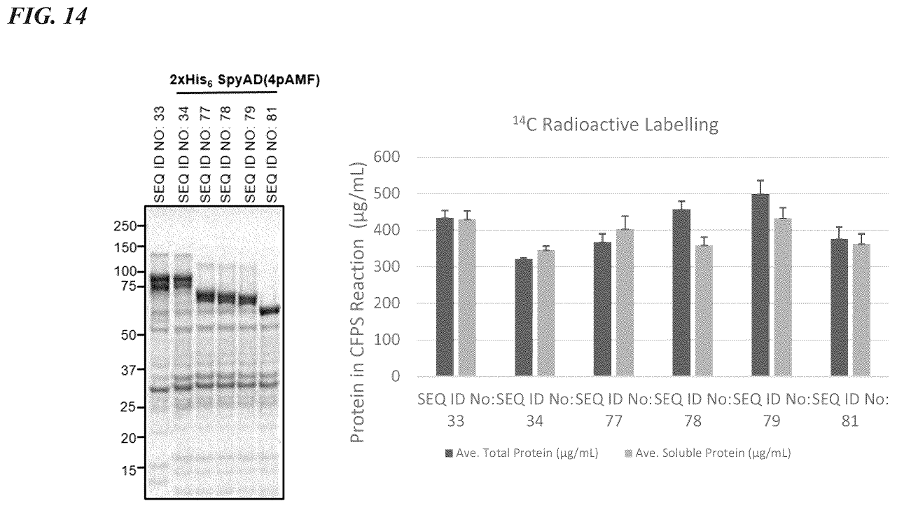

FIG. 14 shows the autoradiography results for the expression of native SpyAD, SpyAD(4pAMF), and the four fragments, as well as an expression titer of total and soluble proteins in each of the samples. Notably, the native SpyAD and SpyAD(4pAMF) expression mixture has 6 bands of contaminating fragments between 50 and 75 kDa, whereas the SpyAD fragments of SEQ ID NOs: 77-79, and 81 show fewer bands within the same region. The improved protein expression levels in the CFPS reactions result in higher recovery yield. This simplifies the purification procedure for the polypeptides and results in cleaner material to carry forward through conjugation to derivatized polysaccharide.

### Biological Example 1: Active Immunization of Mice with Polysaccharide or Polysaccharide Conjugate

Mice are actively immunized prior to being challenged by subdermal and IP injection.

TABLE 4
Sample Immunization Experiment
Timeline - Subdermal and IP Challenges

| Subdermal Challenge - Steps | IP Challenge - Steps |
|---|---|
| Day 1: 1^{st} immunization | Day 1: 1^{st} immunization |
| Day 14: 2^{nd} immunization | Day 14: 2^{nd} immunization |
| Day 28: 3^{rd} immunization | Day 28: 3^{rd} immunization |
| Day 42: infection challenge | Day 41: shave mice |
| Day 49: terminate experiment | Day 42: infection challenge |
| | Day 45: euthanize mice and collect lesions |

1^{st}, 2^{nd}, and 3^{rd} Immunizations—Subdermal and IP Challenges: Antigen/adjuvant mixtures are prepared by combining 50 μL alum (Alhydrogel) with 10 μg antigen(s) or 5 μg of conjugate and mixing rigorously to allow antigens to adsorb onto the alum. Each antigen/adjuvant mixture is drawn into 1 mL syringes fitted with 26½ gauge needles. Each mouse is anesthetized with inhaled isoflurane and injected with 100 μL of the prepared vaccine into the hind leg muscle.

Preparation of Mice for Challenge—Subdermal Challenge: Mice are anesthetized with isoflurane. The backs of the mice are shaved with an electric razor, with care taken not to nick the skin. Hair depilation cream is applied to the shaved backs and is allowed to sit for a few minutes before thoroughly wiping them clean with damp paper towels. The mice are patted dry and allowed to recover from isoflurane treatment.

Preparation of Materials for Challenge—Subdermal and IP Challenges: A GAS culture is grown to mid-logarithmic phase. The cell concentration is adjusted with sterile phosphate buffered saline, serially diluting and plating bacteria onto agar to confirm bacterial dose. For Subdermal Challenge, the targeted CFU per 10 μL per mouse is 1×10⁶, and the bacteria is drawn into 500 μL Hamilton syringes fitted with 26½ gauge needles. For IP Challenge on day 35, the targeted CFU per 100 μL per mouse is 1×10⁷, and the bacteria is drawn into 1 mL syringes fitted with 26½ gauge needles. The mice are anesthetized with inhaled isoflurane and then injected with 200 μL of M1 89155 bacteria into the peritoneal cavity. The mice are allowed to recover from the anesthetic in normal air. Survival of the mice is tracked over the course of 1 week, checking multiple times per day.

Subdermal Challenge and Lesion Collection: For subdermal challenges on day 35, the mice are anesthetized with inhaled isoflurane and then injected with 10 μL of GAS bacteria into the shaved backs using a repeat dispenser for the Hamilton syringe. Lesion sizes are tracked daily over the course of 3 days by photographing isoflurane-anesthetized mice alongside a ruler. Prior to lesion collection on day 3, sterile 2 mL screw cap tubes with 1.0 mm silica beads and 1 mL PBS for each skin lesion were prepared. The weights of each tube were recorded. On day 3, when lesions were fully developed, the mice are euthanized with CO₂ and cervical dislocation. Using clean surgical instruments, each skin lesion is cut out and placed into the pre-weighed tubes. Tube weights are recorded for tissue mass calculations. The tubes are placed into a MagnaLyser bead beater, and the tubes are beat at 6000 rpm for 60 s. The tubes are then placed onto ice to cool for 60 s before repeating the beating cycle. Samples are serially diluted, and the lysate is placed onto agar to quantify bacterial burden.

Two parallel experiments are performed in which in one set of animals were bled throughout the experiment in order to test for antigen-specific antibody titers post-vaccination. In the other arm, the mice are not bled during the course of the experiment. Both set of animals are challenged similarly in the end to perform the lesion size & CFU/mg analysis

### Biological Example 2: Polypeptide Antigens or Non-GAS Carrier Polypeptides, and their Polypeptide-Polysaccharide Conjugates, are Assessed for Immunogenicity in Murine Models

Polypeptide antigens or non-GAS carrier polypeptides, and their polysaccharide conjugates, of Synthetic Examples 2 and 3, are assessed in murine models by methods as described above in Biological Example 1. Mice are immunized on days 0, 7, and 14, followed by a terminal bleed on day 21 post-sacrifice.

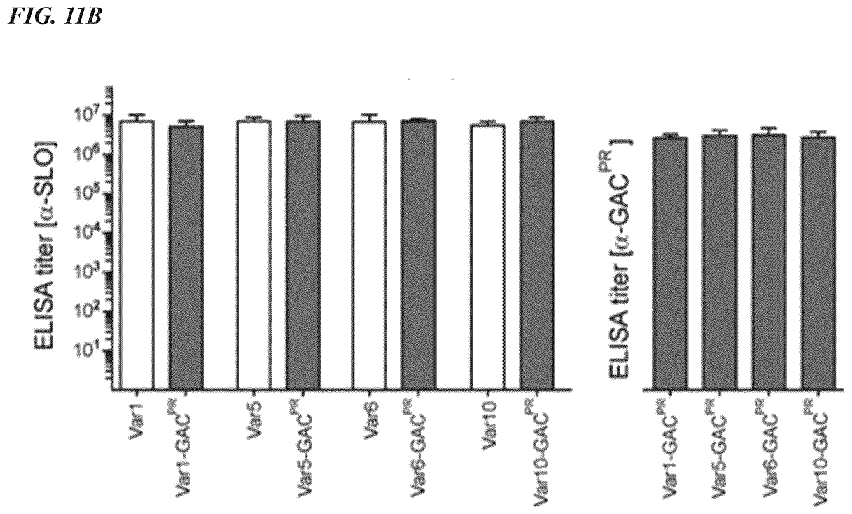

5 μg each of the polysaccharide conjugates are used to immunize mice. The antibody titers against the polypeptide and the polysaccharide are measured after the terminal bleed. The long polysaccharides produced according to the methods of Example 1 may be used as coating antigens in ELISA analysis of these immunogenicity experiments. FIG. 11A shows ELISAs after 5 μg each of the polysaccharide conjugates of pAMF-substituted SLO(ΔC101)-var1, SLO(ΔC101)-var5, SLO(ΔC101)-var6, and SLO(ΔC101)-var10 (corresponding to SEQ ID NO: 55, 59, 60, and 64) were used to immunize mice. These polypeptide-polysaccharide conjugates used short polysaccharides, but the first plot of FIG. 11A shows antibody titers measured after the terminal bleed, where the ELISA utilized a long polysaccharide (produced by the methods of Example 1) as the coating antigen. The second plot of FIG. 11A shows the antibody titers in an ELISA utilizing the polypeptide coating antigen.

The above experiment was repeated with a different cohort of mice, and antibody titers (>10⁶) against SLO were recorded when antisera from protein alone or conjugate group was analyzed, as shown in FIG. 11B. This confirms that careful selection of pAMF incorporation residues for site-selective conjugation does not disrupt critical immunogenic epitopes on SLO as a carrier protein. Finally, robust antibody responses (>10⁶) were also recorded against GAC^{PR}(FIG. 11B). Overall, this shows that CFPS provides an effective platform for expressing and purifying nnAA-containing pathogen-specific protein antigens, which can be successfully utilized to generate immunogenic conjugates.

### Biological Example 3: Stability of Polysaccharide and Polypeptide-Polysaccharide Conjugates

Experiments are performed to measure the stability of the polysaccharides of Example 1 and their conjugates. Polysaccharide or polypeptide-polysaccharide conjugates are held at −20° C., 5° C., and 25° C. for at least 6 months. Samples are taken at 6, 12, and 24 months, and each is analyzed for pH and molecular weight to determine stability.

### Biological Example 4: Comparison of SLO Variant Conjugates to Coadministration of SLO Variant and eCRM197-Polysaccharide Conjugate in an M1 GAS Challenge

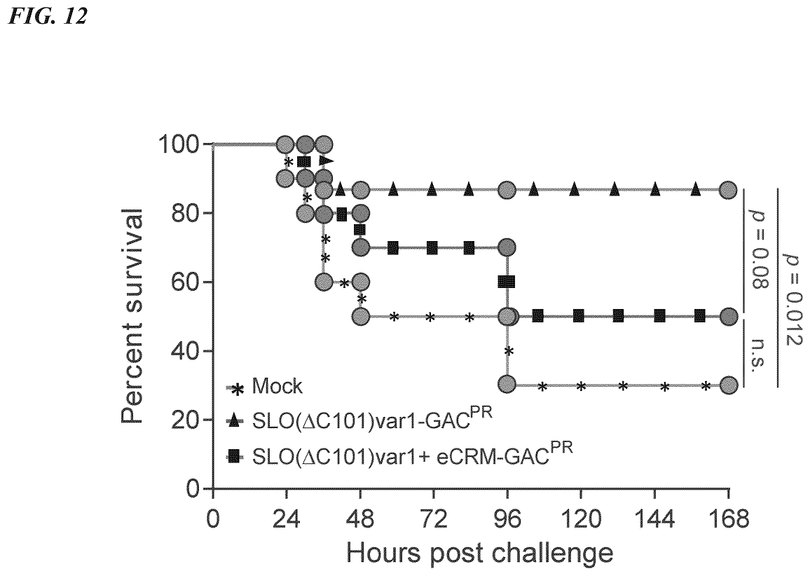

Mice (N=10 per group) were immunized with mock, SLO(ΔC101)var1-GAC^{PR} conjugate or a combination vaccine [SLO(ΔC101)var1+eCRM-GAC^{PR}]. Wild-type female CD-1 mice (Charles River) were immunized every 14 days for a total of 3 doses starting at age of 5 weeks. Intramuscular immunizations delivered consisted of 100 μl total volume per mouse per dose, including 50 μl of Alhydrogel 2% aluminum hydroxide adjuvant (Invivogen), prepared per manufacturer's instructions. 14 days after the final immunization, mice were infected with 1×10⁷ CFU M1 89155 GAS by i.p. injection and tracked for survival. Statistics of Kaplan-Meier survival curves were calculated using log-rank Mantel-Cox test. As shown in FIG. 12, unlike the combination vaccine group, immunization with SLO(ΔC101)var1-GAC^{PR} alone provided significantly higher protection (p=0.012) in comparison to the mock group. This shows that unlike conventional methods of generating conjugates using non-relevant carrier proteins, site-specific conjugation of GAC^{PR} to CFPS generated GAS specific protein antigens provides better protection in vivo.

### Biological Example 5: Evaluation of Immunogenicity of SpyAD-PS Conjugates Using Immunogenic Compositions in NZW Rabbits

Test articles (shown in Table 5) were prepared containing various amounts of a SpyAD-PS conjugate in combination with an equivalent amount of polypeptide antigens C5a (SEQ ID NO: 30) and SLO (SEQ ID NO: 50). Conjugates CNJ-AE, CNJ-AF, and CNJ-AG (see Synthetic Examples 6 and 7) were tested.

TABLE 4
Sample Immunization Experiment
Timeline - Subdermal and IP Challenges

| Test Article | Arm | No. of Antigens |
|---|---|---|
| 0.062 μg conjugate (CNJ-AE) | 1 | 3 |
| and of each protein antigen | | |
| 0.25 μg conjugate (CNJ-AE) | 2 | 3 |
| and of each protein antigen | | |
| 1.0 μg conjugate (CNJ-AE) | 3 | 3 |
| and of each protein antigen | | |
| 4.0 μg conjugate (CNJ-AE) | 4 | 3 |
| and of each protein antigen | | |
| 0.062 μg conjugate (CNJ-AG) | 5 | 3 |
| and of each protein antigen | | |
| 0.25 μg conjugate (CNJ-AG) | 6 | 3 |
| and of each protein antigen | | |
| 1.0 μg conjugate (CNJ-AG) | 7 | 3 |
| and of each protein antigen | | |
| 4.0 μg conjugate (CNJ-AG) | 8 | 3 |
| and of each protein antigen | | |
| 0.25 μg conjugate (CNJ-AF) | 9 | 3 |
| and of each protein antigen | | |

All test articles were formulated in a buffer containing 5 mM sodium succinate pH 5.8, 150 mM sodium chloride, and 0.02% polysorbate 80. 31.2 μg of aluminum phosphate was present in each dose. The conjugates were dosed intramuscularly in female New Zealand white rabbits, 0.25 mL/dose bilateral (0.125 mL/limb). Doses were administered on days 0, 21, and 42, with blood collected on days −1, 14, 35, and 56. Ten rabbits were dosed per arm. Titers against each antigen were measured by ELISA using SLO, C5A, SpyAD, SpyAD-GAC, and eCRM-GAC as a coating antigen).

For each of the ELISA experiments, coating solution containing the antigen of interest was diluted with sterile filtered PBS (total of 100 μL), pH 7.4±0.2, and was added to each well at 0.5 μg/mL. The plate was held at 2° C. to 8° C. for 16 to 24 hours in a sealed container. The plate was then washed 3 times with plate washing buffer (350 μL/well), after which the plate was blocked by adding 200 μL of PBS+3% bovine serum albumin (BSA) and incubated for 60 to 65 minutes at room temperature. After washing the plate 3 times with plate washing buffer, 5-fold serial dilutions of serum samples (50 μL) from test article-treated animals were prepared starting at 1:10 concentrations and added to designated wells. Controls (the sample diluent, 1×PBS+3% BSA; commercial normal serum [Jackson ImmunoResearch] negative control; and positive controls serially diluted by 3- or 5-fold dilutions) were also added to designated wells. Test article serum samples were loaded in duplicate and controls were loaded in singles, preferably on one plate, and incubated 60 to 65 minutes at 35° C. to 39° C. The plate was washed 6 times with plate washing buffer, donkey anti-rabbit IgG (H+L) peroxidase conjugated was added, and the plate was incubated for 60 to 65 minutes at room temperature and then washed again 6 times. ABTS substrate was added and the plate was incubated 30 minutes at room temperature. The plate was read at 415 and 570 nm.

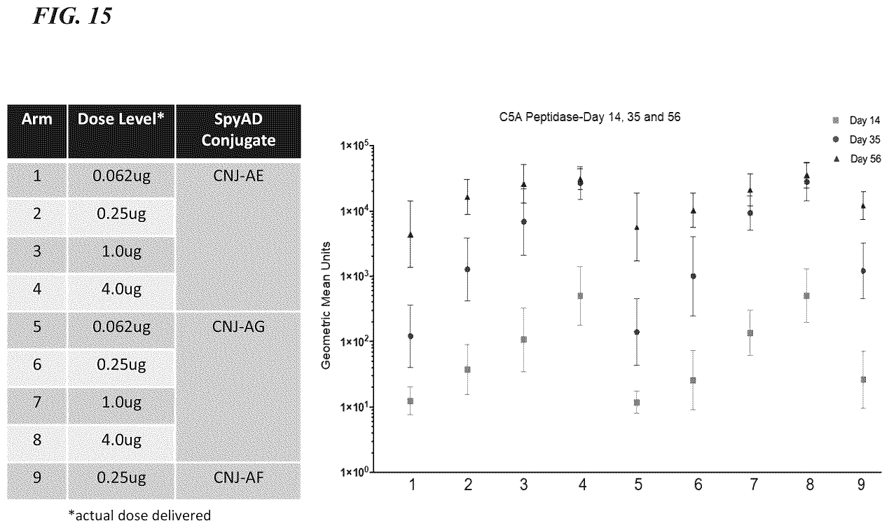

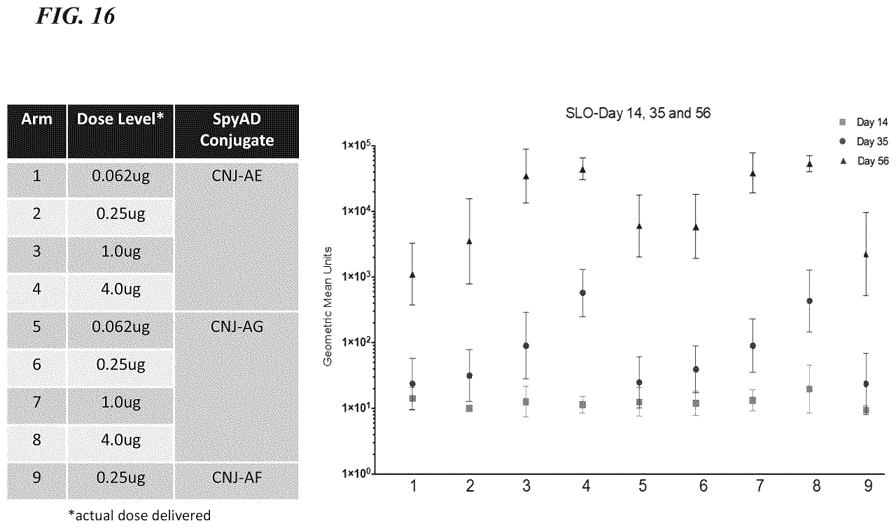

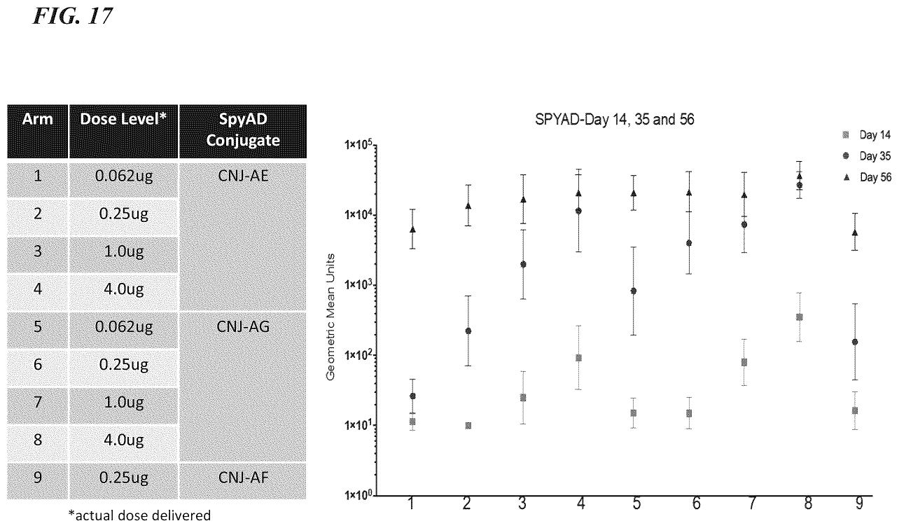

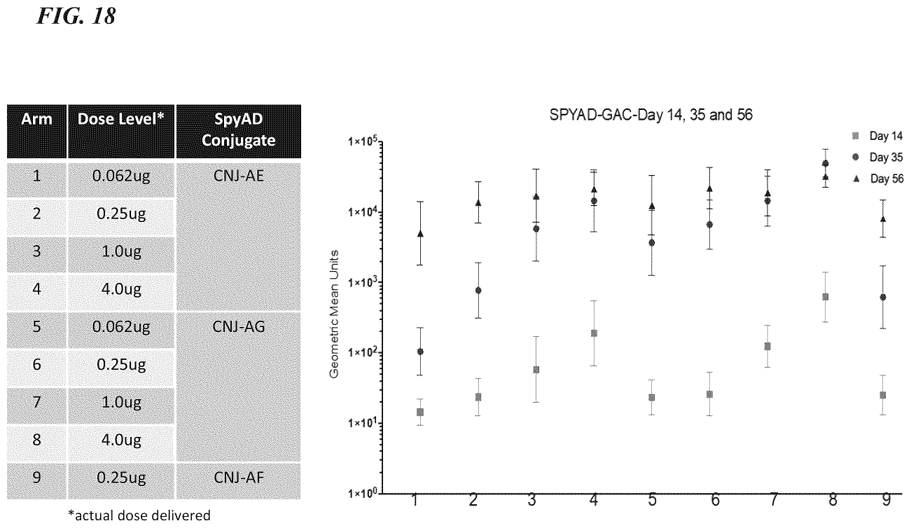

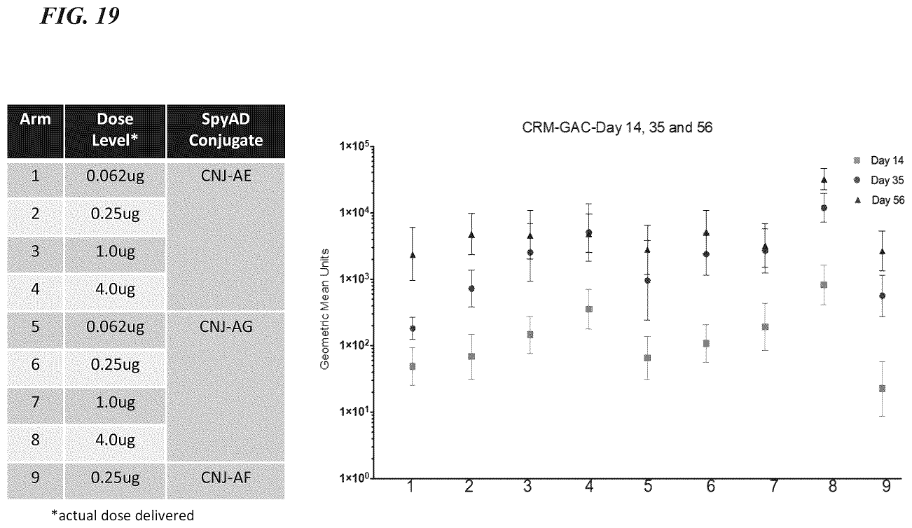

The results of each ELISA shown in FIGS. 15, 16, 17, 18, and 19 (C5a, SLO, SpyAD, SpyAD-GAC, and eCRM-GAC coating antigens, respectively), show that high molecular weight SpyAD-GAC conjugate (CNJ-AG) elicit stronger titers against both SpyAD and GAC than low molecular weight conjugates (CNJ-AE and CNJ-AF). In some cases, the high molecular weight conjugate elicits titers 3-30× higher than the low molecular weight conjugate (CNJ-AE) at the equivalent dose at day 35 (e.g., 0.062 and 0.25 μg, arms 6 vs. 2).

The stability of the conjugates is measured in various buffers to assess changes in mass recovery, conjugate molecular weight, and particle formation upon storage at 25° C. or upon undergoing multiple freeze/thaw cycles. Four such buffers are (i) 20 mM potassium phosphate pH 7.4, 10% sorbitol; (ii) 20 mM potassium phosphate pH 7.4, 10% sorbitol, 10 mM sodium chloride; (iii) 20 mM potassium phosphate pH 7.4, 10% sorbitol, 10 mM sodium chloride, 0.02% polysorbate 80; and (iv) 20 mM tris(hydroxymethyl)aminomethane pH 8.0, 10% sorbitol, 50 mM sodium chloride.

In vivo immunogenicity studies are also performed, as above, with conjugates of varying molecular weight, at one or two doses, and before and after being subjected to accelerated stability (e.g., higher temperatures than what might be used for a manufactured vaccine product) conditions or freeze/thaw cycles. For example, conjugates are below 250 kDa, between 250 and 400 kDa, and/or over 400 kDa. These experiments further assess the effect of conjugation conditions on yield and immunogenicity, assess how subjecting the conjugates described here to free/thaw cycles may results in a change in immunogenicity, and assess both of these variable at one or more dose levels.

### Biological Example 6: Human Phase I and Phase IIA Clinical Trial Design

Initial Phase I clinical studies will be a randomized, placebo-controlled, ascending dose study in healthy adults 18-29 years of age (N=96) (Table 3). Objectives of this initial clinical study will be safety, dose response, and immunogenicity (IgG antibody). Since many individuals at this age range will have pre-existing exposure and immunity to GAS, baseline immunity will be fully evaluated to understand the impact of pre-existing immunity on vaccine responses. IgG response to each vaccine component and OPK antibody titer against a diverse panel of contemporary GAS isolates of different M serotypes will be evaluated.

TABLE 5
Phase I Clinical Trial Groups
Phase I Clinical Trial Design

| Group | N | Treatment | Dose | Schedule |
|---|---|---|---|---|
| 1A | 24 | GAS vaccine | Low | Day 0, 28 |
| 1B | 8 | Placebo | | Day 0, 28 |
| 2A | 24 | GAS vaccine | Mid | Day 0, 28 |
| 2B | 8 | Placebo | | Day 0, 28 |
| 3A | 24 | GAS vaccine | High | Day 0, 28 |
| 3B | 8 | Placebo | | Day 0, 28 |

The Phase 2A clinical study will be a randomized, placebo controlled, multi-center study to evaluate the vaccine in successive cohorts of individuals from 10 to 17 years of age, followed by children 5 to 9 years old (N=96) (Table 4). The objectives of this study will be safety, immunogenicity (IgG antibody responses and opsonophagocytic activity of serum), and an evaluation of preliminary efficacy (incidence of GAS pharyngitis). Each patient will be monitored for 12 months to determine the incidence of strep pharyngitis in the treatment groups.

TABLE 6
Phase IIA Clinical Trial Groups
Phase IIA Clinical Trial Design

| N | Age (yr) | Treatment | Dose | Schedule |
|---|---|---|---|---|
| 36 | 10-17 | GAS vaccine | Determined by PI | Day 0, 28 |
| 12 | 10-17 | Placebo | | Day 0, 28 |
| 36 | 5-9 | GAS vaccine | Mid | Day 0, 28 |
| 12 | 5-9 | Placebo | | Day 0, 28 |

1. A polypeptide-polysaccharide conjugate comprising:
(a) a GAS polypeptide antigen or a non-GAS carrier polypeptide comprising at least one non-natural amino acid (nnAA), wherein the nnAA comprises a click chemistry reactive group; and
(b) a purified cell wall polysaccharide or a peptidoglycan-bound capsular polysaccharide with a molecular weight of at least about 10 kDa to at least about 40 kDa.

2. The polypeptide-polysaccharide conjugate of claim 1, wherein the purified cell wall polysaccharide lacks an immunodominant N-acetyl Glucosamine (GlcNAc) side chain.

3. The polypeptide-polysaccharide conjugate of claims 1-2, wherein the GAS polypeptide antigen is a full-length GAS polypeptide antigen or a fragment of a full-length GAS polypeptide antigen.

4. The polypeptide-polysaccharide conjugate of any one of claims 1-3, wherein the cell wall polysaccharide is a GAS polysaccharide, or a variant thereof.

5. The polypeptide-polysaccharide conjugate of claim 4, wherein between about 8 mol % and about 20 mol % of the polysaccharide repeat units of the GAS polysaccharide, or a variant thereof, are derivatized by a linker.

6. The polypeptide-polysaccharide conjugate of claims 4-5, wherein between about 10 mol % and about 20 mol % of the polysaccharide repeat units of the GAS polysaccharide, or a variant thereof, are derivatized by a linker.

7. The polypeptide-polysaccharide conjugate of claims 4-6, wherein between about 10 mol % and about 18 mol % of the polysaccharide repeat units of the GAS polysaccharide, or a variant thereof, are derivatized by a linker.

8. The polypeptide-polysaccharide conjugate of any one of claims 1-6, wherein the average molecular weight is between about 185 kDa and about 700.

9. The polypeptide-polysaccharide conjugate of any one of claims 1-7, wherein the average molecular weight is between about 200 kDa and about 700 kDa.

10. The polypeptide-polysaccharide conjugate of any one of claims 1-9, wherein the average molecular weight of the polypeptide-polysaccharide conjugate is between about 300 kDa and about 600 kDa.

11. The polypeptide-polysaccharide conjugate of any one of claims 1-10, wherein the average molecular weight of the polypeptide-polysaccharide conjugate is between about 400 kDa and about 500 kDa.

12. The polypeptide-polysaccharide conjugate of any one of claims 1-11, wherein the at least one nnAA is substituted for a lysine, a leucine, an isoleucine, or an arginine in the GAS polypeptide antigen or the non-GAS carrier polypeptide.

13. The polypeptide-polysaccharide conjugate of any one of claims 1-12, wherein the at least one nnAA is selected from 2-amino-3-(4-azidophenyl)propanoic acid (pAF), 2-amino-4-azidobutanoic acid, 2-azido-3-phenylpropionic acid, 2-amino-3-azidopropanoic acid, 2-amino-3-(4-(azidomethyl)phenyl)propanoic acid (pAMF), 2-amino-3-(5-(azidomethyl)pyridin-2-yl)propanoic acid, 2-amino-3-(4-(azidomethyl)pyridin-2-yl)propanoic acid, 2-amino-3-(6-(azidomethyl)pyridin-3-yl)propanoic acid, and 2-amino-5-azidopentanoic acid.

14. The polypeptide-polysaccharide conjugate of any one of claims 1-13, wherein the at least one nnAA is pAMF.

15. The polypeptide-polysaccharide conjugate of any one of claims 1-14, wherein the GAS polypeptide antigen is selected from C5a peptidase, streptolysin O (SLO), SpyAD, Sib35, and Sfb1.

16. The polypeptide-polysaccharide conjugate of any one of claims 1-15, wherein the GAS polypeptide antigen is a SpyAD polypeptide, or a fragment thereof.

17. The polypeptide-polysaccharide conjugate of claim 16, wherein the SpyAD polypeptide comprises an amino acid sequence that is at least 95% identical to SEQ ID NO: 33.

18. The polypeptide-polysaccharide conjugate of claim 16, wherein the SpyAD polypeptide is at least 95% identical to SEQ ID NO: 33.

19. The polypeptide-polysaccharide conjugate of any one of claims 16-18, wherein the SpyAD polypeptide comprises a pAMF substitution at positions K64, K287, K386, and K657 of SEQ ID NO: 33.

20. The polypeptide-polysaccharide conjugate of any one of claims 16-19, wherein the SpyAD polypeptide comprises the amino acid sequence of SEQ ID NO: 34.

21. The polypeptide-polysaccharide conjugate of any one of claims 16-19, wherein the SpyAD polypeptide has the amino acid sequence of SEQ ID NO: 34.

22. The polypeptide-polysaccharide conjugate of any one of claims 16-19, wherein the SpyAD polypeptide comprises the amino acid sequence of SEQ ID NO: 77, SEQ ID NO: 78, SEQ ID NO: 79, SEQ ID NO: 81, SEQ ID NO: 82, or SEQ ID NO: 83.

23. The polypeptide-polysaccharide conjugate of any one of claims 16-19, wherein the SpyAD polypeptide has the amino acid sequence of SEQ ID NO: 77, SEQ ID NO: 78, SEQ ID NO: 79, SEQ ID NO: 81, SEQ ID NO: 82, or SEQ ID NO: 83.

24. The polypeptide-polysaccharide conjugate of any one of claim 1-2 or 4-14, wherein the non-GAS carrier polypeptide is selected from ADI, ferritin, Protein D, and eCRM197.

25. The polypeptide-polysaccharide conjugate of claim 24, wherein the non-GAS carrier polypeptide is eCRM197.

26. The polypeptide-polysaccharide conjugate of claim 25, wherein the eCRM197 has the sequence of SEQ ID NO: 25.

27. The polypeptide-polysaccharide conjugate of any one of claims 1-13, wherein the GAS polypeptide antigen or a non-GAS carrier polypeptide comprises or consists comprises or consists of the amino acid sequence of a polypeptide listed in Table 1.

28. The polypeptide-polysaccharide conjugate of any one of claims 1-13, wherein the GAS polypeptide antigen or a non-GAS carrier polypeptide comprises or consists comprises or consists of the amino acid sequence of a polypeptide listed in Table 1A.

29. An immunogenic composition comprising:
(a) a Group A Streptococcus (GAS) C5a peptidase polypeptide antigen;
(b) a GAS streptolysin O (SLO) polypeptide antigen; and
(c) a polypeptide-polysaccharide conjugate comprising
a Streptococcus pyogenes Adhesion and Division (SpyAD) conjugate polypeptide, or a fragment thereof, comprising at least one non-natural amino acid (nnAA), wherein the at least one nnAA comprises a click chemistry reactive group, and
a GAS polysaccharide, or a variant thereof, that lacks an immunodominant N-acetyl Glucosamine (GlcNAc) side chain;
wherein between about 8 mol % and about 20 mol % of the polysaccharide repeat units of the GAS polysaccharide, or a variant thereof, are derivatized by a linker; and
wherein the average molecular weight of the polypeptide-polysaccharide conjugate is between about 185 kDa and about 700 kDa.

30. The immunogenic composition of claim 29, wherein the C5a peptidase polypeptide antigen comprises the amino acid sequence of SEQ ID NO: 29 or SEQ ID NO: 30.

31. The immunogenic composition of claim 29 or 30, wherein the C5a peptidase polypeptide antigen comprises an amino acid sequence that is at least 95% identical to SEQ ID NO: 29 or SEQ ID NO: 30.

32. The immunogenic composition of any one of claims 29-31, wherein the C5a peptidase polypeptide antigen has the amino acid sequence of SEQ ID NO: 29 or SEQ ID NO: 30.

33. The immunogenic composition of any one of claims 29-32, wherein the SLO polypeptide antigen comprises the amino acid sequence of SEQ ID NO: 31, SEQ ID NO: 32, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53.

34. The immunogenic composition of claims 29-33, wherein the SLO antigen comprises an amino acid sequence that is at least 95% identical to SEQ ID NO: 31, SEQ ID NO: 32, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53.

35. The immunogenic composition of any one of claims 29-34, wherein the SLO polypeptide antigen has the amino acid sequence of SEQ ID NO: 31, SEQ ID NO: 32, SEQ ID NO: 44, SEQ ID NO: 45, SEQ ID NO: 46, SEQ ID NO: 47, SEQ ID NO: 48, SEQ ID NO: 49, SEQ ID NO: 50, SEQ ID NO: 51, SEQ ID NO: 52, or SEQ ID NO: 53.

36. The immunogenic composition of any one of claims 29-35, wherein the at least one nnAA is selected from 2-amino-3-(4-azidophenyl)propanoic acid (pAF), 2-amino-4-azidobutanoic acid, 2-azido-3-phenylpropionic acid, 2-amino-3-azidopropanoic acid, 2-amino-3-(4-(azidomethyl)phenyl)propanoic acid (pAMF), 2-amino-3-(5-(azidomethyl)pyridin-2-yl)propanoic acid, 2-amino-3-(4-(azidomethyl)pyridin-2-yl)propanoic acid, 2-amino-3-(6-(azidomethyl)pyridin-3-yl)propanoic acid, and 2-amino-5-azidopentanoic acid.

37. The immunogenic composition of any one of claims 29-36, wherein the at least one nnAA is pAMF.

38. The immunogenic composition of any one of claims 29-37, wherein the SpyAD conjugate polypeptide, or a fragment thereof, comprises an amino acid sequence that is at least 95% identical to SEQ ID NO: 33.

39. The immunogenic composition of any one of claims 29-38, wherein the SpyAD conjugate polypeptide comprises an amino acid sequence that is a fragment of SEQ ID NO: 33.

40. The immunogenic composition of any one of claims 29-38, wherein the SpyAD conjugate polypeptide has an amino acid sequence that is a fragment of SEQ ID NO: 33.

41. The immunogenic composition of any one of claims 29-40, wherein the SpyAD conjugate polypeptide comprises a pAMF substitution at positions K64, K287, K386, and K657 of SEQ ID NO: 33.

42. The immunogenic composition of any one of claims 29-41, wherein the SpyAD conjugate polypeptide comprises the amino acid sequence of SEQ ID NO: 34, SEQ ID NO: 77, SEQ ID NO: 78, SEQ ID NO: 79, SEQ ID NO: 81, SEQ ID NO: 82, or SEQ ID NO: 83.

43. The immunogenic composition of any one of claims 29-42, wherein the SpyAD conjugate polypeptide comprises an amino acid sequence that is at least 95% identical to SEQ ID NO: 34, SEQ ID NO: 77, SEQ ID NO: 78, SEQ ID NO: 79, SEQ ID NO: 81, SEQ ID NO: 82, or SEQ ID NO: 83.

44. The immunogenic composition of any one of claims 29-43, wherein the SpyAD conjugate polypeptide has the amino acid sequence of SEQ ID NO: 34, SEQ ID NO: 77, SEQ ID NO: 78, SEQ ID NO: 79, SEQ ID NO: 81, SEQ ID NO: 82, or SEQ ID NO: 83.

45. The immunogenic composition of any one of claims 29-44, wherein the C5a peptidase polypeptide antigen comprises an amino acid sequence that is at least 95% identical to SEQ ID NO: 30; the SLO polypeptide antigen comprises an amino acid sequence that is at least 95% identical to SEQ ID NO: 53; and the SpyAD conjugate polypeptide comprises an amino acid sequence that is at least 95% identical to SEQ ID NO: 34.

46. The immunogenic composition of any one of claims 29-45 wherein the C5a peptidase polypeptide antigen comprises the amino acid sequence of SEQ ID NO: 30; the SLO polypeptide antigen comprises the amino acid sequence of SEQ ID NO: 53; and the SpyAD conjugate polypeptide comprises the amino acid sequence of SEQ ID NO: 34.

47. The immunogenic composition of any one of claims 29-46, wherein the C5a peptidase polypeptide antigen has the amino acid sequence of SEQ ID NO: 30; the SLO polypeptide antigen has the amino acid sequence of SEQ ID NO: 53; and the SpyAD conjugate polypeptide has the amino acid sequence of SEQ ID NO: 34.

48. The immunogenic composition of any one of claims 29-47, wherein between about 15 mol % and about 20 mol % of the polysaccharide repeat units of the GAS polysaccharide, or a variant thereof, are derivatized by a linker.

49. The immunogenic composition of any one of claims 29-48, wherein the linker, prior to reaction with the click chemistry reactive group of the nnAA, comprises a structure of Formula I: 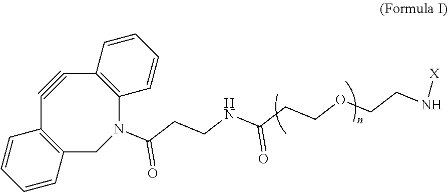
wherein, X is at least one polysaccharide repeat unit of a GAS polysaccharide, or a fragment thereof, and n is at least 1.

50. The immunogenic composition of any one of claims 29-49, wherein the SpyAD conjugate polypeptide, or fragment thereof, is linked to the GAS polysaccharide according to Formula II: 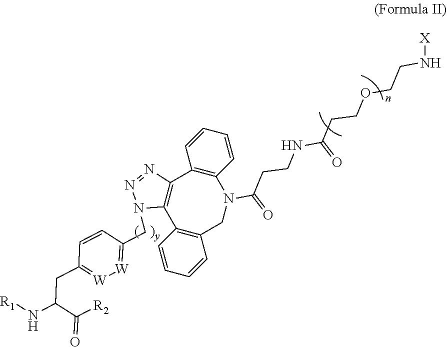
wherein,
R₁ is H, formyl, or at least one amino acid of the SpyAD conjugate polypeptide;
R₂ is OH or at least one amino acid of the SpyAD conjugate polypeptide;
W is CH or N;
y is at least 1;
n is at least 1; and
X is at least one polysaccharide repeat unit of a GAS polysaccharide or variant thereof.

51. The immunogenic composition of any one of claims 29-50, wherein the GAS polysaccharide, or a variant thereof, has a molecular weight of at least about 10 kDa to at least about 40 kDa.

52. The immunogenic composition of any one of claims 29-51, wherein the average molecular weight of the polypeptide-polysaccharide conjugate is between about 185 kDa and about 700.

53. The immunogenic composition of any one of claims 29-52, wherein the average molecular weight of the polypeptide-polysaccharide conjugate is between about 200 kDa and about 700 kDa.

54. The immunogenic composition of any one of claims 29-53, wherein the average molecular weight of the polypeptide-polysaccharide conjugate is between about 300 kDa and about 700 kDa.

55. The immunogenic composition of any one of claims 29-54, wherein the average molecular weight of the polypeptide-polysaccharide conjugate is between about 300 kDa and about 600 kDa.

56. The immunogenic composition of any one of claims 29-55 further comprising less than about 60% free GAS polysaccharide, or a variant thereof.

57. The immunogenic composition of any one of claims 29-56 further comprising less than about 50% free GAS polysaccharide, or a variant thereof.

58. The immunogenic composition of any one of claims 29-57 further comprising less than about 25% free GAS polysaccharide, or a variant thereof.

59. The immunogenic composition of any one of claims 29-58 further comprising less than about 15% free GAS polysaccharide, or a variant thereof.

60. The immunogenic composition of any one of claims 29-59 further comprising less than about 10% free GAS polysaccharide, or a variant thereof.

61. A method of inducing a protective immune response against a Group A Streptococcus (GAS) bacterium in a subject comprising administering the immunogenic composition of any one of claims 29-60 to the subject.

62. The method of claim 61, wherein the immunogenic composition induces an antibody response in the subject against the Group A Streptococcus (GAS) bacterium and does not induce an antibody response in the subject against human tissue.

63. The use of the immunogenic composition of any one of claims 29-60 in the manufacture of a medicament for inducing a protective immune response against a GAS bacterium in a subject.

64. Use of the immunogenic composition of any one of claims 29-60 for inducing a protective immune response against a GAS bacterium in a subject.

65. A process for purifying cell wall polysaccharides or peptidoglycan-bound capsular polysaccharides from a bacterial cell, the process comprising:
(a) hydrolyzing the bacterial cell in a solution comprising base and a reducing agent to form a lysate comprising polysaccharide; and
(b) incubating the lysate comprising polysaccharide with a muralytic enzyme to form a free polysaccharide solution.

66. The process according to claim 65, wherein the bacterial cell is aPseudomonas bacterial cell, a Streptococcus bacterial cell, a Staphylococcus bacterial cell, a Neisseria bacterial cell, a Haemophilus bacterial cell, a Listeria bacterial cell, a Enterococcus bacterial cell, or a Clostridium bacterial cell.

67. The process according to claim 65 or 66, wherein the bacterial cell is selected from of Pseudomonas aeruginosa, Streptococcus viridans, Streptococcus mutans, and Streptococcus pyogenes.

68. The process according to claim 67, wherein the Streptococcus pyogenes bacterial cell is of a serotype selected from M1, M2, M3, M4, M5, M6, M9, M11, M12, M13, M18, M22, M25, M28, M62, M71, M72, M74, M75, M77, M80, M81, M83, M87, M89, or M92.

69. The process according to claim 68, wherein the Streptococcus pyogenes bacterial cell produces a polysaccharide or a variant thereof that lacks an immunodominant N-acetyl Glucosamine (GlcNAc) side chain.

70. The process according to claim 65, for purifying a peptidoglycan-bound capsular polysaccharide from a bacterial cell.

71. The process according to any one of claims 65-70, wherein the base of step (a) is NaOH, KOH, or LiOH.

72. The process according to any one of claims 65-71, wherein the base of step (a) is NaOH.

73. The process according to any one of claims 65-72, wherein the concentration of base is between about 2M to about 8M.

74. The process according to any one of claims 65-73, wherein the solution comprising base and a reducing agent is about pH 14.

75. The process according to any one of claims 65-72, wherein the reducing agent is sodium borohydride, sodium cyanoborohydride, sodium triacetoxyborohydride, dithiothreitol, or beta-mercaptoethanol.

76. The process according to any one of claims 65-73, wherein the reducing agent is sodium borohydride.

77. The process according to any one of claims 65-76, wherein the concentration of the reducing agent is between about 1 mM and 500 mM.

78. The process according to any one of claims 65-75, wherein step (a) further comprises incubating the solution between about 30° C. and about 100° C.

79. The process according to any one of claims 65-78, wherein step (a) further comprises incubating the solution for between about 0.5 to about 20 hours.

80. The process according to any one of claims 65-79, wherein step (a) further comprises one or more pH adjustment steps.

81. The process according to claim 80, wherein the one or more pH adjustment steps are independently selected from:
(i) raising the lysate comprising polysaccharide pH, or
(ii) lowering the lysate comprising polysaccharide pH.

82. The process according to claim 81, comprising lowering the lysate comprising polysaccharide pH to between about 3 and 7.0.

83. The process according to claim 81 or 82, comprising lowering the lysate comprising polysaccharide pH to about 6.5.

84. The process according to claim 81 or 82, comprising lowering the lysate comprising polysaccharide pH to between about 3 and about 4.

85. The process according to claim 80 or 81, comprising
(i) lowering the lysate comprising polysaccharide pH to between about 5.5 and 7.0;
(ii) lowering the lysate comprising polysaccharide pH to about 3; and
(iii) raising the lysate comprising polysaccharide pH to between about 5.5 and 7.0.

86. The process according to any one of claims 80-85, wherein the lysate comprising polysaccharide is incubated at about room temperature (r.t.) after the one or more pH adjustment steps.

87. The process according to any one of claims 81-84, wherein the lysate comprising polysaccharide is incubated at between about 4° C. and about 30° C. after the one or more pH adjustment steps.

88. The process according to any one of claims 65-87, further comprising removing solids from the lysate comprising polysaccharide.

89. The process according to claim 88, wherein removing solids from the lysate comprising polysaccharide comprises filtration, centrifugation, or a combination thereof.

90. The process according to claim 89, wherein the filtration comprises depth filtration, tangential flow filtration (TFF), sterile filtration, or a combination of the foregoing.

91. The process according to claim 89 or 90, wherein the filtration comprises depth filtration followed by TFF.

92. The process according to claim 88 or 89, wherein solids are removed from the lysate comprising polysaccharide by centrifugation.

93. The process according to any one of claims 65-92, wherein the muralytic enzyme of step (b) is mutanolysin, lysozyme, or a bacteriophage hydrolase.

94. The process according to any one of claims 65-93, wherein step (b) further comprises incubating with a protease.

95. The process according to claim 94, wherein the protease is proteinase K, trypsin, chymotrypsin, endoproteinase Asp-N, endoproteinase Arg-C, endoproteinase Glu-C, endoproteinase Lys-C, pepsin, thermolysin, elastase, papain, substilisin, clostripain, carboxypeptidase A, carboxypeptidase B, carboxypeptidase P, carboxypeptidase Y, cathepsin C, acylamino-acid releasing enzyme, or pyroglutamate.

96. The process according to any one of claims 65-95, wherein step (b) further comprises warming the lysate comprising polysaccharide with the muralytic enzyme to between about 30° C. and about 65° C.

97. The process according to any one of claims 94-96, wherein the lysate comprising polysaccharide with the protease is warmed to between about 45° C. and 55° C.

98. The process according to claim 96 or 97, wherein the lysate is warmed between about 6 and about 20 hours.

99. The process according to any one of claims 65-98, wherein the free polysaccharide solution of step (b) is further purified to reduce the concentration of nucleic acids, enzymes, host cell proteins (HCPs), or a combination of the foregoing.

100. The process according to any one of claims 65-99, wherein the free polysaccharide solution of step (b) is further purified by precipitation.

101. The process according to any one of claims 65-100, wherein the free polysaccharide solution of step (b) is treated with cetyltrimethylammonium bromide (CTAB).

102. The process according to claim 101, wherein the concentration of CTAB in the free polysaccharide solution is about 0.1% to about 10%.

103. The process according to claim 101 or 102, the concentration of CTAB is between about 0.5% and about 3%.

104. The process according to any one of claims 101-103, wherein the free polysaccharide solution of step (b) is treated with potassium iodide (KI).

105. The process according to claim 104, wherein the concentration of KI in the free polysaccharide solution is between about 20 mM to about 400 mM.

106. The process according to any one of claims 65-105, wherein the free polysaccharide solution is further purified by filtration, centrifugation, chromatography, or a combination of the foregoing.

107. The process according to claim 106, wherein the filtration comprises depth filtration, tangential flow filtration (TFF), sterile filtration, or a combination of the foregoing.

108. The process according to claim 106, wherein the chromatography comprises hydrophobic interaction chromatography (HIC), anion-exchange chromatography (AEX), ceramic hydroxyapatite-type chromatography, or cation exchange chromatography (CEX).


SEQUENCE LISTING (83 sequences)

SEQ ID NO: 1 - protein, 962 aa, synthetic construct
  REGION 1..962: note = C5a [90-1035 fragment] - WT w/ leader
MHHHHHHGSGENLYFQGKTADTPVTSKATIRDLNDPSQVKTLQEKAGKGAGTVVAVIDAGFDKNHEAWRLTDKTKARYQSKEDLEKAKKEHGITYGEWVNDKVAYYHDYSKDGKTAVDQEHGTHVSGILSGNAPSETKEPYRLEGAMPEAQLLLMRVEIVNGLADYARNYAQAIRDAVNLGAKVINMSFGNAALAYANLPDETKKAFDYAKSKGVSIVTSAGNDSSFGGKTRLPLADHPDYGVVGTPAAADSTLTVASYSPDKQLTETATVKTADKQDKEMPVLSTNRFEPNKAYDYAYANRGMKEDDFKDVKGKIALIERGDIDFKDKIANAKKAGAVGVLIYDNQDKGFPIELPNVDQMPAAFISRKDGLLLKDNPKKTITFNATPKVLPTASGTKLSRFSSWGLTADGNIKPDIAAPGQDILSSVANNKYAKLSGTSMSAPLVAGIMGLLQKQYETQYPDMTPSERLDLAKKVLMSSATALYDEDEKAYFSPRQQGAGAVDAKKASAATMYVTDKDNTSSKVHLNNVSDTFEVTVTVHNKSDKPQELYYQATVQTDKVDGKHFALAPKALYETSWQKITIPANSSKQVTVPIDASRFSKDLLAQMKNGYFLEGFVRFKQDPTKEELMSIPYIGFRGDFGNLSALEKPIYDSKDGSSYYHEANSDAKDQLDGDGLQFYALKNNFTALTTESNPWTIIKAVKEGVENIEDIESSEITETIFAGTFAKQDDDSHYYIHRHANGKPYAAISPNGDGNRDYVQFQGTFLRNAKNLVAEVLDKEGNVVWTSEVTEQVVKNYNNDLASTLGSTRFEKTRWDGKDKDGKVVANGTYTYRVRYTPISSGAKEQHTDFDVIVDNTTPEVATSATFSTEDRRLTLASKPKTSQPVYRERIAYTYMDEDLPTTEYISPNEDGTFTLPEEAETMEGATVPLKMSDFTYVVEDMAGNITYTPVTKLLEGHSNK

SEQ ID NO: 2 - protein, 945 aa, Streptococcus pyogenes
KTADTPVTSKATIRDLNDPSQVKTLQEKAGKGAGTVVAVIDAGFDKNHEAWRLTDKTKARYQSKEDLEKAKKEHGITYGEWVNDKVAYYHDYSKDGKTAVDQEHGTHVSGILSGNAPSETKEPYRLEGAMPEAQLLLMRVEIVNGLADYARNYAQAIRDAVNLGAKVINMSFGNAALAYANLPDETKKAFDYAKSKGVSIVTSAGNDSSFGGKTRLPLADHPDYGVVGTPAAADSTLTVASYSPDKQLTETATVKTADKQDKEMPVLSTNRFEPNKAYDYAYANRGMKEDDFKDVKGKIALIERGDIDFKDKIANAKKAGAVGVLIYDNQDKGFPIELPNVDQMPAAFISRKDGLLLKDNPKKTITFNATPKVLPTASGTKLSRFSSWGLTADGNIKPDIAAPGQDILSSVANNKYAKLSGTSMSAPLVAGIMGLLQKQYETQYPDMTPSERLDLAKKVLMSSATALYDEDEKAYFSPRQQGAGAVDAKKASAATMYVTDKDNTSSKVHLNNVSDTFEVTVTVHNKSDKPQELYYQATVQTDKVDGKHFALAPKALYETSWQKITIPANSSKQVTVPIDASRFSKDLLAQMKNGYFLEGFVRFKQDPTKEELMSIPYIGFRGDFGNLSALEKPIYDSKDGSSYYHEANSDAKDQLDGDGLQFYALKNNFTALTTESNPWTIIKAVKEGVENIEDIESSEITETIFAGTFAKQDDDSHYYIHRHANGKPYAAISPNGDGNRDYVQFQGTFLRNAKNLVAEVLDKEGNVVWTSEVTEQVVKNYNNDLASTLGSTRFEKTRWDGKDKDGKVVANGTYTYRVRYTPISSGAKEQHTDFDVIVDNTTPEVATSATFSTEDRRLTLASKPKTSQPVYRERIAYTYMDEDLPTTEYISPNEDGTFTLPEEAETMEGATVPLKMSDFTYVVEDMAGNITYTPVTKLLEGHSNK

SEQ ID NO: 3 - protein, 962 aa, synthetic construct
  REGION 1..962: note = C5a [90-1035 fragment] D131A/S513A mutant w/ leader
MHHHHHHGSGENLYFQGKTADTPVTSKATIRDLNDPSQVKTLQEKAGKGAGTVVAVIAAGFDKNHEAWRLTDKTKARYQSKEDLEKAKKEHGITYGEWVNDKVAYYHDYSKDGKTAVDQEHGTHVSGILSGNAPSETKEPYRLEGAMPEAQLLLMRVEIVNGLADYARNYAQAIRDAVNLGAKVINMSFGNAALAYANLPDETKKAFDYAKSKGVSIVTSAGNDSSFGGKTRLPLADHPDYGVVGTPAAADSTLTVASYSPDKQLTETATVKTADKQDKEMPVLSTNRFEPNKAYDYAYANRGMKEDDFKDVKGKIALIERGDIDFKDKIANAKKAGAVGVLIYDNQDKGFPIELPNVDQMPAAFISRKDGLLLKDNPKKTITFNATPKVLPTASGTKLSRFSSWGLTADGNIKPDIAAPGQDILSSVANNKYAKLSGTAMSAPLVAGIMGLLQKQYETQYPDMTPSERLDLAKKVLMSSATALYDEDEKAYFSPRQQGAGAVDAKKASAATMYVTDKDNTSSKVHLNNVSDTFEVTVTVHNKSDKPQELYYQATVQTDKVDGKHFALAPKALYETSWQKITIPANSSKQVTVPIDASRFSKDLLAQMKNGYFLEGFVRFKQDPTKEELMSIPYIGFRGDFGNLSALEKPIYDSKDGSSYYHEANSDAKDQLDGDGLQFYALKNNFTALTTESNPWTIIKAVKEGVENIEDIESSEITETIFAGTFAKQDDDSHYYIHRHANGKPYAAISPNGDGNRDYVQFQGTFLRNAKNLVAEVLDKEGNVVWTSEVTEQVVKNYNNDLASTLGSTRFEKTRWDGKDKDGKVVANGTYTYRVRYTPISSGAKEQHTDFDVIVDNTTPEVATSATFSTEDRRLTLASKPKTSQPVYRERIAYTYMDEDLPTTEYISPNEDGTFTLPEEAETMEGATVPLKMSDFTYVVEDMAGNITYTPVTKLLEGHSNK

SEQ ID NO: 4 - protein, 945 aa, synthetic construct
  REGION 1..945: note = C5a [90-1035 fragment] D131A/S513A mutant w/o leader
KTADTPVTSKATIRDLNDPSQVKTLQEKAGKGAGTVVAVIAAGFDKNHEAWRLTDKTKARYQSKEDLEKAKKEHGITYGEWVNDKVAYYHDYSKDGKTAVDQEHGTHVSGILSGNAPSETKEPYRLEGAMPEAQLLLMRVEIVNGLADYARNYAQAIRDAVNLGAKVINMSFGNAALAYANLPDETKKAFDYAKSKGVSIVTSAGNDSSFGGKTRLPLADHPDYGVVGTPAAADSTLTVASYSPDKQLTETATVKTADKQDKEMPVLSTNRFEPNKAYDYAYANRGMKEDDFKDVKGKIALIERGDIDFKDKIANAKKAGAVGVLIYDNQDKGFPIELPNVDQMPAAFISRKDGLLLKDNPKKTITFNATPKVLPTASGTKLSRFSSWGLTADGNIKPDIAAPGQDILSSVANNKYAKLSGTAMSAPLVAGIMGLLQKQYETQYPDMTPSERLDLAKKVLMSSATALYDEDEKAYFSPRQQGAGAVDAKKASAATMYVTDKDNTSSKVHLNNVSDTFEVTVTVHNKSDKPQELYYQATVQTDKVDGKHFALAPKALYETSWQKITIPANSSKQVTVPIDASRFSKDLLAQMKNGYFLEGFVRFKQDPTKEELMSIPYIGFRGDFGNLSALEKPIYDSKDGSSYYHEANSDAKDQLDGDGLQFYALKNNFTALTTESNPWTIIKAVKEGVENIEDIESSEITETIFAGTFAKQDDDSHYYIHRHANGKPYAAISPNGDGNRDYVQFQGTFLRNAKNLVAEVLDKEGNVVWTSEVTEQVVKNYNNDLASTLGSTRFEKTRWDGKDKDGKVVANGTYTYRVRYTPISSGAKEQHTDFDVIVDNTTPEVATSATFSTEDRRLTLASKPKTSQPVYRERIAYTYMDEDLPTTEYISPNEDGTFTLPEEAETMEGATVPLKMSDFTYVVEDMAGNITYTPVTKLLEGHSNK

SEQ ID NO: 5 - protein, 510 aa, synthetic construct
  REGION 1..510: note = SLO [79-571] fragment w/ leader
MHHHHHHGSGENLYFQGAPKEMPLESAEKEEKKSEDKKKSEEDHTEEINDKIYSLNYNELEVLAKNGETIENFVPKEGVKKADKFIVIERKKKNINTTPVDISIIDSVTDRTYPAALQLANKGFTENKPDAVVTKRNPQKIHIDLPGMGDKATVEVNDPTYANVSTAIDNLVNQWHDNYSGGNTLPARTQYTESMVYSKSQIEAALNVNSKILDGTLGIDFKSISKGEKKVMIAAYKQIFYTVSANLPNNPADVFDKSVTFKELQRKGVSNEAPPLFVSNVAYGRTVFVKLETSSKSNDVEAAFSAALKGTDVKTNGKYSDILENSSFTAVVLGGDAAEHNKVVTKDFDVIRNVIKDNATFSRKNPAYPISYTSVFLKNNKIAGVNNRTEYVETTSTEYTSGKINLSHQGAYVAQYEILWDEINYDDKGKEVITKRRWDNNWYSKTSPFSTVIPLGANSRNIRIMARECTGLAWEWWRKVIDERDVKLSKEINVNISGSTLSPYGSITYK

SEQ ID NO: 6 - protein, 493 aa, Streptococcus pyogenes
APKEMPLESAEKEEKKSEDKKKSEEDHTEEINDKIYSLNYNELEVLAKNGETIENFVPKEGVKKADKFIVIERKKKNINTTPVDISIIDSVTDRTYPAALQLANKGFTENKPDAVVTKRNPQKIHIDLPGMGDKATVEVNDPTYANVSTAIDNLVNQWHDNYSGGNTLPARTQYTESMVYSKSQIEAALNVNSKILDGTLGIDFKSISKGEKKVMIAAYKQIFYTVSANLPNNPADVFDKSVTFKELQRKGVSNEAPPLFVSNVAYGRTVFVKLETSSKSNDVEAAFSAALKGTDVKTNGKYSDILENSSFTAVVLGGDAAEHNKVVTKDFDVIRNVIKDNATFSRKNPAYPISYTSVFLKNNKIAGVNNRTEYVETTSTEYTSGKINLSHQGAYVAQYEILWDEINYDDKGKEVITKRRWDNNWYSKTSPFSTVIPLGANSRNIRIMARECTGLAWEWWRKVIDERDVKLSKEINVNISGSTLSPYGSITYK

SEQ ID NO: 7 - protein, 510 aa, synthetic construct
  REGION 1..510: note = SLO [79-571 fragment] - W535 mutant w/ leader
MHHHHHHGSGENLYFQGAPKEMPLESAEKEEKKSEDKKKSEEDHTEEINDKIYSLNYNELEVLAKNGETIENFVPKEGVKKADKFIVIERKKKNINTTPVDISIIDSVTDRTYPAALQLANKGFTENKPDAVVTKRNPQKIHIDLPGMGDKATVEVNDPTYANVSTAIDNLVNQWHDNYSGGNTLPARTQYTESMVYSKSQIEAALNVNSKILDGTLGIDFKSISKGEKKVMIAAYKQIFYTVSANLPNNPADVFDKSVTFKELQRKGVSNEAPPLFVSNVAYGRTVFVKLETSSKSNDVEAAFSAALKGTDVKTNGKYSDILENSSFTAVVLGGDAAEHNKVVTKDFDVIRNVIKDNATFSRKNPAYPISYTSVFLKNNKIAGVNNRTEYVETTSTEYTSGKINLSHQGAYVAQYEILWDEINYDDKGKEVITKRRWDNNWYSKTSPFSTVIPLGANSRNIRIMARECTGLAAEWWRKVIDERDVKLSKEINVNISGSTLSPYGSITYK

SEQ ID NO: 8 - protein, 493 aa, synthetic construct
  REGION 1..493: note = SLO [79-571 fragment] - W535 mutant w/o leader
APKEMPLESAEKEEKKSEDKKKSEEDHTEEINDKIYSLNYNELEVLAKNGETIENFVPKEGVKKADKFIVIERKKKNINTTPVDISIIDSVTDRTYPAALQLANKGFTENKPDAVVTKRNPQKIHIDLPGMGDKATVEVNDPTYANVSTAIDNLVNQWHDNYSGGNTLPARTQYTESMVYSKSQIEAALNVNSKILDGTLGIDFKSISKGEKKVMIAAYKQIFYTVSANLPNNPADVFDKSVTFKELQRKGVSNEAPPLFVSNVAYGRTVFVKLETSSKSNDVEAAFSAALKGTDVKTNGKYSDILENSSFTAVVLGGDAAEHNKVVTKDFDVIRNVIKDNATFSRKNPAYPISYTSVFLKNNKIAGVNNRTEYVETTSTEYTSGKINLSHQGAYVAQYEILWDEINYDDKGKEVITKRRWDNNWYSKTSPFSTVIPLGANSRNIRIMARECTGLAAEWWRKVIDERDVKLSKEINVNISGSTLSPYGSITYK

SEQ ID NO: 9 - protein, 834 aa, synthetic construct
  REGION 1..834: note = SpyAD [33-849 fragment] WT w/ leader
MHHHHHHGSGENLYFQGQVKADDRASGETKASNTHDDSLPKPETIQEAKATIDAVEKTLSQQKAELTELATALTKTTAEINHLKEQQDNEQKALTSAQEIYTNTLASSEETLLAQGAEHQRELTATETELHNAQADQHSKETALSEQKASISAETTRAQDLVEQVKTSEQNIAKLNAMISNPDAITKAAQTANDNTKALSSELEKAKADLENQKAKVKKQLTEELAAQKAALAEKEAELSRLKSSAPSTQDSIVGNNTMKAPQGYPLEELKKLEASGYIGSASYNNYYKEHADQIIAKASPGNQLNQYQDIPADRNRFVDPDNLTPEVQNELAQFAAHMINSVRRQLGLPPVTVTAGSQEFARLLSTSYKKTHGNTRPSFVYGQPGVSGHYGVGPHDKTIIEDSAGASGLIRNDDNMYENIGAFNDVHTVNGIKRGIYDSIKYMLFTDHLHGNTYGHAINFLRVDKHNPNAPVYLGFSTSNVGSLNEHFVMFPESNIANHQRFNKTPIKAVGSTKDYAQRVGTVSDTIAAIKGKVSSLENRLSAIHQEADIMAAQAKVSQLQGKLASTLKQSDSLNLQVRQLNDTKGSLRTELLAAKAKQAQLEATRDQSLAKLASLKAALHQTEALAEQAAARVTALVAKKAHLQYLRDFKLNPNRLQVIRERIDNTKQDLAKTTSSLLNAQEALAALQAKQSSLEATIATTEHQLTLLKTLANEKEYRHLDEDIATVPDLQVAPPLTGVKPLSYSKIDTTPLVQEMVKETKQLLEASARLAAENTSLVAEALVGQTSEMVASNAIVSKITSSITQPSSKTSYGSGSSTTSNLISDVDESTQR

SEQ ID NO: 10 - protein, 817 aa, Streptococcus pyogenes
QVKADDRASGETKASNTHDDSLPKPETIQEAKATIDAVEKTLSQQKAELTELATALTKTTAEINHLKEQQDNEQKALTSAQEIYTNTLASSEETLLAQGAEHQRELTATETELHNAQADQHSKETALSEQKASISAETTRAQDLVEQVKTSEQNIAKLNAMISNPDAITKAAQTANDNTKALSSELEKAKADLENQKAKVKKQLTEELAAQKAALAEKEAELSRLKSSAPSTQDSIVGNNTMKAPQGYPLEELKKLEASGYIGSASYNNYYKEHADQIIAKASPGNQLNQYQDIPADRNRFVDPDNLTPEVQNELAQFAAHMINSVRRQLGLPPVTVTAGSQEFARLLSTSYKKTHGNTRPSFVYGQPGVSGHYGVGPHDKTIIEDSAGASGLIRNDDNMYENIGAFNDVHTVNGIKRGIYDSIKYMLFTDHLHGNTYGHAINFLRVDKHNPNAPVYLGFSTSNVGSLNEHFVMFPESNIANHQRFNKTPIKAVGSTKDYAQRVGTVSDTIAAIKGKVSSLENRLSAIHQEADIMAAQAKVSQLQGKLASTLKQSDSLNLQVRQLNDTKGSLRTELLAAKAKQAQLEATRDQSLAKLASLKAALHQTEALAEQAAARVTALVAKKAHLQYLRDFKLNPNRLQVIRERIDNTKQDLAKTTSSLLNAQEALAALQAKQSSLEATIATTEHQLTLLKTLANEKEYRHLDEDIATVPDLQVAPPLTGVKPLSYSKIDTTPLVQEMVKETKQLLEASARLAAENTSLVAEALVGQTSEMVASNAIVSKITSSITQPSSKTSYGSGSSTTSNLISDVDESTQR

SEQ ID NO: 11 - protein, 834 aa, synthetic construct
  REGION 1..834: note = SpyAD [33-849 fragment] nnAA (e.g. pAMF) K64/K287/K386/K657 mutant w/ leader
  VARIANT 49: note = any non-natural amino acid (e.g pAMF)
  VARIANT 272: note = any non-natural amino acid (e.g pAMF)
  VARIANT 371: note = any non-natural amino acid (e.g pAMF)
  VARIANT 642: note = any non-natural amino acid (e.g pAMF)
MHHHHHHGSGENLYFQGQVKADDRASGETKASNTHDDSLPKPETIQEAXATIDAVEKTLSQQKAELTELATALTKTTAEINHLKEQQDNEQKALTSAQEIYTNTLASSEETLLAQGAEHQRELTATETELHNAQADQHSKETALSEQKASISAETTRAQDLVEQVKTSEQNIAKLNAMISNPDAITKAAQTANDNTKALSSELEKAKADLENQKAKVKKQLTEELAAQKAALAEKEAELSRLKSSAPSTQDSIVGNNTMKAPQGYPLEELKXLEASGYIGSASYNNYYKEHADQIIAKASPGNQLNQYQDIPADRNRFVDPDNLTPEVQNELAQFAAHMINSVRRQLGLPPVTVTAGSQEFARLLSTSYKXTHGNTRPSFVYGQPGVSGHYGVGPHDKTIIEDSAGASGLIRNDDNMYENIGAFNDVHTVNGIKRGIYDSIKYMLFTDHLHGNTYGHAINFLRVDKHNPNAPVYLGFSTSNVGSLNEHFVMFPESNIANHQRFNKTPIKAVGSTKDYAQRVGTVSDTIAAIKGKVSSLENRLSAIHQEADIMAAQAKVSQLQGKLASTLKQSDSLNLQVRQLNDTKGSLRTELLAAKAKQAQLEATRDQSLAKLASLKAALHQTEALAEQAAARVTALVAKXAHLQYLRDFKLNPNRLQVIRERIDNTKQDLAKTTSSLLNAQEALAALQAKQSSLEATIATTEHQLTLLKTLANEKEYRHLDEDIATVPDLQVAPPLTGVKPLSYSKIDTTPLVQEMVKETKQLLEASARLAAENTSLVAEALVGQTSEMVASNAIVSKITSSITQPSSKTSYGSGSSTTSNLISDVDESTQR

SEQ ID NO: 12 - protein, 817 aa, synthetic construct
  REGION 1..817: note = SpyAD [33-849 fragment] nnAA (e.g. pAMF) K64/K287/K386/K657 mutant w/o leader
  VARIANT 31: note = any non-natural amino acid (e.g pAMF)
  VARIANT 32: note = any naturally occurring amino acid
  VARIANT 255: note = any non-natural amino acid (e.g pAMF)
  VARIANT 354: note = any non-natural amino acid (e.g pAMF)
  VARIANT 625: note = any non-natural amino acid (e.g pAMF)
QVKADDRASGETKASNTHDDSLPKPETIQEAXATIDAVEKTLSQQKAELTELATALTKTTAEINHLKEQQDNEQKALTSAQEIYTNTLASSEETLLAQGAEHQRELTATETELHNAQADQHSKETALSEQKASISAETTRAQDLVEQVKTSEQNIAKLNAMISNPDAITKAAQTANDNTKALSSELEKAKADLENQKAKVKKQLTEELAAQKAALAEKEAELSRLKSSAPSTQDSIVGNNTMKAPQGYPLEELKXLEASGYIGSASYNNYYKEHADQIIAKASPGNQLNQYQDIPADRNRFVDPDNLTPEVQNELAQFAAHMINSVRRQLGLPPVTVTAGSQEFARLLSTSYKXTHGNTRPSFVYGQPGVSGHYGVGPHDKTIIEDSAGASGLIRNDDNMYENIGAFNDVHTVNGIKRGIYDSIKYMLFTDHLHGNTYGHAINFLRVDKHNPNAPVYLGFSTSNVGSLNEHFVMFPESNIANHQRFNKTPIKAVGSTKDYAQRVGTVSDTIAAIKGKVSSLENRLSAIHQEADIMAAQAKVSQLQGKLASTLKQSDSLNLQVRQLNDTKGSLRTELLAAKAKQAQLEATRDQSLAKLASLKAALHQTEALAEQAAARVTALVAKXAHLQYLRDFKLNPNRLQVIRERIDNTKQDLAKTTSSLLNAQEALAALQAKQSSLEATIATTEHQLTLLKTLANEKEYRHLDEDIATVPDLQVAPPLTGVKPLSYSKIDTTPLVQEMVKETKQLLEASARLAAENTSLVAEALVGQTSEMVASNAIVSKITSSITQPSSKTSYGSGSSTTSNLISDVDESTQR

SEQ ID NO: 13 - protein, 427 aa, synthetic construct
  REGION 1..427: note = ADI [full length] WT w/ leader
MHHHHHHGSGENLYFQGTAQTPIHVYSEIGKLKKVLLHRPGKEIENLMPDYLERLLFDDIPFLEDAQKEHDAFAQALRDEGIEVLYLETLAAESLVTPEIREAFIDEYLSEANIRGRATKKAIRELLMAIEDNQELIEKTMAGVQKSELPEIPASEKGLTDLVESNYPFAIDPMPNLYFTRDPFATIGTGVSLNHMFSETRNRETLYGKYIFTHHPIYGGGKVPMVYDRNETTRIEGGDELVLSKDVLAVGISQRTDAASIEKLLVNIFKQNLGFKKVLAFEFANNRKFMHLDTVFTMVDYDKFTIHPEIEGDLRVYSVTYDNEELHIVEEKGDLAELLAANLGVEKVDLIRCGGDNLVAAGREQWNDGSNTLTIAPGVVVVYNRNTITNAILESKGLKLIKIHGSELVRGRGGPRCMSMPFEREDI

SEQ ID NO: 14 - protein, 410 aa, Streptococcus pyogenes
TAQTPIHVYSEIGKLKKVLLHRPGKEIENLMPDYLERLLFDDIPFLEDAQKEHDAFAQALRDEGIEVLYLETLAAESLVTPEIREAFIDEYLSEANIRGRATKKAIRELLMAIEDNQELIEKTMAGVQKSELPEIPASEKGLTDLVESNYPFAIDPMPNLYFTRDPFATIGTGVSLNHMFSETRNRETLYGKYIFTHHPIYGGGKVPMVYDRNETTRIEGGDELVLSKDVLAVGISQRTDAASIEKLLVNIFKQNLGFKKVLAFEFANNRKFMHLDTVFTMVDYDKFTIHPEIEGDLRVYSVTYDNEELHIVEEKGDLAELLAANLGVEKVDLIRCGGDNLVAAGREQWNDGSNTLTIAPGVVVVYNRNTITNAILESKGLKLIKIHGSELVRGRGGPRCMSMPFEREDI

SEQ ID NO: 15 - protein, 427 aa, synthetic construct
  REGION 1..427: note = ADI [full length] D277A mut w/ leader
MHHHHHHGSGENLYFQGTAQTPIHVYSEIGKLKKVLLHRPGKEIENLMPDYLERLLFDDIPFLEDAQKEHDAFAQALRDEGIEVLYLETLAAESLVTPEIREAFIDEYLSEANIRGRATKKAIRELLMAIEDNQELIEKTMAGVQKSELPEIPASEKGLTDLVESNYPFAIDPMPNLYFTRDPFATIGTGVSLNHMFSETRNRETLYGKYIFTHHPIYGGGKVPMVYDRNETTRIEGGDELVLSKDVLAVGISQRTDAASIEKLLVNIFKQNLGFKKVLAFEFANNRKFMHLATVFTMVDYDKFTIHPEIEGDLRVYSVTYDNEELHIVEEKGDLAELLAANLGVEKVDLIRCGGDNLVAAGREQWNDGSNTLTIAPGVVVVYNRNTITNAILESKGLKLIKIHGSELVRGRGGPRCMSMPFEREDI

SEQ ID NO: 16 - protein, 410 aa, synthetic construct
  REGION 1..410: note = ADI [full length] D277A mut w/o leader
TAQTPIHVYSEIGKLKKVLLHRPGKEIENLMPDYLERLLFDDIPFLEDAQKEHDAFAQALRDEGIEVLYLETLAAESLVTPEIREAFIDEYLSEANIRGRATKKAIRELLMAIEDNQELIEKTMAGVQKSELPEIPASEKGLTDLVESNYPFAIDPMPNLYFTRDPFATIGTGVSLNHMFSETRNRETLYGKYIFTHHPIYGGGKVPMVYDRNETTRIEGGDELVLSKDVLAVGISQRTDAASIEKLLVNIFKQNLGFKKVLAFEFANNRKFMHLATVFTMVDYDKFTIHPEIEGDLRVYSVTYDNEELHIVEEKGDLAELLAANLGVEKVDLIRCGGDNLVAAGREQWNDGSNTLTIAPGVVVVYNRNTITNAILESKGLKLIKIHGSELVRGRGGPRCMSMPFEREDI

SEQ ID NO: 17 - protein, 427 aa, synthetic construct
  REGION 1..427: note = ADI [full length] WT nnAA (e.g. pAMF) K15/K193/K316 w/ leader
  VARIANT 31: note = any non-natural amino acid (e.g pAMF)
  VARIANT 209: note = any non-natural amino acid (e.g pAMF)
  VARIANT 332: note = any non-natural amino acid (e.g pAMF)
MHHHHHHGSGENLYFQGTAQTPIHVYSEIGXLKKVLLHRPGKEIENLMPDYLERLLFDDIPFLEDAQKEHDAFAQALRDEGIEVLYLETLAAESLVTPEIREAFIDEYLSEANIRGRATKKAIRELLMAIEDNQELIEKTMAGVQKSELPEIPASEKGLTDLVESNYPFAIDPMPNLYFTRDPFATIGTGVSLNHMFSETRNRETLYGXYIFTHHPIYGGGKVPMVYDRNETTRIEGGDELVLSKDVLAVGISQRTDAASIEKLLVNIFKQNLGFKKVLAFEFANNRKFMHLDTVFTMVDYDKFTIHPEIEGDLRVYSVTYDNEELHIVEEXGDLAELLAANLGVEKVDLIRCGGDNLVAAGREQWNDGSNTLTIAPGVVVVYNRNTITNAILESKGLKLIKIHGSELVRGRGGPRCMSMPFEREDI

SEQ ID NO: 18 - protein, 410 aa, synthetic construct
  REGION 1..410: note = ADI [full length] WT nnAA (e.g. pAMF) K15/K193/K316 w/o leader
  VARIANT 14: note = any non-natural amino acid (e.g pAMF)
  VARIANT 192: note = any non-natural amino acid (e.g pAMF)
  VARIANT 315: note = any non-natural amino acid (e.g pAMF)
TAQTPIHVYSEIGXLKKVLLHRPGKEIENLMPDYLERLLFDDIPFLEDAQKEHDAFAQALRDEGIEVLYLETLAAESLVTPEIREAFIDEYLSEANIRGRATKKAIRELLMAIEDNQELIEKTMAGVQKSELPEIPASEKGLTDLVESNYPFAIDPMPNLYFTRDPFATIGTGVSLNHMFSETRNRETLYGXYIFTHHPIYGGGKVPMVYDRNETTRIEGGDELVLSKDVLAVGISQRTDAASIEKLLVNIFKQNLGFKKVLAFEFANNRKFMHLDTVFTMVDYDKFTIHPEIEGDLRVYSVTYDNEELHIVEEXGDLAELLAANLGVEKVDLIRCGGDNLVAAGREQWNDGSNTLTIAPGVVVVYNRNTITNAILESKGLKLIKIHGSELVRGRGGPRCMSMPFEREDI

SEQ ID NO: 19 - protein, 427 aa, synthetic construct
  REGION 1..427: note = ADI [full length] D277A nnAA (e.g. pAMF) K15/K193/K316 w/ leader
  VARIANT 31: note = any non-natural amino acid (e.g pAMF)
  VARIANT 209: note = any non-natural amino acid (e.g pAMF)
  VARIANT 332: note = any non-natural amino acid (e.g pAMF)
MHHHHHHGSGENLYFQGTAQTPIHVYSEIGXLKKVLLHRPGKEIENLMPDYLERLLFDDIPFLEDAQKEHDAFAQALRDEGIEVLYLETLAAESLVTPEIREAFIDEYLSEANIRGRATKKAIRELLMAIEDNQELIEKTMAGVQKSELPEIPASEKGLTDLVESNYPFAIDPMPNLYFTRDPFATIGTGVSLNHMFSETRNRETLYGXYIFTHHPIYGGGKVPMVYDRNETTRIEGGDELVLSKDVLAVGISQRTDAASIEKLLVNIFKQNLGFKKVLAFEFANNRKFMHLATVFTMVDYDKFTIHPEIEGDLRVYSVTYDNEELHIVEEXGDLAELLAANLGVEKVDLIRCGGDNLVAAGREQWNDGSNTLTIAPGVVVVYNRNTITNAILESKGLKLIKIHGSELVRGRGGPRCMSMPFEREDI

SEQ ID NO: 20 - protein, 410 aa, synthetic construct
  REGION 1..410: note = ADI [full length] D277A nnAA (e.g. pAMF) K15/K193/K316 w/o leader
  VARIANT 14: note = any non-natural amino acid (e.g pAMF)
  VARIANT 192: note = any non-natural amino acid (e.g pAMF)
  VARIANT 315: note = any non-natural amino acid (e.g pAMF)
TAQTPIHVYSEIGXLKKVLLHRPGKEIENLMPDYLERLLFDDIPFLEDAQKEHDAFAQALRDEGIEVLYLETLAAESLVTPEIREAFIDEYLSEANIRGRATKKAIRELLMAIEDNQELIEKTMAGVQKSELPEIPASEKGLTDLVESNYPFAIDPMPNLYFTRDPFATIGTGVSLNHMFSETRNRETLYGXYIFTHHPIYGGGKVPMVYDRNETTRIEGGDELVLSKDVLAVGISQRTDAASIEKLLVNIFKQNLGFKKVLAFEFANNRKFMHLATVFTMVDYDKFTIHPEIEGDLRVYSVTYDNEELHIVEEXGDLAELLAANLGVEKVDLIRCGGDNLVAAGREQWNDGSNTLTIAPGVVVVYNRNTITNAILESKGLKLIKIHGSELVRGRGGPRCMSMPFEREDI

SEQ ID NO: 21 - protein, 184 aa, synthetic construct
  REGION 1..184: note = hFerritin [full length] WT w/ leader
MHHHHHHGSGSSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLKHD

SEQ ID NO: 22 - protein, 174 aa, Homo sapiens
SSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLKHD

SEQ ID NO: 23 - protein, 184 aa, synthetic construct
  REGION 1..184: note = hFerritin [full length] nnAA (e.g. pAMF) mutant I5 w/ leader
  VARIANT 14: note = any non-natural amino acid (e.g pAMF)
MHHHHHHGSGSSQXRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLKHD

SEQ ID NO: 24 - protein, 174 aa, synthetic construct
  REGION 1..174: note = hFerritin [full length] nnAA (e.g. pAMF) mutant I5 w/o leader
  VARIANT 4: note = any non-natural amino acid (e.g pAMF)
SSQXRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLKHD

SEQ ID NO: 25 - protein, 536 aa, synthetic construct
  REGION 1..536: note = eCRM - nnAA (e.g. pAMF) mutant
  VARIANT 34: note = any non-natural amino acid (e.g pAMF)
  VARIANT 213: note = any non-natural amino acid (e.g pAMF)
  VARIANT 245: note = any non-natural amino acid (e.g pAMF)
  VARIANT 265: note = any non-natural amino acid (e.g pAMF)
  VARIANT 386: note = any non-natural amino acid (e.g pAMF)
  VARIANT 527: note = any non-natural amino acid (e.g pAMF)
MGADDVVDSSKSFVMENFSSYHGTKPGYVDSIQXGIQKPKSGTQGNYDDDWKEFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRNSVGSSLSCINLDWDVIRDXTKTKIESLKEHGPIKNKMSESPNKTVSEEKAXQYLEEFHQTALEHPELSELXTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHXTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSXLSLFFEIKS

SEQ ID NO: 26 - protein, 427 aa, synthetic construct
  REGION 1..427: note = ADI (mut2) [full length] D277A nnAA (e.g. pAMF) mutant w/ leader
  VARIANT 139: note = any non-natural amino acid (e.g pAMF)
  VARIANT 263: note = any non-natural amino acid (e.g pAMF)
  VARIANT 332: note = any non-natural amino acid (e.g pAMF)
MHHHHHHGSGENLYFQGTAQTPIHVYSEIGKLKKVLLHRPGKEIENLMPDYLERLLFDDIPFLEDAQKEHDAFAQALRDEGIEVLYLETLAAESLVTPEIREAFIDEYLSEANIRGRATKKAIRELLMAIEDNQELIEXTMAGVQKSELPEIPASEKGLTDLVESNYPFAIDPMPNLYFTRDPFATIGTGVSLNHMFSETRNRETLYGKYIFTHHPIYGGGKVPMVYDRNETTRIEGGDELVLSKDVLAVGISQRTDAASIEXLLVNIFKQNLGFKKVLAFEFANNRKFMHLATVFTMVDYDKFTIHPEIEGDLRVYSVTYDNEELHIVEEXGDLAELLAANLGVEKVDLIRCGGDNLVAAGREQWNDGSNTLTIAPGVVVVYNRNTITNAILESKGLKLIKIHGSELVRGRGGPRCMSMPFEREDI

SEQ ID NO: 27 - protein, 411 aa, synthetic construct
  REGION 1..411: note = ADI (mut2) [full length] D277A nnAA (e.g. pAMF) mutant w/o leader, with G overhang from cleavage
  VARIANT 123: note = any non-natural amino acid (e.g pAMF)
  VARIANT 247: note = any non-natural amino acid (e.g pAMF)
  VARIANT 316: note = any non-natural amino acid (e.g pAMF)
GTAQTPIHVYSEIGKLKKVLLHRPGKEIENLMPDYLERLLFDDIPFLEDAQKEHDAFAQALRDEGIEVLYLETLAAESLVTPEIREAFIDEYLSEANIRGRATKKAIRELLMAIEDNQELIEXTMAGVQKSELPEIPASEKGLTDLVESNYPFAIDPMPNLYFTRDPFATIGTGVSLNHMFSETRNRETLYGKYIFTHHPIYGGGKVPMVYDRNETTRIEGGDELVLSKDVLAVGISQRTDAASIEXLLVNIFKQNLGFKKVLAFEFANNRKFMHLATVFTMVDYDKFTIHPEIEGDLRVYSVTYDNEELHIVEEXGDLAELLAANLGVEKVDLIRCGGDNLVAAGREQWNDGSNTLTIAPGVVVVYNRNTITNAILESKGLKLIKIHGSELVRGRGGPRCMSMPFEREDI

SEQ ID NO: 28 - protein, 157 aa, synthetic construct
  REGION 1..157: note = Sfb1 repeat region with leader
MHHHHHHGSGVETEDTKEPGVLMGGQSESVEFTKDTQTGMSGQTTPQVETEDTKEPGVLMGGQSESVEFTKDTQTGMSGQTASQVETEDTKEPGVLMGGQSESVEFTKDTQTGMSGQTTPQVETEDTKEPGVLMGGQSESVEFTKDTQTGMSGFSET

SEQ ID NO: 29 - protein, 946 aa, synthetic construct
  REGION 1..946: note = C5a [90-1035 fragment] WT w/o leader, with G overhang from cleavage
GKTADTPVTSKATIRDLNDPSQVKTLQEKAGKGAGTVVAVIDAGFDKNHEAWRLTDKTKARYQSKEDLEKAKKEHGITYGEWVNDKVAYYHDYSKDGKTAVDQEHGTHVSGILSGNAPSETKEPYRLEGAMPEAQLLLMRVEIVNGLADYARNYAQAIRDAVNLGAKVINMSFGNAALAYANLPDETKKAFDYAKSKGVSIVTSAGNDSSFGGKTRLPLADHPDYGVVGTPAAADSTLTVASYSPDKQLTETATVKTADKQDKEMPVLSTNRFEPNKAYDYAYANRGMKEDDFKDVKGKIALIERGDIDFKDKIANAKKAGAVGVLIYDNQDKGFPIELPNVDQMPAAFISRKDGLLLKDNPKKTITFNATPKVLPTASGTKLSRFSSWGLTADGNIKPDIAAPGQDILSSVANNKYAKLSGTSMSAPLVAGIMGLLQKQYETQYPDMTPSERLDLAKKVLMSSATALYDEDEKAYFSPRQQGAGAVDAKKASAATMYVTDKDNTSSKVHLNNVSDTFEVTVTVHNKSDKPQELYYQATVQTDKVDGKHFALAPKALYETSWQKITIPANSSKQVTVPIDASRFSKDLLAQMKNGYFLEGFVRFKQDPTKEELMSIPYIGFRGDFGNLSALEKPIYDSKDGSSYYHEANSDAKDQLDGDGLQFYALKNNFTALTTESNPWTIIKAVKEGVENIEDIESSEITETIFAGTFAKQDDDSHYYIHRHANGKPYAAISPNGDGNRDYVQFQGTFLRNAKNLVAEVLDKEGNVVWTSEVTEQVVKNYNNDLASTLGSTRFEKTRWDGKDKDGKVVANGTYTYRVRYTPISSGAKEQHTDFDVIVDNTTPEVATSATFSTEDRRLTLASKPKTSQPVYRERIAYTYMDEDLPTTEYISPNEDGTFTLPEEAETMEGATVPLKMSDFTYVVEDMAGNITYTPVTKLLEGHSNK

SEQ ID NO: 30 - protein, 946 aa, synthetic construct
  REGION 1..946: note = C5a [90-1035 fragment] D131A/S513A w/o leader, with G overhang from cleavage
GKTADTPVTSKATIRDLNDPSQVKTLQEKAGKGAGTVVAVIAAGFDKNHEAWRLTDKTKARYQSKEDLEKAKKEHGITYGEWVNDKVAYYHDYSKDGKTAVDQEHGTHVSGILSGNAPSETKEPYRLEGAMPEAQLLLMRVEIVNGLADYARNYAQAIRDAVNLGAKVINMSFGNAALAYANLPDETKKAFDYAKSKGVSIVTSAGNDSSFGGKTRLPLADHPDYGVVGTPAAADSTLTVASYSPDKQLTETATVKTADKQDKEMPVLSTNRFEPNKAYDYAYANRGMKEDDFKDVKGKIALIERGDIDFKDKIANAKKAGAVGVLIYDNQDKGFPIELPNVDQMPAAFISRKDGLLLKDNPKKTITFNATPKVLPTASGTKLSRFSSWGLTADGNIKPDIAAPGQDILSSVANNKYAKLSGTAMSAPLVAGIMGLLQKQYETQYPDMTPSERLDLAKKVLMSSATALYDEDEKAYFSPRQQGAGAVDAKKASAATMYVTDKDNTSSKVHLNNVSDTFEVTVTVHNKSDKPQELYYQATVQTDKVDGKHFALAPKALYETSWQKITIPANSSKQVTVPIDASRFSKDLLAQMKNGYFLEGFVRFKQDPTKEELMSIPYIGFRGDFGNLSALEKPIYDSKDGSSYYHEANSDAKDQLDGDGLQFYALKNNFTALTTESNPWTIIKAVKEGVENIEDIESSEITETIFAGTFAKQDDDSHYYIHRHANGKPYAAISPNGDGNRDYVQFQGTFLRNAKNLVAEVLDKEGNVVWTSEVTEQVVKNYNNDLASTLGSTRFEKTRWDGKDKDGKVVANGTYTYRVRYTPISSGAKEQHTDFDVIVDNTTPEVATSATFSTEDRRLTLASKPKTSQPVYRERIAYTYMDEDLPTTEYISPNEDGTFTLPEEAETMEGATVPLKMSDFTYVVEDMAGNITYTPVTKLLEGHSNK

SEQ ID NO: 31 - protein, 494 aa, synthetic construct
  REGION 1..494: note = SLO [79-571 fragment] WT w/o leader, with G overhang from cleavage
GAPKEMPLESAEKEEKKSEDKKKSEEDHTEEINDKIYSLNYNELEVLAKNGETIENFVPKEGVKKADKFIVIERKKKNINTTPVDISIIDSVTDRTYPAALQLANKGFTENKPDAVVTKRNPQKIHIDLPGMGDKATVEVNDPTYANVSTAIDNLVNQWHDNYSGGNTLPARTQYTESMVYSKSQIEAALNVNSKILDGTLGIDFKSISKGEKKVMIAAYKQIFYTVSANLPNNPADVFDKSVTFKELQRKGVSNEAPPLFVSNVAYGRTVFVKLETSSKSNDVEAAFSAALKGTDVKTNGKYSDILENSSFTAVVLGGDAAEHNKVVTKDFDVIRNVIKDNATFSRKNPAYPISYTSVFLKNNKIAGVNNRTEYVETTSTEYTSGKINLSHQGAYVAQYEILWDEINYDDKGKEVITKRRWDNNWYSKTSPFSTVIPLGANSRNIRIMARECTGLAWEWWRKVIDERDVKLSKEINVNISGSTLSPYGSITYK

SEQ ID NO: 32 - protein, 494 aa, synthetic construct
  REGION 1..494: note = SLO [79-571 fragment] W535A w/o leader, with G overhang from cleavage
GAPKEMPLESAEKEEKKSEDKKKSEEDHTEEINDKIYSLNYNELEVLAKNGETIENFVPKEGVKKADKFIVIERKKKNINTTPVDISIIDSVTDRTYPAALQLANKGFTENKPDAVVTKRNPQKIHIDLPGMGDKATVEVNDPTYANVSTAIDNLVNQWHDNYSGGNTLPARTQYTESMVYSKSQIEAALNVNSKILDGTLGIDFKSISKGEKKVMIAAYKQIFYTVSANLPNNPADVFDKSVTFKELQRKGVSNEAPPLFVSNVAYGRTVFVKLETSSKSNDVEAAFSAALKGTDVKTNGKYSDILENSSFTAVVLGGDAAEHNKVVTKDFDVIRNVIKDNATFSRKNPAYPISYTSVFLKNNKIAGVNNRTEYVETTSTEYTSGKINLSHQGAYVAQYEILWDEINYDDKGKEVITKRRWDNNWYSKTSPFSTVIPLGANSRNIRIMARECTGLAAEWWRKVIDERDVKLSKEINVNISGSTLSPYGSITYK

SEQ ID NO: 33 - protein, 818 aa, synthetic construct
  REGION 1..818: note = SpyAD [33-849 fragment] WT w/o leader, with G overhang from cleavage
GQVKADDRASGETKASNTHDDSLPKPETIQEAKATIDAVEKTLSQQKAELTELATALTKTTAEINHLKEQQDNEQKALTSAQEIYTNTLASSEETLLAQGAEHQRELTATETELHNAQADQHSKETALSEQKASISAETTRAQDLVEQVKTSEQNIAKLNAMISNPDAITKAAQTANDNTKALSSELEKAKADLENQKAKVKKQLTEELAAQKAALAEKEAELSRLKSSAPSTQDSIVGNNTMKAPQGYPLEELKKLEASGYIGSASYNNYYKEHADQIIAKASPGNQLNQYQDIPADRNRFVDPDNLTPEVQNELAQFAAHMINSVRRQLGLPPVTVTAGSQEFARLLSTSYKKTHGNTRPSFVYGQPGVSGHYGVGPHDKTIIEDSAGASGLIRNDDNMYENIGAFNDVHTVNGIKRGIYDSIKYMLFTDHLHGNTYGHAINFLRVDKHNPNAPVYLGFSTSNVGSLNEHFVMFPESNIANHQRFNKTPIKAVGSTKDYAQRVGTVSDTIAAIKGKVSSLENRLSAIHQEADIMAAQAKVSQLQGKLASTLKQSDSLNLQVRQLNDTKGSLRTELLAAKAKQAQLEATRDQSLAKLASLKAALHQTEALAEQAAARVTALVAKKAHLQYLRDFKLNPNRLQVIRERIDNTKQDLAKTTSSLLNAQEALAALQAKQSSLEATIATTEHQLTLLKTLANEKEYRHLDEDIATVPDLQVAPPLTGVKPLSYSKIDTTPLVQEMVKETKQLLEASARLAAENTSLVAEALVGQTSEMVASNAIVSKITSSITQPSSKTSYGSGSSTTSNLISDVDESTQR

SEQ ID NO: 34 - protein, 818 aa, synthetic construct
  REGION 1..818: note = SpyAD [33-849 fragment] nnAA (e.g. pAMF) mutant w/o leader, with G overhang from cleavage
  VARIANT 33: note = any non-natural amino acid (e.g pAMF)
  VARIANT 256: note = any non-natural amino acid (e.g pAMF)
  VARIANT 355: note = any non-natural amino acid (e.g pAMF)
  VARIANT 626: note = any non-natural amino acid (e.g pAMF)
GQVKADDRASGETKASNTHDDSLPKPETIQEAXATIDAVEKTLSQQKAELTELATALTKTTAEINHLKEQQDNEQKALTSAQEIYTNTLASSEETLLAQGAEHQRELTATETELHNAQADQHSKETALSEQKASISAETTRAQDLVEQVKTSEQNIAKLNAMISNPDAITKAAQTANDNTKALSSELEKAKADLENQKAKVKKQLTEELAAQKAALAEKEAELSRLKSSAPSTQDSIVGNNTMKAPQGYPLEELKXLEASGYIGSASYNNYYKEHADQIIAKASPGNQLNQYQDIPADRNRFVDPDNLTPEVQNELAQFAAHMINSVRRQLGLPPVTVTAGSQEFARLLSTSYKXTHGNTRPSFVYGQPGVSGHYGVGPHDKTIIEDSAGASGLIRNDDNMYENIGAFNDVHTVNGIKRGIYDSIKYMLFTDHLHGNTYGHAINFLRVDKHNPNAPVYLGFSTSNVGSLNEHFVMFPESNIANHQRFNKTPIKAVGSTKDYAQRVGTVSDTIAAIKGKVSSLENRLSAIHQEADIMAAQAKVSQLQGKLASTLKQSDSLNLQVRQLNDTKGSLRTELLAAKAKQAQLEATRDQSLAKLASLKAALHQTEALAEQAAARVTALVAKXAHLQYLRDFKLNPNRLQVIRERIDNTKQDLAKTTSSLLNAQEALAALQAKQSSLEATIATTEHQLTLLKTLANEKEYRHLDEDIATVPDLQVAPPLTGVKPLSYSKIDTTPLVQEMVKETKQLLEASARLAAENTSLVAEALVGQTSEMVASNAIVSKITSSITQPSSKTSYGSGSSTTSNLISDVDESTQR

SEQ ID NO: 35 - protein, 411 aa, synthetic construct
  REGION 1..411: note = ADI [full length] WT w/o leader, with G overhang from cleavage
GTAQTPIHVYSEIGKLKKVLLHRPGKEIENLMPDYLERLLFDDIPFLEDAQKEHDAFAQALRDEGIEVLYLETLAAESLVTPEIREAFIDEYLSEANIRGRATKKAIRELLMAIEDNQELIEKTMAGVQKSELPEIPASEKGLTDLVESNYPFAIDPMPNLYFTRDPFATIGTGVSLNHMFSETRNRETLYGKYIFTHHPIYGGGKVPMVYDRNETTRIEGGDELVLSKDVLAVGISQRTDAASIEKLLVNIFKQNLGFKKVLAFEFANNRKFMHLDTVFTMVDYDKFTIHPEIEGDLRVYSVTYDNEELHIVEEKGDLAELLAANLGVEKVDLIRCGGDNLVAAGREQWNDGSNTLTIAPGVVVVYNRNTITNAILESKGLKLIKIHGSELVRGRGGPRCMSMPFEREDI

SEQ ID NO: 36 - protein, 411 aa, synthetic construct
  REGION 1..411: note = DI [full length] D277A w/o leader, with G overhang from cleavage
GTAQTPIHVYSEIGKLKKVLLHRPGKEIENLMPDYLERLLFDDIPFLEDAQKEHDAFAQALRDEGIEVLYLETLAAESLVTPEIREAFIDEYLSEANIRGRATKKAIRELLMAIEDNQELIEKTMAGVQKSELPEIPASEKGLTDLVESNYPFAIDPMPNLYFTRDPFATIGTGVSLNHMFSETRNRETLYGKYIFTHHPIYGGGKVPMVYDRNETTRIEGGDELVLSKDVLAVGISQRTDAASIEKLLVNIFKQNLGFKKVLAFEFANNRKFMHLATVFTMVDYDKFTIHPEIEGDLRVYSVTYDNEELHIVEEKGDLAELLAANLGVEKVDLIRCGGDNLVAAGREQWNDGSNTLTIAPGVVVVYNRNTITNAILESKGLKLIKIHGSELVRGRGGPRCMSMPFEREDI

SEQ ID NO: 37 - protein, 411 aa, synthetic construct
  REGION 1..411: note = ADI [full length] nnAA (e.g. pAMF) mutant w/o leader, with G overhang from cleavage
  VARIANT 15: note = any non-natural amino acid (e.g pAMF)
  VARIANT 193: note = any non-natural amino acid (e.g pAMF)
  VARIANT 316: note = any non-natural amino acid (e.g pAMF)
GTAQTPIHVYSEIGXLKKVLLHRPGKEIENLMPDYLERLLFDDIPFLEDAQKEHDAFAQALRDEGIEVLYLETLAAESLVTPEIREAFIDEYLSEANIRGRATKKAIRELLMAIEDNQELIEKTMAGVQKSELPEIPASEKGLTDLVESNYPFAIDPMPNLYFTRDPFATIGTGVSLNHMFSETRNRETLYGXYIFTHHPIYGGGKVPMVYDRNETTRIEGGDELVLSKDVLAVGISQRTDAASIEKLLVNIFKQNLGFKKVLAFEFANNRKFMHLDTVFTMVDYDKFTIHPEIEGDLRVYSVTYDNEELHIVEEXGDLAELLAANLGVEKVDLIRCGGDNLVAAGREQWNDGSNTLTIAPGVVVVYNRNTITNAILESKGLKLIKIHGSELVRGRGGPRCMSMPFEREDI

SEQ ID NO: 38 - protein, 411 aa, synthetic construct
  REGION 1..411: note = ADI (mut5) [full length] D277A nnAA (e.g. pAMF) mutant w/o leader, with G overhang from cleavage
  VARIANT 15: note = any non-natural amino acid (e.g pAMF)
  VARIANT 193: note = any non-natural amino acid (e.g pAMF)
  VARIANT 316: note = any non-natural amino acid (e.g pAMF)
GTAQTPIHVYSEIGXLKKVLLHRPGKEIENLMPDYLERLLFDDIPFLEDAQKEHDAFAQALRDEGIEVLYLETLAAESLVTPEIREAFIDEYLSEANIRGRATKKAIRELLMAIEDNQELIEKTMAGVQKSELPEIPASEKGLTDLVESNYPFAIDPMPNLYFTRDPFATIGTGVSLNHMFSETRNRETLYGXYIFTHHPIYGGGKVPMVYDRNETTRIEGGDELVLSKDVLAVGISQRTDAASIEKLLVNIFKQNLGFKKVLAFEFANNRKFMHLATVFTMVDYDKFTIHPEIEGDLRVYSVTYDNEELHIVEEXGDLAELLAANLGVEKVDLIRCGGDNLVAAGREQWNDGSNTLTIAPGVVVVYNRNTITNAILESKGLKLIKIHGSELVRGRGGPRCMSMPFEREDI

SEQ ID NO: 39 - protein, 184 aa, synthetic construct; identical to SEQ ID NO: 21
  REGION 1..184: note = hFL [full length] WT w/ leader

SEQ ID NO: 40 - protein, 174 aa, Homo sapiens; identical to SEQ ID NO: 22

SEQ ID NO: 41 - protein, 184 aa, synthetic construct; identical to SEQ ID NO: 23
  REGION 1..184: note = hFL [full length] nnAA (e.g. pAMF) mutant w/ leader
  VARIANT 14: note = any non-natural amino acid (e.g pAMF)

SEQ ID NO: 42 - protein, 174 aa, synthetic construct; identical to SEQ ID NO: 24
  REGION 1..174: note = hFL [full length] nnAA (e.g. pAMF) mutant w/o leader
  VARIANT 4: note = any non-natural amino acid (e.g pAMF)

SEQ ID NO: 43 - protein, 535 aa, synthetic construct
  REGION 1..535: note = eCRM-nnAA (e.g. pAMF) mutant
  VARIANT 33: note = any non-natural amino acid (e.g pAMF)
  VARIANT 212: note = any non-natural amino acid (e.g pAMF)
  VARIANT 244: note = any non-natural amino acid (e.g pAMF)
  VARIANT 264: note = any non-natural amino acid (e.g pAMF)
  VARIANT 385: note = any non-natural amino acid (e.g pAMF)
  VARIANT 526: note = any non-natural amino acid (e.g pAMF)
GADDVVDSSKSFVMENFSSYHGTKPGYVDSIQXGIQKPKSGTQGNYDDDWKEFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRNSVGSSLSCINLDWDVIRDXTKTKIESLKEHGPIKNKMSESPNKTVSEEKAXQYLEEFHQTALEHPELSELXTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHXTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSXLSLFFEIKS

SEQ ID NO: 44 - protein, 483 aa, synthetic construct
  REGION 1..483: note = SLO(deltaC11) W535A w/o leader, with G overhang from cleavage
GAPKEMPLESAEKEEKKSEDKKKSEEDHTEEINDKIYSLNYNELEVLAKNGETIENFVPKEGVKKADKFIVIERKKKNINTTPVDISIIDSVTDRTYPAALQLANKGFTENKPDAVVTKRNPQKIHIDLPGMGDKATVEVNDPTYANVSTAIDNLVNQWHDNYSGGNTLPARTQYTESMVYSKSQIEAALNVNSKILDGTLGIDFKSISKGEKKVMIAAYKQIFYTVSANLPNNPADVFDKSVTFKELQRKGVSNEAPPLFVSNVAYGRTVFVKLETSSKSNDVEAAFSAALKGTDVKTNGKYSDILENSSFTAVVLGGDAAEHNKVVTKDFDVIRNVIKDNATFSRKNPAYPISYTSVFLKNNKIAGVNNRTEYVETTSTEYTSGKINLSHQGAYVAQYEILWDEINYDDKGKEVITKRRWDNNWYSKTSPFSTVIPLGANSRNIRIMARECTGLAAEWWRKVIDERDVKLSKEINVNISGS

SEQ ID NO: 45 - protein, 473 aa, synthetic construct
  REGION 1..473: note = SLO(deltaC21) fragment W535A w/o leader, with G overhang from cleavage
GAPKEMPLESAEKEEKKSEDKKKSEEDHTEEINDKIYSLNYNELEVLAKNGETIENFVPKEGVKKADKFIVIERKKKNINTTPVDISIIDSVTDRTYPAALQLANKGFTENKPDAVVTKRNPQKIHIDLPGMGDKATVEVNDPTYANVSTAIDNLVNQWHDNYSGGNTLPARTQYTESMVYSKSQIEAALNVNSKILDGTLGIDFKSISKGEKKVMIAAYKQIFYTVSANLPNNPADVFDKSVTFKELQRKGVSNEAPPLFVSNVAYGRTVFVKLETSSKSNDVEAAFSAALKGTDVKTNGKYSDILENSSFTAVVLGGDAAEHNKVVTKDFDVIRNVIKDNATFSRKNPAYPISYTSVFLKNNKIAGVNNRTEYVETTSTEYTSGKINLSHQGAYVAQYEILWDEINYDDKGKEVITKRRWDNNWYSKTSPFSTVIPLGANSRNIRIMARECTGLAAEWWRKVIDERDVKLS

SEQ ID NO: 46 - protein, 463 aa, synthetic construct
  REGION 1..463: note = SLO(deltaC31) fragment W535A w/o leader, with G overhang from cleavage
GAPKEMPLESAEKEEKKSEDKKKSEEDHTEEINDKIYSLNYNELEVLAKNGETIENFVPKEGVKKADKFIVIERKKKNINTTPVDISIIDSVTDRTYPAALQLANKGFTENKPDAVVTKRNPQKIHIDLPGMGDKATVEVNDPTYANVSTAIDNLVNQWHDNYSGGNTLPARTQYTESMVYSKSQIEAALNVNSKILDGTLGIDFKSISKGEKKVMIAAYKQIFYTVSANLPNNPADVFDKSVTFKELQRKGVSNEAPPLFVSNVAYGRTVFVKLETSSKSNDVEAAFSAALKGTDVKTNGKYSDILENSSFTAVVLGGDAAEHNKVVTKDFDVIRNVIKDNATFSRKNPAYPISYTSVFLKNNKIAGVNNRTEYVETTSTEYTSGKINLSHQGAYVAQYEILWDEINYDDKGKEVITKRRWDNNWYSKTSPFSTVIPLGANSRNIRIMARECTGLAAEWWRK

SEQ ID NO: 47 - protein, 453 aa, synthetic construct
  REGION 1..453: note = SLO(deltaC41) fragment of SLO(deltaC11) w/o leader, with G overhang from cleavage
GAPKEMPLESAEKEEKKSEDKKKSEEDHTEEINDKIYSLNYNELEVLAKNGETIENFVPKEGVKKADKFIVIERKKKNINTTPVDISIIDSVTDRTYPAALQLANKGFTENKPDAVVTKRNPQKIHIDLPGMGDKATVEVNDPTYANVSTAIDNLVNQWHDNYSGGNTLPARTQYTESMVYSKSQIEAALNVNSKILDGTLGIDFKSISKGEKKVMIAAYKQIFYTVSANLPNNPADVFDKSVTFKELQRKGVSNEAPPLFVSNVAYGRTVFVKLETSSKSNDVEAAFSAALKGTDVKTNGKYSDILENSSFTAVVLGGDAAEHNKVVTKDFDVIRNVIKDNATFSRKNPAYPISYTSVFLKNNKIAGVNNRTEYVETTSTEYTSGKINLSHQGAYVAQYEILWDEINYDDKGKEVITKRRWDNNWYSKTSPFSTVIPLGANSRNIRIMAREC

SEQ ID NO: 48 - protein, 443 aa, synthetic construct
  REGION 1..443: note = SLO(deltaC51) fragment of SLO(deltaC11) w/o leader, with G overhang from cleavage
GAPKEMPLESAEKEEKKSEDKKKSEEDHTEEINDKIYSLNYNELEVLAKNGETIENFVPKEGVKKADKFIVIERKKKNINTTPVDISIIDSVTDRTYPAALQLANKGFTENKPDAVVTKRNPQKIHIDLPGMGDKATVEVNDPTYANVSTAIDNLVNQWHDNYSGGNTLPARTQYTESMVYSKSQIEAALNVNSKILDGTLGIDFKSISKGEKKVMIAAYKQIFYTVSANLPNNPADVFDKSVTFKELQRKGVSNEAPPLFVSNVAYGRTVFVKLETSSKSNDVEAAFSAALKGTDVKTNGKYSDILENSSFTAVVLGGDAAEHNKVVTKDFDVIRNVIKDNATFSRKNPAYPISYTSVFLKNNKIAGVNNRTEYVETTSTEYTSGKINLSHQGAYVAQYEILWDEINYDDKGKEVITKRRWDNNWYSKTSPFSTVIPLGANS

SEQ ID NO: 49 - protein, 433 aa, synthetic construct
  REGION 1..433: note = SLO(deltaC61) fragment of SLO(deltaC11) w/o leader, with G overhang from cleavage
GAPKEMPLESAEKEEKKSEDKKKSEEDHTEEINDKIYSLNYNELEVLAKNGETIENFVPKEGVKKADKFIVIERKKKNINTTPVDISIIDSVTDRTYPAALQLANKGFTENKPDAVVTKRNPQKIHIDLPGMGDKATVEVNDPTYANVSTAIDNLVNQWHDNYSGGNTLPARTQYTESMVYSKSQIEAALNVNSKILDGTLGIDFKSISKGEKKVMIAAYKQIFYTVSANLPNNPADVFDKSVTFKELQRKGVSNEAPPLFVSNVAYGRTVFVKLETSSKSNDVEAAFSAALKGTDVKTNGKYSDILENSSFTAVVLGGDAAEHNKVVTKDFDVIRNVIKDNATFSRKNPAYPISYTSVFLKNNKIAGVNNRTEYVETTSTEYTSGKINLSHQGAYVAQYEILWDEINYDDKGKEVITKRRWDNNWYSKTSPF

SEQ ID NO: 50 - protein, 423 aa, synthetic construct
  REGION 1..423: note = SLO(deltaC71) fragment of SLO(deltaC11) w/o leader, with G overhang from cleavage
GAPKEMPLESAEKEEKKSEDKKKSEEDHTEEINDKIYSLNYNELEVLAKNGETIENFVPKEGVKKADKFIVIERKKKNINTTPVDISIIDSVTDRTYPAALQLANKGFTENKPDAVVTKRNPQKIHIDLPGMGDKATVEVNDPTYANVSTAIDNLVNQWHDNYSGGNTLPARTQYTESMVYSKSQIEAALNVNSKILDGTLGIDFKSISKGEKKVMIAAYKQIFYTVSANLPNNPADVFDKSVTFKELQRKGVSNEAPPLFVSNVAYGRTVFVKLETSSKSNDVEAAFSAALKGTDVKTNGKYSDILENSSFTAVVLGGDAAEHNKVVTKDFDVIRNVIKDNATFSRKNPAYPISYTSVFLKNNKIAGVNNRTEYVETTSTEYTSGKINLSHQGAYVAQYEILWDEINYDDKGKEVITKRRWD

SEQ ID NO: 51 - protein, 413 aa, synthetic construct
  REGION 1..413: note = SLO(deltaC81) fragment of SLO(deltaC11) w/o leader, with G overhang from cleavage
GAPKEMPLESAEKEEKKSEDKKKSEEDHTEEINDKIYSLNYNELEVLAKNGETIENFVPKEGVKKADKFIVIERKKKNINTTPVDISIIDSVTDRTYPAALQLANKGFTENKPDAVVTKRNPQKIHIDLPGMGDKATVEVNDPTYANVSTAIDNLVNQWHDNYSGGNTLPARTQYTESMVYSKSQIEAALNVNSKILDGTLGIDFKSISKGEKKVMIAAYKQIFYTVSANLPNNPADVFDKSVTFKELQRKGVSNEAPPLFVSNVAYGRTVFVKLETSSKSNDVEAAFSAALKGTDVKTNGKYSDILENSSFTAVVLGGDAAEHNKVVTKDFDVIRNVIKDNATFSRKNPAYPISYTSVFLKNNKIAGVNNRTEYVETTSTEYTSGKINLSHQGAYVAQYEILWDEINYDDKG

SEQ ID NO: 52 - protein, 403 aa, synthetic construct
  REGION 1..403: note = SLO(deltaC91) fragment of SLO(deltaC11) w/o leader, with G overhang from cleavage
GAPKEMPLESAEKEEKKSEDKKKSEEDHTEEINDKIYSLNYNELEVLAKNGETIENFVPKEGVKKADKFIVIERKKKNINTTPVDISIIDSVTDRTYPAALQLANKGFTENKPDAVVTKRNPQKIHIDLPGMGDKATVEVNDPTYANVSTAIDNLVNQWHDNYSGGNTLPARTQYTESMVYSKSQIEAALNVNSKILDGTLGIDFKSISKGEKKVMIAAYKQIFYTVSANLPNNPADVFDKSVTFKELQRKGVSNEAPPLFVSNVAYGRTVFVKLETSSKSNDVEAAFSAALKGTDVKTNGKYSDILENSSFTAVVLGGDAAEHNKVVTKDFDVIRNVIKDNATFSRKNPAYPISYTSVFLKNNKIAGVNNRTEYVETTSTEYTSGKINLSHQGAYVAQYEIL

SEQ ID NO: 53 - protein, 393 aa, synthetic construct
  REGION 1..393: note = SLO(deltaC101) fragment of W535A w/o leader, with G overhang from cleavage
GAPKEMPLESAEKEEKKSEDKKKSEEDHTEEINDKIYSLNYNELEVLAKNGETIENFVPKEGVKKADKFIVIERKKKNINTTPVDISIIDSVTDRTYPAALQLANKGFTENKPDAVVTKRNPQKIHIDLPGMGDKATVEVNDPTYANVSTAIDNLVNQWHDNYSGGNTLPARTQYTESMVYSKSQIEAALNVNSKILDGTLGIDFKSISKGEKKVMIAAYKQIFYTVSANLPNNPADVFDKSVTFKELQRKGVSNEAPPLFVSNVAYGRTVFVKLETSSKSNDVEAAFSAALKGTDVKTNGKYSDILENSSFTAVVLGGDAAEHNKVVTKDFDVIRNVIKDNATFSRKNPAYPISYTSVFLKNNKIAGVNNRTEYVETTSTEYTSGKINLSHQ

SEQ ID NO: 54 - protein, 393 aa, synthetic construct
  REGION 1..393: note = SLO(deltaC101) fragment of SLO(deltaC11) w/o leader, with G overhang from cleavage
  VARIANT 21: note = any non-natural amino acid (e.g pAMF)
  VARIANT 35: note = any non-natural amino acid (e.g pAMF)
  VARIANT 74: note = any non-natural amino acid (e.g pAMF)
  VARIANT 112: note = any non-natural amino acid (e.g pAMF)
  VARIANT 195: note = any non-natural amino acid (e.g pAMF)
  VARIANT 246: note = any non-natural amino acid (e.g pAMF)
  VARIANT 280: note = any non-natural amino acid (e.g pAMF)
  VARIANT 298: note = any non-natural amino acid (e.g pAMF)
  VARIANT 330: note = any non-natural amino acid (e.g pAMF)
  VARIANT 387: note = any non-natural amino acid (e.g pAMF)
GAPKEMPLESAEKEEKKSEDXKKSEEDHTEEINDXIYSLNYNELEVLAKNGETIENFVPKEGVKKADKFIVIEXKKKNINTTPVDISIIDSVTDRTYPAALQLANKGFTENXPDAVVTKRNPQKIHIDLPGMGDKATVEVNDPTYANVSTAIDNLVNQWHDNYSGGNTLPARTQYTESMVYSKSQIEAALNVNSXILDGTLGIDFKSISKGEKKVMIAAYKQIFYTVSANLPNNPADVFDKSVTFXELQRKGVSNEAPPLFVSNVAYGRTVFVKLETSSXSNDVEAAFSAALKGTDVXTNGKYSDILENSSFTAVVLGGDAAEHNKVVTXDFDVIRNVIKDNATFSRKNPAYPISYTSVFLKNNKIAGVNNRTEYVETTSTEYTSGXINLSHQ

SEQ ID NO: 55 - protein, 393 aa, synthetic construct
  REGION 1..393: note = SLO(deltaC101) fragment of SLO(deltaC11) - nnAA (e.g. pAMF) mutant 1 - w/o leader, with G overhang from cleavage
  VARIANT 21: note = any non-natural amino acid (e.g pAMF)
  VARIANT 74: note = any non-natural amino acid (e.g pAMF)
  VARIANT 195: note = any non-natural amino acid (e.g pAMF)
  VARIANT 280: note = any non-natural amino acid (e.g pAMF)
GAPKEMPLESAEKEEKKSEDXKKSEEDHTEEINDKIYSLNYNELEVLAKNGETIENFVPKEGVKKADKFIVIEXKKKNINTTPVDISIIDSVTDRTYPAALQLANKGFTENKPDAVVTKRNPQKIHIDLPGMGDKATVEVNDPTYANVSTAIDNLVNQWHDNYSGGNTLPARTQYTESMVYSKSQIEAALNVNSXILDGTLGIDFKSISKGEKKVMIAAYKQIFYTVSANLPNNPADVFDKSVTFKELQRKGVSNEAPPLFVSNVAYGRTVFVKLETSSXSNDVEAAFSAALKGTDVKTNGKYSDILENSSFTAVVLGGDAAEHNKVVTKDFDVIRNVIKDNATFSRKNPAYPISYTSVFLKNNKIAGVNNRTEYVETTSTEYTSGKINLSHQ

SEQ ID NO: 56 - protein, 393 aa, synthetic construct
  REGION 1..393: note = SLO(deltaC101) fragment of SLO(deltaC11) - nnAA (e.g. pAMF) mutant 2 - w/o leader, with G overhang from cleavage
  VARIANT 35: note = any non-natural amino acid (e.g pAMF)
  VARIANT 112: note = any non-natural amino acid (e.g pAMF)
  VARIANT 246: note = any non-natural amino acid (e.g pAMF)
  VARIANT 298: note = any non-natural amino acid (e.g pAMF)
GAPKEMPLESAEKEEKKSEDKKKSEEDHTEEINDXIYSLNYNELEVLAKNGETIENFVPKEGVKKADKFIVIERKKKNINTTPVDISIIDSVTDRTYPAALQLANKGFTENXPDAVVTKRNPQKIHIDLPGMGDKATVEVNDPTYANVSTAIDNLVNQWHDNYSGGNTLPARTQYTESMVYSKSQIEAALNVNSKILDGTLGIDFKSISKGEKKVMIAAYKQIFYTVSANLPNNPADVFDKSVTFXELQRKGVSNEAPPLFVSNVAYGRTVFVKLETSSKSNDVEAAFSAALKGTDVXTNGKYSDILENSSFTAVVLGGDAAEHNKVVTKDFDVIRNVIKDNATFSRKNPAYPISYTSVFLKNNKIAGVNNRTEYVETTSTEYTSGKINLSHQ

SEQ ID NO: 57 - protein, 393 aa, synthetic construct
  REGION 1..393: note = SLO(deltaC101) fragment of SLO(deltaC11) - nnAA (e.g. pAMF) mutant 3 - w/o leader, with G overhang from cleavage
  VARIANT 74: note = any non-natural amino acid (e.g pAMF)
  VARIANT 195: note = any non-natural amino acid (e.g pAMF)
  VARIANT 280: note = any non-natural amino acid (e.g pAMF)
  VARIANT 330: note = any non-natural amino acid (e.g pAMF)
GAPKEMPLESAEKEEKKSEDKKKSEEDHTEEINDKIYSLNYNELEVLAKNGETIENFVPKEGVKKADKFIVIEXKKKNINTTPVDISIIDSVTDRTYPAALQLANKGFTENKPDAVVTKRNPQKIHIDLPGMGDKATVEVNDPTYANVSTAIDNLVNQWHDNYSGGNTLPARTQYTESMVYSKSQIEAALNVNSXILDGTLGIDFKSISKGEKKVMIAAYKQIFYTVSANLPNNPADVFDKSVTFKELQRKGVSNEAPPLFVSNVAYGRTVFVKLETSSXSNDVEAAFSAALKGTDVKTNGKYSDILENSSFTAVVLGGDAAEHNKVVTXDFDVIRNVIKDNATFSRKNPAYPISYTSVFLKNNKIAGVNNRTEYVETTSTEYTSGKINLSHQ

SEQ ID NO: 58 - protein, 393 aa, synthetic construct
  REGION 1..393: note = SLO(deltaC101) fragment of SLO(deltaC11) - nnAA (e.g. pAMF) mutant 4 - w/o leader, with G overhang from cleavage
  VARIANT 74: note = any non-natural amino acid (e.g pAMF)
  VARIANT 195: note = any non-natural amino acid (e.g pAMF)
  VARIANT 298: note = any non-natural amino acid (e.g pAMF)
  VARIANT 387: note = any non-natural amino acid (e.g pAMF)
GAPKEMPLESAEKEEKKSEDKKKSEEDHTEEINDKIYSLNYNELEVLAKNGETIENFVPKEGVKKADKFIVIEXKKKNINTTPVDISIIDSVTDRTYPAALQLANKGFTENKPDAVVTKRNPQKIHIDLPGMGDKATVEVNDPTYANVSTAIDNLVNQWHDNYSGGNTLPARTQYTESMVYSKSQIEAALNVNSXILDGTLGIDFKSISKGEKKVMIAAYKQIFYTVSANLPNNPADVFDKSVTFKELQRKGVSNEAPPLFVSNVAYGRTVFVKLETSSKSNDVEAAFSAALKGTDVXTNGKYSDILENSSFTAVVLGGDAAEHNKVVTKDFDVIRNVIKDNATFSRKNPAYPISYTSVFLKNNKIAGVNNRTEYVETTSTEYTSGXINLSHQ

SEQ ID NO: 59 - protein, 393 aa, synthetic construct
  REGION 1..393: note = SLO(deltaC101) fragment of SLO(deltaC11) - nnAA (e.g. pAMF) mutant 5 - w/o leader, with G overhang from cleavage
  VARIANT 35: note = any non-natural amino acid (e.g pAMF)
  VARIANT 195: note = any non-natural amino acid (e.g pAMF)
  VARIANT 280: note = any non-natural amino acid (e.g pAMF)
  VARIANT 387: note = any non-natural amino acid (e.g pAMF)
GAPKEMPLESAEKEEKKSEDKKKSEEDHTEEINDXIYSLNYNELEVLAKNGETIENFVPKEGVKKADKFIVIERKKKNINTTPVDISIIDSVTDRTYPAALQLANKGFTENKPDAVVTKRNPQKIHIDLPGMGDKATVEVNDPTYANVSTAIDNLVNQWHDNYSGGNTLPARTQYTESMVYSKSQIEAALNVNSXILDGTLGIDFKSISKGEKKVMIAAYKQIFYTVSANLPNNPADVFDKSVTFKELQRKGVSNEAPPLFVSNVAYGRTVFVKLETSSXSNDVEAAFSAALKGTDVKTNGKYSDILENSSFTAVVLGGDAAEHNKVVTKDFDVIRNVIKDNATFSRKNPAYPISYTSVFLKNNKIAGVNNRTEYVETTSTEYTSGXINLSHQ

SEQ ID NO: 60 - protein, 393 aa, synthetic construct
  REGION 1..393: note = SLO(deltaC101) fragment of SLO(deltaC11) - nnAA (e.g. pAMF) mutant 6 - w/o leader, with G overhang from cleavage
  VARIANT 21: note = any non-natural amino acid (e.g pAMF)
  VARIANT 112: note = any non-natural amino acid (e.g pAMF)
  VARIANT 280: note = any non-natural amino acid (e.g pAMF)
GAPKEMPLESAEKEEKKSEDXKKSEEDHTEEINDKIYSLNYNELEVLAKNGETIENFVPKEGVKKADKFIVIERKKKNINTTPVDISIIDSVTDRTYPAALQLANKGFTENXPDAVVTKRNPQKIHIDLPGMGDKATVEVNDPTYANVSTAIDNLVNQWHDNYSGGNTLPARTQYTESMVYSKSQIEAALNVNSKILDGTLGIDFKSISKGEKKVMIAAYKQIFYTVSANLPNNPADVFDKSVTFKELQRKGVSNEAPPLFVSNVAYGRTVFVKLETSSXSNDVEAAFSAALKGTDVKTNGKYSDILENSSFTAVVLGGDAAEHNKVVTKDFDVIRNVIKDNATFSRKNPAYPISYTSVFLKNNKIAGVNNRTEYVETTSTEYTSGKINLSHQ

SEQ ID NO: 61 - protein, 393 aa, synthetic construct
  REGION 1..393: note = SLO(deltaC101) fragment of SLO(deltaC11) - nnAA (e.g. pAMF) mutant 7 - w/o leader, with G overhang from cleavage
  VARIANT 21: note = any non-natural amino acid (e.g pAMF)
  VARIANT 35: note = any non-natural amino acid (e.g pAMF)
  VARIANT 112: note = any non-natural amino acid (e.g pAMF)
  VARIANT 246: note = any non-natural amino acid (e.g pAMF)
GAPKEMPLESAEKEEKKSEDXKKSEEDHTEEINDXIYSLNYNELEVLAKNGETIENFVPKEGVKKADKFIVIERKKKNINTTPVDISIIDSVTDRTYPAALQLANKGFTENXPDAVVTKRNPQKIHIDLPGMGDKATVEVNDPTYANVSTAIDNLVNQWHDNYSGGNTLPARTQYTESMVYSKSQIEAALNVNSKILDGTLGIDFKSISKGEKKVMIAAYKQIFYTVSANLPNNPADVFDKSVTFXELQRKGVSNEAPPLFVSNVAYGRTVFVKLETSSKSNDVEAAFSAALKGTDVKTNGKYSDILENSSFTAVVLGGDAAEHNKVVTKDFDVIRNVIKDNATFSRKNPAYPISYTSVFLKNNKIAGVNNRTEYVETTSTEYTSGKINLSHQ

SEQ ID NO: 62 - protein, 393 aa, synthetic construct
  REGION 1..393: note = SLO(deltaC101) fragment of SLO(deltaC11) - nnAA (e.g. pAMF) mutant 8 - w/o leader, with G overhang from cleavage
  VARIANT 21: note = any non-natural amino acid (e.g pAMF)
  VARIANT 74: note = any non-natural amino acid (e.g pAMF)
  VARIANT 195: note = any non-natural amino acid (e.g pAMF)
GAPKEMPLESAEKEEKKSEDXKKSEEDHTEEINDKIYSLNYNELEVLAKNGETIENFVPKEGVKKADKFIVIEXKKKNINTTPVDISIIDSVTDRTYPAALQLANKGFTENKPDAVVTKRNPQKIHIDLPGMGDKATVEVNDPTYANVSTAIDNLVNQWHDNYSGGNTLPARTQYTESMVYSKSQIEAALNVNSXILDGTLGIDFKSISKGEKKVMIAAYKQIFYTVSANLPNNPADVFDKSVTFKELQRKGVSNEAPPLFVSNVAYGRTVFVKLETSSKSNDVEAAFSAALKGTDVKTNGKYSDILENSSFTAVVLGGDAAEHNKVVTKDFDVIRNVIKDNATFSRKNPAYPISYTSVFLKNNKIAGVNNRTEYVETTSTEYTSGKINLSHQ

SEQ ID NO: 63 - protein, 393 aa, synthetic construct
  REGION 1..393: note = SLO(deltaC101) fragment of SLO(deltaC11) - nnAA (e.g. pAMF) mutant 9 - w/o leader, with G overhang from cleavage
  VARIANT 35: note = any non-natural amino acid (e.g pAMF)
  VARIANT 195: note = any non-natural amino acid (e.g pAMF)
  VARIANT 298: note = any non-natural amino acid (e.g pAMF)
GAPKEMPLESAEKEEKKSEDKKKSEEDHTEEINDXIYSLNYNELEVLAKNGETIENFVPKEGVKKADKFIVIERKKKNINTTPVDISIIDSVTDRTYPAALQLANKGFTENKPDAVVTKRNPQKIHIDLPGMGDKATVEVNDPTYANVSTAIDNLVNQWHDNYSGGNTLPARTQYTESMVYSKSQIEAALNVNSXILDGTLGIDFKSISKGEKKVMIAAYKQIFYTVSANLPNNPADVFDKSVTFKELQRKGVSNEAPPLFVSNVAYGRTVFVKLETSSKSNDVEAAFSAALKGTDVXTNGKYSDILENSSFTAVVLGGDAAEHNKVVTKDFDVIRNVIKDNATFSRKNPAYPISYTSVFLKNNKIAGVNNRTEYVETTSTEYTSGKINLSHQ

SEQ ID NO: 64 - protein, 393 aa, synthetic construct
  REGION 1..393: note = SLO(deltaC101) fragment of SLO(deltaC11) - nnAA (e.g. pAMF) mutant 10 - w/o leader, with G overhang from cleavage
  VARIANT 35: note = any non-natural amino acid (e.g pAMF)
  VARIANT 246: note = any non-natural amino acid (e.g pAMF)
  VARIANT 330: note = any non-natural amino acid (e.g pAMF)
GAPKEMPLESAEKEEKKSEDKKKSEEDHTEEINDXIYSLNYNELEVLAKNGETIENFVPKEGVKKADKFIVIERKKKNINTTPVDISIIDSVTDRTYPAALQLANKGFTENKPDAVVTKRNPQKIHIDLPGMGDKATVEVNDPTYANVSTAIDNLVNQWHDNYSGGNTLPARTQYTESMVYSKSQIEAALNVNSKILDGTLGIDFKSISKGEKKVMIAAYKQIFYTVSANLPNNPADVFDKSVTFXELQRKGVSNEAPPLFVSNVAYGRTVFVKLETSSKSNDVEAAFSAALKGTDVKTNGKYSDILENSSFTAVVLGGDAAEHNKVVTXDFDVIRNVIKDNATFSRKNPAYPISYTSVFLKNNKIAGVNNRTEYVETTSTEYTSGKINLSHQ

SEQ ID NO: 65 - protein, 393 aa, synthetic construct
  REGION 1..393: note = SLO(deltaC101) fragment of SLO(deltaC11) - nnAA (e.g. pAMF) mutant 10 - w/o leader, with G overhang from cleavage
  VARIANT 21: note = any non-natural amino acid (e.g pAMF)
  VARIANT 74: note = any non-natural amino acid (e.g pAMF)
  VARIANT 195: note = any non-natural amino acid (e.g pAMF)
  VARIANT 280: note = any non-natural amino acid (e.g pAMF)
  VARIANT 330: note = any non-natural amino acid (e.g pAMF)
GAPKEMPLESAEKEEKKSEDXKKSEEDHTEEINDKIYSLNYNELEVLAKNGETIENFVPKEGVKKADKFIVIEXKKKNINTTPVDISIIDSVTDRTYPAALQLANKGFTENKPDAVVTKRNPQKIHIDLPGMGDKATVEVNDPTYANVSTAIDNLVNQWHDNYSGGNTLPARTQYTESMVYSKSQIEAALNVNSXILDGTLGIDFKSISKGEKKVMIAAYKQIFYTVSANLPNNPADVFDKSVTFKELQRKGVSNEAPPLFVSNVAYGRTVFVKLETSSXSNDVEAAFSAALKGTDVKTNGKYSDILENSSFTAVVLGGDAAEHNKVVTXDFDVIRNVIKDNATFSRKNPAYPISYTSVFLKNNKIAGVNNRTEYVETTSTEYTSGKINLSHQ

SEQ ID NO: 66 - protein, 393 aa, synthetic construct
  REGION 1..393: note = SLO(deltaC101) fragment of SLO(deltaC11) - nnAA (e.g. pAMF) mutant 10 - w/o leader, with G overhang from cleavage
  VARIANT 35: note = any non-natural amino acid (e.g pAMF)
  VARIANT 112: note = any non-natural amino acid (e.g pAMF)
  VARIANT 246: note = any non-natural amino acid (e.g pAMF)
  VARIANT 298: note = any non-natural amino acid (e.g pAMF)
  VARIANT 387: note = any non-natural amino acid (e.g pAMF)
GAPKEMPLESAEKEEKKSEDKKKSEEDHTEEINDXIYSLNYNELEVLAKNGETIENFVPKEGVKKADKFIVIERKKKNINTTPVDISIIDSVTDRTYPAALQLANKGFTENXPDAVVTKRNPQKIHIDLPGMGDKATVEVNDPTYANVSTAIDNLVNQWHDNYSGGNTLPARTQYTESMVYSKSQIEAALNVNSKILDGTLGIDFKSISKGEKKVMIAAYKQIFYTVSANLPNNPADVFDKSVTFXELQRKGVSNEAPPLFVSNVAYGRTVFVKLETSSKSNDVEAAFSAALKGTDVXTNGKYSDILENSSFTAVVLGGDAAEHNKVVTKDFDVIRNVIKDNATFSRKNPAYPISYTSVFLKNNKIAGVNNRTEYVETTSTEYTSGXINLSHQ

SEQ ID NO: 67 - protein, 393 aa, synthetic construct
  REGION 1..393: note = SLO(deltaC101) fragment of SLO(deltaC11) - nnAA (e.g. pAMF) mutant 10 - w/o leader, with G overhang from cleavage
  VARIANT 35: note = any non-natural amino acid (e.g pAMF)
  VARIANT 74: note = any non-natural amino acid (e.g pAMF)
  VARIANT 195: note = any non-natural amino acid (e.g pAMF)
  VARIANT 280: note = any non-natural amino acid (e.g pAMF)
  VARIANT 330: note = any non-natural amino acid (e.g pAMF)
GAPKEMPLESAEKEEKKSEDKKKSEEDHTEEINDXIYSLNYNELEVLAKNGETIENFVPKEGVKKADKFIVIEXKKKNINTTPVDISIIDSVTDRTYPAALQLANKGFTENKPDAVVTKRNPQKIHIDLPGMGDKATVEVNDPTYANVSTAIDNLVNQWHDNYSGGNTLPARTQYTESMVYSKSQIEAALNVNSXILDGTLGIDFKSISKGEKKVMIAAYKQIFYTVSANLPNNPADVFDKSVTFKELQRKGVSNEAPPLFVSNVAYGRTVFVKLETSSXSNDVEAAFSAALKGTDVKTNGKYSDILENSSFTAVVLGGDAAEHNKVVTXDFDVIRNVIKDNATFSRKNPAYPISYTSVFLKNNKIAGVNNRTEYVETTSTEYTSGKINLSHQ

SEQ ID NO: 68 - protein, 393 aa, synthetic construct
  REGION 1..393: note = SLO(deltaC101) fragment of SLO(deltaC11) - nnAA (e.g. pAMF) mutant 10 - w/o leader, with G overhang from cleavage
  VARIANT 21: note = any non-natural amino acid (e.g pAMF)
  VARIANT 74: note = any non-natural amino acid (e.g pAMF)
  VARIANT 195: note = any non-natural amino acid (e.g pAMF)
  VARIANT 280: note = any non-natural amino acid (e.g pAMF)
  VARIANT 330: note = any non-natural amino acid (e.g pAMF)
  VARIANT 387: note = any non-natural amino acid (e.g pAMF)
GAPKEMPLESAEKEEKKSEDXKKSEEDHTEEINDKIYSLNYNELEVLAKNGETIENFVPKEGVKKADKFIVIEXKKKNINTTPVDISIIDSVTDRTYPAALQLANKGFTENKPDAVVTKRNPQKIHIDLPGMGDKATVEVNDPTYANVSTAIDNLVNQWHDNYSGGNTLPARTQYTESMVYSKSQIEAALNVNSXILDGTLGIDFKSISKGEKKVMIAAYKQIFYTVSANLPNNPADVFDKSVTFKELQRKGVSNEAPPLFVSNVAYGRTVFVKLETSSXSNDVEAAFSAALKGTDVKTNGKYSDILENSSFTAVVLGGDAAEHNKVVTXDFDVIRNVIKDNATFSRKNPAYPISYTSVFLKNNKIAGVNNRTEYVETTSTEYTSGXINLSHQ

SEQ ID NO: 69 - protein, 393 aa, synthetic construct
  REGION 1..393: note = SLO(deltaC101) fragment of SLO(deltaC11) - nnAA (e.g. pAMF) mutant 10 - w/o leader, with G overhang from cleavage
  VARIANT 35: note = any non-natural amino acid (e.g pAMF)
  VARIANT 74: note = any non-natural amino acid (e.g pAMF)
  VARIANT 112: note = any non-natural amino acid (e.g pAMF)
  VARIANT 246: note = any non-natural amino acid (e.g pAMF)
  VARIANT 298: note = any non-natural amino acid (e.g pAMF)
  VARIANT 387: note = any non-natural amino acid (e.g pAMF)
GAPKEMPLESAEKEEKKSEDKKKSEEDHTEEINDXIYSLNYNELEVLAKNGETIENFVPKEGVKKADKFIVIEXKKKNINTTPVDISIIDSVTDRTYPAALQLANKGFTENXPDAVVTKRNPQKIHIDLPGMGDKATVEVNDPTYANVSTAIDNLVNQWHDNYSGGNTLPARTQYTESMVYSKSQIEAALNVNSKILDGTLGIDFKSISKGEKKVMIAAYKQIFYTVSANLPNNPADVFDKSVTFXELQRKGVSNEAPPLFVSNVAYGRTVFVKLETSSKSNDVEAAFSAALKGTDVXTNGKYSDILENSSFTAVVLGGDAAEHNKVVTKDFDVIRNVIKDNATFSRKNPAYPISYTSVFLKNNKIAGVNNRTEYVETTSTEYTSGXINLSHQ

SEQ ID NO: 70 - protein, 393 aa, synthetic construct
  REGION 1..393: note = SLO(deltaC101) fragment of SLO(deltaC11) - nnAA (e.g. pAMF) mutant 10 - w/o leader, with G overhang from cleavage
  VARIANT 21: note = any non-natural amino acid (e.g pAMF)
  VARIANT 35: note = any non-natural amino acid (e.g pAMF)
  VARIANT 112: note = any non-natural amino acid (e.g pAMF)
  VARIANT 246: note = any non-natural amino acid (e.g pAMF)
  VARIANT 298: note = any non-natural amino acid (e.g pAMF)
  VARIANT 387: note = any non-natural amino acid (e.g pAMF)
GAPKEMPLESAEKEEKKSEDXKKSEEDHTEEINDXIYSLNYNELEVLAKNGETIENFVPKEGVKKADKFIVIERKKKNINTTPVDISIIDSVTDRTYPAALQLANKGFTENXPDAVVTKRNPQKIHIDLPGMGDKATVEVNDPTYANVSTAIDNLVNQWHDNYSGGNTLPARTQYTESMVYSKSQIEAALNVNSKILDGTLGIDFKSISKGEKKVMIAAYKQIFYTVSANLPNNPADVFDKSVTFXELQRKGVSNEAPPLFVSNVAYGRTVFVKLETSSKSNDVEAAFSAALKGTDVXTNGKYSDILENSSFTAVVLGGDAAEHNKVVTKDFDVIRNVIKDNATFSRKNPAYPISYTSVFLKNNKIAGVNNRTEYVETTSTEYTSGXINLSHQ

SEQ ID NO: 71 - protein, 393 aa, synthetic construct
  REGION 1..393: note = SLO(deltaC101) fragment of SLO(deltaC11) - nnAA (e.g. pAMF) mutant 10 - w/o leader, with G overhang from cleavage
  VARIANT 35: note = any non-natural amino acid (e.g pAMF)
  VARIANT 74: note = any non-natural amino acid (e.g pAMF)
  VARIANT 112: note = any non-natural amino acid (e.g pAMF)
  VARIANT 195: note = any non-natural amino acid (e.g pAMF)
  VARIANT 280: note = any non-natural amino acid (e.g pAMF)
  VARIANT 330: note = any non-natural amino acid (e.g pAMF)
  VARIANT 387: note = any non-natural amino acid (e.g pAMF)
GAPKEMPLESAEKEEKKSEDKKKSEEDHTEEINDXIYSLNYNELEVLAKNGETIENFVPKEGVKKADKFIVIEXKKKNINTTPVDISIIDSVTDRTYPAALQLANKGFTENXPDAVVTKRNPQKIHIDLPGMGDKATVEVNDPTYANVSTAIDNLVNQWHDNYSGGNTLPARTQYTESMVYSKSQIEAALNVNSXILDGTLGIDFKSISKGEKKVMIAAYKQIFYTVSANLPNNPADVFDKSVTFKELQRKGVSNEAPPLFVSNVAYGRTVFVKLETSSXSNDVEAAFSAALKGTDVKTNGKYSDILENSSFTAVVLGGDAAEHNKVVTXDFDVIRNVIKDNATFSRKNPAYPISYTSVFLKNNKIAGVNNRTEYVETTSTEYTSGXINLSHQ

SEQ ID NO: 72 - protein, 393 aa, synthetic construct
  REGION 1..393: note = SLO(deltaC101) fragment of SLO(deltaC11) - nnAA (e.g. pAMF) mutant 10 - w/o leader, with G overhang from cleavage
  VARIANT 21: note = any non-natural amino acid (e.g pAMF)
  VARIANT 74: note = any non-natural amino acid (e.g pAMF)
  VARIANT 112: note = any non-natural amino acid (e.g pAMF)
  VARIANT 246: note = any non-natural amino acid (e.g pAMF)
  VARIANT 298: note = any non-natural amino acid (e.g pAMF)
  VARIANT 330: note = any non-natural amino acid (e.g pAMF)
  VARIANT 387: note = any non-natural amino acid (e.g pAMF)
GAPKEMPLESAEKEEKKSEDXKKSEEDHTEEINDKIYSLNYNELEVLAKNGETIENFVPKEGVKKADKFIVIEXKKKNINTTPVDISIIDSVTDRTYPAALQLANKGFTENXPDAVVTKRNPQKIHIDLPGMGDKATVEVNDPTYANVSTAIDNLVNQWHDNYSGGNTLPARTQYTESMVYSKSQIEAALNVNSKILDGTLGIDFKSISKGEKKVMIAAYKQIFYTVSANLPNNPADVFDKSVTFXELQRKGVSNEAPPLFVSNVAYGRTVFVKLETSSKSNDVEAAFSAALKGTDVXTNGKYSDILENSSFTAVVLGGDAAEHNKVVTXDFDVIRNVIKDNATFSRKNPAYPISYTSVFLKNNKIAGVNNRTEYVETTSTEYTSGXINLSHQ

SEQ ID NO: 73 - protein, 393 aa, synthetic construct
  REGION 1..393: note = SLO(deltaC101) fragment of SLO(deltaC11) - nnAA (e.g. pAMF) mutant 10 - w/o leader, with G overhang from cleavage
  VARIANT 35: note = any non-natural amino acid (e.g pAMF)
  VARIANT 112: note = any non-natural amino acid (e.g pAMF)
  VARIANT 195: note = any non-natural amino acid (e.g pAMF)
  VARIANT 280: note = any non-natural amino acid (e.g pAMF)
  VARIANT 298: note = any naturally occurring amino acid
  VARIANT 330: note = any non-natural amino acid (e.g pAMF)
  VARIANT 387: note = any non-natural amino acid (e.g pAMF)
GAPKEMPLESAEKEEKKSEDKKKSEEDHTEEINDXIYSLNYNELEVLAKNGETIENFVPKEGVKKADKFIVIERKKKNINTTPVDISIIDSVTDRTYPAALQLANKGFTENXPDAVVTKRNPQKIHIDLPGMGDKATVEVNDPTYANVSTAIDNLVNQWHDNYSGGNTLPARTQYTESMVYSKSQIEAALNVNSXILDGTLGIDFKSISKGEKKVMIAAYKQIFYTVSANLPNNPADVFDKSVTFKELQRKGVSNEAPPLFVSNVAYGRTVFVKLETSSXSNDVEAAFSAALKGTDVXTNGKYSDILENSSFTAVVLGGDAAEHNKVVTXDFDVIRNVIKDNATFSRKNPAYPISYTSVFLKNNKIAGVNNRTEYVETTSTEYTSGXINLSHQ

SEQ ID NO: 74 - protein, 393 aa, synthetic construct
  REGION 1..393: note = SLO(deltaC101) fragment of SLO(deltaC11) - nnAA (e.g. pAMF) mutant 10 - w/o leader, with G overhang from cleavage
  VARIANT 21: note = any non-natural amino acid (e.g pAMF)
  VARIANT 35: note = any non-natural amino acid (e.g pAMF)
  VARIANT 74: note = any non-natural amino acid (e.g pAMF)
  VARIANT 112: note = any non-natural amino acid (e.g pAMF)
  VARIANT 195: note = any non-natural amino acid (e.g pAMF)
  VARIANT 246: note = any non-natural amino acid (e.g pAMF)
  VARIANT 280: note = any non-natural amino acid (e.g pAMF)
  VARIANT 298: note = any non-natural amino acid (e.g pAMF)
GAPKEMPLESAEKEEKKSEDXKKSEEDHTEEINDXIYSLNYNELEVLAKNGETIENFVPKEGVKKADKFIVIEXKKKNINTTPVDISIIDSVTDRTYPAALQLANKGFTENXPDAVVTKRNPQKIHIDLPGMGDKATVEVNDPTYANVSTAIDNLVNQWHDNYSGGNTLPARTQYTESMVYSKSQIEAALNVNSXILDGTLGIDFKSISKGEKKVMIAAYKQIFYTVSANLPNNPADVFDKSVTFXELQRKGVSNEAPPLFVSNVAYGRTVFVKLETSSXSNDVEAAFSAALKGTDVXTNGKYSDILENSSFTAVVLGGDAAEHNKVVTKDFDVIRNVIKDNATFSRKNPAYPISYTSVFLKNNKIAGVNNRTEYVETTSTEYTSGKINLSHQ

SEQ ID NO: 75 - protein, 393 aa, synthetic construct
  REGION 1..393: note = SLO(deltaC101) fragment of SLO(deltaC11) - nnAA (e.g. pAMF) mutant 10 - w/o leader, with G overhang from cleavage
  VARIANT 21: note = any non-natural amino acid (e.g pAMF)
  VARIANT 74: note = any non-natural amino acid (e.g pAMF)
  VARIANT 112: note = any non-natural amino acid (e.g pAMF)
  VARIANT 195: note = any non-natural amino acid (e.g pAMF)
  VARIANT 246: note = any non-natural amino acid (e.g pAMF)
  VARIANT 280: note = any non-natural amino acid (e.g pAMF)
  VARIANT 330: note = any non-natural amino acid (e.g pAMF)
  VARIANT 387: note = any non-natural amino acid (e.g pAMF)
GAPKEMPLESAEKEEKKSEDXKKSEEDHTEEINDKIYSLNYNELEVLAKNGETIENFVPKEGVKKADKFIVIEXKKKNINTTPVDISIIDSVTDRTYPAALQLANKGFTENXPDAVVTKRNPQKIHIDLPGMGDKATVEVNDPTYANVSTAIDNLVNQWHDNYSGGNTLPARTQYTESMVYSKSQIEAALNVNSXILDGTLGIDFKSISKGEKKVMIAAYKQIFYTVSANLPNNPADVFDKSVTFXELQRKGVSNEAPPLFVSNVAYGRTVFVKLETSSXSNDVEAAFSAALKGTDVKTNGKYSDILENSSFTAVVLGGDAAEHNKVVTXDFDVIRNVIKDNATFSRKNPAYPISYTSVFLKNNKIAGVNNRTEYVETTSTEYTSGXINLSHQ

SEQ ID NO: 76 - protein, 393 aa, synthetic construct
  REGION 1..393: note = SLO(deltaC101) fragment of SLO(deltaC11) - nnAA (e.g. pAMF) mutant 10 - w/o leader, with G overhang from cleavage
  VARIANT 35: note = any non-natural amino acid (e.g pAMF)
  VARIANT 112: note = any non-natural amino acid (e.g pAMF)
  VARIANT 195: note = any non-natural amino acid (e.g pAMF)
  VARIANT 246: note = any non-natural amino acid (e.g pAMF)
  VARIANT 280: note = any non-natural amino acid (e.g pAMF)
  VARIANT 298: note = any non-natural amino acid (e.g pAMF)
  VARIANT 330: note = any non-natural amino acid (e.g pAMF)
  VARIANT 387: note = any non-natural amino acid (e.g pAMF)
GAPKEMPLESAEKEEKKSEDKKKSEEDHTEEINDXIYSLNYNELEVLAKNGETIENFVPKEGVKKADKFIVIERKKKNINTTPVDISIIDSVTDRTYPAALQLANKGFTENXPDAVVTKRNPQKIHIDLPGMGDKATVEVNDPTYANVSTAIDNLVNQWHDNYSGGNTLPARTQYTESMVYSKSQIEAALNVNSXILDGTLGIDFKSISKGEKKVMIAAYKQIFYTVSANLPNNPADVFDKSVTFXELQRKGVSNEAPPLFVSNVAYGRTVFVKLETSSXSNDVEAAFSAALKGTDVXTNGKYSDILENSSFTAVVLGGDAAEHNKVVTXDFDVIRNVIKDNATFSRKNPAYPISYTSVFLKNNKIAGVNNRTEYVETTSTEYTSGXINLSHQ

SEQ ID NO: 77 - protein, 683 aa, synthetic construct
  REGION 1..683: note = SpyAD [33-714 fragment] nnAA (e.g., pAMF) mutant w/o leader
  VARIANT 33: note = any non-natural amino acid (e.g pAMF)
  VARIANT 256: note = any non-natural amino acid (e.g pAMF)
  VARIANT 355: note = any non-natural amino acid (e.g pAMF)
  VARIANT 626: note = any non-natural amino acid (e.g pAMF)
GQVKADDRASGETKASNTHDDSLPKPETIQEAXATIDAVEKTLSQQKAELTELATALTKTTAEINHLKEQQDNEQKALTSAQEIYTNTLASSEETLLAQGAEHQRELTATETELHNAQADQHSKETALSEQKASISAETTRAQDLVEQVKTSEQNIAKLNAMISNPDAITKAAQTANDNTKALSSELEKAKADLENQKAKVKKQLTEELAAQKAALAEKEAELSRLKSSAPSTQDSIVGNNTMKAPQGYPLEELKXLEASGYIGSASYNNYYKEHADQIIAKASPGNQLNQYQDIPADRNRFVDPDNLTPEVQNELAQFAAHMINSVRRQLGLPPVTVTAGSQEFARLLSTSYKXTHGNTRPSFVYGQPGVSGHYGVGPHDKTIIEDSAGASGLIRNDDNMYENIGAFNDVHTVNGIKRGIYDSIKYMLFTDHLHGNTYGHAINFLRVDKHNPNAPVYLGFSTSNVGSLNEHFVMFPESNIANHQRFNKTPIKAVGSTKDYAQRVGTVSDTIAAIKGKVSSLENRLSAIHQEADIMAAQAKVSQLQGKLASTLKQSDSLNLQVRQLNDTKGSLRTELLAAKAKQAQLEATRDQSLAKLASLKAALHQTEALAEQAAARVTALVAKXAHLQYLRDFKLNPNRLQVIRERIDNTKQDLAKTTSSLLNAQEALAALQAKQSSLEAT

SEQ ID NO: 78 - protein, 668 aa, synthetic construct
  REGION 1..668: note = SpyAD [33-699 fragment] nnAA (e.g., pAMF) mutant w/o leader
  VARIANT 33: note = any non-natural amino acid (e.g pAMF)
  VARIANT 256: note = any non-natural amino acid (e.g pAMF)
  VARIANT 355: note = any non-natural amino acid (e.g pAMF)
  VARIANT 626: note = any non-natural amino acid (e.g pAMF)
GQVKADDRASGETKASNTHDDSLPKPETIQEAXATIDAVEKTLSQQKAELTELATALTKTTAEINHLKEQQDNEQKALTSAQEIYTNTLASSEETLLAQGAEHQRELTATETELHNAQADQHSKETALSEQKASISAETTRAQDLVEQVKTSEQNIAKLNAMISNPDAITKAAQTANDNTKALSSELEKAKADLENQKAKVKKQLTEELAAQKAALAEKEAELSRLKSSAPSTQDSIVGNNTMKAPQGYPLEELKXLEASGYIGSASYNNYYKEHADQIIAKASPGNQLNQYQDIPADRNRFVDPDNLTPEVQNELAQFAAHMINSVRRQLGLPPVTVTAGSQEFARLLSTSYKXTHGNTRPSFVYGQPGVSGHYGVGPHDKTIIEDSAGASGLIRNDDNMYENIGAFNDVHTVNGIKRGIYDSIKYMLFTDHLHGNTYGHAINFLRVDKHNPNAPVYLGFSTSNVGSLNEHFVMFPESNIANHQRFNKTPIKAVGSTKDYAQRVGTVSDTIAAIKGKVSSLENRLSAIHQEADIMAAQAKVSQLQGKLASTLKQSDSLNLQVRQLNDTKGSLRTELLAAKAKQAQLEATRDQSLAKLASLKAALHQTEALAEQAAARVTALVAKXAHLQYLRDFKLNPNRLQVIRERIDNTKQDLAKTTSSLLNAQE

SEQ ID NO: 79 - protein, 653 aa, synthetic construct
  REGION 1..653: note = SpyAD [33-684 fragment] nnAA (e.g., pAMF) mutant w/o leader
  VARIANT 33: note = any non-natural amino acid (e.g pAMF)
  VARIANT 256: note = any non-natural amino acid (e.g pAMF)
  VARIANT 355: note = any non-natural amino acid (e.g pAMF)
  VARIANT 626: note = any non-natural amino acid (e.g pAMF)
GQVKADDRASGETKASNTHDDSLPKPETIQEAXATIDAVEKTLSQQKAELTELATALTKTTAEINHLKEQQDNEQKALTSAQEIYTNTLASSEETLLAQGAEHQRELTATETELHNAQADQHSKETALSEQKASISAETTRAQDLVEQVKTSEQNIAKLNAMISNPDAITKAAQTANDNTKALSSELEKAKADLENQKAKVKKQLTEELAAQKAALAEKEAELSRLKSSAPSTQDSIVGNNTMKAPQGYPLEELKXLEASGYIGSASYNNYYKEHADQIIAKASPGNQLNQYQDIPADRNRFVDPDNLTPEVQNELAQFAAHMINSVRRQLGLPPVTVTAGSQEFARLLSTSYKXTHGNTRPSFVYGQPGVSGHYGVGPHDKTIIEDSAGASGLIRNDDNMYENIGAFNDVHTVNGIKRGIYDSIKYMLFTDHLHGNTYGHAINFLRVDKHNPNAPVYLGFSTSNVGSLNEHFVMFPESNIANHQRFNKTPIKAVGSTKDYAQRVGTVSDTIAAIKGKVSSLENRLSAIHQEADIMAAQAKVSQLQGKLASTLKQSDSLNLQVRQLNDTKGSLRTELLAAKAKQAQLEATRDQSLAKLASLKAALHQTEALAEQAAARVTALVAKXAHLQYLRDFKLNPNRLQVIRERIDNTK

SEQ ID NO: 80 - protein, 840 aa, synthetic construct
  REGION 1..840: note = SpyAD [33-849 fragment] nnAA (e.g ., pAMF)mutant w/ long leader
  VARIANT 55: note = any non-natural amino acid (e.g pAMF)
  VARIANT 278: note = any non-natural amino acid (e.g pAMF)
  VARIANT 377: note = any non-natural amino acid (e.g pAMF)
  VARIANT 648: note = any non-natural amino acid (e.g pAMF)
MHHHHHHHHHHHHGSGENLYFQGQVKADDRASGETKASNTHDDSLPKPETIQEAXATIDAVEKTLSQQKAELTELATALTKTTAEINHLKEQQDNEQKALTSAQEIYTNTLASSEETLLAQGAEHQRELTATETELHNAQADQHSKETALSEQKASISAETTRAQDLVEQVKTSEQNIAKLNAMISNPDAITKAAQTANDNTKALSSELEKAKADLENQKAKVKKQLTEELAAQKAALAEKEAELSRLKSSAPSTQDSIVGNNTMKAPQGYPLEELKXLEASGYIGSASYNNYYKEHADQIIAKASPGNQLNQYQDIPADRNRFVDPDNLTPEVQNELAQFAAHMINSVRRQLGLPPVTVTAGSQEFARLLSTSYKXTHGNTRPSFVYGQPGVSGHYGVGPHDKTIIEDSAGASGLIRNDDNMYENIGAFNDVHTVNGIKRGIYDSIKYMLFTDHLHGNTYGHAINFLRVDKHNPNAPVYLGFSTSNVGSLNEHFVMFPESNIANHQRFNKTPIKAVGSTKDYAQRVGTVSDTIAAIKGKVSSLENRLSAIHQEADIMAAQAKVSQLQGKLASTLKQSDSLNLQVRQLNDTKGSLRTELLAAKAKQAQLEATRDQSLAKLASLKAALHQTEALAEQAAARVTALVAKXAHLQYLRDFKLNPNRLQVIRERIDNTKQDLAKTTSSLLNAQEALAALQAKQSSLEATIATTEHQLTLLKTLANEKEYRHLDEDIATVPDLQVAPPLTGVKPLSYSKIDTTPLVQEMVKETKQLLEASARLAAENTSLVAEALVGQTSEMVASNAIVSKITSSITQPSSKTSYGSGSSTTSNLISDVDESTQR

SEQ ID NO: 81 - protein, 562 aa, synthetic construct
  REGION 1..562: note = SpyAD [33-594 fragment] nnAA (e.g., pAMF)mutant w/long leader
  VARIANT 32: note = any non-natural amino acid (e.g pAMF)
  VARIANT 255: note = any non-natural amino acid (e.g pAMF)
  VARIANT 354: note = any non-natural amino acid (e.g pAMF)
QVKADDRASGETKASNTHDDSLPKPETIQEAXATIDAVEKTLSQQKAELTELATALTKTTAEINHLKEQQDNEQKALTSAQEIYTNTLASSEETLLAQGAEHQRELTATETELHNAQADQHSKETALSEQKASISAETTRAQDLVEQVKTSEQNIAKLNAMISNPDAITKAAQTANDNTKALSSELEKAKADLENQKAKVKKQLTEELAAQKAALAEKEAELSRLKSSAPSTQDSIVGNNTMKAPQGYPLEELKXLEASGYIGSASYNNYYKEHADQIIAKASPGNQLNQYQDIPADRNRFVDPDNLTPEVQNELAQFAAHMINSVRRQLGLPPVTVTAGSQEFARLLSTSYKXTHGNTRPSFVYGQPGVSGHYGVGPHDKTIIEDSAGASGLIRNDDNMYENIGAFNDVHTVNGIKRGIYDSIKYMLFTDHLHGNTYGHAINFLRVDKHNPNAPVYLGFSTSNVGSLNEHFVMFPESNIANHQRFNKTPIKAVGSTKDYAQRVGTVSDTIAAIKGKVSSLENRLSAIHQEADIMAAQAKVSQLQGKLASTLKQSDSLNLQV

SEQ ID NO: 82 - protein, 625 aa, synthetic construct
  REGION 1..625: note = SpyAD [33-657 fragment] nnAA (e.g., pAMF)mutant w/long leader
  VARIANT 32: note = any non-natural amino acid (e.g pAMF)
  VARIANT 255: note = any non-natural amino acid (e.g pAMF)
  VARIANT 354: note = any non-natural amino acid (e.g pAMF)
  VARIANT 625: note = any non-natural amino acid (e.g pAMF)
QVKADDRASGETKASNTHDDSLPKPETIQEAXATIDAVEKTLSQQKAELTELATALTKTTAEINHLKEQQDNEQKALTSAQEIYTNTLASSEETLLAQGAEHQRELTATETELHNAQADQHSKETALSEQKASISAETTRAQDLVEQVKTSEQNIAKLNAMISNPDAITKAAQTANDNTKALSSELEKAKADLENQKAKVKKQLTEELAAQKAALAEKEAELSRLKSSAPSTQDSIVGNNTMKAPQGYPLEELKXLEASGYIGSASYNNYYKEHADQIIAKASPGNQLNQYQDIPADRNRFVDPDNLTPEVQNELAQFAAHMINSVRRQLGLPPVTVTAGSQEFARLLSTSYKXTHGNTRPSFVYGQPGVSGHYGVGPHDKTIIEDSAGASGLIRNDDNMYENIGAFNDVHTVNGIKRGIYDSIKYMLFTDHLHGNTYGHAINFLRVDKHNPNAPVYLGFSTSNVGSLNEHFVMFPESNIANHQRFNKTPIKAVGSTKDYAQRVGTVSDTIAAIKGKVSSLENRLSAIHQEADIMAAQAKVSQLQGKLASTLKQSDSLNLQVRQLNDTKGSLRTELLAAKAKQAQLEATRDQSLAKLASLKAALHQTEALAEQAAARVTALVAKX

SEQ ID NO: 83 - protein, 635 aa, synthetic construct
  REGION 1..635: note = SpyAD [33-667 fragment] nnAA (e.g., pAMF)mutant w/long leader
  VARIANT 32: note = any non-natural amino acid (e.g pAMF)
  VARIANT 255: note = any non-natural amino acid (e.g pAMF)
  VARIANT 354: note = any non-natural amino acid (e.g pAMF)
  VARIANT 625: note = any non-natural amino acid (e.g pAMF)
QVKADDRASGETKASNTHDDSLPKPETIQEAXATIDAVEKTLSQQKAELTELATALTKTTAEINHLKEQQDNEQKALTSAQEIYTNTLASSEETLLAQGAEHQRELTATETELHNAQADQHSKETALSEQKASISAETTRAQDLVEQVKTSEQNIAKLNAMISNPDAITKAAQTANDNTKALSSELEKAKADLENQKAKVKKQLTEELAAQKAALAEKEAELSRLKSSAPSTQDSIVGNNTMKAPQGYPLEELKXLEASGYIGSASYNNYYKEHADQIIAKASPGNQLNQYQDIPADRNRFVDPDNLTPEVQNELAQFAAHMINSVRRQLGLPPVTVTAGSQEFARLLSTSYKXTHGNTRPSFVYGQPGVSGHYGVGPHDKTIIEDSAGASGLIRNDDNMYENIGAFNDVHTVNGIKRGIYDSIKYMLFTDHLHGNTYGHAINFLRVDKHNPNAPVYLGFSTSNVGSLNEHFVMFPESNIANHQRFNKTPIKAVGSTKDYAQRVGTVSDTIAAIKGKVSSLENRLSAIHQEADIMAAQAKVSQLQGKLASTLKQSDSLNLQVRQLNDTKGSLRTELLAAKAKQAQLEATRDQSLAKLASLKAALHQTEALAEQAAARVTALVAKXAHLQYLRDFK